SEQUENCE LISTING (265 sequences)

SEQ ID NO: 1 - DNA, 529 bp, Physcomitrella patens ssp. patens
  misc_feature (524)..(528): n is a, c, g, or t
ggttcatatatagcagtgccgaagcttttctagggtttacagctccgcaatcgttcgtttcccttagctgctgcgtgatccgccaggtccaggaagtcggaagagatggcgtcggaagaaattggaaagacgccggagtgggtgatggagaggcagcaggcgctccagagggtgcacaagctgacccatctgaaaggtccgcgcgacagaatcacgtccgtgatcatcccgggtgctttggctgccatcgggctgtcgctgatgggtcggggagtgtaccacctggcaacagggcaaggtttgaaggaatgagatcccggtagaggacggacgagtgggcgtgcttagttgtagtcgtaattagaggctccgagcgccatggcggtagggggccgtgcgagggtgccgcaaataagaggtgtggataagcgagtgtgcgggatggttcggggtgccgcgtcccttctttggatgatgaatgcaaattgtgccgtggaaatgggatgagatattgcctccaaaannnnna

SEQ ID NO: 2 - protein, 68 aa, Physcomitrella patens ssp. patens
MASEEIGKTPEWVMERQQALQRVHKLTHLKGPRDRITSVIIPGALAAIGLSLMGRGVYHLATGQGLKE

SEQ ID NO: 3 - DNA, 502 bp, Solanum lycopersicum
atcggccgaattgatcgtcttcagctttctctccgtcgcttgccaagtgagttcatctgcaaaccctaggatgtcagaagaagcacctttctatcccagagaaaagcttgttgagaagcaaaagttttaccaaagcgtccacaagcacacatacttgaaaggtcgttttgacaaggtcacctcagtggccattccagctgctttggctgcttctgctttgtttatgattgggagagggatctacaacatgtctcatggcatagggaagaaggaataaatagcggctactgctcgactattgttcctatggtggctgaatttgaaacgacctgtttgttcttttgttgattttcaattatcagtagctaatactagtcacttggatgctgatattaaaacatgccgatttatggcatatgctatcagtactccgcagcataataacacattatctcctctgtttaggggttctgcatatttgtattaaacggtgtttgtgtgg

SEQ ID NO: 4 - protein, 68 aa, Solanum lycopersicum
MSEEAPFYPREKLVEKQKFYQSVHKHTYLKGRFDKVTSVAIPAALAASALFMIGRGIYNMSHGIGKKE

SEQ ID NO: 5 - DNA, 759 bp, Hordeum vulgare
gacccaacaacccccctcaccctctcgtccccctcttcttccctgcccttcctcgtcccttccaggtcacgacgctacccccaaatccccagcctccagatccacccgccgccgccggcaaccccgtcgcttcccggcccctgcgcgccccagccagccaggatggcgcacgaagaggcaccattttacccacgtgagaagcttgttcagaagcagcagtatttccagaacttgagcaagcatatccaccttaaaggccgttacgatgcggtcatctccgttgccattccccttgcgcttgctggctccagcttgttcatgattggtcgtgggatctacaacatgtctaacgggatcgggaaaaaggagtgaatttctgtcggttttgctagtatctcaggaagcggcatggaagcgatactgcccaagctatgatgccatcttcgtttgcaaataatattgtcagagaaagactgagttcatttccagttctcttttgttggtatttgtggatatttgatgtcagcaaattgatgctaaactggcaatgatgattctataacattggcatcattatcctttctgttaattgtaaaaattatgttctacgtttcctttcaactgatgttttgtttgtgcattcatgacatgatttcttgtctgtgaattcatgagtttgtttgtatggtacgcagcaagttcctacgtggtttgaggtcctgaatatactggctaatatgagtccatcgggattatata

SEQ ID NO: 6 - protein, 69 aa, Hordeum vulgare
MAHEEAPFYPREKLVQKQQYFQNLSKHIHLKGRYDAVISVAIPLALAGSSLFMIGRGIYNMSNGIGKKE

SEQ ID NO: 7 - DNA, 525 bp, Populus trichocarpa
actagtttaattaaattaatcccccccccccggtagcctcctcttctctgctctctctcagcatccaaaccctaaccagattttcttgagcatcattcagctaggatgacaacagaagcacctttccgaccaagggagaagctcgttgagcaccagaaatatttccaaagcattcacaagcacacatatttgaagggacctcttgataaggttacctctgttgccattccaatagcattcgcagccacctcactttttcttattgggcgagggatctataacatgtctcatgggattggaaagaaggaatgaggaggctgtttgtgtaatgatcactgttatgtttttactgctcatgttttgaaggattattcgcttatgcatgtgaccagtattatttttaaatttgttaattaataattgcatagttgctggcttgcagctaccaatggaagtttaaacttcgccatcgatgcctttgttttctatttggcttttaatgatacatgattgaatttcagggtt

SEQ ID NO: 8 - protein, 68 aa, Populus trichocarpa
MTTEAPFRPREKLVEHQKYFQSIHKHTYLKGPLDKVTSVAIPIAFAATSLFLIGRGIYNMSHGIGKKE

SEQ ID NO: 9 - DNA, 2194 bp, Oryza sativa
aatccgaaaagtttctgcaccgttttcaccccctaactaacaatatagggaacgtgtgctaaatataaaatgagaccttatatatgtagcgctgataactagaactatgcaagaaaaactcatccacctactttagtggcaatcgggctaaataaaaaagagtcgctacactagtttcgttttccttagtaattaagtgggaaaatgaaatcattattgcttagaatatacgttcacatctctgtcatgaagttaaattattcgaggtagccataattgtcatcaaactcttcttgaataaaaaaatctttctagctgaactcaatgggtaaagagagagattttttttaaaaaaatagaatgaagatattctgaacgtattggcaaagatttaaacatataattatataattttatagtttgtgcattcgtcatatcgcacatcattaaggacatgtcttactccatcccaatttttatttagtaattaaagacaattgacttatttttattatttatcttttttcgattagatgcaaggtacttacgcacacactttgtgctcatgtgcatgtgtgagtgcacctcctcaatacacgttcaactagcaacacatctctaatatcactcgcctatttaatacatttaggtagcaatatctgaattcaagcactccaccatcaccagaccacttttaataatatctaaaatacaaaaaataattttacagaatagcatgaaaagtatgaaacgaactatttaggtttttcacatacaaaaaaaaaaagaattttgctcgtgcgcgagcgccaatctcccatattgggcacacaggcaacaacagagtggctgcccacagaacaacccacaaaaaacgatgatctaacggaggacagcaagtccgcaacaaccttttaacagcaggctttgcggccaggagagaggaggagaggcaaagaaaaccaagcatcctccttctcccatctataaattcctccccccttttcccctctctatataggaggcatccaagccaagaagagggagagcaccaaggacacgcgactagcagaagccgagcgaccgccttctcgatccatatcttccggtcgagttcttggtcgatctcttccctcctccacctcctcctcacagggtatgtgcctcccttcggttgttcttggatttattgttctaggttgtgtagtacgggcgttgatgttaggaaaggggatctgtatctgtgatgattcctgttcttggatttgggatagaggggttcttgatgttgcatgttatcggttcggtttgattagtagtatggttttcaatcgtctggagagctctatggaaatgaaatggtttagggatcggaatcttgcgattttgtgagtaccttttgtttgaggtaaaatcagagcaccggtgattttgcttggtgtaataaagtacggttgtttggtcctcgattctggtagtgatgcttctcgatttgacgaagctatcctttgtttattccctattgaacaaaaataatccaactttgaagacggtcccgttgatgagattgaatgattgattcttaagcctgtccaaaatttcgcagctggcttgtttagatacagtagtccccatcacgaaattcatggaaacagttataatcctcaggaacaggggattccctgttcttccgatttgctttagtcccagaattttttttcccaaatatcttaaaaagtcactttctggttcagttcaatgaattgattgctacaaataatgcttttatagcgttatcctagctgtagttcagttaataggtaatacccctatagtttagtcaggagaagaacttatccgatttctgatctccatttttaattatatgaaatgaactgtagcataagcagtattcatttggattattttttttattagctctcaccccttcattattctgagctgaaagtctggcatgaactgtcctcaattttgttttcaaattcacatcgattatctatgcattatcctcttgtatctacctgtagaagtttctttttggttattccttgactgcttgattacagaaagaaatttatgaagctgtaatcgggatagttatactgcttgttcttatgattcatttcctttgtgcagttcttggtgtagcttgccactttcaccagcaaagttc

SEQ ID NO: 10 - DNA, 53 bp, Artificial sequence
  primer prm18880
ggggacaagtttgtacaaaaaagcaggcttaaacaatggcgtcggaagaaatt

SEQ ID NO: 11 - DNA, 49 bp, Artificial sequence
  primer prm18881
ggggaccactttgtacaagaaagctgggtgtcctctaccgggatctcat

SEQ ID NO: 12 - DNA, 56 bp, Artificial sequence
  primer prm18882
ggggacaagtttgtacaaaaaagcaggcttaaacaatgtcagaagaagcacctttc

SEQ ID NO: 13 - DNA, 50 bp, Artificial sequence
  primer prm18883
ggggaccactttgtacaagaaagctgggttagccgctatttattccttct

SEQ ID NO: 14 - DNA, 51 bp, Artificial sequence
  primer prm18884
ggggacaagtttgtacaaaaaagcaggcttaaacaatggcgcacgaagagg

SEQ ID NO: 15 - DNA, 50 bp, Artificial sequence
  primer prm18885
ggggaccactttgtacaagaaagctgggtaaccgacagaaattcactcct

SEQ ID NO: 16 - DNA, 56 bp, Artificial sequence
  primer prm18886
ggggacaagtttgtacaaaaaagcaggcttaaacaatgacaacagaagcacctttc

SEQ ID NO: 17 - DNA, 50 bp, Artificial sequence
  primer prm18887
ggggaccactttgtacaagaaagctgggtatcattacacaaacagcctcc

SEQ ID NO: 18 - DNA, 824 bp, Medicago truncatula
gaaacctccagaacctgaatttataaataaacccaaaatcccagaaagattgagtgaagagtaccttagaatagccatgtcggcgttggcgaggtttttgcagaggcgattcatttcaacccaatcattccatcatgatcgccaccccatctttcaagcatcctctgggcattcttctatcaatgcaatattaaacggacgtggaattctcaaaaggggggtctcgacacagacaaatctaaatcaaaatatctgtgaagatgtaaaaatcagtgaagctgacaccttgaagtctggtgtgaataacgtccctacatccatgagcattaccgaggactctgccatcaagggttctgctggttttagtgtgaaagtatcctcaagacatgatcttgctatggttttcacctgcaaggtctgtgaaacaaggtcggtaaagacgttttgtcgcgaatcttatgagaaaggagttgtaatagcaaggtgcgggggatgtaataatcttcacttgattgcagatcaccgtggatggtttggtgaaaaaggaactgttgaggacttcctggctgctcatggagaaaaagttaaaagagggtcaattgatacactgaatgcgacatttgaagatataactggaaaacaatcttcgaagggtacaatttcaccaaatatataagttgcattagggtagcactgagatattggaagggtaaggggatgtaataatttttgctatttgtttttgaggaaacaattgttggtgtttgtaaactcgtatttttattactgtctcttgattattccgatattaaaaagtgtcattc

SEQ ID NO: 19 - protein, 199 aa, Medicago truncatula
MSALARFLQRRFISTQSFHHDRHPIFQASSGHSSINAILNGRGILKRGVSTQTNLNQNICEDVKISEADTLKSGVNNVPTSMSITEDSAIKGSAGFSVKVSSRHDLAMVFTCKVCETRSVKTFCRESYEKGVVIARCGGCNNLHLIADHRGWFGEKGTVEDFLAAHGEKVKRGSIDTLNATFEDITGKQSSKGTISPNI

SEQ ID NO: 20 - protein, 10 aa, Artificial sequence
  Motif 1
FTCKVCETRS

SEQ ID NO: 21 - protein, 31 aa, Artificial sequence
  Motif 2
CRESYEKGVVVARCGGCNNLHLIADHLGWFG

SEQ ID NO: 22 - protein, 9 aa, Artificial sequence
  Motif 3
KRGSXDTLN

SEQ ID NO: 23 - protein, 7 aa, Artificial sequence
  Motif 4
TLEDLAG

SEQ ID NO: 24 - DNA, 53 bp, Artificial sequence
  primer prm11653
ggggacaagtttgtacaaaaaagcaggcttaaacaatgtcggcgttggcgagg

SEQ ID NO: 25 - DNA, 54 bp, Artificial sequence
  primer prm11654
ggggaccactttgtacaagaaagctgggtcccttccaatatctcagtgctaccc

SEQ ID NO: 26 - DNA, 2194 bp, Oryza sativa; identical to SEQ ID NO: 9

SEQ ID NO: 27 - protein, 170 aa, Arabidopsis thaliana
MANTAAGWSPVLAPIYSPVNTKPINFHFSASFYKPPRPFYKQQNPISALHRSKTTRVIEVVTPKQRNRSFSVFGSLADDSKLNPDEESNDSAEVASIDIKLPRRSLQVEFTCNSCGERTKRLINRHAYEKGLVFVQCAGCLKHHKLVDNLGLIVEYDFRETSKDLGTDHV

SEQ ID NO: 28 - protein, 223 aa, Arabidopsis thaliana
MIKKASFIVLRFQNFTENRSVEFLLSLRLSMAARLLALRRALSLFSNQQHRFPLSQVSTEQLSLSNSLFSRSHVYGRLFQRQLSVIREANEASVTNVCNSSNSATESAKVPSPATPSEEMMVKYKSQLKINPRHDFMMVFTCKVCDTRSMKMASRESYENGVVVVRCGGCDNLHLIADRRGWFGEPGSVEDFLASQGEEFKKGSMDSLNLTPEDLAGGKISTE

SEQ ID NO: 29 - protein, 212 aa, Arabidopsis thaliana
MEATSLSSAATIISSSSSPLSIFSPKKRTDSSPPPRIVRLSNKKEDKDYDPQHSESNSSSLFRNRTLSNDEAMGLVLSAASVKGWTTGSGMEGPSLPAKTDTDTVSTFPWSLFTKSPRRRMRVAFTCNVCGQRTTRAINPHAYTDGTVFVQCCGCNVFHKLVDNLNLFHEVKYYVSSSSFDYTDAKWDVSGLNLFDDEDDDNAGDSNDVFPL

SEQ ID NO: 30 - protein, 188 aa, Glycine max
MAARMLQRRFISIFSRQTHHPITQESWYSPTSAILNSYGFHQRGVMTHTNPIKPVCEDVENNEADTLKSSPNPDEVATSISVNETSSIKFSAKSSLKTSSRHDLAMVFTCKVCETRSIKTVCRESYEKGVVVARCGGCNNLHLIADHLGWFGEPGSIEDFLASRGEEGKRGSGDTLNLTLEDLAGRKP

SEQ ID NO: 31 - protein, 191 aa, Hordeum vulgare
MAAGRFLPLAGRRIIAALSQPSAPSSRGIFFPSPATAGLRSLQTIIEASSNASDERHHDPEDHKTDTPPQPASVPAAAESSFMVRDASSLKISPRHDMAMIFTCKVCETRSVKMASRDSYDNGVVVARCGGCNNLHLMADRLGWFGQPGSIEDFLAAQGQDVKKGDTDTFSFTLEDLAGSQVKSKEPSGEN

SEQ ID NO: 32 - protein, 188 aa, Oryza sativa
MATRFLPLVRRGLAGVLNQSPAPASTRGFLFPAPVTAGIRSLQTIMEASNNASDDRNQDIEDSKTDTVPATVPSSDSGFKVRDTSNLKISPRHDLAMIFTCKVCETRSMKMASKESYEKGVVVARCGGCNNFHLIADRLGWFGEPGSIEDFLAEQGEEVKKGSTDTLNFTLEDLVGSQANDKGPSDKK

SEQ ID NO: 33 - protein, 199 aa, Populus trichocarpa
MAAARNTLQLRRLLSALAHNNQPFTSSLNKEHSWKLLPSASSLFTRNDFYGRGLQTLAKPANQANEESENHENGLKPNCSSANAPAQVNSNEGSATTYSSLSNLKTSPRHDLAMIFTCKVCETRSVKTVCRESYEKGVVVARCGGCNNLHLIADHLGWFGQPGSIEEILAARGEEVKKGSADTFNLTLEDLAGKKIFKE

SEQ ID NO: 34 - protein, 191 aa, Triticum aestivum
MAAGRFLPLAGRRIIAALSQPSAPSSRGIFFPSTATAGLRSLQTIIEAGSNASNERRHDPEDHKTGTPPPPASVPAAAESSFKVRDASTLKISPRHDMAMIFTCKVCETRSVKMASRDSYDNGVVVARCGGCNNLHLMADRLGWFGQPGSIEDFLAEQGQDVKKGDTDTLSFTLEDLAGSQVKSKEPSGEK

SEQ ID NO: 35 - protein, 191 aa, Triticum aestivum; identical to SEQ ID NO: 34

SEQ ID NO: 36 - protein, 109 aa, Triticum aestivum
MIFTCKVCETRSVKMASRDSYDNGVVVARCGGCNNLHLMADRLGWFGQPGSIEDFLAEQGQDVKKGDTDTLSFTLGGLGRVSRSNPRNLPGENKPCCCNILGFWAQQQL

SEQ ID NO: 37 - protein, 187 aa, Zea mays
MATTRLLPLLRRRLAAAIAGSPAPYSLRGPSFPAPAAAGLRSLLKAAGASDTATEPQDQQHSETTPPPASVPTPESGLKVRDTSNLKISPRHDLAMIFTCKVCETRSMKMASRDSYENGVVVVRCGGCNNLHLMADRLGWFGEPGSIEDFLATQGEEVKKGSTDTISFTLDDLAGSQVSSKGPSEQN

SEQ ID NO: 38 - protein, 211 aa, Zea mays
MESVASAAIATTSRSLPLPFSSAPVHRRRRAAFLPVAASKRHDDDKEAAKGSSSEPRREPTSLAPYGLSISPLSKDAAMGLVVSAATGSGWTTGSGMEGPPTASKAGGAGRPEVSTLPWSLFTKSPRRRMRVAFTCNVCGQRTTRAINPHAYTDGTVFVQCCGCNVFHKLVDNLNLFHEMKCYVGPDFRYEGDAPFNYLDRNEDGDSIFPR

SEQ ID NO: 39 - DNA, 513 bp, Arabidopsis thaliana
atggcgaatactgccgccggttggtctccggttttggctccaatctattctccggtaaacacaaagccaatcaattttcacttctcagcttctttctacaagcctcctcgtccattttacaagcagcaaaaccctatatcggctctacacaggtcgaaaactactcgtgtgatagaggtagtaacaccaaagcaaaggaatcgttctttttctgtttttggatcactcgctgatgattctaagttaaacccagatgaagaatcaaatgattccgcagaggtagcttctatagatattaagctaccgagaagaagtttgcaagtggaatttacttgcaattcatgtggagaaagaactaagcggcttatcaataggcatgcctatgaaaagggccttgtctttgttcaatgtgcagggtgtctaaagcatcataaactggttgacaatcttggtctcattgttgagtatgatttccgggaaacctccaaggatttgggtaccgatcacgtttga

SEQ ID NO: 40 - DNA, 868 bp, Arabidopsis thaliana
acttgaccataacgaaaactaactctaatgattaaaaaggcttcttttattgtgctccgattccaaaatttcacggaaaatagaagcgtcgagttcctcctctccctcaggttgtcaatggccgctaggttacttgctttgagacgcgctttgtctcttttcagcaaccaacaacatcgttttcctttgtctcaagtctcaacagagcagttgtcgctatcaaactcactcttcagcagaagtcatgtttatggaagattatttcagagacagttatctgtaatccgtgaggcaaatgaagcttctgtaaccaatgtctgcaactcgtcaaactctgctactgaatcggccaaagttccctcccctgcgacgccctctgaggaaatgatggtgaagtacaagtcccagttaaaaataaacccgaggcatgacttcatgatggtcttcacttgcaaggtctgtgatacaagatctatgaagatggcgagccgagaatcatatgaaaacggcgttgtggtggtacgatgtggagggtgtgataatctacatttgattgcagaccgtcgtggttggtttggagaaccaggaagcgtggaggacttccttgcttctcaaggggaagaattcaagaaaggatccatggattctcttaacctaactcctgaagatttagctggaggaaagatttctactgaataaggagtgatcttctttgcttttgctacttaaccattatggaacagaacttcaccttatgttccagtattataattgtttcttgtagttcactgtttggtaacatttaaatgcagaagatgtttaatacaattatgtgtttggaatgttccatataggttgagatgttcca

SEQ ID NO: 41 - DNA, 813 bp, Arabidopsis thaliana
tctctcagagaagtaaaaacaaaatttcgttctgtgtgaagcctcttcttcttcgatcaaccatggaagctacctctctaagctctgcagcaacaatcatctcctcctcatcttccccactctccatattctctccaaagaagcgaacagactcatcacctcccccgagaatcgtccgtctctcgaacaagaaggaagacaaagattacgatccgcaacattccgaatcgaactcatcaagcctcttccggaatcgaactctctccaatgatgaagcaatgggactggtgttgagtgcagcttcggttaaaggatggacaaccggttccggtatggaaggaccgtctctaccggctaaaaccgatacagacacggtttccacatttccatggtcattattcactaaatcgcctcgtaggcgaatgcgtgttgctttcacttgtaacgtatgtgggcaaagaactacaagagctattaatcctcatgcttacactgatggcactgtcttcgtgcagtgttgtggatgtaatgtgtttcataagctggtcgataatctcaacttgtttcatgaggttaagtattatgtgagcagctcgagcttcgattacaccgatgctaagtgggatgttagcggcttgaatcttttcgatgatgaggatgatgataatgctggtgatagcaatgatgtctttcctttgtaaagaacctttctacaatatctttgttatatatctgtatatagctaccttgtcatatcatcttggtgtgaataacatttggaagtaataaattggtcatatagctcatgttctg

SEQ ID NO: 42 - DNA, 616 bp, Glycine max
ctgggcataacaccacattacattggcttgggagctatggcggcaaggatgttgcagaggcgattcatttcaatcttctcaagacaaacccatcaccccattactcaagaatcttggtattctcctaccagtgcaatattaaacagttatggattccatcaacggggggtcatgacccatacaaatccaatcaaacctgtctgtgaagatgtagagaataatgaagcagacaccttaaaatcaagtccaaacccagatgaagttgctacatctattagtgttaatgaaacctcttctataaagttttctgccaagtccagtttgaagacatcctcaaggcatgatcttgctatggttttcacctgcaaggtctgtgaaacaagatccattaagacggtttgtcgtgaatcatatgagaaaggtgtggtggtggcaagatgtggggggtgtaataaccttcacctgattgcagatcaccttgggtggtttggtgaaccaggaagcattgaggacttcctggcttctcgtggagaagaagggaaaagagggtcaggtgacacactgaaccttacattggaagatttagcaggaaggaaaccttgaaaggtacaatttg

SEQ ID NO: 43 - DNA, 936 bp, Hordeum vulgare
  misc_feature (935)..(935): n is a, c, g, or t
ctcgcctccttctttcttcgccggcaagaagaagacgcgctcccctcccctccgtcgccggccgacacgccatggccgccggccggtttcttccgctggcgggccgccgcatcatcgcggccctatcccagccgtccgccccctcttcccgcggaattttcttcccttcgcctgcgaccgcaggcttgaggtccctccagacgatcatcgaagcaagcagcaacgcatcggacgagcgtcaccatgacccggaggatcacaagaccgacaccccgccgcagccagcttcggtcccggcagcagcggagtcgagcttcatggtcagagacgcatcgagcctgaagatctcaccgaggcatgacatggcgatgatcttcacttgcaaggtctgcgagacgaggtccgtgaagatggcgagccgtgactcgtacgacaacggggtggtggtcgcacgctgcgggggctgcaacaacctgcacctgatggcagacaggctcggctggtttggccagccggggagcatcgaggacttcctggcggcgcaggggcaggacgtgaagaaaggcgacacagatactttcagcttcaccctggaggacttggccgggtctcaggtcaaatcgaaggaaccttctggtgaaaattaggccttgctgtgataccttggccttctggtccagcagcagctataaaactgtcacctccttaccgaaactttgcgagttattcgctcagtttcatggcttctcaagtggagtacaagctgttaagttcaacttagattaaagctgcaatagtgaagtgaattttctgtagtggactacccacactctagtattttgtgctttatcagttcttgtccaagcatgtttccgagagaacaaagatattgagatgtgaccttctgaacctctatctagttttactggaatatnt

SEQ ID NO: 44 - DNA, 567 bp, Oryza sativa
atggccactcggtttctgcctctggtgcgccgcggccttgccggcgtcctgaatcaatcgcccgcgcccgcgtccacccgaggattcttgtttcctgcacctgtgactgctggcataagatctctgcaaactatcatggaagcaagcaataatgcttcagatgaccgtaaccaggacatagaggattccaaaaccgacaccgtgccagctacggtcccttcatcggattccggcttcaaagttagagatacatcaaacttgaagatctcaccgagacacgacctcgccatgatctttacgtgcaaggtttgcgagaccaggtctatgaagatggcgagcaaggaatcatatgagaaaggagtggtggtcgctcgttgcggcggctgcaataatttccacctgatcgcggataggcttggctggtttggggagccaggaagcatcgaagactttctagccgaacaaggagaggaggtgaagaaaggctcaacagatactcttaacttcactcttgaggacttggttgggtctcaggctaatgataagggcccttctgataaaaaatag

SEQ ID NO: 45 - DNA, 710 bp, Populus trichocarpa
cagagctgcgagcttggacaccattctttcatggcggcagctagaaacacgttgcagctgaggcgattgctctctgctcttgcccataataatcaacccttcacctcttctcttaataaagaacatagctggaagcttcttccttctgcaagttcactcttcaccaggaatgatttttatggaagagggctgcagactctagcaaaaccagccaaccaagctaatgaggagtcggaaaatcatgaaaatggtttgaagcccaattgcagctcagccaatgctcccgcccaagtgaacagtaatgagggttctgctacaacttattcttctttatccaacttgaaaacctctccaaggcatgatcttgccatgatctttacttgcaaggtctgcgagacaagatctgtcaagacagtttgtcgtgaatcatatgaaaaaggtgtggtggtggcacggtgtggtggttgcaataacctgcacctgattgcagaccatcttggatggtttggacagcctggaagcattgaggaaatcctggctgctcgaggggaggaagtgaaaaaagggtctgccgatacatttaatttaacacttgaagatctagctggaaagaaaatcttcaaagagtgaattcagctgccatgtaacatcattctagtgacttttttttcttctcaatatggcattttctggctgaactctcatcgata

SEQ ID NO: 46 - DNA, 1164 bp, Triticum aestivum
tgccgctgtactgtgccgctcgcctctttctttcttcgccggcaacaagaagaagatgacgcgctcccctcccctccgtcgccggccgacacgccatggccgccggccggtttctgccgctggcgggccgccgcatcatcgcggccctgtcccagccgtccgccccctcttcccgtggaattttcttcccttctactgcgaccgcaggcttgaggtccctccaaacgatcatcgaagcaggcagcaacgcgtcaaatgagcgtcgccatgacccggaagatcacaagaccggcaccccgccgccgccagcttcggtccctgcagcagcggagtcgagcttcaaggtcagagacgcgtcgaccctgaagatctcgccgaggcacgacatggccatgatcttcacgtgcaaggtctgcgagacgaggtccgtgaagatggccagccgggactcgtacgacaacggggtggtggtcgcccgctgcgggggctgcaacaacctgcacctgatggcagacaggctcggctggtttggccagccggggagcatcgaggacttcctggcggagcaggggcaggacgtgaagaaaggcgacacggatactctcagcttcaccctggaggacttggccgggtctcaggtcaaatccaaggaaccttctggtgaaaaataggccttactgtaatatcttggccttctggtccagcagcagctataataaaactgtcacctccttaccgaaactttgcgaattattcgctcagtttcatggcttctcaagtggagtacaagctgctaagttctagatttaagctgccatagtgaagtgaattttctgtagtggactacccacactctagtaatttgtgctttatcctttcttgtccagcatgtttttcggagagaagagaacaaagatattgagatgtgactttctgaacctctagtttaactggaatatggcgttcaaaaaaaaaaaaagccgggcgctctaaagtttcctcaaggggccaagcttaccgtaccagcttcttgtacaagtgttcctatgtgggtctatttaagctagccctggcggcgtttaaccgtctgactggaaactgttactgggatttgtgaggacctactttgggggtaattttggcaactcctccggattaacctta

SEQ ID NO: 47 - DNA, 1173 bp, Triticum aestivum
  misc_feature (26)..(26): n is a, c, g, or t
taccgctgtactgtaccgtacgcctnctttctttcttcgccggcaacaagaagaagaagacgcgctcccctcccctccgtcgccggccgacacgccatggccgccggccggtttctgccgctggcgggccgccgcatcatcgcggccctgtcccagccgtccgccccctcttcccgtggaattttcttcccttctactgcgaccgcaggcttgaggtccctccaaacgatcatcgaagcaggcagcaacgcgtcaaatgagcgtcgccatgacccggaagatcacaagaccggcaccccgccgccgccagcttcggtccctgcagcagcggagtcgagcttcaaggtcagagacgcgtcgaccctgaagatctcgccgaggcacgacatggccatgatcttcacgtgcaaggtctgcgagacgaggtccgtgaagatggccagccgggactcgtacgacaacggggtggtggtcgcccgctgcgggggctgcaacaacctgcacctgatggcagacaggctcggctggtttggccagccggggagcatcgaggacttcctggcggagcaggggcaggacgtgaagaaaggcgacacggatactctcagcttcaccctggaggacttggccgggtctcaggtcaaatccaaggaaccttctggtgaaaaataggccttactgtaatatcttggccttctggtccagcagcagctataataaaactgtcacctccttaccgaaactttgcgaattattcgctcagtttcatggcttctcaagtggagtacaagctgcntagttctagatttaagctgcaatagtgaagtgaattttctgtagtggactacccacactctagtaatttgtgctttatcctttcttggtcaggcatgtttttccgagagaagagaacaagatattgagatgtgaccttctgaactctagtttactgggataatgcgtcaaaaaaaaaaaaagggcggcgctctagagtatctcgagggccaaagcttcgcgtacccgcttctggtacaggtgtcctatggggatctatatagctaggacggccgtgtttaaaagtttgactggaaatggatactgggatctgggaaggacttatttgggggtgcttttgggcaactcctccgaatttagccccggtaaattatttaa

SEQ ID NO: 48 - DNA, 590 bp, Triticum aestivum
  misc_feature (418)..(418): n is a, c, g, or t
aatgatcttcacgtgcaaggtctgcgagacgaggtccgtgaagatggccagccgggactcgtacgacaacggggtggtggtcgcccgctgcgggggctgcaacaacctgcacctgatggcggacaggctcggctggtttggccagccggggagcatcgaggacttcctggcggagcaggggcaggacgtgaagaaaggcgacacggatactctcagcttcaccctgggaggacttggccgggtctcaaggtcaaatccaaggaaccttcctggtgaaaataagccttgctgctgtaatatcttgggcttctgggcccagcagcagctataataaaactgtcacctcccttaacgaaacttttgcgaactattcgctcatttcaaagcttctcaaagtggattataactgttaattccnactanattaaactgcaatatgaattgattcctggtatggactaccacacccnatatttgtgcttanccttctgcccaatcattttccgtcaaagaacaagtatganattgacctctaactcanttaactggatatgngtcttttttaagtcaatatatataatgttcnctat

SEQ ID NO: 49 - DNA, 1056 bp, Zea mays
agacagacgcaggcggcaggcagcggcgcagcgcaccgcttcttcctcttctatctctcatctacagccttcgctgcgccgccatggccaccacccgcttgctgccgctgctccgacgccgcctcgccgccgcaatcgccggatcgcctgctccctactccctccgaggaccctcatttcctgcaccagcagctgcagggctaaggtccctcctaaaagctgctggagcgagcgatactgcaacagaaccccaggaccaacagcattccgaaacaactcccccgccggcttctgtcccgacaccggagtccggtctcaaagtcagggacacctccaacctgaagatctcaccaaggcatgacctcgccatgatctttacgtgcaaggtgtgcgagaccaggtccatgaagatggccagcagggactcgtacgagaacggagtcgtggtcgtgcggtgcggtggctgcaacaacctccacctcatggcggacaggcttggctggtttggggagccagggagcattgaggacttcctagcgacgcaaggggaggaggtgaagaaaggttcgacagatactatcagctttactttggacgacttggctgggtctcaggtcagttctaaggggccttccgaacaaaattaatatgatagtgtttggtccagtaagaacctgcagaagcctctctttactataaagaagacgcacatgtcacctgtgtgttgaagagaagaaaaaagcgcctctagaagcctaccttaactgttgcacctgtagttctgcttaacttcatggcttttcatgtgtagctttcgagcccatcaaatacgcgatgttgtgattctattgtagtgtagctatttcctataccaaaaactggatccaggagagcctacataaattgatggactgcccgcatttctgatcatgtagtcagttgtgtgcctgcattcattcacggattgcttgtggcgcctcttacagatggttgttatgactttaaacttgtgctggaagctggtgaagagctacagatttcatgattaaaaaaaaa

SEQ ID NO: 50 - DNA, 961 bp, Zea mays
cagaagaagaaaaacatctcacgccacgcctgctccagcacctcgctctccgctccgttggccccatggagtccgtcgcgtccgccgcgatcgccaccacctcccgctctctcccgctccccttctcttctgccccggtccaccgccggcgccgtgccgccttcctccccgttgccgcctccaagcgtcacgacgacgacaaggaggccgcgaaagggtccagctcggaaccacggcgcgagcctaccagcctcgcgccgtacggactctccatctcgccactctccaaggacgcggccatggggctggtggtgagcgctgccacggggagcggctggacgacgggatcggggatggagggcccgccgacggcgagcaaagccggtggggctggcaggccggaggtgtcgacgctgccttggtccctcttcacgaaatcaccgcggcggcgcatgcgggtggccttcacctgcaacgtgtgcgggcagcgtacgaccagggccatcaatcctcatgcctacaccgatggaactgtgtttgttcagtgctgcggttgcaacgtgttccataagttggtcgacaacctgaacctgtttcatgagatgaagtgctatgttggcccagatttccgctacgaaggggatgctccattcaactaccttgacagaaacgaggatggcgacagtatcttccctcgctaaagcctcccttatgttgcagacttgcagtacccaaaaacaaatgatcggtcctgttctaatttgttcgtgtagctgtacttaataagcagatgtaccttcatgaacctgtaagactagtttatcatacgcatagatgaccagtcactagaatctacactggagttgtaatgtcggtgaccagttagtgtaattttcaattgcctgtcaacttttgggcatataataaaaaatatgacctgcatttcgtctaaaaaaaaaaa

SEQ ID NO: 51 - DNA, 1577 bp, Populus trichocarpa
aagaagtggtatagatcataggaggaggcaatgggagctaggtgctccaaattatcactctgttggtggccttcccatctcaaatcaaatctcaactattcctctgatcttgagaatggggagttattgcctggtgggtttagagagtacagtttggagcagctaagagctgccacgtcagggttcagttcggacaacatagtatcagaacacggagagaaagctccgaatgtagtttacagaggaaagcttcaagaagatgatcgctggattgctgttaaacgctttaacaagtctgcttggcctgattctcgccaattccttgaggaggctagagcagtggggcagttaaggaatgaaagattggccaatttgatagggtgttgctgtgaaggagaggagaggttacttgttgctgagtttatgcctaatgagactctctctaagcatctttttcattgggagaatcagccgatgaaatgggctatgaggttgagagtggctctttatttagctcaagctttggagtactgtagtagtaaaggaagggcattgtaccatgactttaatgcatatagaattttgtttgaccaggatggtaacccgaggctctcctgctttggcctgatgaagaacagtagagatggaaagagctacagtacaaatttggcattcacccctcccgagtacttgagaactggaagagtgacaccggagagcgtggtttatagctttggcaccctattacttgatcttctcagtggaaaacatatccctccaagccatgcacttgaccttatacgcggaaaaaattttctgatgctgatggactcttgtttggagggtcatttttcaaacgatgatggaactgaacttgtgcgtttagcttcacgttgcttacagtttgaagctcgtgagaggcccaatgcaaaatctcttgtcactgctctcactcctcttctaaaagatactcaggttccatcctatattttgatgggtattccacatggaactgaatccccaaagcaaacaatgtcattgacacctctaggggaagcttgctcaagactggatcttactgcaatacatgaaatgctggaaaaggtgggatacaatgatgatgagggaattgcaaatgagctttccttccaaatgtggacagatcagatacaggaaacgctgaattgtaagaaacgtggtgatgctgcttttcgagctaaagattttaacgctgccattgattgttatactcaatttatcgatggcgggaccatggtatctccaactgtatttgctagacgctgtttgtgctacttgataagtgacttgccacaacaagctcttggagatgctatgcaagctcaagcagtttctcccgagtggcccactgccttctatcttcaagctgcttccctctttagcctcgggatggacactgatgcacaggaaactctaaaagatggctcatctttagaagctaaaaatcatggaaactgaaaatgtatagcctttcgtatttatttttttccttttaaacttgcgcactccatgtattcatctctatttgtttccttttg

SEQ ID NO: 52 - protein, 488 aa, Populus trichocarpa
MGARCSKLSLCWWPSHLKSNLNYSSDLENGELLPGGFREYSLEQLRAATSGFSSDNIVSEHGEKAPNVVYRGKLQEDDRWIAVKRFNKSAWPDSRQFLEEARAVGQLRNERLANLIGCCCEGEERLLVAEFMPNETLSKHLFHWENQPMKWAMRLRVALYLAQALEYCSSKGRALYHDFNAYRILFDQDGNPRLSCFGLMKNSRDGKSYSTNLAFTPPEYLRTGRVTPESVVYSFGTLLLDLLSGKHIPPSHALDLIRGKNFLMLMDSCLEGHFSNDDGTELVRLASRCLQFEARERPNAKSLVTALTPLLKDTQVPSYILMGIPHGTESPKQTMSLTPLGEACSRLDLTAIHEMLEKVGYNDDEGIANELSFQMWTDQIQETLNCKKRGDAAFRAKDFNAAIDCYTQFIDGGTMVSPTVFARRCLCYLISDLPQQALGDAMQAQAVSPEWPTAFYLQAASLFSLGMDTDAQETLKDGSSLEAKNHGN

SEQ ID NO: 53 - DNA, 2171 bp, Hordeum vulgare
agacccatctccccaccccccgctttcggtttcttggaaatgggggcgccgccgtggaggctgctgtgctgctgctgctgccgcgagtccgatcgcaatggggtggacgacctcaagctcaagcccgacgcagccgatggggaggtggcggcgggggactggtacgacctcccgccattccaggagttcaccttccagcagctgcgcctcgccacctcgggcttcgccgccgagaacatcatctccgaaagcggcgacaaggcgcctaacgtcgtctacaagggcaagctcgacgcccagcgccggatcgctgtcaagcgcttcagccgctctgcctggcccgacccacgccagttcatggaagaagctaagtctgttggccagctccggaacaaaagaatcgtaaatttgcttggttgttgctgtgaagccgatgaaagattgcttgttgctgagtacatgcccaatgacacactggcgaagcatctgttccattgggagtcacaggcaatggtatggcccatgagattacgggttgttctgtatcttgccgaggctttagactactgcgtaagcaaggagcgggctctctatcatgatcttaatgcatatagagttctgtttgatgatgactgcaaccctaggctttcatgtttcggcctaatgaagaacagtcgagatggcaaaagttacagcacaaatttggcattcactcctcctgaatatatgaggactggaagaataactccggaaagtgtcatatacagctttggtacattgttgttggatgttcttagtgggaagcatattcctcctagccatgctcttgacctgattcgtgatcgaaacttcagtatgctcatagactcctgtttagagggccaattttcaaatgaagaaggaacagaactgatgcgtttagcttcaaggtgcctgcattatgaaccacgagagcggcctaatgtaagatctttggttcttgcactggcttctcttcagaaggatgttgagtccccatcttacgatctgatggataagccccgtggtggtgcatttactcttcaatcaattcatctttctcctcttgctgaagctttctccagaaaggatcttactgcaatacatgaacacctagaaacagctggctataaagatgatgagggaacagcaaatgagctctcatttcagatgtggactaatcaaatgcaagctactattgactcaaagaagaagggtgacactgcatttcgacaaaaggattttagcatggccattgattgctactctcagttcattgatgttggtaccatggtttcaccaacaatttatgcgaggcgttgcttgtcatatctcatgaatgacatgccacaacaagctctggatgatgcagtgcaggctctggcgatatttcctacatggccaactgcattttatcttcaggctgcggccttattttcgttaggaaaagaaaacgaagctcgagaagcactcaaggatggttcggctgtggagacaaggagcaaggggcattgaagatggatagctgaacatcaggtgctctcatttggacataatttgttggagacaacagcagttgttaatctggcttaggcgcatggggactgtcagttagccttgtgatatacatatacaattggtggtgtatatacgaaaaacatgcatagatagaattgacccgagagagggaaagacagaggagataccagcggttaataaatgtacatcccacatggagctaacaaggggaaaggaggtgagcctgttcatccaggtcaccctgccacataaaagaggagatatagaaaggaagaaaaggtggccatctgttctgtcaaacatcattatcacccatcagatcacgttagtgttggtaatagattgtagggtttgtagagcaagtggtgtttggtctttttgctcttgtcttcgttctcagctcagccaggcactgtgatttgggtcatatattagtaatgatttggtattgttaggagtgtagttgagagaagatcgatgagttctcctgtaatgattcaatctttgggtacagtgtgggctttgtatatttgtgagaaggctctattcggaacagatcatgtgctgctactttgttggtcccataaggcataagccatgaaatggttggtggc

SEQ ID NO: 54 - protein, 497 aa, Hordeum vulgare
MGAPPWRLLCCCCCRESDRNGVDDLKLKPDAADGEVAAGDWYDLPPFQEFTFQQLRLATSGFAAENIISESGDKAPNVVYKGKLDAQRRIAVKRFSRSAWPDPRQFMEEAKSVGQLRNKRIVNLLGCCCEADERLLVAEYMPNDTLAKHLFHWESQAMVWPMRLRVVLYLAEALDYCVSKERALYHDLNAYRVLFDDDCNPRLSCFGLMKNSRDGKSYSTNLAFTPPEYMRTGRITPESVIYSFGTLLLDVLSGKHIPPSHALDLIRDRNFSMLIDSCLEGQFSNEEGTELMRLASRCLHYEPRERPNVRSLVLALASLQKDVESPSYDLMDKPRGGAFTLQSIHLSPLAEAFSRKDLTAIHEHLETAGYKDDEGTANELSFQMWTNQMQATIDSKKKGDTAFRQKDFSMAIDCYSQFIDVGTMVSPTIYARRCLSYLMNDMPQQALDDAVQALAIFPTWPTAFYLQAAALFSLGKENEAREALKDGSAVETRSKGH

SEQ ID NO: 55 - DNA, 2194 bp, Oryza sativa; identical to SEQ ID NO: 9

SEQ ID NO: 56 - DNA, 53 bp, Artificial sequence
  primer prm18890
ggggacaagtttgtacaaaaaagcaggcttaaacaatgggagctaggtgctcc

SEQ ID NO: 57 - DNA, 51 bp, Artificial sequence
  primer prm18891
ggggaccactttgtacaagaaagctgggtggctatacattttcagtttcca

SEQ ID NO: 58 - DNA, 1911 bp, Arabidopsis thaliana
gtaaaacctctgtaccagttagagtctctcttgttctctctctgggtttaatagataacataacaacaaacaacagctggtgaggaaaaatctcagcaatggcgctacgaacactctcaacgtttccttctcttcctcgtcgccacacaacgacgagacgtgaacccaatctcaccgtcatttaccgtaatccgacgacatcaatcgtctgtaaatcaatagctaattcagaaccaccagtttcactctcggaacgagatggatttgcggcggctgctccaacccctggagaaaggttcctggagaaccaacgagctcatgaagctcagaaagtagtgaagaaagagatcaaaaaggagaagaagaaaaagaaagaggagattattgcacggaaagttgttgatacctcagtctcatgttgttacggctgcggagctccgttacaaacttccgacgtcgattctccgggatttgtcgatttggttacttatgaattgaagaagaagcatcaccagttaagaactatgatatgtggaagatgtcagctattgtcacatggacatatgattacagcagttggtggtaatggaggttatccaggtgggaaacaatttgtatcagctgatgaacttcgtgagaaactttctcatttacgccatgagaaagctttgattgttaaattggttgatatagtggattttaatggaagctttttagctcgtgttcgtgatttagttggagctaatccgattatacttgttataactaagattgatcttcttccaaaaggaacggatatgaattgtatcggggattgggttgtggaagtgaccatgaggaaaaagcttaatgtcttgagtgtccatctcacaagttcaaagtccctggatggagttagcggagttgcatcagagatccagaaggagaaaaagggacgagatgtctacattctgggtgcagctaacgtagggaagtcagcattcatcaatgctttgctgaaaacgatggccgaaagggatcctgttgcagcagcggcacaaaagtacaaaccaattcaatctgctgtccctggaaccaccttgggtccaattcagatcaacgctttcgtcggaggagagaagttgtatgacacaccgggtgtgcacctacaccacaggcaagcagctgtcgttcattcagatgatttacccgcccttgctcctcaaaatcgtctcagaggccaatctttcgatatttcaactttgccaactcaatcgtcaagtagtcccaagggtgagagcttaaacggttatacatttttctggggaggtctcgttaggattgacatcttgaaggctctaccggaaacatgtttcacattctatggaccaaaagctcttgagattcatgcagtaccaaccaaaacagcgactgccttttacgaggcaaaactgggtgtgcttctaacacctccatcagggaaaaatcagatgcaggagtggaaagggttacaatctcaccggttacttcaaatcgaaatcaacgatgcaaaaagaccggctagtgatgtggcaatatcagggttaggatggatttcaattgaaccaatccgcaaaacacgaggaactgaaccgagagatctcaatgaagcagagcatgagatacatatttgtgtcagtgtgccaaaaccagttgaagtttttcttcgaccaacattgccaattggtacttcaggtactgaatggtatcagtatcgtgagttaaccgataaggaagaagaagtaagacccaaatggtacttttgaatttttttttgtttagatgcaaaaatatcggcttagtatcacaaaccagagttatatgtaacaaaatagtaaattgtttgtaatgaaatctacttaagtaacatagcattcaaggaattaaacatct

SEQ ID NO: 59 - protein, 561 aa, Arabidopsis thaliana
MALRTLSTFPSLPRRHTTTRREPNLTVIYRNPTTSIVCKSIANSEPPVSLSERDGFAAAAPTPGERFLENQRAHEAQKVVKKEIKKEKKKKKEEIIARKVVDTSVSCCYGCGAPLQTSDVDSPGFVDLVTYELKKKHHQLRTMICGRCQLLSHGHMITAVGGNGGYPGGKQFVSADELREKLSHLRHEKALIVKLVDIVDFNGSFLARVRDLVGANPIILVITKIDLLPKGTDMNCIGDWVVEVTMRKKLNVLSVHLTSSKSLDGVSGVASEIQKEKKGRDVYILGAANVGKSAFINALLKTMAERDPVAAAAQKYKPIQSAVPGTTLGPIQINAFVGGEKLYDTPGVHLHHRQAAVVHSDDLPALAPQNRLRGQSFDISTLPTQSSSSPKGESLNGYTFFWGGLVRIDILKALPETCFTFYGPKALEIHAVPTKTATAFYEAKLGVLLTPPSGKNQMQEWKGLQSHRLLQIEINDAKRPASDVAISGLGWISIEPIRKTRGTEPRDLNEAEHEIHICVSVPKPVEVFLRPTLPIGTSGTEWYQYRELTDKEEEVRPKWYF

SEQ ID NO: 60 - protein, 50 aa, Artificial sequence
  motif 5
LTEAPVPGTTLGIIRIXGVLGGGAKMYDTPGLLHPYQLTMRLNREEQKLV

SEQ ID NO: 61 - protein, 50 aa, Artificial sequence
  motif 6
LLQPPIGEERVXELGKWXEREVKVSGESWDRSSVDIAIAGLGWFSVGLKG

SEQ ID NO: 62 - protein, 50 aa, Artificial sequence
  motif 7
KLVDIVDFNGSFLARVRDLAGANPIILVITKVDLLPRDTDLNCVGDWVVE

SEQ ID NO: 63 - protein, 50 aa, Artificial sequence
  motif 8
TYELKKKHHQLRTVLCGRCQLLSHGHMITAVGGHGGYPGGKQFVSAEELR

SEQ ID NO: 64 - protein, 50 aa, Artificial sequence
  motif 9
KMYDTPGLLHPYQLSMRLNREEQKMVEIRKELKPRTYRIKAGQSVHIGGL

SEQ ID NO: 65 - protein, 50 aa, Artificial sequence
  motif 10
RLQPPIGEERVAELGKWEEREVKVSGTSWDVSSVDIAIAGLGWFGVGLKG

SEQ ID NO: 66 - protein, 6 aa, Artificial sequence
  motif 11
CYGCGA

SEQ ID NO: 67 - protein, 13 aa, Artificial sequence
  motif 12
KLVDVVDFNGSFL

SEQ ID NO: 68 - protein, 15 aa, Artificial sequence
  motif 13
VYILGSANVGKSAFI

SEQ ID NO: 69 - protein, 11 aa, Artificial sequence
  motif 14
YDTPGVHLHHR

SEQ ID NO: 70 - protein, 10 aa, Artificial sequence
  motif 15
DVAISGLGWI

SEQ ID NO: 71 - DNA, 2194 bp, Oryza sativa; identical to SEQ ID NO: 9

SEQ ID NO: 72 - DNA, 54 bp, Artificial sequence
  primer prm09511
ggggacaagtttgtacaaaaaagcaggcttaaacaatggcgctacgaacactct

SEQ ID NO: 73 - DNA, 50 bp, Artificial sequence
  primer prm09512
ggggaccactttgtacaagaaagctgggttaagccgatatttttgcatct

SEQ ID NO: 74 - protein, 547 aa, Medicago truncatula
MALKTLSTFLTPLSLPNPKFPQIHSKPCLILCEFSRPSKSRLPEGTGAAAPSPGEKFLERQQSFEPTKLIPKQNNSKKKEKPLKASISVASCYGCGAPLQTSDNDAPGFVHSETYELKKKHHQLKTVLCGRCQLLSHGEMITAVGGHGGYSGGKQFITAEDLRQKLSHLRDAKALIVKLVDVVDFNGSFLSRVRDLAGANPIIMVVTKVDLLPRDTDFNCVGDWVVEAITRKKLNVLSVHLTSSKSLVGITGVISEIQKEKKGRDVYILGSANVGKSAFINALLKTMSYNDPVAAAAQRYKPVQSAVPGTTLGPIQINAFFGGGKLYDTPGVHLHHRQTAVVPSEDLSSLAPKSRLRGLSFPSSQVLSDNTNKGASTVNGLNGFSIFWGGLVRIDVLKALPETCLTFYGPKRMPIHMVPTEKADEFYQKELGVLLTPPSGREKAEHWRGLDSERKLQIKFEDAERPACDIAISGLGWLSVEPVGRSHRFSQQNAIDTTGELLLAVHVPKPVEIFTRPPLPVGKAGAEWYEYAELTDKEQEMRPKWYF

SEQ ID NO: 75 - protein, 547 aa, Oryza sativa
MAAPPLLSLSQRLLFLSLSLPKPQLAPNPSSFSPTRAASTAPPPPEGAGPAAPSRGDRFLGTQLAAEAAARVLAPEDAERRRRRREKRKALARKPSAAACYGCGAPLQTADEAAPGYVHPATYDLKKRHHQLRTVLCGRCKLLSHGHMITAVGGHGGYPGGKQFVSADQLRDKLSYLRHEKALIIKLVDIVDFNGSFLARVRDFAGANPIILVITKVDLLPRDTDLNCIGDWVVEAVVKKKLNVLSVHLTSSKSLVGVTGVISEIQQEKKGRDVYILGSANVGKSAFISAMLRTMAYKDPVAAAAQKYKPIQSAVPGTTLGPIQIEAFLGGGKLYDTPGVHLHHRQAAVIHADDLPSLAPQSRLRARCFPANDTDVGLSGNSLFWGGLVRIDVVKALPRTRLTFYGPKKLKINMVPTTEADEFYEREVGVTLTPPAGKEKAEGWVGLQGVRELQIKYEESDRPACDIAISGLGWVAVEPLGVPSSNPDESAEEEDNESGELHLRVHVPKPVEIFVRPPLPVGKAASQWYRYQELTEEEEELRPKWHY

SEQ ID NO: 76 - protein, 564 aa, Populus trichocarpa
MAPKSLSAFLFPLSLPHNLTYSTPKFLRIYTKPSPILCKSQQTPTATAHSSVSIPDQDGTGAAAPSRGDQFLERQKSFEAAKLVMKEVKKSKRREKGKALKLNTAVASCYGCGAPLHTLDPDAPGFVDPDTYELKKRHRQLRTVLCGRCRLLSHGHMITAVGGNGGYSGGKQFVSADELREKLSHLRHEKALIVKLVDVVDFNGSFLARLRDLVGANPIILVVTKVDLLPRDTDLNCVGDWVVEATTKKKLSVLSVHLTSSKSLVGIAGVVSEIQREKKGRDVYILGSANVGKSAFISALLKTMALRDPAAAAARKYKPIQSAVPGTTLGPIQIDAFLGGGKLYDTPGVHLHHRQAAVVHSEDLPALAPRSRLKGQSFPNSKVASENRMAEKIQSNGLNGFSIFWGGLVRVDILKVLPETCLTFYGPKALQIHVVPTDKADEFYQKELGVLLTPPTGKERAQDWRGLELEQQLQVKFEEVERPASDVAISGLGWIAVEPVSKSLRRSDINLEETIKELHLAVHVPKPVEVFVRPPLPVGKAGAQWYQYRELTEKEEELRPKWHY

SEQ ID NO: 77 - protein, 546 aa, Sorghum bicolor
MASPHLPFLSFPKTLPPPPPPLKPHAHRTSLAVAAAPAPPPAPPDGAGPAAPTRGDRFLGRQLATEAAARVLAPDDADRRRRRKEKRRALSRKPSGLASCYGCGAPLQTAEEAAPGYVDPDTYELKKRHHQLRTVLCGRCKLLSHGHMVTAVGGHGGYPGGKQFVSAEQLREKLSYLRHEKALIVKLVDIVDFNGSFLARVRDFAGANPIILVITKVDLLPRDTDLNCIGDWVVESVVKKKLNVLSVHLTSSKSLVGITGVISEIQQEKKGRDVYILGSANVGKSAFISAMLRTMAYKDPVAAAAQKYKPIQSAVPGTTLGPIQIEAFLGGGKLYDTPGVHLHHRQAAVIHADDLPSLAPQSRLKGRCFPANDTDVELSGNSLFWAGLVRIDVVKALPRARLTFYGPKKLKINMVPTTEADQFYETEVGVTLTPPTGKERAEGWQGLQGVRELKIKYEERDRPACDIAISGLGWISVEPSGVPSNSSDDNVEEEYDGGELHLVVHVPKPVEVFVRPPLPVGKAASQWYQYQELTEEEEELRPKWHY

SEQ ID NO: 78 - protein, 599 aa, Selaginella moellendorffii
MPVFSASALVSPSAFSTSRWLAANIVASSSERKNVKSFAKKTLQGDSIVIEIADKKRLDRFGARHEKTRQETQYKAGDSRKFKGPADPKESTKEEVSSGYVPLPSRGDKFLEEQKVRDQALVEKLAAKREKKKGKSQVVKLKSLEPCCYGCGAVLQYTQENTPGYINAETYELKKKHHQLKSVLCSRCQLMCHGKLIPAVGGYGIYGREKGFVTAEELRAQLAHIREERVLVLKLVDIVDFSGSFLTRVRDLVGNNPIVLVATKVDLLPEGTDLAAVGDWIVESTQRKKLNVISVHLTSAKYFMGITNIVKEIHRERQGRDVYILGAANVGKSAFISSLLKEMAARDPIAAVARKRKPVQSVLPGTTVGPISIDAFASGGSMYDTPGVHLHHRIETAISPDDLPSLFPARRLRGYSIFSEALKQAEKDEVISNVQDLTGTTMFWGGIARIDVLKAPQNTRLTFYASAALRVHKVLTSEADEFYKRELGKTLVPPSDERASAWPGLDHRNKFTFDYDDTRPVGDIAISGLGWMRMEFLQTESGVEDSLELEVYVPRGIEVFRRPAIPVGANTHSWYSFSELTAEQEKTRPRLYYSEHRGL

SEQ ID NO: 79 - protein, 562 aa, Vitis vinifera
MALKPLTSVFLSPLSLPYSPSNPTPKFSSFYTKPTPISCQTQAHQQAAPTSDPYRPESDGLGAAAPTRGDLFLEHHQSVAASEVVFNANKKKKKVKFSGSWKASAASACYGCGAPLQTLETDAPGYVDPETYELKKKHRQLRTVLCGRCRLLSHGQMITAVGGNGGYSGGKQFISAEELREKLSHLRHEKALIVKLVDIVDFNGSFLAHVRDLAGANPIILVVTKVDLLPKETDLNCVGDWVVEATMKKKLNVLSVHLTSSKSLVGISGVASEIQKEKKGRNVYILGSANVGKSAFINALLKMMAQRDPAAAAAQRYKPIQSAVPGTTLGPIQIDAFLGGGKLYDTPGVHLHHRQAAVVHSEDLPALAPRSRLRGQCFPVLAFDDSTLSRIKSNGLNGFSIFWGGLVRIDIVKVLPQTRLTFYGPKALNIHMVPTDKADEFYQKELGVLLTPPTGKQRAEDWLGLETERQLQIKFEDSDRPACDLAISGLGWIAVEPIGRSLRTSDSDLEETAEQLQLSIQVPKPVEIFVRPPIPVGKGGGEWYQYRELTEKEVEVRPQWYF

SEQ ID NO: 80 - protein, 733 aa, Chlamydomonas reinhardtii
MRAAVGRDALAAGAAVASPCSTSGRAALLRPLVVAAAPGFRGQASGAAAAAAVPSPSPSPLLAGASSSSPSCSPSCYSQQRQASLLSRRWSSISSTSHRPVATAASGRGDGATVADGAAGSSPASSSSPPRPSAADLSAASAQLLSDDQLRAAGLRLPSHCCGCGMRLQRRDAEAPGYFIIPARLFEPKRDPDADEDGFGRAGRGRGGAGAGAEAGGELGELMKAARQEMDADAEADAYDDVGLVRADEEPDVLCQRCFSLKHSGKVKVQAAETALPDFDLGKKVGRKIHLQKDRRAVVLCVVDMWDFDGSLPRAALRSLLPPGVTSEAAAPEDLKFSLMVAANKFDLLPPQATPARVQQWVRLRLKQAGLPPPDKVFLVSAAKGTGVKDMVQDVRQALGYRGDLWVVGAQNAGKSSLIAAMKRLAGTAGKGEPTIAPVPGTTLGLLQVPGLPLGPKHRAFDTPGVPHGHQLTSRLGLEDVKQVLPSKPLKGRTYRLAPGNTLLIGGGLARLDVVSSPGATLYLTVFVSHHVNLHLGKTEGAEERLPRLVEGGLLTPPDDPARAEQLPPLVPLDVEVEGTDWRRSTVDVAIAGLGWVGVGCAGRAGFRLWTLPGVAVTTHAALIPDMAEMFERPGVSSLLPKAQTRAHAAVKEKKAERAERRGGAGGDGGDGGGGGGEGRVVSRGERGWEAAGAVPAVGRSGGGGGGGRGGRGGGRGGRGRGGRSSGGRGGNS

SEQ ID NO: 81 - protein, 648 aa, Chlorella
MRLAAQKLQLVASRLAGCRTSRAGAASFNAVQRAAHRVAGRAPREAAARWPARRPVARASAAHEAGPDGSTSRPGYEADLQLPTHCSGCGVELQQEEPEAPGFFQVPKRLLEQLAAEGDLDGAGLEEDDSELVFDDVGLEADEAGAEAAAGQEQAGVAGEAAAGPGEVQEAASTSGRDPEEEAKWAAFDEMVESWLGGSKPARVEVASYAEQEEGQGTGGSSVLCARCFSLRHYGSVKSEAAEAELPAFDFERRVGLKIQLQKFRRSVVLCVVDVADFDGSLPRQALRSILPPDLQQGPLDVGRPLPLGFRLLVAVNKADLLPKQVTPARLEKWVRRRMAQAGLPRPSAVHVVSSTKQRGVRELLSDLQAAVGVRGDVWVVGAQNAGKSSLINAMRQVARLPRDKDVTTAPLPGTTLGMLRVTGLLPTGCKMLDTPGVPHAHQLSGHLTADEMRMVLPRRQLKPRTFRIGAGQTVMIGGLARVDVVDSPGATLYLSVFASDEIVCHLGKTETAEERYAMHAGGKLCPPLGGEQRMAAFPPLRPTEVTAEGDSWKASSKDVAIAGLGWVGVGVSGTAALRVWAPPGVAVTTHDALVPDYARDLERPGFGVALTEVGKNRREEEARQFKAAKQQQRKGRQGAKRAAAAGS

SEQ ID NO: 82 - protein, 604 aa, Ostreococcus lucimarinus
MPTATTRASGASVAARAQRTTTTTTTAAGTRWGRTGGSQRRGRAATARARAVGTGTPSVCPGCGVGLQREDANAPGYYVTPRRALEAAAAAEERNDEDDAEEASEAFEFEDGDDDVDDDAIDETYVPPGFELMDEENVSGLDAEEAAARLDALNSLFDDDEDDEATKRRAKKKRGPPTVVCARCFALRTSGRVKNAAAEVLLPSFDFARVVGDSFERLTGEGRAVVLLMVDLLDFDGSFPVDAIDVIEPYVEKGVVDVLLVANKVDLMPTQCTRTRLTSFVRRRSKDFGLSRCAGVHLVSAKAGMGVAILAQQLEDMLDRGKEVYVVGAQNAGKSSLINRLSQRYGGPGEEDGGPIASPLPGTTLGMVKLPALLPNSSDVYDTPGLLQPFQLSSRLNGDEMKVVLPNKRVTPRTYRIEVGGTIHIGGLARIDVLESPQRTLYLTVWASNKVATHYARTTKGADTFLEKHGGTKMTPPIGEARMRQFGAWGSRVVNIYGEDWQASTRDISIAGLCWIGVGCNGNASFKIWTHEGVQVVTREALVPDMAKSLMSPGFSFENVGGDSSNKRPNDRANRQRGRGGGGGGGGRGGRGGRGGRGGRSRSS

SEQ ID NO: 83 - protein, 542 aa, Ostreococcus RCC809
MGVASVCPGCGVGLQSEDKNAPGFFVMPKKVLEAASARAEDEDEDEGGEEAFELDETFEFGEDDDDFDDEDIDETYVPPGFELADEENVSALSAEEAEARLDALNSLFADEEDEDDEATKRRAKKKKGPPAVVCARCFALRTSGRVKNEAVEILLPSFDFSRVIGDRFERLTTKGSAVVLLMVDLLDFDGSFPVDAIDVIEPYSEEGVVDVLLVANKVDLMPVQCTRTRLTSFVRRRAKDFGLSRCAGVHLVSAKAGMGVQIFADQLEKLLDRGKEVYVVGAQNAGKSSLINRLSKRYGGPGEEDGGPIASPLPGTTLGMVKLPSLLPNGSDVYDTPGLLQPFQLSSRLNGEEMKIVLPNKRVTPRTYRIEVGGTIHIGGLARIDLLESPQRTLYLTVWASNKVPTHYARSSKGADAFLEKHGGTKMTPPVGELRMQQFGKWGSRIVNVYGEDWKSSTRDISIAGLCWIGVGCDGNASFRVWTHEGVQVVTREALVPDMDKSLMSPGFSFENVGGGSSNKRPNDRANRQRGRGGGGGRGRSR

SEQ ID NO: 84 - protein, 509 aa, Ostreococcus taurii
MLAAADADADADAEDEEAFDFDPDDDDFDDDDIDETLTLPGYELAPLVDAEDAEAKLDAFNALFDEDDEGTKRRAKKKKKGPPVIVCARCFALRTSGRVKNEAGESLLPSFDFERVIGDRFNRLREKNSAVVLLMVDLIDYDGSFPVDAVDVIEPYVQKGVLEVLLVANKVDLMPAQCTRTRLTSFVRQRSKDFGLSRCSGVHLVSAKAGMGMEILANQLEEMLDRGKEVYVVGAQNAGKSSLINRLSSKYGGPGEEDGGPIASPLPGTTLGMVKLASLLPNGSDVYDTPGLLQPFQLSARLTGEEMKMVLPNKRLTPRTYRIQVGGTIHIGALARIDLLESPQRTLYLTVWASNKVPTHYSTSAKAADTFLEKHAGTKMTPPLGQERMQQFGQWGSRLVNVYGEDWQKSTRDISIAGLCWIGVGCNGNASFRVWTHEGVQVVTREALVPDMDKQLMSPGFSFENVGGGSSGSNKKPNERANRQRGIGGGGGGRGGERGGGRGRSGSKR

SEQ ID NO: 85 - protein, 722 aa, Volvox carteri
MPTAGCCPEPVNGHATLTSHVTYSLAYREIQVTFKLVQSRTSPAERIDNFARILNPTFTTQGESPWATGVAPLEWVILKLDFGSLLPALEHGTHNPVLNCILTNFYYGISSTACGVPFVSAVFRRASVTQPAGAMRSCAPCGPTCRSSTIRAAWRLGSKTVVPPHILSLAPTLLLPQFRHHFATEKPALVAASAAEPAASTESNLGDVGEPRGPRGARGRRPVNTIGTSSASVAPPSAADLAAANLLSDEALRAMGIKLPSHCCGCGMKLQRQDERAPGFFTIPARLLEPPRGAAGPAAAGEDAGEVPVVRRELGNWGGGEDRHDEVEFDDVGALGADEPDVLCQRCYWLTHAGKLKSYEGEAALPTFDLSKKVGRKIHLQKDRKAVVLCVVDLWDFDGSLPRQAISALLPPGSGDEAPQELKFKLMVAANKFDLLPSVATVPRVQQWVRTRLKQAGLPHADKVFMVSAAKGLGVKDMDIRQALGFRGDLWVVGAQNAGKSSLIRAMKRLAGTDGKGDPTVAPVPGTTLGLLQVPGIPLGPKHRTFDTPGVPHTHQLTSHLNPEVVKKPGHSVLLGAGLARVDVVSAPGQTLYLTVFVSAHVNLHMGKTEGADDKVKSLTQNGLLSPPESPEEVAALPKWQPVEVEVEGTDWSRSTVDVAVAGLGWVGVGCRGKAHLRFWTLPGVAVTTHAALIPDYAKEFEKKGVSTLLPRTPKKQQARKV

SEQ ID NO: 86 - protein, 404 aa, Emiliania huxleyi
MRAHRFRLVTSAALAASLEDPRALEAEAARRGQPGAGFEMLGSYGGGPAGRPAGSAPLQAAIEMPRGFCCGCGVRFQANDEAAPGYLPASVLQQRLAPREAVCQRCHSLRYQNRLPSDGLRVGGGVQGADDPDAASHAELRPAHFRALIRSLRSKQCVVVCLVDLFDFHGSLVPELPSIVGEDSPLMLVDLLPKGIHQPAVERWVRAECRRASLPHLHSLDLVSARTGAGMPQLTTSHLPGTTLGFVKTAQLGGRHALYDTPGLVLPNQLTTRLTADELAAVVPKRRGQPVSLRLEEGRSLLLGGLARLDLVAGRPFLFTAYLSDAVTLHPTATAKAAEVRRKHAGGVLTPPASLERLEALGELEAQHELREHELRVEGRGWGEAAVDVVFPGLGWIAVTGSSG

SEQ ID NO: 87 - protein, 710 aa, Phaeodactylum tricornutum
MRTNFALSTRCFASSSDNHDEEEQRDSPKQRSKRSQTNRSKKFKIAESIDQSKIDKLAQAFDELARKEGFDSSTARFADDVTFEDKFDDDSFLDDDDDNNKDKVGNLHLDASMFSLSDFIDKSEEDGGNPTDQDDEDYLDFGADIDMSIEARIAAAKRDMDLGRVSAPPDMRSSRREVTAADLRKLGFRTEANPFGNDETPRKERFQLVTNSMSCSACGSDFQCHNEDRPGYLPPEKFATQTALGKIEQMQKLQDKAEKAEWTPEDEIEWLIQTQGKKDPNKEMQEVPQIDVDSLAGEMGLDLVELSKKMVICKRCHGLQNFGKVQDSLRPGWTKEPLLSQEKFRELLRPIKEKPAVIVALVDLFDFSGSVLPELDEIAGENPVILAANKADLLPSEMGRVRAESWVRRELEYLGVKSLAGMRGAVRLVSCKTGAGINDLLEKARGLAEEIDGDIYVVGAANAGKSTLLNFVLGQDKVNRSPGKARAGNRNAFKGAVTTSPLPGTTLKFIKVDLGGGRSLYDTPGLLVLGTVTQLLTPEELKIVVPKKPIEPVTLRLSTGKCVLVGGLARIELIGDSRPFMFTFFVANEIKLHPTDIERADEFVLKHAGGMLTPPLAPGPKRMEEIGEFEDHIVDIQGAGWKEAAADISLTGLGWVAVTGAGTAQVKISVPKGIGVSVRPPLMPFDIWKVASKYTGSRAVNYNFSLSVDI

SEQ ID NO: 88 - protein, 644 aa, Arabidopsis thaliana
MVVLISSTVTICNVKPKLEDGNFRVSRLIHRPEVPFFSGLSNEKKKKCAVSVMCLAVKKEQVVQSVESVNGTIFPKKSKNLIMSEGRDEDEDYGKIICPGCGIFMQDNDPDLPGYYQKRKVIANNLEGDEHVENDELAGFEMVDDDADEEEEGEDDEMDDEIKNAIEGSNSESESGFEWESDEWEEKKEVNDVELDEKKKRVSKTERKKIAREEAKKDNYDDVTVCARCHSLRNYGQVKNQAAENLLPDFDFDRLISTRLIKPMSNSSTTVVVMVVDCVDFDGSFPKRAAKSLFQVLQKAENDPKGSKNLPKLVLVATKVDLLPTQISPARLDRWVRHRAKAGGAPKLSGVYMVSARKDIGVKNLLAYIKELAGPRGNVWVIGAQNAGKSTLINALSKKDGAKVTRLTEAPVPGTTLGILKIGGILSAKAKMYDTPGLLHPYLMSLRLNSEERKMVEIRKEVQPRSYRVKAGQSVHIGGLVRLDLVSASVETIYITIWASHSVSLHLGKTENAEEIFKGHSGLRLQPPIGENRASELGTWEEKEIQVSGNSWDVKSIDISVAGLGWLSLGLKGAATLALWTYQGIDVTLREPLVIDRAPYLERPGFWLPKAITEVLGTHSSKLVDARRRKKQQDSTDFLSDSVA

SEQ ID NO: 89 - protein, 616 aa, Medicago truncatula
MAILFSTIALPSTNVTSKLSILNNTSHSHALRHFSGNTTKRFHKASSFIAFAVKNNPTIRKTTPRRDSRNPLLSEGRDEDEALGPICPGCGIFMQDNDPNLPGFYQQKEVKIETFSEEDYELDDEEDDGEEEDNGSIDDESDWDSEELEAMLLGEENDDKVDLDGFTHAGVGYGNVTEEVLERAKKKKVSKAEKKRMAREAEKVKEEVTVCARCHSLRNYGQVKNYMAENLIPDFDFDRLITTRLMNPAGSGSSTVVVMVVDCVDFDGSFPRTAVKSLFKALEGMQENTKKGKKLPKLVLVATKVDLLPSQVSPTRLDRWVRHRASAGGAPKLSAVYLVSSRKDLGVRNVLSFVKDLAGPRGNVWVIGAQNAGKSTLINAFAKKEGAKVTKLTEAPVPGTTLGILRIAGILSAKAKMFDTPGLLHPYLLSMRLNREEQKMAGQAIHVGGLARLDLIEASVQTMYVTVWASPNVSLHMGKIENANEIWNNHVGVRLQPPIGNDRAAELGTWKEREVKVSGSSWDVNCMDVSIAGLGWFSLGIQGEATMKLWTNDGIEITLREPLVLDRAPSLEKPGFWLPKAISEVIGNQTKLEAQRRKKLEDEDTEYMGASIEISA

SEQ ID NO: 90 - protein, 681 aa, Oryza sativa
MAKPLLLPATVAAAAAARLPSRLAVGAAPPFRVLPFFLCPPPQSRSLSFSPVSAVSTAGKRGRSPPPPPSPVISEGRDDEDAAVGRPVCPGCGVFMQDADPNLPGFFKNPSRLSDDEMGEDGSPPLAAEPDGFLGDDEEDGAPSESDLAAELDGLDSDLDEFLEEEDENGEDGAEMKADIDAKIDGFSSDWDSDWDEEMEDEEEKWRKELDGFTPPGVGYGKITEETLERWKKEKLSKSERKRRAREAKKAEAEEDAAVVCARCHSLRNYGHVKNDKAENLIPDFDFDRFISSRLMKRSAGTPVIVMVADCADFDGSFPKRAAKSLFKALEGRGTSKLSETPRLVLVGTKVDLLPWQQMGVRLEKWVRGRAKAFGAPKLDAVFLISVHKDLSVRNLISYVKELAGPRSNVWVIGAQNAGKSTLINAFAKKQGVKITRLTEAAVPGTTLGILRITGVLPAKAKMYDTPGLLHPYIMSMRLNSEERKMVEIRKELRPRCFRVKAGQSVHIGGLTRLDVLKASVQTIYITVWASPSVSLHLGKTENAEELRDKHFGIRLQPPIRPERVAELGHWTERQIDVSGVSWDVNSMDIAISGLGWYSLGLKGNATVAVWTFDGIDVTRRDAMILHRAQFLERPGFWLPIAIANAIGEETRKKNERRKKAEQRDDLLLEESAEDDVEVLI

SEQ ID NO: 91 - protein, 666 aa, Populus trichocarpa
MAVLLSTVAVTKPRLKLFNNNGITQEISSIPINIFTGLSLENKKHKKRLCLVNFVAKNQTSIETKQRGHAKIGPRRGGKDLVLSEGREEDENYGPICPGCGVFMQDKDPNLPGYYKKREVIVERNEVVEEGGEEEYVVDEFEDGFEGDEEKLEDAVEGKLEKSDGKEGNLETWAGFDLDSDEFEPFLEDEEGDDSDLDGFIPAGVGYGNITEEIIEKQRRKKEQKKVSKAERKRLARESKKEKDEVTVCARCHSLRNYGQVKNQTAENLIPDFDFDRLITTRLMKPSGSGNVTVVVMVVDCVDFDGSFPKRAAQSLFKALEGVKDDPRTSKKLPKLVLVGTKVDLLPSQISPTRLDRWVRHRARAAGAPKLSGVYLVSSCKDVGVRNLLSFIKELAGPRGNVWVIGAQNAGKSTLINALAKKGGAKVTKLTEAPVPGTTVGILRIGGILSAKAKMYDTPGLLHPYLMSMRLNRDEQKMVEIRKELQPRTYRVKAGQTIHVGGLLRLDLNQASVQTIYVTVWASPNVSLHIGKMENADEFWKNHIGVRLQPPTGEDRASELGKWEEREIKVSGTSWDANSIDISIAGLGWFSVGLKGEATLTLWTYDGIEITLREPLVLDRAPFLERPGFLLPKAISDAIGNQTKLEAKIRKKLQESSLDFLSEVST

SEQ ID NO: 92 - protein, 666 aa, Sorghum bicolor
MAAKPLLPIAAAAARLPFRLLSPSAPPPRGLPLLSPPFLPQRRSLSASAVPTGRRSRPPAPVISEGRDDEEAAVGRPVCPGCGVFMQDADPNLPGFFKNPSRSSQDETGGGGEVLLAAADTDAFLEDEKEGVVAEDALDAELEGLDSDIDEFLEDFEDGDEEDDGSPVKGATDIDAFASDWDSDWEEMEEDEDEKWRKELDGFTPPGVGYGNITEETIQRLKKEKLSKSERKRQAREAKRAEAEEDSALVCSRCHSLRNYGLVKNDKAENLIPDFDFDRFISSRVMKRSAGTPVIVMVVDCADFDGSFPKRAAKSLFEALEGRRNSKVSETPRLVLVGTKVDLLPWQQMGVRLDRWVRGRAKAFGAPKLDAVFLISVHRDLAVRNLISYIKESAGPRSNVWVIGAQNAGKSTLINAFAKKQGVKITRLTEAAVPGTTLGILRVTGVLPAKAKMYDTPGLLHPYIMAMRLNNEERKMVEIRKELRPRSFRVKVGQSVHIGGLTRLDVLKSSAQTIYVTVWASSNVPLHLGKTENADELREKHFGIRLQPPIGPERVNELGHWTERHIEVSGASWDVNSMDIAVSGLGWYSLGLKGTATVSLWTFEGIGVTERDAMILHRAQFLERPGFWLPIAIANALGEETRKKNEKRKAEQRRREEEELLLEEIV

SEQ ID NO: 93 - protein, 597 aa, Vitis vinifera
MRKNSRKNDIKFSFVALSVKSKYTIQETQKNNWKNPRKVGGNPILSEGKDEDESYGQICPGCGVYMQDEDPNLPGYYQKRKLTLTEMPEGQEDMEGSDGEESNLGTEDGNEFDWDSDEWESELEGEDDDLDLDGFAPAGVGYGNITEETINKRKKKRVSKSEKKRMAREAEKEREEVTVCARCHSLRNYGQVKNQMAENLIPDFDFDRLIATRLMKPTGTADATVVVMVVDCVDFDGSFPKRAAKSLFKALEGSRVGAKVSRKLPKLVLVATKVDLLPSQISPTRLDRWVRNRAKAGGAPKLSGVYLVSARKDLGVRNLLSFIKELAGPRGNVWVIGSQNAGKSTLINTFAKREGVKLTKLTEAAVPGTTLGILRIGGILSAKAKMYDTPGLLHPYLMSMRLNRDEQKMAEIRKELQPRTYRMKAGQAVHVGGLMRLDLNQASVETIYVTIWASPNVSLHMGKIENADEIWRKHVGVRLQPPVRVDRVSEIGKWEEQEIKVSGASWDVNSIDIAVAGLGWFSLGLKGEATLALWTYDGIEVILREPLVLDRAPFLERPGFWLPKAISDAIGNQSKLEAEARKRDQEESTKSLSEMST

SEQ ID NO: 94 - protein, 668 aa, Zea mays
MATKPFLSIPAAAVARLPFRLLCSAAPPPRLLPFFPQPFLLQRRSLSASTVPAGRRSSPPAPVISEGRDDEDAAVGRPVCPGCGVFMQDEDPNLPGFFKNPSRSSQDETGGSGEVLLAADTDAFLEEEDDNDDRRVADDASDAELEGLDSDIDEFLEEFDKGDEDDGLPVKSATDTDAFASDWDSDWEEMEEDEDEKWRKELDGFTLPGVGYGNITEETIERMKKEKLSKSQRKRQAREAKRAEAEEDSALVCSRCHSLRNYGLVKNDKAENLIPDFDFDRFISSRLMKRSAGTPVIVMVVDCADFDGSFPKRAAKSLFEALEGRRNSKASETPRLVLVGTKVDLLPWQQMGVRLDKWVRGRAKALGAPKLDGVFLISVHRDLAVRNLITYIKESAGPRSNVWVIGAQNAGKSTLINAFAKKQGVKITRLTEAAVPGTTLGILRVTGVLPAKAKMYDTPGLLHPYIMAMRLNNEERKMVEIRKEMRPRSFRVKVGQSVHIGGLARLDVLKSSVQTIYITVWASSNVPLHLGKTENSDELRDKHFGIRLQPPIGPERVDELGHWTGRSIEVSGASWDVNSMDIAVSGLGWYSLGLKGTATVSLWTFEGIGVTERDAMILHRAQFLERPGFWLPIAIANAIGEETRKKNEKRKAEQRRREEEELLLEEMV

SEQ ID NO: 95 - protein, 597 aa, Arabdidopsis thaliana
MLSKAARELSSSKLKPLFALHLSSFKSSIPTKPNPSPPSYLNPHHFNNISKPPFLRFYSSSSSSNLLPLNRDGNYNDTTSITISVCPGCGVHMQNSNPKHPGFFIKPSTEKQRNDLNLRDLTPISQEPEFIDSIKRGFIIEPISSSDLNPRDDEPSDSRPLVCARCHSLRHYGRVKDPTVENLLPDFDFDHTVGRRLGSASGARTVVLMVVDASDFDGSFPKRVAKLVSRTIDENNMAWKEGKSGNVPRVVVVVTKIDLLPSSLSPNRFEQWVRLRAREGGLSKITKLHFVSPVKNWGIKDLVEDVAAMAGKRGHVWAVGSQNAGKSTLINAVGKVVGGKVWHLTEAPVPGTTLGIIRIEGVLPFEAKLFDTPGLLNPHQITTRLTREEQRLVHISKELKPRTYRIKEGYTVHIGGLMRLDIDEASVDSLYVTVWASPYVPLHMGKKENAYKTLEDHFGCRLQPPIGEKRVEELGKWVRKEFRVSGTSWDTSSVDIAVSGLGWFALGLKGDAILGVWTHEGIDVFCRDSLLPQRAHTFEDSGFTVSKIVAKADRNFNQIHKEETQKKRKPNKSFSDSVSDRDNSREVSQPSDILPTM

SEQ ID NO: 96 - protein, 605 aa, Glycine max
MLVARSLSPSKLKPLFYLSILCECQNHFHSSLIPYSKPHLQNFPKFYPQPSTNLFRFFSSQPADSTEKQNLPLSREGNYDEVNSQSLHVCPGCGVYMQDSNPKHPGYFIKPSEKDLSYRLYNNLEPVAQEPEFSNTVKRGIVIEPEKLDDDDANLIRKPEKPVVCARCHSLRHYGKVKDPTVENLLPDFDFDHTVGRKLASASGTRSVVLMVVDVVDFDGSFPRKVAKLVSKTIEDHSAAWKQGKSGNVPRVVLVVTKIDLLPSSLSPTRLEHWIRQRAREGGINKVSSLHMVSALRDWGLKNLVDNIVDLAGPRGNVWAVGAQNAGKSTLINSIGKYAGGKITHLTEAPVPGTTLGIVRVEGVFSSQAKLFDTPGLLHPYQITTRLMREEQKLVHVGKELKPRTYRIKAGHSIHIAGLVRLDIEETPLDSIYVTVWASPYLPLHMGKIENACKMFQDHFGCQLQPPIGEKRVQELGNWVRREFHVSGNSWESSSVDIAVAGLGWFAFGLKGDAVLGVWTYEGVDAVLRNALIPYRSNTFEIAGFTVSKIVSQSDQALNKSKQRNDKKAKGIDSKAPTSFKEKLRNVRGPYIALPAMSERERQRR

SEQ ID NO: 97 - protein, 604 aa, Oryza sativa
MLSRARRLHPTLQRILRPVPPPAHPPPPPSPPHRPVFSQTPKPFFPFLRRHLSTKPPPPQAPPEKSLAPAKVSSDPPAVSANGLCPGCGIAMQSSDPSLPGFFSLPSPKSPDYRARLAPVTADDTRISASLKSGHLREGEAAAAASSSSAAVGVGVEVEKEGKKENKVVVCARCHSLRHYGVVKRPEAEPLLPDFDFVAAVGPRLASPSGARSLVLLLADASDFDGSFPRAVARLVAAAGEAHGSDWKHGAPANLPRALLVVTKLDLLPTPSLSPDDVHAWAHSRARAGAGGDLRLAGVHLVSAARGWGVRDLLDHVRQLAGSRGNVWAVGARNVGKSTLLNAIARCSGIEGGPTLTEAPVPGTTLDVIQVDGVLGSQAKLFDTPGLLHGHQLTSRLTREEQKLVRVSKEMRPRTYRLKPGQSVHIGGLVRLDIEELTVGSVYVTVWASPLVPLHMGKTENAAAMVKDHFGLQLQPPIGQQRVNELGKWVRKQFKVSGNSWDVNSKDIAIAGLGWFGIGLKGEAVLGLWTYDGVDVVSRNSLVHERATIFEEAGFTVSKIVSQADSMANRLKNPKKINKKKDNKANSSPSTDPESSNPVEAVDA

SEQ ID NO: 98 - protein, 597 aa, Sorghum bicolor
MLSRARRLHPAVRRFLLPNTPAPSRPAPLPPQHSASAQTSKTFSILFRRHLCSSPPAPPPSTSPPPAVVSSDLPAVRVNEVCPGCGISMQSSDPALPGFFLLPSAKSPDYRARLAPVTTDDTRISASLKSGHLREDLEPSGSDKPAAAAAEMADSKGEGKVLVCARCHSLRHYGRVKHPDAERLLPDFDFVAAVGPRLASPSGARSLVLLLADASDFDGSFPRAVARLVAAAGEAHSADWKHGAPANLPRALLVVTKLDLLPTPSLSPDDVHAWAHSRARAGAGSDLRLAGVHLVSAARGWGVRDLLEHVRELAGTRGNVWAVGARNVGKSTLLNAIARCSGIAGRPTLTEAPVPGTTLDVIKLDGVLGAQAKLFDTPGLLHGHQLTSRLTSEEMKLVQVRKEMSPRTYRIKTGQSIHIGGLVRLDVEELTVGSIYVTVWAAPLVPLHMGKTENAAALMKEHFGLQLQPPIGQEQVKELGKWVRKQFKVSGNSWDMNSKDIAIAGIGWFGIGLKGEAVLGLWTYDGVDVISRSSLVHERASIFEEAGFTVSQIVSKADSMTNKLKSTKKPNKKKERTKSASPLTKPEASEPASNIDA

SEQ ID NO: 99 - protein, 566 aa, Vitis vinifera
MIVRKFSASKLKHLLPLSVFTHSSTNLSLSPFSSNPISKTLNPNPHFLFSHSKLRPFSSSQSKPSLPFTRDGNFDETLSQSLFICPGCGVQMQDSDPVQPGYFIKPSQKDPNYRSRIDRRPVAEEPEISDSLKKGLLKPVVCARCHSLRHYGKVKDPTVENLLPEFDFDHTVGRRLVSTSGTRSVVLMVVDASDFDGSFPKRVAKMVSTTIDENYTAWKMGKSGNVPRVVLVVTKIDLLPSSLSPTRFEHWVRQRAREGGANKLTSVHLVSSVRDWGLKNLVDDIVQLVGRRGNVWAIGAQNAGKSTLINSIGKHAGGKLTHLTEAPVPGTTLGIVRVEGVLTGAAKLFDTPGLLNPHQITTRLTGEEQKLVHVSKELKPRTYRIKAGHSVHIAGLARLDVEELSVDTVYITVWASPYLPLHMGKTENACTMVEDHFGRQLQPPIGERRVKELGKWERKEFRVSGTSWDSSSVDVAVAGLGWFAVGLKGEAVLGVWTYDGVDLILRNSLLPYRSQNFEVAGFTVSKIVSKADQASNKSGQSQKRRKSSDPKAAAHCLPSPLTANAG

SEQ ID NO: 100 - protein, 560 aa, Chlorella
MIPAVVDFPQQQQQQQQRQPPQQEQPQQGQEREQAAAAGRRQDPLQEQDQLQQAQELERRRRRTGFTDKALLTPEELRQKLKVVQQQRALVVLLVDLLDASGSILGKVRELVGNNPIMLVGTKADLLPAGADGAQVAAWLQAAAAFKRIAAVSVHLVSSRTGAGVPEAVGAIRRERRGRDVFVMGAANVGKSAFIRALMKDMCRMGSRQFDPQALSRGRYLPVESAMPGTTLELIPMENKQLHPRRRLRPYVPPSPGELLQVTAAACSMPARPRDAGGAAAGAGAGAAAAAAAGPSCHVATYWWGGLAKLQLLSCPPDTELVFYGPQALLVEASVEAADPAGDAAAASEGDASASEGEGPGGDVGAGRRRGGGAASGGSGMGGGWPGLGGEEVAEEPHGFGAGSVMRRGGLRPCKTLHIKCGAGGSGARQAVADIAVSGVPGWVAVHASAGRGHTVQVRVWTPPGVEVFSRPPLPVPSPLVEPGAPDAWLPPRAAATPGGTLEQQQQEEPAGAAPATAAAAAAAPSAAAAQVTEVQGAGEAEEGRGQRVRQRPSSVDDWW

SEQ ID NO: 101 - protein, 365 aa, Emiliania huxleyi
MILLPLLLALPPLGLRRPAPLARRCAPPVAAEALVPRNRVACYGCGAELQADVAGSPGYMEPERYKMKRKRRQLRESLCDRCRRLSSGEILPAVVEGRLKRPSGAAVGEEGRGITTPEALRGVLLPLRERPALIALLVDLTDVAGTLLPRVRELVGGNPILLIGTKLDLLPRGTEPERVADWLSGAARKIGGVVDVHLVSSKAAPPRLSVGSVGSVGTPTSGLAGTSFFWGGLARIDVVSAPPALRLTFCTGGSRLRLHECPTAEAAEAHAARAGIEWTPPQDAASAAELGELQLARTARLRLTPCEQAADLAISGLGWVSVGCLPTLQQGALEATLAVWVPRGVEVFVRPPMPVGGLPTVGSEA

SEQ ID NO: 102 - protein, 460 aa, Ostreococcus taurii
MLARTSTRASTARARARSSRSSNAGARAPGERAARRHRARTRASNDPAATTATARERARCYGCGVGVQTRSNDVAGYVDVATYERKATHGQWDMMLCARCAKLSNGAYVNAVEGQGGVKASPGLITPKELRDQLKPIREKKALVVKVVDATDFHGSFLKKVRDVVGGNPIVLVVTKIDLLGNAVDHDALERWVAKEAETRRLTLAGIALVSSRRGSGMREAVLQMMRERNGRDVYVIGAANVGKSSFIRAAMEELRSAGNYFAPTKRLPVASAMPGTTLGVIPLKAFEGKGVLFDTPGVFLHHRLNSLLSAEDLSEMKLGSSLKKFVPPTPECAEPPGFASFKGYSLYWGSFVRVDVLECPPNVTFGFFGPKSTRVSLMKTADVPETISGQEEAALRLVQEIDFLPPMHVDGPLVDLSVSGLGGWIRVEKTSGRGDGPIRAHIYGIRGLEVFARDVMPTA

SEQ ID NO: 103 - protein, 714 aa, Phaeodactylum tricornutum
MLRSIRTGIRLGASPRKGLTAMKLQQPTPVFPASSVVTTIDRYNNQQYLYQTATAAFSWSAQQHLSAGPFLLAELGRSYTFLSSKLAPTATVSRRFAVAAKSPKSKKKGSSKKKQQSPVQKQPSSSKGKKHPGAPGTISSSSKIATRKPNKAGGSPPRGVKGRVVLQQHSAGKRISNAVPKLCSGCGTQVVSAKVSGRRSNNTDSANITGTRLVGGEDTMEHTSSLSKRIQKKTRYMDVGDYATRPMDSFLCSRCQSLQRNDIWGAYDALRDIEPKVFSEQLRFIVARRKFGMCIMVVDATDPEHTVVKHLRRTIGSIPVILVINKIDLLPRCSESDVMNITRRIEAMSGVRFTSVFDVSATNGVGLVRLAESILLQLGGRDVFVIGTANVGKSSLVKTLSPLIAESVYLKGQNRFAVKRRATIKNLKVTGSNLPGTTLQAVRVPCFPSDSHALWDTPGVISPRALQYKIFPAHLMEPLTRPEAIPIPASRNGLKVSLREGQSLLIEASWMGKDEENTKGIWDEDEETCVLGRIDVVQAKHHINAQAFLHPSLRLRVVPTSRAPDRATIPSFHIARVKERIFEATRNEVRGLADEYSLPLLPFLTETAPDGRFVAGYKEFVSASGRYVMDVSFASLGWVGFIDSNQYGVIPYCVEGSIFSKRRSLYPFNLAESVHSQEYTEQIPDHLDERAVKRQLSIAANEGRHTSNKVRQRF

SEQ ID NO: 104 - DNA, 1644 bp, Medicago truncatula
atggcgcttaaaaccctatccactttcttaacccctctttctctcccaaaccccaaattccctcaaattcactccaaaccttgtctcattctctgcgaattctctcgtccttccaaatcacgcttaccagaaggcaccggagccgctgctccgtcaccaggcgagaagttcctcgaacgccagcagtcatttgaaccaaccaaactcatccccaaacagaacaacagtaaaaagaaagagaagcctcttaaagcttccatttccgtagcttcttgctatggctgtggcgctcctttacaaacttctgataatgacgctcctggatttgtccactccgaaacctatgaattgaagaagaaacatcaccagcttaaaactgttctatgtgggcggtgccagcttttgtctcatggtgaaatgataactgctgttggaggacatggaggatactctggcgggaaacagttcattactgcagaagatcttcgacaaaaattgtctcatttgcgtgatgccaaagctctaattgtcaaattggttgatgttgttgacttcaatggcagttttttgtcccgagtgcgagatcttgctggtgctaatccaataatcatggttgtgactaaggttgatctccttccaagagatactgattttaattgtgttggggattgggttgtagaggctatcacaagaaagaaactaaatgttctcagtgtccatctcacaagttcaaaatcattggtaggaataactggagtgatatcagaaatccagaaagagaagaagggaagagatgtttacattctgggttcagcaaatgttggtaaatctgctttcatcaacgccttattaaagacaatgtcatataatgatccagtggctgcagctgcacaaagatacaaaccagtacagtctgctgttcctggaactaccttagggccaattcaaattaatgctttttttggaggagggaaactgtatgacactccaggagttcatctccaccacaggcaaactgcagttgttccttccgaagatctatcctcccttgctcctaaaagccgactgaggggcctatctttcccgagttcacaagtactttccgacaatacaaacaaaggtgcttcaacagtaaatggcttgaatggattttcaatattttggggaggtcttgttagaattgatgtcttgaaggctctaccggaaacatgtttaactttttacgggcctaagaggatgccaattcatatggtgcccacggagaaagcagatgaattttatcagaaagaacttggagttctgctaaccccaccaagtggaagagagaaggctgagcactggagaggacttgactcagaacgtaaattgcaaataaaatttgaagatgctgaaaggccagcttgtgatattgctatatcaggtctaggatggctttctgttgagccggttggcaggtcacacagattctcacaacaaaatgcaatagacactacaggcgaattgcttttagccgtacatgtccccaaacctgttgagatttttacgaggccaccattaccagtaggcaaggctggggcagagtggtacgagtatgcagaattaacggataaagaacaggaaatgagaccaaaatggtacttttga

SEQ ID NO: 105 - DNA, 1644 bp, Oryza sativa
atggcggcgcctcctctgctgagcctgagccagcggctactcttcctctccctctccctccccaagccacagctcgcccccaacccctcctctttctcccccacgcgcgccgcctccaccgccccgccacctccggaaggggcgggccccgccgcgccatcccgcggggaccgcttcctcggcacccagctcgcggccgaggccgccgcccgcgtcctcgctccggaggacgccgagaggcgccgccgccgccgggagaagcgcaaggccctcgcgcggaagccctccgccgccgcctgctacggctgcggcgcgccgctgcagacggccgacgaggccgcgcccggctacgtccaccccgccacctacgacctgaagaagagacaccatcagctgagaaccgtgctatgtgggagatgcaagctcttgtctcatggccacatgatcactgctgttggtggccatggcggctatcctggagggaagcagttcgtttccgcggaccaactcagggacaagctctcctaccttcgtcatgagaaagctttgattatcaagctggttgacatagttgacttcaatgggagcttcctggcgcgtgtgcgcgattttgctggtgctaatcctattatactagtcatcacaaaggttgatctccttcctagagatacagatttgaattgcattggcgactgggttgttgaggcagttgtcaagaagaagctcaacgtacttagtgtccatttgacaagctcaaagtcactcgttggcgtcactggggttatatcagagattcagcaggaaaagaagggccgagatgtatatatactgggttcagcaaatgttgggaaatctgcatttataagtgctatgctaaggacgatggcatataaggatccagtggcagctgcagctcaaaaatacaagccgatacaatctgctgttcctggaacgacccttggtcctattcaaattgaagcatttttaggaggcgggaaattatatgatacacctggagtccatcttcaccaccggcaagcagcagttatccatgctgatgatctgccttctcttgcaccacaaagtcgtctaagggcacggtgttttcctgctaatgatacagatgttggattgagtgggaattcattattctggggtggactagtccgtattgatgttgtcaaggctcttccacgcacacgactgacgttctatggacccaagaagctaaagattaatatggtcccaaccacagaagcagatgaattttatgagagagaagttggagttacattgactcccccagctggcaaagagaaggctgaaggatgggttggtctgcagggtgttcgtgagttgcagataaaatacgaagagtctgatagacctgcttgtgacattgcaatttctggtctcgggtgggttgcggttgagccacttggtgtgccatcaagcaacccagatgagagtgctgaggaagaagacaatgagagtggtgaactgcatttgagagtacatgttcccaagcctgttgagatctttgtccgacctccattgcctgttggtaaagcagcatcgcaatggtacagataccaggagttgaccgaggaagaagaggagttgagacctaaatggcattactga

SEQ ID NO: 106 - DNA, 1805 bp, Populus trichocarpa
aaccctgtcttctccgcttaacggtcatccatggcacctaaatccctctccgcatttctctttccactctctctcccccataatctcacatactccacccctaaattccttagaatttacaccaaaccctctcccatcctttgcaaatcacagcaaacgccaacagcgacagcccactcctctgtttccatacccgaccaggatggcaccggggcagctgctccttcccgaggagaccagttccttgagcgtcaaaaatcgtttgaggctgctaagttggtaatgaaagaggtgaagaagagtaagagaagagagaaagggaaggctttgaagctcaatacggctgttgctagttgttatggatgtggagctccgttgcataccttggatcctgatgctccgggttttgtcgacccggatacttatgaattgaagaagagacaccgccaacttagaacagttctttgtggaaggtgcaggcttttatctcatgggcacatgataactgctgttggtggaaatggcgggtattccggtgggaagcagtttgtttcagccgatgagcttcgtgaaaagctgtctcatttgcggcacgagaaagccttgattgtcaaattggttgatgttgtggacttcaatggcagctttttggctcgcttgcgtgatcttgttggtgccaatccaataatactagttgtgactaaggttgatctccttcctagggacactgatcttaattgtgttggtgattgggttgtagaggccaccacaaagaaaaagcttagtgttttgagtgtccatctcaccagctccaaatcattagttgggattgctggagttgtgtcagaaattcaaagggagaaaaagggccgagatgtttacattctgggttcagctaatgttgggaaatctgcattcatcagtgctttactgaaaacaatggcacttcgggatccagctgctgctgctgctcgaaaatacaaaccaatacagtcggctgttcctggaacaaccttaggtccaattcagattgacgctttccttggaggagggaaattatatgacacacccggagttcatctccaccatagacaagctgcagtggttcattcagaagatttacctgctcttgcccctcgaagtcgtctcaagggtcaatcttttcctaactctaaggtggcctctgaaaacaggatggcagaaaaaatccaatccaatggcttgaatggattttcaattttttggggaggtcttgtaagagttgatatcttgaaggttctccccgaaacatgcttaacattttatggccccaaggctctgcagattcatgtagtacccactgataaagctgatgagttttaccagaaagaacttggagttctattgacacctccaactggaaaagagagagcacaagattggagaggacttgaattagagcagcagttgcaagtaaaattcgaggaagtggaaaggcctgctagtgatgtagctatatcgggtctcggatggattgctgtggaaccggtaagcaaatcacttaggcggtcggatataaatttggaagaaactatcaaagaactgcatttagctgtgcatgtaccaaagccagtggaggtttttgtccggcctcctttaccagtaggcaaggctggagcacagtggtatcagtatcgagagttgacagagaaagaagaagaattgagaccaaaatggcactattagtggctgtgcctctttgatgtggtctgtgcaatcaatgtgcactgttggatagataaatgtcatatttcattacaaatttt

SEQ ID NO: 107 - DNA, 1925 bp, Sorghum bicolor
atggcgtcgccgcaccttcccttcctctccttccccaaaaccctaccgccaccacctccaccgctcaagccccacgcccaccgcacctccctcgccgtcgccgctgctcctgctcccccgcccgccccgcctgacggcgcggggcccgccgcgcccacgcgtggcgaccgcttcctcggccgccagctggccaccgaggccgccgcgcgcgtgctcgcgcccgacgacgccgacaggcgccgccgacgcaaggagaagcgccgggcactgtcgcggaagccctccggcctcgcctcttgctacgggtgcggcgccccgctgcagacggcggaggaggccgcgccgggatacgtagaccccgacacgtacgaactgaaaaagaggcaccaccaactgagaaccgttctatgtggaaggtgcaagctgctctctcatggccacatggtcactgctgttggtggccacggcggttatcctgggggcaagcagtttgtttctgcggaacagctcagggagaagctgtcatacctccgtcacgagaaagcactgatagtcaaattggttgacatcgttgacttcaatgggagtttcctggcacgagtacgtgactttgctggtgcaaatccgattattcttgtgataacaaaggttgatctccttcccagagacactgatttgaattgcataggcgactgggttgtcgagtcagttgtcaagaagaagcttaatgtccttagtgtccatttgacaagctcaaagtcactggtcggtatcacaggggttatatcagagattcaacaggaaaagaagggccgagatgtatatatactgggttctgcaaatgttgggaaatctgcatttatcagtgcaatgctaagaacaatggcatacaaggatccggtggcagcggcagctcaaaaatacaagcctatacagtctgctgttcctggaacaacccttggccctattcaaattgaagcatttttaggcggagggaaattgtatgatacacctggagtccaccttcaccataggcaggcagcagttatccatgctgatgatctgccttctcttgcaccacaaagtcgtttgaaagggcgatgttttcccgctaatgatacagatgttgaattgagtgggaattcattattctgggctgggctagtccgcattgatgttgtcaaggctcttccacgcgcacggctgacattctatgggcccaagaagctaaagattaatatggtcccgacaacagaagcagatcaattttacgagactgaagttggagttacattgactccaccaactggtaaggagagagctgaaggatggcaagggcttcaaggtgttcgcgagttgaagataaagtatgaggaacgtgacaggcctgcttgtgacatcgcgatctctgggcttgggtggatttctgtggagccatcaggcgtgccctcaaacagctctgatgacaatgtcgaggaagaatacgatggcggtgagctgcatctggtagtacatgtacccaaaccggttgaggtcttcgtccgccccccattgcctgttggtaaagcagcgtcgcaatggtaccagtatcaagagctcacagaggaagaagaggagttgaggcctaaatggcactactgatgctgctctggttctctacctatttctctagcagtagtacagttttgtgtaatccaaatatttagcacaaagttatatgctatgatgagatgatgttggcacgactagtgttaggatcaggtgataaggtggattcagaaaacaaattttgaataaatgtctctagtgtctatctagcacatgcttcagaaggccatgaggaaaagaatcctccagtacgtgctgtacatgtactctaaatgaaggagcttaccaacaagccatcgaagagattcataacaatt

SEQ ID NO: 108 - DNA, 3207 bp, Selaginella moellendorffii
atgccagtattcagcgcgtccgcgttggtttcacccagcgcattctcgacctcgcgctggcttgctgcgaatatcgtggctagcagctccgagaggaagaatgtgaagtctttcgccaagaagactttgcaaggtgattcaatcgtgattgagatcgctgataaaaagcgattggatcgttttggagcaagacacgagaagaccagacaagaaacacagtacaaggctggcgattctcggaagtttaaaggaccagctgatccaaaggaatcgaccaaagaggaggtttcgtcagggtatgttccgcttccaagcaggggtgacaagtttttggaggagcagaaagtcagagaccaagctctggtagagaaactggccgcaaagcgtgaaaagaagaaggggaagtcacaggtggtcaagctaaaatctctcgagccttgctgctatggttgcggtgcagttctccagtatacgcaagaaaacactcctggatacatcaatgccgagacgtatgaattgaaaaagaagcaccatcagctaaaatctgtgctctgtagcagatgccaactgatgtgccatggaaagcttatacctgccgtcggtggctacggcatctatggacgcgagaaaggttttgtgacggcggaggaattgcgtgcgcaattggcacatatacgcgaggaaagagtattggtgttgaaactcgttgacattgtggatttcagtggcagttttcttacccgtgttcgcgatctcgtcgggaataatccaattgttttggtggcgaccaaggtcgatcttcttcctgaaggtacagacttggcagctgttggcgactggattgtagagtctacacaacgaaagaaactgaatgtaatcagtgtgcatttgacgagcgcgaagtacttcatgggcattacaaatattgtcaaagagattcaccgcgaaagacagggacgtgatgtctatatactgggagcagccaacgtcggtaaatccgcattcatcagctctctgctcaaagaaatggccgctagagatccaattgcagcggtagcaaggaaacgcaagccagttcagtcagttttacctggcactacagtcggcccaatctcaatcgatgcttttgctagtggagggagcatgtacgatactcctggggttcaccttcaccaccgtattgagactgctatctccccagacgatcttccatcactttttccagctcgtcgtcttcgaggctattccatattttcagaagctctgaagcaagcggagaaggacgaggtgatatcgaacgtacaggaccttaccggcactactatgttctggggaggcatcgcaaggatcgatgtcttgaaggctcctcaaaacacccggttgacgttctatgcctcagcagcactgcgggtacacaaagtcctcacatccgaagctgacgagttctataaaagagaacttggaaagactctagtcccaccatccgatgagagagcttcggcatggcccggtttagatcaccgaaacaagttcactttcgattacgatgacaccagacctgtcggagacattgcgatatcgggtctcggctggatgagaatggagttcctccaaacggaaagtggagttgaagactccttggaactggaggtttatgtcccccggggaattgaggttttccgtcggccggctattccagtgggtgccaacacgcactcatggtactcgttctcagagttgacggcggagcaggagaaaacgaggccgaggctctactacagtgaacacaggggattgtagtttgcagttagtacttgcggcgatacagtgtagcgacaaacattccgctcagaagagttcccagcaaccaggattcctgtgatggtcccgaagtccgacgtgtccgtggatggactctccagacaagcagcctcatccaagagcacgtcacacctccacgcgagttcctcatgctgtccaacgcgaagaggagcaacgaagagcaaagaagaggagcaaggagcaattacgcacagaaagtctcatgcttcgccgagaagcgtttgttcttcgtgaaaccagttatggccagatagcagcagcatcgcttttgtggatccaatctctcgggagatgctcagtcgccaacatatttactcgggaattctctacagctttggtccctagagatccatctccggagatttccatgtcgtcgcttcagcgatcacgagctctgcccagctccgaattcctgcaacagaaggatcatgcttccaattactgtgtggagaagcctcagaatgcagccgctagtttgaggaagatcttttgttctcagcaagttcagcaaacgaggctcgcacccgcggaaagaatcaagcagggtttccagagattcaagcaagagacatacaaccaaaaaccagagctcttcagccaattggccacaggacaacatcccaagttcatggtgatcgcttgctccgattctagagtttgtcccacaacaattctggggttccagccaggggaagcatttgtcgttcgcaacattgcaaacatggtgcctcctccggaacaggctggctatccaggaacgagcgcagctcttgaatacgcagtcacggctctcaaggtcgagaacattttggtgatcggacatagtagatgcggcggcatcaaggctctcatgacacaaaaagaaaacacaaacaaatggagttcgttcattgaggactgggtcgaaatcggacgtccagctcgtgccgtgacgctcgccgcagcagcccagcagcaagttgagcaccaatgtacaaaatgcgagaaggaatccgtgaatgtgtcgctggcaaaccttcttgccttccctttcatcaaggaagcggtctccagcggcacgcttgctctccatggcggctactacaactttgtggagggctcgttcgagtactggtggtacggaattgacgggaagagcgaggtgtccaagttctagatctcctggaggtcagtttgttatttgattgatcgattggaagcgtgatcatcgtcactcctgcttggaaagccattctgccctccattccaaaggggatatcaagacgtgatcgactttccagccacattcttgcatctacaatgaaagagaataagaaacgattgtgatgatttactgcttaccatggcatagagcttgttaaagtt

SEQ ID NO: 109 - DNA, 1689 bp, Vitis vinifera
atggcacttaaacccctcacctccgtcttcctctctcctctgtcccttccctacagcccctcaaaccccacccccaaattctccagtttttacacaaaaccaactcccatctcatgccaaacccaagcccatcaacaagcagcccctacctccgacccttaccgtccagaatccgatggtttaggagcagcagccccgacccgaggtgacctattcctcgagcatcatcaatccgtggctgcttccgaggtcgtgttcaatgcgaataaaaagaagaagaaggtgaagtttagtgggtcttggaaggcttctgctgcgagtgcttgttatggttgcggagctccattgcagactttggaaactgatgctcctggttatgttgatccggaaacgtatgaattgaagaagaaacaccgccagcttagaactgttctttgtgggaggtgccggcttttgtctcatgggcaaatgattactgctgttggtggaaatggaggttattctggtgggaagcagtttatttcagctgaggagctccgagagaagttatctcacctgagacatgagaaagccttaattgttaaactggttgatattgtggacttcaatggaagttttttggctcatgttcgtgatcttgctggtgctaatccaataatattagttgtaacaaaggttgatctccttcctaaagagactgatcttaattgtgtgggtgattgggttgttgaggcgaccatgaagaagaagcttaatgttctgagtgtccatctgacaagttcaaaatctctggttggaatttctggagttgcatcagaaattcaaaaggagaaaaagggtcggaacgtgtacattctgggctcagctaatgttggaaaatctgcattcatcaatgctctactaaagatgatggcccaaagggatccagctgctgcagcagcacaaagatacaagccgatacaatctgctgttcctggaactaccttaggtccaattcaaattgatgctttcctaggtggagggaaattatatgacacacctggcgttcatcttcatcataggcaagctgctgttgttcattctgaagacctacctgcccttgctcctcgaagtcgcctcaggggccaatgctttcctgtactggcctttgatgatagtacattgagcagaattaaatctaatggactgaatgggttttcaatattctggggaggtcttgtgagaattgatattgtgaaggttctcccacagacaagattgacattctacgggcctaaggcattaaatattcatatggtgccgactgacaaagcagatgaattttaccagaaagaacttggagttcttttgacaccaccaactggaaaacagagagcagaagactggttagggcttgaaacagagcgccaactgcaaataaaatttgaagatagcgacaggcctgcatgtgatttggcaatctcgggcctaggatggattgctgttgaaccaataggcagatcactcagaacttctgattcagatttagaagaaactgccgaacaactgcagttatccattcaagttccgaagccggtggagatatttgtaaggcctccaattccagtggggaagggtggaggagagtggtaccagtaccgggaattgactgagaaagaagtggaagtgagaccacaatggtatttctga

SEQ ID NO: 110 - DNA, 2202 bp, Chlamydomonas reinhardtii
atgcgcgccgccgtcggtcgcgacgctcttgccgcgggcgcagcggtggcgtctccgtgcagcaccagcggccgcgccgccctgctacggccgctggtcgtggcagcggcccccggctttcgtggccaagcgagcggtgcagccgctgctgccgcagtgcccagccctagcccgagcccgctgctcgctggggcctcctcctcgtccccctcgtgctccccctcgtgctacagccagcagcgccaggccagcttgctcagccggcgttggtccagcatcagctccacctcgcaccgccccgtggctactgccgccagcggccgcggcgacggcgcaaccgtcgccgacggcgccgctggctcctcccccgcctcctcgtcttcccctccgcggccgtccgccgccgacctgtccgccgccagcgcgcagctgctgagcgacgaccagctgcgggcggcggggctgcggctgccgtcgcactgctgcggctgcggcatgcggctgcagcgccgggacgcggaggcgcccggctatttcatcattcccgcccgcctgtttgagcccaagcgggacccggacgcggacgaggacggcttcggacgggccggccgcggccggggcggcgccggcgctggcgcggaggccggtggcgagctgggggagctgatgaaggcggcgcgtcaggagatggacgcggatgcggaggccgacgcgtacgacgacgtgggtctggtgcgtgcggacgaggagccggacgtgctgtgccagcggtgcttctcgctcaagcactcgggcaaggtcaaggtgcaggcggcggagacggcgctgccggattttgacctgggcaagaaggtgggccgcaagatccacctgcagaaggaccggcgcgcagtggtgctgtgcgtggtggacatgtgggacttcgacggctcactgccgcgcgcggcgctcaggtcgctgctgcccccgggcgtgacttccgaggccgccgcgcccgaggacctcaagttcagcctcatggtggcggccaacaagttcgacctgctgccgccgcaggccacacccgcacgagtgcagcaatgggtgcggctgcggctcaagcaggccggcctgccgccgccggacaaggtgttcctggtcagtgcggccaagggcacaggcgtcaaggacatggtgcaggacgtgcggcaggcgttgggctaccgcggcgacctgtgggtggtgggcgcccagaacgcgggcaagagctccctcatcgccgccatgaagcggctggcggggacggcgggcaagggcgagcccaccatcgcgccagtgcccggcaccacgctgggcctgctgcaggtgccggggctgccgctggggcccaagcaccgcgccttcgacacgcccggcgtgccgcacggccaccaactcaccagccgcctggggctggaggacgtcaagcaggtgctgccctccaagccgctcaaggggcgcacctaccgcctggcgcctggcaacaccctgctcataggcgggggcctggccaggctggacgtggtgtccagccccggcgccacgctgtacctgaccgtgttcgtcagtcaccacgtcaacctgcacctgggcaagacggagggcgccgaggagcggctgcctcggctggtggagggcggcctgctgacgccgcccgacgacccggcgcgcgccgagcagctgccgccgctggtgccgctggacgtggaggtggagggcacggactggcgcaggagcactgtggacgtcgccattgcggggctgggctgggtgggcgtgggctgcgcgggccgggcgggcttccggctgtggacgctgccgggcgtggcggtgacgacgcacgcggcgctgattccggatatggcggagatgtttgagcggccgggggtgtccagcctgctgcccaaggcgcagacgcgcgcgcacgcggccgtgaaggagaagaaggcggagcgggccgagcggcggggaggcgctgggggtgatgggggcgatggaggagggggtggtggtgagggccgtgtggtgagcaggggcgagcgcggctgggaggcggcgggggcggtgccggctgtgggcaggtcgggcggcggaggcggtggtggtcggggagggcgcggcggtgggcgtggcggcagggggcgcggcgggaggtcgtcaggcggccgcggtggcaacagctga

SEQ ID NO: 111 - DNA, 1947 bp, Chlorella
atgcgcctcgctgcacaaaagctgcagctggtagccagccggctggcgggctgccgcaccagtagggcaggagctgccagtttcaatgccgtgcagcgtgctgctcacagggtggctggccgggcgccgcgggaagcggcggcgcggtggcccgcccgccggcccgtggcgagggccagcgcggcacacgaggcgggcccagacggcagcaccagccggccgggctacgaggcagacctgcagctgcccacccactgctccggctgcggcgtggagctgcagcaggaggagccggaggcccccggcttcttccaggtgcccaagcgcctgctggagcagctggcagcggagggcgacctggatggcgctgggcttgaggaggacgacagcgagcttgtgtttgacgacgtggggctcgaggctgatgaggccggcgccgaggcggcggccggccaggagcaggcgggcgtggcgggcgaggcggcggctgggccgggggaggtgcaggaggcggcgagcacgtctgggcgggacccagaggaggaggccaagtgggctgcctttgatgagatggtggagagctggctgggcggctccaagccagcgcgcgtggaggtggccagctatgcggagcaggaggagggccagggcacgggcgggtccagcgtgctgtgcgcgcgctgcttctcgctgcggcactacgggtctgtgaagagcgaggccgcggaggcggagctgccggcctttgactttgagcgcagggtgggcctcaagatccagctccagaagttcaggcgctcggtggtgctctgcgtggtggatgtggcagacttcgacggctcgctgccgcgccaggcgctgcgcagcatcctgccgccggacctgcagcaggggccgctggatgtggggcgaccgctgccgctgggcttccgcctgctggtggccgtcaacaaggcagacctgctgcccaagcaggtcacgcccgcacgcctggagaagtgggtgcgcaggcgcatggcgcaggcaggcctgcccaggcctagcgccgtgcatgtcgtgagcagcaccaagcagcgcggcgtgcgggagctgctgtcagacctgcaggcggcggtgggcgtgcgcggcgacgtgtgggtggtgggcgcgcagaacgcgggcaagagctccctgatcaacgccatgcgccaggtggcgcgcctgcccagggacaaagacgtcaccacggcgccgctgccgggcaccacgctgggcatgctgcgagtgacgggcctgctgcccaccggctgcaaaatgctcgacacgcccggcgtgccgcacgcgcaccagctgtccggccacctgaccgccgacgagatgcgcatggtgctgccccgccgccagctcaagccccgcactttccgcatcggggccggccagacggtcatgattggcgggctggctcgcgtggatgttgtggacagccccggcgccaccctctacctctccgtctttgccagcgacgagattgtgtgccacctgggcaagactgagaccgcggaggagcggtacgccatgcacgccggcggcaagctgtgccccccactgggcggcgagcagcgcatggcggccttcccaccgctgcggcccaccgaggtgacggcggagggcgactcgtggaaggccagtagcaaggatgtggccatagcaggcctgggctgggtgggggtgggcgtgtctggcaccgcggcgctgcgcgtgtgggcgccgccgggtgtggcggtcaccacccacgacgcgctggtccccgactatgctcgggatctggagcgcccaggctttggtgtggcgctgacggaggtggggaagaaccggcgggaggaggaggcgcggcagttcaaggccgccaagcagcagcagcgcaaggggcggcagggggccaagagggcggcggcggccggcagctag

SEQ ID NO: 112 - DNA, 1833 bp, Ostreococcus lucimarinus
atgccgacggcgacgacgcgcgcgagcggcgcgagcgtcgcggcgcgcgcgcagcggacgacgacgacgacgacgacggcggcggggacgcgatggggacggacgggcgggagccagcgacgggggcgcgcggcgacggcgcgcgcgcgcgcggtggggacgggaacgccgagcgtgtgcccggggtgcggggtcgggctgcagcgcgaggacgcgaacgcgccggggtactacgtgacgccgagacgcgcgctggaggcggcggcggcggcggaagagaggaacgacgaggacgacgcggaggaagcgagcgaggcgttcgagttcgaggacggcgacgacgatgtggacgacgacgcgatcgacgagacgtacgtgccgccggggttcgagttgatggatgaagaaaacgtgagcgggttggacgccgaggaggcggcggcgcggttggacgcgttgaattctttgtttgacgacgacgaggacgacgaggcgacgaaacgacgggcgaagaaaaagcgtggaccgccgacggtggtgtgcgcgcggtgcttcgcgctgcgaacgagcggacgggtgaagaacgcggcggcggaggtactgttgccgtcgttcgatttcgcgcgcgtcgtcggcgatagtttcgagcggttgacgggcgaaggccgcgccgtggttttactcatggtcgatttactggatttcgacggatcgtttccggtggatgccatcgacgtcatcgagccgtacgtggagaagggcgtggtggacgtcttgctcgtggcgaacaaggtggacttgatgcccacgcagtgcacgcgcacgcgcttgacttcgttcgtgcgacggcggtcgaaggatttcgggctttcgcgatgcgcgggcgtgcacttggtgagcgccaaagcggggatgggggtggcgattttggcgcaacagctcgaagacatgctcgatcgagggaaagaggtgtacgtcgtcggcgcgcaaaacgcgggtaagagttcgttgatcaaccgcttgagtcaaaggtacggcggcccgggtgaagaagacggaggcccgatcgcgagtccgcttcctgggacgacgctcgggatggtgaagctcccggcgctgttgccgaacagttcagacgtctacgacacgcccggattgttgcaaccgtttcaactctcttcgcgattgaacggcgatgagatgaaggtcgttttaccgaacaagcgtgtcacgccgcgcacgtatcgcatcgaagtcggtggcacgattcacataggtggtttagcgcgcatcgacgtcttggaatcgccgcaacgcacgctatacctcaccgtgtgggcgtcgaacaaggtcgccacgcactacgcgcgcacgacaaagggagcggacacgtttctcgagaagcacggagggacgaagatgacgcctccgatcggagaggctcgcatgagacagtttggcgcgtgggggtcacgcgtcgtgaacatctatggcgaagactggcaagcgtcgacgcgagacatctccatcgccgggctctgttggatcggcgttgggtgcaatgggaacgcttcgttcaagatttggacgcacgagggcgtgcaagtcgtcactcgcgaagcgttagttcccgacatggccaagagtttaatgtcgcccggtttttcctttgaaaacgtcggcggcgattcgtcaaacaagcgtccgaacgatcgcgcgaatcggcaacgcggtcgaggcggcggcggcggcggcggcggtcgaggcggtcgaggcggtcgaggcggtcgaggcggtcgatcgcggtcgtcatagcggtcaatcaataaaagt

SEQ ID NO: 113 - DNA, 1629 bp, Ostreococcus RCC809
atgggggtggcgagcgtgtgtccgggatgcggggtgggattgcagagcgaggataagaacgcgccggggtttttcgtgatgcccaaaaaggttttggaggcggcgagcgcgcgcgccgaggacgaggacgaggacgagggcggggaggaggcgtttgaactcgacgaaacgtttgaattcggtgaggatgacgacgatttcgacgacgaggacatcgacgagacgtacgtgccgcccgggtttgagctggcggatgaagagaacgtgagcgcgttgagcgcagaggaggcggaggcacggttggatgcgttgaattcgttattcgcggacgaagaggacgaggacgacgaggcgacgaagagacgggcgaagaagaaaaagggtccgccggcggtggtgtgcgctcggtgcttcgcgttgagaacgagcggacgggtgaagaacgaggcggtggagattttattgccgtcgtttgatttctctcgcgtcatcggcgatcgattcgaacgacttacaacaaaagggagcgccgtggtgttactcatggtggatttgttggatttcgacggatcgtttccggtcgacgccatcgacgtcatcgagccgtattcggaggagggcgtcgtcgacgtgctcctggtggcgaacaaggttgatttgatgccggtacagtgcacgcgcacgcgtctgacgtccttcgttcgacgtcgcgcgaaggatttcggtctgtcacgatgcgcgggcgtgcacttggtcagtgccaaggcgggcatgggtgtgcaaatttttgccgaccaactcgaaaagttgctggataggggcaaagaggtgtacgtcgttggcgcccaaaacgccgggaagagttctctcatcaatcgtctgagcaagcgttacggcggtcctggtgaggaagacggcggtccgatcgcgagcccgctgcccgggacgacgctcgggatggtgaaacttccgtcgctcttgcccaacggctcggacgtgtacgacacgccgggattgttgcagccgtttcagctgtcgtctcgcttaaacggtgaagagatgaagattgttttaccgaacaagcgcgtgacgccgcgcacatatcgcatcgaggtcggaggaacgattcacatcggcggtttggctcgcatcgacctcttggagtctccgcagcgcacgctctacctcaccgtgtgggcgtccaacaaagtgcccacgcactacgcgcgatcatccaagggcgcggacgctttcctcgagaagcacggtggtacgaaaatgacgccgccggtcggcgaacttcgcatgcaacagttcggtaagtggggttcgcgcatcgtcaacgtatacggagaggattggaagtcgtcgacgcgcgacatctcgatcgcgggtttatgctggatcggcgtcgggtgcgatggaaacgcgtcgtttcgcgtgtggacacacgagggcgtgcaagtggtcacgcgcgaggcgttagttccggacatggataagagcctcatgtcgcccggcttttcgttcgaaaacgtcggcggcggttcgtccaacaaacgccccaacgaccgcgcgaacagacagcgaggtcgcggcggcggcggcgggcgcggacgatcgagatag

SEQ ID NO: 114 - DNA, 1602 bp, Ostreococcus taurii
tgcccgggatgtggggtgggattgcaagacgttgatgcgaacgcgccggggttttacgtgacgccgaagaagatgttggcggcggcggacgcggacgcggacgcggacgcggaagacgaggaggcgttcgacttcgatccggacgatgatgattttgacgacgatgatatcgatgagacgctgacgcttccggggtacgagttggcgcctttggtcgacgcggaggacgcggaggcgaaattggacgcgttcaacgcgctgttcgacgaggacgacgagggaacgaaacgaagggcgaagaagaagaagaagggtccgccggtcatagtgtgcgcgcggtgtttcgcgctgcgaacgagcggtagggtgaagaacgaggcgggggaatcgcttttgccgtctttcgacttcgagcgcgtcatcggggacaggtttaatcgactgagggaaaagaatagcgcggtcgttttactcatggtggacttgatcgattacgacggttcgtttccggtcgacgccgtagatgtcatcgaaccgtacgtgcaaaagggtgtgctggaagtcctgctcgttgcgaacaaagtggacttaatgccggcgcagtgcacgcgaacgcgcttaacctccttcgttcgtcagcgatcgaaagatttcggcctttcacgatgctcgggcgtgcacttggtgagcgcaaaggcaggaatgggaatggaaatcttggcgaaccaactcgaggagatgctcgacagagggaaggaggtgtacgtcgtcggggcgcaaaacgctggcaagagctcactcatcaatcgactgagctcgaaatacggtggaccgggcgaagaggacggtggtccgatcgcgagtccgctcccagggacgacgctcggcatggtcaagctcgcgagcttgctccccaatggctcggacgtgtacgacactcctgggttgttgcaaccgttccagctgtcggctcggctcacgggcgaagagatgaagatggtgctcccgaacaagcgcttaacgcctcggacgtaccgtatccaggtcggtggaaccattcatataggtgctttggcgcgaatagatctgttggagtctccgcagcgcacgctgtacctcacggtgtgggcatccaacaaagtcccgacccactactcgacgtcagccaaggcggcggacactttcctggagaaacacgctgggacgaagatgactcctccgcttgggcaagaacgcatgcagcagttcggtcagtggggctcgcgtttggtgaacgtctacggtgaagactggcagaaatcgacgcgagacatctccatcgctgggttgtgttggatcggcgtcggctgcaacggtaacgcttcgttccgtgtgtggacgcacgagggcgtgcaagtggtcactcgcgaggcgctcgtgccggatatggataaacagttgatgtcacccgggttctcgttcgaaaacgtcggcggtgggtcgtcgggatctaacaaaaaaccaaacgagcgtgcaaacagacagagaggcatcggtggtggaggaggcgggcgtggtggcgaacgcggtggtggacgaggtcgaagtggttcgaaacgatga

SEQ ID NO: 115 - DNA, 2169 bp, Volvox carteri
atgccaactgccggctgctgtccagagccggtgaacggccatgcgacactgacatcacatgtgacatattcgttagcatatcgtgagatccaggtcactttcaagcttgtgcaatcgcgcacaagccctgccgaacgcattgataattttgccaggattctgaatccaactttcacgacgcagggcgagtcgccctgggcgacgggcgtcgccccgctagaatgggttatcctgaagctcgattttgggtcgttattaccagctctcgaacacggcacgcataatccagtccttaattgtattttaacaaacttctattacggtatatcttccactgcctgcggcgttccctttgtgtcagccgtcttcaggagagcatccgtcacgcagccggccggcgccatgcgctcctgcgcaccctgcgggcccacctgccgctcatcaacaatacgcgcagcatggcgtcttggatccaaaaccgtcgtccctccacatatcctttcacttgctccgacgcttctccttcctcagtttcggcatcattttgcaactgagaagccagctcttgtggcggcttctgccgcagaaccagcagccagcacagaatcaaatttaggggacgttggcgaaccccgaggaccccgtggcgctcgcggccgacgacccgttaacactatcggcacgagctccgcctccgtcgcacccccaagcgctgcagacctcgcagcagcaaacttgctgagcgacgaggcgctgcgcgcaatgggcatcaagctacccagtcattgctgcggctgcggcatgaagctgcagcggcaggacgagcgagctccagggttctttactatcccagccaggcttttggagccgccccggggggcggcgggtcccgcagcggcgggtgaagatgcgggggaggtccctgtggtgagaagggagttggggaattggggaggaggggaggaccggcacgatgaggtggagttcgacgacgtgggggcgctgggtgcggatgagcccgatgtgctgtgtcagcgttgctactggctcacgcacgccgggaagctcaagtcgtacgagggggaggcggcgctgccgacattcgatctgagcaagaaggtgggccgcaagatccacctacaaaaggaccggaaggcggtggtgttgtgtgtggtggacctctgggatttcgacggctcgttaccccgccaagctatcagtgcgttgcttcccccgggcagcggtgatgaggccccccaggagctgaaattcaaactgatggtggcggcgaacaaattcgatttgctgccgtccgtcgccacggtgccccgtgtccagcaatgggttcgtacacggctcaagcaggcaggtctcccccacgctgacaaggtgttcatggtcagcgccgccaaggggctcggcgtcaaggacatggatatccgtcaggctctggggttccgaggtgacctctgggtggtgggggcgcagaacgcggggaagagctccctcatccgggccatgaagcggttggcggggacagacggcaagggtgacccaaccgttgcacctgttccgggaacaaccttggggctccttcaggtccccggaatacccctgggccccaaacaccgcacgtttgacacgccgggtgtgccgcacacccatcagctcaccagccacctcaaccccgaggtcgtcaaaaagcccgggcactcggtcttgctgggcgcaggtctggcgcgagtggatgtggtttcggcgccggggcaaaccctgtacctgactgtgttcgtatctgcgcacgtcaacttgcatatgggcaagactgaaggtgcggacgacaaggtgaaatcactgacgcaaaacggtttgttatcgcctccggagtcgccggaagaggttgcagcgttgcccaaatggcagccggtggaggtcgaggtggaaggcacggactggtctagaagcacggtggacgtggcggtagcgggccttggctgggtgggtgtgggttgccgcggcaaggctcatctgcgtttctggacgctgcccggggtagcggtcaccacacatgcggctctcataccggactacgccaaggagtttgagaagaagggcgtgtcaacgctgttgccgaggacgccgaagaagcagcaggcgaggaaggtctga

SEQ ID NO: 116 - DNA, 1215 bp, Emiliania huxleyi
atgcgcgcccaccgcttccgtctcgtcacctcggccgcgctggctgcctcgctcgaggacccgcgcgcgctggaggcggaggcggcgcggcgcggccagccaggcgccggctttgagatgcttggcagctacggaggcgggccggcgggccggcctgcagggagcgcaccgctgcaagcggcgatcgagatgccgcgcggcttctgttgcggctgcggcgtccgcttccaggcgaacgacgaggccgcgccaggctatctgccggcgtccgtgctgcagcagaggcttgcgccgagggaggcggtgtgccagcgctgccactctttgcgctaccagaaccggctgccgtcggatggcttgcgtgtgggcggcggcgtgcagggcgccgacgacccggatgcagcgtcacacgcggagctgcggccggcgcacttccgcgcgctgatccgatcgctgcggtcgaagcagtgcgtcgtcgtctgcctggtcgacctcttcgacttccacggctcgctcgtgccagagctgccctcgatcgtgggcgaggactcccccctcatgctcgtcgacctcctacccaagggcatccaccagccagcggtcgagcggtgggtgcgcgccgagtgccgccgcgcctcgctgccgcacctccactccctcgacctcgtctcggcgaggacgggcgcgggcatgccgcagctcacgacctcgcacctgcccgggaccacgctcggcttcgtcaagacggcgcagctcggagggcggcacgcgctgtacgacacgcccggcctcgtcctgcccaaccagctcaccacgcgcctcacggcggacgagctcgccgccgtcgtgccgaagcggcgcggccagcccgtctcgcttcggctcgaggagggccgctccctcctcctcggaggactcgcgaggctcgacctcgtcgccggccgccccttcctcttcaccgcctatctgagcgacgcggtcaccctgcacccgaccgccaccgcaaaggccgccgaggtgcggcgcaagcacgcgggcggcgtcctcacgccgcccgcctctctcgaacgcctcgaggcgctcggcgagctcgaggcgcagcacgagctccgcgagcacgagctccgcgtcgaggggcgcggctggggcgaggcggcggtcgacgtcgtcttccccggcctgggctggatcgctgtcaccggctcgagcggctag

SEQ ID NO: 117 - DNA, 2133 bp, Phaeodactylum tricornutum
atgcgaacgaattttgctttgtcgacgcgctgctttgcttcttcatccgacaaccatgacgaagaggaacaacgagactctccgaaacaaagatccaaacgcagccaaactaatcggtccaagaaattcaaaattgctgaatcaatcgaccagagcaaaatagataagctagcacaagcattcgatgaactcgctcggaaggaaggcttcgactcgtcaacagcacgctttgccgacgatgtgacgttcgaggacaagtttgacgacgattcgtttctggacgatgacgatgataacaacaaagataaagtgggaaacttgcacctagatgcatccatgttcagtttaagtgactttatagataagagtgaggaagatggcggcaatccaaccgatcaagatgacgaggactaccttgattttggtgcagacattgacatgagtatagaagcaaggattgccgctgccaaacgggatatggatctcggtcgagtcagcgcccctcccgatatgagatcctcgcgcagggaggtaactgcagccgaccttcgcaaacttggatttcgaaccgaggcaaacccattcggcaacgacgaaactccacggaaggagcgcttccagttggtaacaaactccatgtcgtgctccgcctgtggatcggactttcaatgccacaacgaagatcggcccggatatctgcctcctgaaaagttcgctacgcaaacagcacttggaaaaatagaacagatgcaaaagttgcaggataaagcagaaaaagcggaatggacacctgaagatgagattgaatggttgattcagactcagggcaaaaaggatccgaacaaagaaatgcaggaggtgccccagatcgatgttgattctttggcaggggaaatgggccttgacctcgtagagctttccaaaaagatggttatttgcaagcgctgtcacggtctgcaaaactttggaaaagtgcaagattccctccgacctgggtggacgaaggagccactgttgtcgcaggagaaatttcgtgaattgttaaggccaatcaaggaaaagccggcagttatcgttgcattggtcgatctttttgatttttcggggtctgtgctccctgagcttgatgaaatcgctggtgaaaaccctgtaattcttgcggccaacaaggcggatcttcttccaagtgaaatgggacgcgtgcgagctgagagttgggttcgacgcgagctcgaataccttggagtcaagtcgttggccggtatgagaggagcagttcggcttgtcagctgcaagactggagctgggattaatgatttgctggagaaagcaagaggattagccgaggaaatcgacggcgacatatacgtcgtcggggctgcaaatgcaggaaaaagtacgcttttgaattttgttctaggtcaggacaaggtgaacagatcacccggaaaagcacgagcaggcaacaggaatgccttcaagggcgcggtgacgacaagtccactgccaggcacaacgcttaagttcatcaaagtcgatttaggcggcggtcgaagtctatatgacactcctggtcttctggtattaggcactgtgacacagttactgacccccgaagagctgaagatagttgttcccaaaaagccaattgaacctgtcaccctccggctctctaccggaaagtgcgttctagttggaggattggcccgcatcgagttaatcggcgactcaagaccctttatgttcacattttttgttgctaatgagatcaagctccaccctactgacatagagagagccgatgagttcgttctaaagcacgctggtggcatgttgactccaccgctagcacccggaccaaaacgtatggaagagattggagaatttgaagatcacatcgtggatatccagggtgctggctggaaagaagctgctgctgatatcagtcttaccggactaggatgggtggccgttacaggagcagggacagcgcaagtaaaaataagtgttccgaaaggtattggtgtatcggtgcggcctccgcttatgcctttcgatatctggaaagttgcatcgaagtataccggaagtcgagctgtaaattataacttttctctttcagttgatatctag

SEQ ID NO: 118 - DNA, 2077 bp, Arabidopsis thaliana
agaagtgacacgctctcaaacgaaatggtggttttgatttcaagtacagtgacgatttgcaatgttaaaccaaagcttgaagacggaaactttcgcgttagccggttgatacacagacccgaggttccatttttctcaggattgagtaatgagaagaagaagaaatgtgcagtttcggttatgtgtttagctgtgaagaaagaacaagttgttcaaagcgtggagagtgttaacgggacgatttttccgaagaaatcaaaaaatcttatcatgagcgaaggaagagatgaagatgaggactatgggaagattatttgtccaggttgtgggatttttatgcaggacaatgatccagatttacccggatattatcagaagagaaaggtcattgcgaataacttggaaggtgatgaacatgtggaaaatgatgagcttgctgggtttgaaatggttgatgatgatgctgatgaggaggaggaaggggaagatgatgaaatggatgatgagatcaagaatgcaatagaaggtagcaactctgaaagtgagagtgggtttgaatgggaatcagatgagtgggaagaaaagaaggaagtgaatgatgttgaattggatgagaagaagaaacgggtttccaaaacagagaggaagaagatagctagagaggaggcaaagaaagacaattatgatgatgtgactgtgtgtgctcgttgccattctctgaggaattatggccaggtgaagaatcaggctgcagagaatctcttacccgattttgatttcgataggttgatctcaactagactgatcaaaccgatgagtaactccagcactacagttgtagtcatggttgttgattgtgtagactttgatggttcgtttcccaaacgagctgccaagtctctgtttcaagtgcttcaaaaagctgaaaatgatcctaagggtagcaaaaacctcccaaaacttgtacttgttgcaacaaaagtagacttacttcctacacagatttcaccagctcggttagaccgatgggtgcgccaccgtgccaaggctggaggagcacctaagctaagtggggtttatatggttagtgctcgcaaagatattggtgttaagaatctgttagcttacattaaagagttggctggtccaagaggaaatgtgtgggttattggagctcagaacgcggggaaatctactttgattaatgccttatccaagaaagatggtgcaaaggtcacgaggctcacggaagctccagttcctggaacaactcttggaatattgaaaattggcggaatattgtctgcaaaggctaagatgtatgacactcccggccttttgcatccctaccttatgtccctgagattgaattcagaggagcggaaaatggtagagataaggaaggaagttcaacctcggagttacagagtcaaggcaggacagtctgttcacattggtggcctggtcaggctagacctcgtttctgcttcagttgaaacaatatacattacaatatgggcatcacatagtgtttcattgcatctaggaaaaacagagaatgccgaagaaatattcaagggccattccggtttacgccttcagccaccaattggagagaacagagcgtctgaattgggaacatgggaagagaaggagattcaggtgtcgggaaatagctgggacgtgaaaagcatagacatttcagtggctggtcttggctggttatccctgggcctcaaaggtgcagcaacactagcattgtggacttatcaggggattgatgtaaccttgagagaaccattggttattgaccgcgcaccatatcttgagcggcctggcttctggttgccaaaagccatcaccgaagtgcttggaacacattctagtaagcttgttgatgctcgtaggaggaagaagcaacaagacagcacagattttctctctgatagtgttgcttagtataacctgtatcgacttattattagctttcatcagtgtagtcattttggaaagtttatattggtttatgtattttaaaacaattttaaatccacatcgactatttatttatttcaat

SEQ ID NO: 119 - DNA, 1986 bp, Medicago truncatula
atggagaatggagctctccggcaagtcgcggccggaaatcgcggaggaatcgtatccgcgagtgatacaatgagacgaaaatggagaaagacgaacctgaacgagtttgttggatattccgttcgaagcaaagccatggctatcttgttctctacaattgcacttccctccacaaacgtcacttccaaactatccatcttaaacaacacttcacattctcacgcacttcgccatttctcaggtaatactactaaacgctttcataaagcttcctcctttattgcttttgctgtgaagaacaaccccaccataagaaaaaccactccaagaagagatagtagaaacccacttttaagtgaaggtagagatgaagatgaagctcttggacccatttgccctggttgtggaattttcatgcaagataatgatccaaatctccctggtttttaccaacaaaaagaggtaaaaattgaaacattttctgaggaggattatgaattagatgatgaagaggatgatggtgaagaagaggataatgggtcaattgatgatgagtctgattgggattctgaggaattggaagctatgttacttggtgaagaaaatgatgataaggttgatttggatgggtttacacatgcaggtgttgggtatggtaatgttactgaggaggttttggagagggctaagaagaagaaggtttcaaaggctgagaagaagagaatggctagggaagctgagaaggtgaaggaggaggttactgtttgtgctaggtgtcattccttgagaaattatgggcaggtgaagaattatatggcggagaatttgataccggattttgatttcgataggttgattactactaggttaatgaatcctgctggtagtggtagttctactgttgttgttatggttgtggattgtgttgattttgatggttctttcccgagaacagctgtgaagtcgttgtttaaggcattggaaggtatgcaggagaatacaaagaagggtaagaaactgccaaagcttgttcttgtggctacaaaggttgatctccttccgtcgcaggtttctccgacgaggttggatagatgggttcggcaccgtgcaagtgctggaggagcgcctaaattaagcgcggtttatttggtcagttctcgaaaggatttaggtgtgaggaatgtgttgtcgtttgtaaaggatttggctggtcctcgtgggaatgtttgggttattggggctcaaaatgctgggaagtctactctgatcaatgcatttgcgaagaaagaaggagccaaagttaccaagctcacggaagctccagttcctgggacgacacttgggatcttgaggattgcaggaattttgtcagctaaggctaagatgtttgatactccagggctcttgcatccatatttattgtcgatgagattgaatcgggaggaacaaaagatggctggacaagccatacatgttggtggcttggcaagacttgacctaattgaagcctctgttcaaacaatgtatgtcactgtttgggcatcaccaaatgtttctctacacatgggaaaaatagaaaatgctaatgagatttggaataatcatgttggcgtcagactgcagcctcccatcggtaatgaccgcgcagctgaactaggtacatggaaagaaagggaagtaaaagtatctggatctagttgggatgtcaactgcatggacgtatcaatagctggcttaggttggttttctttgggtatccaaggtgaagcaaccatgaaattatggaccaatgatggaattgaaataactttgagagaaccattggtacttgaccgggccccgtcccttgaaaaaccaggtttttggttaccaaaggctatatctgaagttattggcaaccaaactaaacttgaagctcaaagaaggaaaaaacttgaagatgaagatacagaatacatgggagcaagtatagagatatctgcatga

SEQ ID NO: 120 - DNA, 2046 bp, Oryza sativa
atggctaaacccctcctcctccccgctaccgtcgcggcggcagcagcagctcgcctcccctcccgcctcgccgtcggcgcggccccgccattccgcgtcctccccttcttcctctgcccgccgcctcagagccgcagcctctccttctcccccgtctccgccgtgtccacggccggcaagcgcggcaggtcgccgccgccgccgccgagcccggtcatcagcgagggcagggatgacgaggacgccgccgtcggccgccccgtctgccccggctgcggcgtgttcatgcaggacgccgaccccaacctgcccggcttcttcaagaacccctcccgcctctccgacgacgagatgggggaagacgggtcgcctcctcttgccgccgagcctgatggatttcttggagacgacgaggaggacggtgcgccgtcggaatctgatcttgccgccgaattggacggtctggacagcgatttggatgaatttcttgaagaagaggatgagaatggagaggatggggcggagatgaaggctgacatagatgccaagatcgatggcttctcgagcgactgggactcggattgggatgaggagatggaagacgaggaggagaaatggaggaaagaactggatggtttcaccccaccgggagttgggtatggaaagatcactgaggagacactcgagagatggaagaaggagaagctgtccaagtccgagaggaaacgccgggcacgggaagccaagaaggccgaggccgaggaggacgccgccgtggtctgtgcccggtgccactcactgaggaattatgggcatgtgaagaatgacaaggctgagaatttgatcccggacttcgatttcgatcggttcatatcgtcccgtctgatgaaacgttcagctggcacaccggttatcgtcatggtagcggattgcgcggactttgacggctcattcccaaagagggctgccaagtcgctgttcaaggcgctcgaggggcggggaacttctaagttgagtgaaacgccaaggcttgttcttgttggaacgaaggtggatttgctgccatggcagcaaatgggagtgaggctggagaagtgggtgagaggccgagctaaggctttcggagcaccaaagctggatgctgttttcttgatcagtgttcataaggatttgtctgtcagaaacttgatctcatatgtcaaggaactagctgggccccgtagcaatgtttgggtgattggtgcacagaatgctgggaaatccactctaattaatgcatttgcaaagaaacaaggtgtcaaaatcacaaggttgactgaggctgctgtgccaggaactacattaggaatcttgagaataacaggtgttttgccagcaaaggctaaaatgtatgacactcctggcttattgcatccatatataatgtcaatgagattaaacagtgaggaacgcaagatggttgaaattcggaaagaactccggccaaggtgcttcagggtgaaggcaggacaatctgtacatattggaggtttaacacgacttgatgtgttaaaagcttcagtccaaactatctacataactgtttgggcatctcctagtgtgtccctccatctggggaagactgaaaatgctgaagaactgcgggacaaacattttggcatcagacttcagccaccgatcaggccagagcgagttgccgaattaggtcactggacggaaagacagattgatgtgtcgggggtcagttgggatgtgaacagtatggatattgctatttcggggttaggctggtattccttgggcctgaaaggaaatgccacagttgcggtgtggactttcgatggcattgatgtgacacggcgtgatgcgatgattcttcaccgagctcagttcctcgaaaggcctggattttggctacccatcgccatcgccaatgctataggtgaggagaccaggaagaagaatgagagaaggaagaaggctgagcaaagagatgatctccttttggaagaaagcgccgaggatgatgtggaggtgctcatatag

SEQ ID NO: 121 - DNA, 2111 bp, Populus trichocarpa
ttggctgctttagagctttgctcggaggaaatggcagttttgttgtcaacagtagcagtgaccaagccaagattgaagctttttaacaacaatggcattacacaagaaatatcttcaatcccaattaatattttcactggattgagtttagagaacaagaaacacaagaagagattatgtttggtaaattttgttgctaagaatcaaacaagcattgaaacaaaacaaagaggtcatgctaaaataggacctagaagaggaggtaaagacttagttttgagtgaaggaagagaagaagatgagaattacggacctatttgtcctggttgtggggtcttcatgcaagataaagacccaaaccttcctggatattataagaaaagagaagttattgttgaaagaaatgaagtagtggaagaggggggtgaggaggagtatgttgtagatgaatttgaagatggttttgaaggtgatgaagagaagttagaggatgccgttgagggtaaacttgagaaaagtgatggaaaggaaggtaatttggaaacatgggccggttttgatttggattctgacgaatttgaaccctttttagaagatgaagagggtgatgattctgacttggatggttttattccagctggggttggatatggtaacattacagaggagataattgagaaacaaaggaggaaaaaggagcagaaaaaggtgtccaaagcagagaggaagaggttggctagggagtctaagaaggaaaaggatgaggttacagtgtgtgctcgatgtcattctttgaggaattatgggcaggtcaagaaccaaacagctgaaaatttgatacctgatttcgattttgataggttgatcacaactaggttgatgaaacctagtggcagtggtaatgttactgttgttgttatggttgttgattgtgttgactttgatggctcatttcctaagcgggcagcacagtccttgttcaaggcattggaaggagtcaaggatgaccctagaacaagtaaaaagttgcctaagcttgttctcgtgggtacaaaggttgatctcctcccttctcaaatttcacctaccagattagatagatgggttaggcaccgtgcgagggctgcaggggcacctaagcttagtggggtttacttagttagttcttgtaaggatgtgggtgtgagaaacttgttatcattcattaaggaattggctggtcctcgagggaatgtgtgggttattggggctcagaatgcaggcaagtctactctaatcaatgcattagccaagaaaggaggtgctaaagtcacaaagcttacagaagctccagttcctgggacgacagttggaattttgagaattggagggattctatcagctaaggcaaagatgtatgacactccaggtcttctacatccatatctaatgtccatgagattgaatagggatgagcagaaaatggttgaaatacgaaaggagctacaacctcgaacatatagagtgaaggcaggacagacaatacatgttggtggcttgttgcgactggatctcaatcaagcatctgtgcaaacaatctatgtcacagtttgggcatcgccaaatgtttctctgcacattgggaagatggaaaatgctgatgagttttggaagaaccatattggtgttcgtttgcagccaccaactggcgaagatcgagcttctgagttaggaaaatgggaagagagggaaatcaaagtaagtggaacaagctgggatgccaatagcattgatatttctatagctggtttaggctggttttctgttggcctcaaaggggaggcaaccctgactttgtggacatatgatggcattgagatcactttgagagaacctttggtccttgaccgagcaccattccttgagagacctggatttttgttgcctaaggcaatatccgatgctattggcaaccaaaccaaactagaagccaaaattaggaaaaagcttcaagaatcgagtctggattttctatccgaggtttctacttaaacgggaaggagatgatcaatgtccctttcaaagttgccttctcaagtaggaaagaagatcagtttgtttctcttctcaaa

SEQ ID NO: 122 - DNA, 2380 bp, Sorghum bicolor
atggctgctaaacccctcctcccaatcgccgcggcggcggctcgccttcccttccgcctcctctccccgtcagctccacctccccgcggcctccccttgctgtccccgccattcctgccccaaaggcgcagcctttccgcctctgccgtacccaccggcaggcgtagcaggccgccggccccggtcatcagcgagggcagggatgacgaggaggccgccgtaggccggcctgtatgtcctggatgcggggtcttcatgcaggatgcggatcctaacctccctggcttcttcaagaacccatcccgcagctcccaggacgagacgggaggaggtggagaagtgctcctggccgccgccgatacggatgcgtttcttgaagatgagaaggagggggtggtggcggaggacgcgttggatgctgaattggagggcctggacagcgatatcgatgagttccttgaagatttcgaggatggggacgaagaggatgatggctcaccggtgaaaggtgccactgatatcgatgcttttgccagcgattgggactctgattgggaggagatggaagaagacgaggatgagaaatggaggaaagaactggacggtttcaccccgccgggagtcggctatgggaacatcactgaggagacgatccagaggctgaagaaagagaagctgtccaagtccgagaggaagcgccaagcgagggaggccaagagggctgaggctgaggaggactcggccctcgtctgtagccggtgccactcgctgaggaattatgggcttgtgaagaatgacaaggctgagaacctgatcccagactttgattttgatcggttcatttcgtctcgggtcatgaagcggtcggctggcacaccggtcatagtcatggtggtggactgtgcagactttgatgggtcgtttccgaagcgagctgccaagtcgttgttcgaggcacttgaaggaaggaggaattcaaaggtgagcgaaacaccgaggcttgttcttgttggtacaaaggtggatttgcttccatggcaacaaatgggtgtccggttggataggtgggttcgtggccgtgctaaggcttttggagcacccaagctagatgctgtgttcttgatcagcgtccacagagatttggctgttagaaacctaatttcgtacatcaaggagtcagcaggacctcggagcaacgtttgggtgattggtgcgcagaatgctgggaaatctacgctgatcaatgcttttgcaaagaaacagggtgttaagatcacaagattgactgaagctgctgtcccgggaacaacactcggcatattgagggtaacaggtgttttacctgcaaaggcaaagatgtacgacactcctggcctgttgcatccttacataatggcaatgagattaaacaatgaggaaaggaagatggttgaaataaggaaagaattgcggccacgatccttcagggtgaaagtaggacaatctgtccatattggaggcttaacacggctggatgtgctaaaatcatcagctcaaactatctatgttactgtttgggcatcttccaatgttcccctccatcttggaaagactgaaaatgctgatgaattgcgagagaaacattttggtatcagacttcagcctccaattggcccagagcgagtcaatgaattgggtcactggacagaaagacatattgaggtttctggggcaagctgggacgtcaacagtatggacattgctgtttctggccttggatggtactccttgggccttaaaggcactgccactgtttccttgtggacatttgagggcattggtgtgacagaacgagatgcgatgattctgcatcgagcccagtttctcgaaaggcctggattttggttacctattgccatcgctaatgctctaggtgaggagacaagaaagaagaacgagaagagaaaggctgagcaaagaagaagagaggaagaagagctccttttggaagaaattgtttagtgatgattctgtagcccacaaagtcaggattcccgtttctcctgtcgacatgggcttttgtctgtcccagttcttgatgtcttttgacataggctgcatcctcttattttttttctttgctcatagatatatatctcaatttttctgtctatttgttttccatgtgttctttgcaaccagttttgttaatgctgcttgtatcttacagattcagttcttgcaacttgaggaagattgatatccttgattgcctattcagttatgtgcaaaaacgagacttttagacatgcagagcagccaatttattatgtactactttttttaactgaaaacaatttattatgtactatttttttgaactgaaaacaatttattatgt

SEQ ID NO: 123 - DNA, 1794 bp, Vitis vinifera
atgagaaaaaatagcaggaagaacgacatcaaattttcatttgttgcattatcagtgaagagcaaatacacaattcaagaaacacagaaaaataattggaaaaacccaagaaaagttggtggaaacccaattttgagtgaaggaaaagatgaggatgagagctatggccaaatttgtcctggttgtggagtttatatgcaagatgaagacccaaatcttcctggttattatcaaaaaagaaagttgactctaacagaaatgccagagggtcaggaggatatggagggaagtgatggggaggaaagcaatttgggaacggaagatggcaatgagtttgattgggattctgatgagtgggaatcggagttggagggtgaagatgacgatctggacttggatggttttgctccagcaggtgttggatatggtaatattacagaggagactattaacaaaagaaaaaagaagagggtctcaaagtctgagaagaagagaatggctagggaggctgagaaagagagggaggaggttacagtttgtgcaaggtgccattctttgaggaattacgggcaagtgaagaaccagatggccgaaaacttaatacccgattttgattttgataggttgattgctacccggttgatgaaacccactgggactgctgatgccacagttgtagttatggtggttgattgtgttgactttgatggttcatttccaaaacgggcagcaaagtctttgttcaaggcattggaggggagcagagttggggcaaaggttagtagaaaattgcctaaacttgttcttgtcgccacaaaagttgatctcctcccatcacaaatttcaccaactagattagatagatgggtacggaatcgggccaaggctggaggtgcacctaagctaagtggggtttatttggttagtgcccggaaggatttgggtgtcagaaatttgttgtcttttatcaaggaattggctggccctcgtggaaatgtgtgggttattgggtctcagaatgcaggtaagtctactcttatcaacacatttgcaaagagagagggtgtgaaactcacaaagcttacagaagctgctgttcctgggacaactcttggaattttgagaattggagggattttgtcagccaaggcgaagatgtatgacaccccagggcttctccatccatatttaatgtccatgagattgaatagggatgagcagaaaatggctgagatacggaaggagctacagcctcggacttataggatgaaggctgggcaggctgttcatgttggtggcttaatgagattagaccttaatcaggcttcagtggaaacaatttatgtcacaatttgggcatcaccaaatgtttctctacacatggggaagatagaaaatgctgatgaaatctggagaaagcatgttggagttaggttgcagcctcctgtcagagtggatcgagtttcagaaataggaaaatgggaagagcaagaaatcaaagtgtctggagcaagctgggatgtgaacagcatagatattgcagtagctggcttgggttggttctcgttgggtctcaaaggtgaagcaacattggcattgtggacatatgatggcattgaggtaattctacgtgaacctttggttcttgatcgagcaccattccttgagagacctgggttttggctaccaaaggctatatctgatgccattggcaatcaatctaaacttgaagctgaagcaaggaaaagggatcaagaggagagtacaaaatccctttcagagatgtctacttga

SEQ ID NO: 124 - DNA, 2373 bp, Zea mays
ttttttttcataataaattgttttcaattcaaaaaatagtacataataaattggctgctctgcatgtctaaacagtcccattttgcacacagctgaataggtaatctaggatatcaattttcctcaagttgcaggaattgaatctgtaagatgcaagcagaactgaaaaaactggttgtaaaacacatggaaaacaaatacacagaaaatttgagatatacataagcaaagaaaaaaaatcacaggatgcagcctatgtcaaaagacatcaagaactaggataaacaaaagcccgtgttgacaagagaaacagaatcctaactttgtgggctgggctacagaatcatcactaaaccatttcttccaaaaggagctcttcttcctctcttcttctttgctcagcctttctcttttcgttcttctttcttgtctcctcacctatagcattagcaatggcaataggtaaccaaaatccaggcctttcgagaaactgggctcggtgcagaatcattgcatcacgttctgtcacaccaatgccctcaaatgtccataaggaaacagtggcagtgcctttaaggcccaaggagtaccatccaaggccagaaacagcaatgtccatactgttgacatcccagcttgccccagacacctcaatagatcttcctgtccagtgacccaattcatcgactcgctctgggccaattggtggctgaagtctgatgccaaaatgtttgtctcgcaattcatcagaattttcagtctttccaagatggaggggaacattggaagatgcccaaacagttatatagatagtttgcactgatgattttagcacatccagccgtgccaagcctccaatatgtacggattgtcctactttcaccctgaaggatcgtggccgcatttctttccttatttcaaccatcttccgttcctcattatttaatctcattgccattatgtaaggatgcaacaggccaggagtgtcatacatctttgcctttgcaggtaaaacacctgttaccctcaatatgcctaatgttgttcccgggacagcagcttcagtcaatcttgtgatcttaacaccctgtttctttgcaaaagcattgatcagcgtagatttcccagcattctgtgcaccaatcacccaaacattgctacgaggtcctgctgactccttgatgtatgtaattaggtttctaacagccaaatctctgtggacgctgatcaagaacacaccatctagcttgggtgctcccaaagccttagcacggccacggacccacttatccaaccgcactcccatttgctgccatggaagcaaatccacctttgtaccaacaagaacaagtctcggcgtttcactcgcctttgaatttctccttccttcaagtgcctcgaacaatgacttggcagctcgcttaggaaacgacccatcgaagtctgcgcagtccaccaccatgacgatgaccggggtaccagctgaccgcttcatgagccgagacgagatgaaccgatcgaaatcaaagtccgggatcaggttctcagccttgtcattcttcacaagcccatagttcctcagcgagtggcaccggctacagacgagggctgaatcctcctcggcctcagcccttttggcctccctcgcctggcgcttcctctgggacttggacagcttctctttcttcatcctctcgatcgtttcctcagtgatgttcccgtacccgacacccggcagggtgaaaccatccagttctttcctccatttctcatcctcgtcttcttccatctcctcccaatcagagtcccaatcgctggcgaaagcatcggtatcagtggcgcttttcaccggtaaaccgtcatcttcgtcccccttatcgaattcttcaaggaactcatcgatatcgctgtccagaccctccagttcagcatccgacgcgtcatccgccaccctccgatcatcattatcatcttcttcttcaagaaacgcatccgtatcggcggccaggagcacttctccacttcctcccgtctcgtcctgggagctgcgggaggggttcttgaagaagccagggaggttgggatcctcatcctgcataaagaccccgcatccaggacatacaggccggccgacggcggcgtcctcgtcatccctgccctcgctgatgaccggggccggaggactgctacgcctgccggcgggtacggtggaggcggaaaggctgcgtctttggagcaggaatggctgggggaagaaagggaggaggcggggaggtggagctgccgagcagaggaggcgaaagggaaggcgagccaccgccgcggcagggattgagaggaagggtttagtagccattctggagctgcagcggc

SEQ ID NO: 125 - DNA, 1914 bp, Arabidopsis thaliana
attacccgtcggagtgaaatgctttcgaaagcagcaagagagctttcatcatcaaagcttaaacctttattcgctcttcatctctcttccttcaaatcttccatacccactaaaccaaacccttctcctccttcatatctcaatccccaccacttcaacaatatctcaaaaccgccatttttgcgtttctactcttcttcttcgtcctctaatctccttccgctaaacagagatgggaattacaacgatacaacttcaatcacaatctccgtttgcccaggttgtggagttcatatgcaaaactcaaacccaaaacatccaggtttcttcatcaaaccatcaacagagaaacagaggaacgatttgaatcttcgtgatctcacacccatctctcaagagcctgaatttatagattcaatcaaacgagggtttatcattgaaccaatcagtagttctgacttaaaccctagagatgatgaaccatcagattcaagaccattggtttgtgctaggtgtcattcacttagacattacgggagagtgaaagatccaacggttgagaatcttcttcctgattttgattttgatcatactgttggtaggagactaggttcagcttctggtgctagaactgttgtgttgatggttgttgatgcttcagatttcgatggttcttttcctaagagggtagctaagcttgtgtcgagaactattgatgagaataatatggcttggaaagaagggaagtctggtaatgtacctagagttgttgttgttgtgactaagattgatttgttaccgagttcgttgtctcctaataggtttgagcaatgggttagattaagagctcgtgaaggtggtttaagtaagattactaagttgcattttgttagtcctgttaagaattgggggattaaggatttggttgaagatgtggctgctatggctgggaagagaggtcatgtttgggctgttggatcgcagaatgccggaaaaagtacgttgattaatgctgttgggaaggttgttggtgggaaagtttggcatttgacggaagctcctgtgccgggaactacgttggggataattaggattgaaggtgttttgccttttgaggctaagttgtttgatactccggggctgttgaatccgcatcagatcactacgaggcttacgagagaggagcagagacttgttcatattagcaaggagcttaaaccaaggacttataggatcaaggaaggttatacggttcacattggtgggctaatgagacttgacattgatgaagcatctgttgattctctatatgtgacagtttgggcgtctccttatgttccacttcacatggggaagaaggagaatgcttacaaaacactcgaggaccatttcggttgtcgattgcagccgccgattggagagaagcgggttgaagagttggggaaatgggttagaaaggaattccgagtgagtggaaccagttgggacacaagttcagtagatatagctgtttcaggtctcggttggtttgcgttaggactaaaaggagacgcgattttaggtgtatggactcacgaggggattgatgtcttctgccgtgactcattgctcccgcaacgagcacacacttttgaagactctggattcactgtctccaagatcgttgccaaagctgatagaaattttaaccaaattcacaaggaggaaacacagaagaaacgaaaacccaacaagtctttttcagattctgtatctgacagagacaatagccgcgaggtgtcacagccttcagatatcttaccaacaatgtgactcttataagttagttaccttttccttggtttgttgaaattacattgaaagcttattttcttcaaagcttatttcattcattgaaaggttcattacatagac

SEQ ID NO: 126 - DNA, 1818 bp, Glycine max
atgcttgtagctcgaagcctctccccttcaaagcttaaaccactcttttatctatcgatcctttgtgaatgccaaaatcatttccactcaagcttaataccatactcaaaacctcatctccaaaacttcccaaaattttatcctcagccatcaactaatctgtttagatttttctcttcacagcctgcagattcaactgagaaacagaatttgcccctctctcgtgaaggtaattacgatgaagtcaattcccaatctcttcatgtttgccctggctgtggggtttatatgcaagattccaaccctaagcaccctggttattttatcaaaccctctgagaaggacttgagttatagattgtataacaatcttgaacccgttgctcaagagcctgagttctctaacactgttaaaaggggaattgttattgaaccagaaaagcttgatgatgatgatgcaaacttgattaggaaaccagagaagccagtggtgtgtgcgcgctgtcattcgttgaggcactatgggaaggtgaaggatcctaccgtggaaaacttgctacctgattttgactttgatcacacggtgggtaggaagttagcatcagctagtgggacccggtctgtggtgctgatggttgtggatgtagtggattttgatgggtcttttccaaggaaggttgcaaagttggtttctaagacaattgaggatcattctgctgcatggaagcagggtaagtcagggaatgtgcctagagtggtgcttgtggtgacgaagattgacttgttgcctagttcattgtcaccaacaaggttggagcattggattaggcagagagcaagagagggtggaattaataaggtttctagtttgcacatggtgagtgcattgagggattgggggctgaagaatcttgtggataatatagttgatttggctggacctagagggaatgtgtgggctgttggagcacagaatgcaggaaagagtactttgataaactctatagggaaatatgctggagggaagattacacatctgactgaagcacctgtgccagggactacactaggcattgttagagtggagggtgttttttcaagtcaagcaaaactgtttgatacacccggccttcttcatccttaccagattacaacgaggttgatgagggaagagcaaaagcttgttcatgtgggcaaggaattgaaacctaggacttacagaattaaggctggtcattcaattcacatagctggtctagtgagattagatattgaagaaactcccttggattctatttacgtcacagtgtgggcatctccttatcttccactacatatgggtaaaatagaaaatgcatgtaaaatgttccaagatcattttgggtgccagttacagccaccaattggagaaaaacgagtacaagaactggggaattgggtgagaagggaattccatgtcagtgggaacagttgggagtcaagttcagtagacattgctgttgctggcctcggttggtttgcctttggacttaaaggagatgcagtgttaggagtttggacttatgaaggagttgatgctgttcttcgcaatgctttaataccctatagatcaaatacttttgaaattgcagggtttactgtgtccaagattgtatcccagtctgaccaagctttaaacaagtcaaagcaacgaaatgacaaaaaggcaaagggaattgactcaaaagcgccaaccagttttaaagaaaagttgagaaacgtaagaggtccttacatagcattgccagccatgagtgagagagagagacagaggagataa

SEQ ID NO: 127 - DNA, 2315 bp, Oryza sativa
gtcgaacagctggtgcgcgtcctctcatgtcgagcacctgaccgccggtcacgtagcggcggcggcggcggcggcgcggcaagatgctctcccgcgcccggcgcctccaccccaccctccagcgaatcctccggccagtcccccctcccgcccatcctcctcctcctccttccccacctcaccgccccgtcttctcccaaacccctaaacccttcttccccttcctccgccgccacctctcgaccaaaccgccgccgccgcaggcgccgccagagaagtcgctggctccggcgaaggtgagctccgatccacctgccgtcagcgcgaatggcctctgcccgggatgcggcatcgcgatgcagtcctcggacccgtcccttccgggcttcttctccctcccttcgccaaaatcccccgactaccgcgcgcgcctcgcccccgtcaccgccgacgacacccgcatctcggcctccctgaagtccggccacctccgggagggcgaggcggcggcggcggcgtcgtcgtcgtcggcggcggtgggggtgggggtggaggtggagaaggaggggaagaaggagaacaaggtggtcgtgtgcgcgcgctgccactcgctgcgccactacggcgtcgtcaagcggcccgaggccgagccgctgctcccggacttcgacttcgtcgccgccgtggggccgcgcctcgcgtcgccctcgggcgccaggtcgctcgtgctgctcctcgccgacgcgtcggacttcgacggctccttcccgcgcgccgtggcgcgcctcgtcgcggccgcgggggaggcccacgggtccgactggaagcacggcgcgccggcgaacctcccgcgcgcgctgctcgtggtcaccaagctcgacctgctccccacgccgtccctgtcccccgacgacgtccacgcgtgggcgcactcccgcgcgcgcgccggcgccggcggcgacctgcgcctcgccggggtgcacctcgtcagcgcggcgcgcgggtggggcgtgcgcgacctgctcgaccacgttcgccagctcgctgggtcgcgtggcaatgtgtgggcagtgggtgcgaggaatgttggcaagtctacactgctcaatgccattgcccggtgctccgggattgaaggcggaccgaccttgacggaggcgccggtgccaggaacgacccttgatgtgatccaggttgatggcgttcttggatcgcaggcgaagctgttcgacacaccgggcttgcttcatggtcaccagctgacatcgaggctgactcgcgaggagcagaagctggttcgagtgagcaaggagatgcggcccaggacatacagattaaagccagggcagtctgtacatattggagggctggtgcgcctggacatcgaagagttaactgtaggatcagtttatgtaacggtatgggcatcaccacttgtcccacttcacatggggaagacggaaaatgctgccgctatggtaaaagaccactttggtttgcaactacagcctcctattggccaacaacgggtaaacgaactaggtaaatgggtgaggaagcagttcaaagtttctgggaacagttgggatgtgaattccaaggatattgcaattgctggtcttggctggtttggaataggtctgaaaggagaagcggtattaggactatggacatatgatggtgtcgatgtcgtctccagaaactcccttgtccatgagagggcaacaatatttgaggaagccgggttcacagtttcgaagattgtctctcaggcggatagcatggcaaataggctaaagaaccctaagaaaataaacaagaagaaggataacaaagccaattcatctccctccacagatccagaatcttcaaatccagttgaggctgtagatgcttaaatgatttctattcctttctaggacaggagttcccgaaggtgaattaagttctatgatgttggcatttggtcagttgaggattgatatacagagccataggttgcacaatttatacttgttcagacttagatagcatgctgctctccgcacaagtctttttttttttccctgggatcatggattttatgtagtcttgttgtgggcttgtaacattaacctatggcttttatgtactcaatgaacttctaccactatggctttggagtttggaccataagtacaattttgatagtcaacttgatgaggagtcagtactgaagaatactcgttgtaatgctgttatggctgaacttctgaaaccggcatctcacagctttgttatgcctgctttgaacacaggaattttacattgatttt

SEQ ID NO: 128 - DNA, 2433 bp, Sorghum bicolor
gcaagcctgtcctctcgagtcgaacacctgaaacccaccgcccgccgatcaccaagcggcggcggcggcggcggcacagcaagatgctatcccgcgcgcggcgcctccatcccgccgtccgccgattcctcctcccaaacacgcctgcaccctcccgtcctgctccgctcccacctcaacacagcgcttccgcccaaacctctaaaaccttctcgatcctcttccgccgccacctctgctcctcaccacccgcgccgccgccgtcgacatcaccgcctccagcggtggtatcttctgacctcccggccgttcgcgtcaatgaagtctgcccgggatgcggaatctccatgcaatcctccgaccccgcgctcccgggcttcttcttgctcccctccgcaaaatcccccgactaccgcgcgcgcctcgcgcccgtcaccaccgacgacactcgaatctccgcctccctcaagtccggtcaccttagggaggacttagagccgtcgggaagcgacaagccggccgcggcggcggctgagatggctgattccaagggagagggaaaggtgttggtatgcgcgcgatgccactccctgcgccactacggccgcgtcaagcatccggacgccgagcgcctcctcccggacttcgacttcgtcgccgccgtcggcccgcgcctcgcgtcgccttccggggccaggtcgctcgtgctgctcctggcggacgcctctgacttcgacggctcgttcccgcgcgccgtcgcgcggttggtggccgcagccggcgaggcccacagcgcggactggaagcacggggccccggccaacctcccacgcgcgctgctcgtggtcaccaagctcgacctgctccccacgccgtcgctgtcccccgacgatgtgcacgcgtgggcgcactcccgcgctcgtgccggtgcaggttcagaccttcggctcgctggggtgcacttggttagcgccgcgcgcggatggggcgtccgcgacctgctcgaacatgtgcgcgagctcgccgggacgcgcggcaatgtctgggccgtgggtgcgcgaaacgttggtaagtcgacgctgctcaatgcgatcgccagatgctctggcatagccgggcgacccaccttgacggaggcgccagttccgggaacgacccttgatgtgattaagctagatggcgttcttggtgctcaagcaaagctgtttgacactcctggacttctccatgggcatcagttgacatctagactgacgagcgaggagatgaagttggttcaagtgagaaaggagatgagtcccagaacttacagaataaagacaggacagtccatacatatcggtggactggtgcgcctggacgttgaagagttaactgtaggatcgatctatgttacagtttgggcagcaccacttgtcccacttcacatgggaaagacagaaaacgcagcagcattgatgaaagaacactttggcttacaactacagcctcccataggccaggagcaggtaaaggagcttggtaaatgggtgaggaaacaattcaaagtttccgggaacagttgggatatgaactctaaggatatagccattgctggtattggctggtttggaattgggctgaaaggagaggcggtgttaggattatggacatatgatggtgttgatgtcatctccaggagctccttagtccatgagagggcttcaatttttgaggaagctggtttcacagtttcacagattgtttctaaggcagatagcatgaccaataagctgaagagcaccaagaagccgaacaagaagaaagagagaacgaaaagtgcttctcccctcacaaagccggaagcttcagaacctgcttccaacatagatgcttgagtgttttcattcagtcctgtgactggagcatcactttggtggtcatgttcgagcccacaatgttctgcattgaccctagaaactgttaattgaattcagaaacagaagctgaatgtacaatgtattttctccagaagaaggaacctgcactcatcgaaggattttctatttttcatagagctccagagtttgaacctttgctaatttgctgagctggagagtcagttaggaaatactctgagatgtcagtcagtcagttatggaagacctatctggagagttagttaattaggaaatactctgtaagatgttttgatgttaacttataaaatctaacatgagtacttgtgttccagcattaaaagggaggtgtagagatattctagtttacatttgatcttatcattcagttataattgtctcttgtaaagttgtagctctgaactttgatacaggttccacagatgtttgttctgtttccttcaattgcctgcattatagattccgtgggcaactgggcatctttctcagaccacagcttgtctagtgatgaaaa

SEQ ID NO: 129 - DNA, 3579 bp, Vitis vinifera
atgatagtgaggaaattctctgcttcaaagctcaagcaccttcttcctctttctgtcttcacacactcatccacaaatctctcattatcacctttttcttcaaaccccatttctaaaaccctaaaccctaatccccactttttattttcacactcaaagctcaggcctttctcttcttcccagtccaaaccctctttgcccttcaccagagatgggaatttcgatgaaaccctatcccaatccctattcatctgccccggttgtggcgtccaaatgcaagattcagacccggttcaacctgggtacttcatcaaaccctcacaaaaggatccaaattatcgctcccggatcgatcgcagacccgttgcggaagagccggagatttctgattcgctgaaaaagggattgcttaagcccgttgtctgtgctcgttgccattcgttgaggcattatgggaaggtgaaggacccaacggtggagaatttgttgccggagtttgattttgatcacactgttgggaggagattggtttcaacctctggaactcggtctgtggttctaatggtggttgatgcttcggattttgatgggtccttcccaaaaagggtggcgaagatggtttctaccaccattgatgagaattatacagcatggaagatgggcaagtctgggaatgtgcctagagtagtccttgtggtgacaaagattgatttattgccttcatctttatcgccaacccggtttgagcattgggttagacagagagcaagagagggaggagcaaataagctaacgagtgtgcatcttgtgagctcagtgagggattggggattgaagaatcttgttgatgatattgttcaattagtcgggcggagagggaatgtgtgggcaattggggcgcaaaatgcagggaagagtacactgatcaattcgatagggaagcatgcaggagggaaacttacacatttgactgaagctccggtgcccggaaccacattgggcattgtcagggttgagggtgtacttactggggcggcaaagttgtttgatacacctggccttttgaatccccatcagataacaacaaggttgaccggggaagagcagaagcttgttcatgttagcaaggagttgaaaccgaggacatacagaatcaaggcaggccattcagttcatatcgccgggcttgcgaggctggatgtagaagaactgtcagtagacacagtttatatcacagtatgggcatctccttatcttccactgcacatggggaagacagaaaatgcatgcacaatggtagaagaccatttcggtcgtcagttacagccaccaattggagagaggcgagtcaaggagcttggaaaatgggagagaaaagaatttcgtgtttctgggaccagttgggattcgagctctgttgatgttgctgttgctggccttggatggtttgcagttggcctcaagggagaggcggttttaggcgtttggacttatgatggagttgaccttatccttcgcaactctctgcttccttatagatcacaaaattttgaagttgctgggtttacagtttcaaaaatcgtctccaaagccgaccaagcttcaaacaagtcagggcaaagccaaaagagaagaaaatcaagtgacccaaaagccgcagcccattgtttgccatcaccattaacagctaatgcaggctgaaatcagaagaagaaacacaccttccaacgccaactagatgaaatcaaaaggataggatttcaagccaaaaaaagccccgggggggatgagagaacaagctacaatctcagctacaggtaatgaaccttccctgtattttttttactagaaaggggaagagatgattgatattttgaagccttttctgctaactatgcgaggctgggatccttgtgtacgtactgggtaagccatggaagtagggaagatgagatacagaaaggcaagttttgtcagtttatagaaaaggcgtttgtgactttcaagctttctcaaattttggaaattccctccttggtgactctttgctagatttttcattgattcttttgagtgtttctcattgttgacttggctcttgcctggatttcttttttcctccataagttttgggttatgaggctgatattaataggaattttgagaagaaaaataaaaaaataatatatattttaaaatatggtaattcaaactacactttgaaggtggaaaagacacttttaagtctgattggtaattattttggagaataattttttgatctcgaaaataaaatttttttttgcttttcttgggaaaacggaaactaaataaagccttaatggggaagttatttttaagaaaacaacttctaaatttagaaatagttgtaaattatcatgattgattgaataaatatttttggaaaaatgtttttatttttattagaaagatcctaatcccacacttggtggttggatacttgatttgatgggacaagactccatttatttttccagtttaatatgctgctttcaagccacgtgcttttttaggtttccattagggtagctgctgcagcctgctgatccggtgataccagtggggtggggttgcaggatgagtaattaatattttttagaaattcaaaattttgtgattggaatatgaaaaagggacatatcaaatgtcaatcactttctcaccaaatatggttctaagtatgaaaaatatagtaagggaaaaaaaaaagttatgggctcctatgggtccatgtttttcacgtgtatttttactaaaattttcttttgagtcgttatttatttttattttattttttaaaaataaaaataaaataagaaagaaaattgactctttatttagaaaaaacatttataaaaatcaaatctaaatctgatgatcaagttacctagagaaggtacgatggtaaacgtagaacctctacaagcatgtataggtaccgtctctattaaattaatcaagggaattgtgacaattaattaattaatcatgaataccaatattaaaggaacgaaataatatacgatgataaaataaaattataaaaatgtacaaaacaaataacaatagaattatgtaaattaatttattgaactaattaattagaaaaaaaagatatttgaaagaattagttttcaaaaaatttcaaatgattttattaaaaacaattttggattagtgatttcaatttatttatttacaaaaaagaagtttcaatttattttcaattaatgatttgattcaatctcctttgtatttaattttcaaaaaaaaaaaaaaagtcatttacacttattgtatcaaaataatttattacaaaattttaatttggtaacaaaaattatttttacttgcttttatcgtaaatgaatgaatttttataattttatttaaaacaaaaaagtattttaatttattttcatttaaaaaaaagtggcttatacaaattattaaaaaaaattatataacttcatttgcaaaaaaaaaatttaattaaaaaataaattttggaaaaactttacttgcaaaacaattttt

SEQ ID NO: 130 - DNA, 1683 bp, Chlorella
atgatccccgcagttgtcgacttcccgcagcagcagcagcagcagcagcagcggcagccgccccagcaggagcagccccagcaggggcaggagcgggagcaggctgccgccgccgggcggcgccaggacccgctgcaggagcaggaccagctgcagcaggcgcaggagctggagcggcggcggcggcgcaccgggttcaccgacaaggcgctgctgactcccgaggagctgcgccagaagctcaaggtggtgcagcagcagcgggcgctggtggtgctcctggtggacctgctggacgcgagcggcagcatcctggggaaagttcgggagctcgtcggcaacaaccccatcatgctggtgggcaccaaggccgacctgctgcccgcgggcgcagacggcgcccaggtggcggcctggctgcaggcggccgccgccttcaagcggatcgccgccgtgtctgtgcacctggtcagcagccgcaccggggcgggcgtgccggaggcggtgggcgcgatccgcagggagcggcgcggcagggatgtgtttgtgatgggggctgccaacgtggggaagagcgccttcatccgagccctcatgaaggacatgtgccgcatgggcagccgccagttcgacccgcaggcgctgagcagggggcggtaccttcccgtggaaagcgcgatgccggggaccacgctggagctgattcccatggagaacaagcagctgcacccgcgccgccgcctgcgcccctacgtgcccccctcccccggcgagctgctgcaagtcactgccgccgcctgctccatgcccgcacgcccgcgagacgccggcggagctgccgctggcgcgggcgcgggtgcggcggcggcggcggcggcggggcccagctgccacgtggccacctactggtggggcggcctggccaagctgcagctgctcagctgcccgcccgacacagagctggtgttctacgggccccaggccctgctggtggaggcttctgtggaagcggcagaccccgccggcgacgccgccgccgccagcgagggcgacgcctccgccagcgagggcgagggcccgggcggtgatgtgggcgcggggcggcggcgtggcggcggcgccgctagcggcggctccgggatgggaggcgggtggccgggccttggaggggaggaggtggcggaggagcctcacggctttggggcggggtcggtgatgcggcggggcgggctgcggccctgcaagaccctgcacatcaagtgtggagcgggtgggagcggggccaggcaggcggtggccgacatcgctgtctcgggcgtccctggctgggtggcggtgcacgccagcgccggcaggggccacacggtgcaagtgcgcgtgtggacccccccgggcgtggaggtcttcagccgcccgccgctgccggtgccctcgcccctggtcgagccaggcgcccccgatgcctggctgcctccgcgggctgccgccacgccgggaggcaccctggagcagcagcagcaggaggagccggcgggggcggcgcctgctacggcagcagcggcagcggcggcgccctcggcagcggcggcgcaagtgaccgaagtgcaaggagcgggggaggcggaggaggggcgggggcagcgggtgcggcagcgacccagctctgttgacgactggtggtga

SEQ ID NO: 131 - DNA, 1218 bp, Emiliania huxleyi
cgcgtccagggcacaagctcaagctggcacaagctctacaagctcaagcaccgcccgggccgcgaaatcgcacagtgacagttcatccaccaggactcgggccccgctccgccgccgttcatgattctcctgcccctgctcctcgccctgcctccgctcggccttcgtcgccccgcgccgctggcgcggcggtgcgcgcctccggtcgcggcagaggcgctcgtgccccggaaccgcgtcgcctgctacggctgcggcgcagagctgcaggccgacgtggctggttcgcccggctacatggagccagagcggtacaagatgaagcgcaagcgccgccagctccgcgagtcgctctgcgaccggtgccgccgcctgagctcgggcgagatcctgccagccgttgtcgagggtcggctcaagcggccgtcgggcgcggcggtcggcgaggaggggagagggatcacgacacccgaggcgctgcgcggcgtcctcctcccgctgcgcgagcggcctgccctcatcgctctcctcgtcgacttgacagacgtggctggcacgctgctcccgcgcgtgcgcgagctcgtgggcggaaacccgatcttgctgatcggcacgaagctcgacctgctgccgcgcggtacggagcctgagcgggtggcggactggctcagcggcgcggcgcgcaagatcggcggcgtcgtcgacgtgcacctcgtctcgtctaaggcggcccctcctcggctgtcggtgggcagcgtgggcagcgtgggcacaccgacgagtggcttggcaggcacctccttcttctggggcggtctcgcgcgcatcgacgtcgtctccgcgccgcccgcgctgcggctcaccttttgcaccggcggctcgcggctgaggctgcacgagtgcccgacggccgaggcggccgaggcgcacgcggcgagggcgggcatcgagtggacccctccgcaggacgccgcttcggcggcggagctgggggagctgcagctggcgcggacggcccggctgcgcctcacgccgtgcgagcaggcggccgacctcgcaatctcggggctggggtgggtctcggtcggatgcctgccgaccctgcagcagggggcgctcgaggcgaccctcgccgtgtgggtgcctcgcggcgtggaggtcttcgtgcgcccgccgatgcccgtgggtgggctgcccactgtcgggagcgaggcgtga

SEQ ID NO: 132 - DNA, 1415 bp, Ostreococcus taurii
atgctcgcgcgcacgtcgacgcgcgcgtccacggcacgggcgcgcgcgcgatcgtcgcgatcgtcgaatgcgggcgcgcgggcgccgggcgagcgagcggcgcgacgccatcgcgcgcggacgcgcgcgtcgaacgacccggcggcgacgacggcgacggcgcgcgagcgcgcgcggtgttacggatgcggcgtcggcgtgcagacgcgatcgaacgacgtcgcggggtacgtcgatgtcgcgacgtacgagcgaaaggcgacgcacggacagtgggacatgatgctgtgcgcgcggtgcgcgaagctgagcaacggcgcgtacgtgaacgcggtggagggccaggggggggtgaaggcgtcgccggggttgatcacgccgaaagagctgcgggatcagttgaaaccgatccgggagaagaaggcgctggtggtgaaagtcgtggacgcgacggatttccacgggagttttttgaaaaaggtgagagacgtcgtcggcgggaacccgattgtgttggtggtgacgaagattgatttattggggaatgcggtcgatcacgacgcgttggagcggtgggtggcgaaagaggcggagacgaggaggctcacgctggcgggaatcgcgctcgtgagctcgagaaggggttcgggaatgcgagaggcggtgctgcagatgatgcgcgaacgcaacggtcgggacgtctacgtcatcggcgccgcgaacgttgggaaaagctcgttcatcagggccgcgatggaagaattgcgttcggctggaaactactttgcgccgacgaaacgattaccagtggcgagcgcgatgccggggacgactctcggggtgatacctctgaaggcgttcgagggaaagggcgtcttgttcgacactccgggggttttcttgcatcacagattgaactctttgctcagcgcagaggatttatcggagatgaaactaggctcatcgcttaagaagttcgtcccacccacacccgagtgcgccgaaccgccgggtttcgcctcgttcaagggatactcgctgtattggggatcgtttgtgcgcgtcgacgttttagagtgtcctccgaacgtgacgttcggcttcttcggacccaaatcaacgcgcgtgagccttatgaaaacggcagacgtgcctgaaacgatttcggggcaggaagaggcggctttgagattggtgcaagagattgacttcttaccgccgatgcacgtggacggtccgctcgtcgacctctcggtgtccggacttggaggttggattcgcgtcgagaagacttcgggcagaggagacgggccaataagagctcatatatacggcattcgtggtttagaagtgttcgctcgcgatgtcatgccgacggcttagaggaaaatgtaatattcagaaattgttttgcc

SEQ ID NO: 133 - DNA, 4535 bp, Phaeodactylum tricornutum
atgagtggaactgcgcccaatttatctacaggcgttagcacttctgacaatgaaaggcggagaatttcgagcaatgatccaggaaaagacgaggtgggcgtccaagacacaattacctttttcaccactgacatcacagctctcaatactttgggcgcttcgatttggacgtatttggctagagccgctgggaaattgcaggctacgattcgtatagctagctttctatttatggggtacggcttttttctctcgcaaactcttttgttcacttcggaagaatgcggcatgacttattcctggcgccgttttcttgagctggatatatcctccattcatcctgtagggcgttctccatatcgactgtacaaattctatgatcagcgcgacccccgacatgaacgctttttacagcaagagagcgtgacgacttcaagaaaggcttccacggactggtgcctaaacgccgccttcccgactgctgttgtgtatattccaggtcacggcggaagttatcagcaaagtcgaagtttgggtgcgcatggaatacagctcacgcgacagcgggatgtgacgcaaaactacgttgtgcaagcgttacaaaagggaatgtggcatggaaacgcgacgcagctggaaaactttgtttatgacgtgtatgctttggattttgctgaagaaggtggtggtatgcatggagattttttggtggatcagagtcggttcgtgtcgaaagcgattcattttttgagcgaagcatgtggcttttccagtatcacagttgtcgcccactccattggtggcatttcgatccgcttagctttagttcgtgatgaaaagctgcgccttttggttacaaatgttattctactaggatcacctcaagcacgcaccgttctagcctgggatccctctttggaaaaaattcagacagaaattgttgaaaatcacgtaaatggtactgcttttgttgccatatcaggcggcctacgcgacgaaatgattcctcccgcagcttgtgaactcgttcctaaagataataacaccttgacacttttggctgttgatatcatgcctaaggaggcgtcaagcccttcgtttggaatggaccatcgcgcaatcgtgtggtgccacaatgttttggtaccactgcggaaaataatttttgctctagtcaggtcggaacgcgatggagaggctgcaccagcaagaataggagcagtacaatcgctgtttgatcgaagtaagacgcaaaactataacactgcacttcaacgtatgatgacgacgtttcggaaagtgcacggaccagtcgccagtttagccatggtaactggtctccttcacaatgccgaattgctactgggtttatttgcttacatctccctgtggagcaaagctggatttgcctcttgcttcaattctcattttggcgttttcagcggacgcaatccgagctactttgttgtggacagcacatcagtcatcagcactgaagccgacgacgttccaaagcaacgggataagttggcgctgggcgtcagcattgttcatgtcatctttggcgtcgtccgtctcttacgtcccaatgattttgccattgaaatgtcaaattcaatcaatattgcattgatcgcctcgatctatccactggctctccgacgcatccataagtttgcacagaaggttggtagctcccgcttttctttcattgaccttgatctattgacgattgtagtggtcccgtttttgggcgctggagaatttgcttatgtgctgtctaaaggctctgtgcaaaggtcaacactaccgatgctagcagcgcctttcctcattcgattggtcttaacctcgagcgacccaagcattccaccgcattcgtctcgaaaacggtatatctcagatgtcatccgcacacttcaggtatgcattctcttggtggttggtcctagagttctacaaacgggatcaggcttggcgtatagttttaatttaccactcggcggactggtgggtatgatgatgtggacggatacgttatggtcattaacgattagcggactaggttagttattgtcaatgcaattgtttttgcgtagcacatccctggattgtatccgaatatgttccatccgtatagtagtaaccgcagaatcaatcacttcatgggaacatggaagcacttgggtccacttatcgtacacctatggcatcaaatcgtttccaaaagcaccgaggtcttggttaaaatctttgtcgtactttattactggtgtgccgtccttcattggctgctatgctcagctgtcgtttaactgccctctcatccaaatggtctggaatttgttctgtatattcctgggaatgaactgattcagcaaggttgaatgggtaaagggagcgccgcttcgaaaaaatagatccctcgacacagtacggaataaccccatactggttactatcgatgaatcctacccatcccagactggcaaaagaaacgtccattacatatcttccggatgcagaaacaaattccttataacccgccacgaatctcccatccggggcagtctcagtcaaaaatggtagtaagggaagcgaatattcatcggccagccccctcacttcgtttctagttgcttcaaaaattcgttcttttactcttgcaatgtgaaatgatgggatggtggctcgatccggagctcttgatgttggaacaacccgaagtctcagagatggatgtaaaaaagcctgtgcatttatatgatgcttcgcctgcactacatctatacggcccaaaacgcaggtctcctcgtcttcgtcccatatgcctttcgtgttttcttcatccttgcccatccagctggcttcgattaagaggctctgtccctcccgtaacgatactttcaacccattccttgaagccggaataggaattgcttctgggcgagtgagtggttccatcaagtgtgcagggaaaattttgtattgaagcgcacgtggactaataacaccgggtgtgtcccacaaagcgtgagagtccgaaggaaaacatggcacgcgaactgcttgcagcgtggtaccgggtagattcgatccggtgactttcaaattcttgatcgtggctctccgttttacagcgaatcgattttgtccctttaaatacaccgattcagcaattaaaggtgacaatgttttcaccaaacttgattttccgacgttggcagtgccgatgacgaacacatctctacctcctagctgcaggagtatgctttcagccaaccgcaccaatccgacgccatttgtagcactaacatcgaagacggatgtaaatcggacgccggacattgcctcaattctccgagttatattcatcacatcactttcgctgcaacgaggcaacagatcaattttgtttatcaccaatatcaccggaatgcttccaatagttctacgcagatgcttaacgacagtgtgttccggatcagtggcatccaccaccattatacacattccaaacttgcgtcgggctacaatgaagcgtagctgctcgctaaagactttgggttcaatatcgcgcaaggcatcgtaggctccccaaatatcatttctttgtaacgattgacagcgactacagagaaaactatccattgggcgtgtcgcataatctccaacatccatgtaacgagttttcttctgtattcttttgcttaaagatgacgtatgctccatggtatcttcgccaccgacaaggcgagttcctgttatgttcgctgagtctgtattgttcgaccttctaccggataccttggctgaaacaacttgtgtcccgcagccagagcaaagtttcggtacagcatttgatattcgtttgccagcactgtgctgctggagaactacccggcctttcactcctcttggaggcgagccgccagctttgttcggtttgcgcgtggcgattttgctcgaggacgaaatggttccgggagcacccgggtgtttcttccctttggagctgctcggttgtttctgtaccggcgactgctgtttctttttactgctgcccttcttttttgactttgggctttttgcagcaacggcaaaacggcgagatactgttgctgtgggggctagtttggacgataggaacgtatacgaccttcctaattccgctagcaaaaagggaccagcggaaagatgttgctgggcggaccaagaaaacgctgccgttgctgtttggtaaaggtattgctgattgttgtagcgatctatcgttgttacgacactgctcgctgggaacacaggagtcggctgttgtaatttcatcgcggtcaagccttttcgcggagaggcgcctagtcgtatccctgttctaatcgatcgcaacatgcttgcataacagctaccctggaaaaaggtgatgggagtgcaaggaaaggacgctagactgtgtcacttgagcttctctagttgtgcatgaggagagaagccagatcaacaaaagaaacttgtttggtagtaagggtgattgaagg

SEQ ID NO: 134 - DNA, 1228 bp, Oryza sativa
gttgaggcgtactaaaattggggaatctcgtgaagtctctcctccctctcagttttcccgttcgtctccaaacctgcgagaggcggctccctcccgccatcccgatttggcttcccgccccaattcccgccgccgcataaaccctccacaaaagggctcctccgccctcgcctctccctccccatctcgcctcgccgctctccaacccatcgccgcggccgcgcgcgattcctcgcctccgcggcgagcctctcctctcttcctccgcccggcggcgttggcgttggcggcggcggcggcgatgagcgcggtgaacataaccaacgtggcggtgctggacaaccccaccgccttcctcaatcccttccagttcgagatctcctacgagtgcctcatccccctcgacgacgatctggagtggaagcttatatatgttggatctgctgaagatgaaaattatgaccaacaattagagagtgtgcttgttggccccgtcaatgttgggacctaccgttttgtactccaggctgacccaccggatccctcaaagatccgtgaagaagacataatcggcgtcactgtgctgctattgacatgttcctacatgggacaggagttcatgagagtaggttactatgtgaacaatgattatgacgatgagcaactgagggaggaacccccggcaaagcttctaatagacagggtgcagaggaacattctggctgacaagccccgagtcaccaagttcccaatcaacttccatcctgaacccagtacgagcgcagggcagcagcagcaggagccacagacagcttcgccggaaaaccacacaggcggtgaaggaagtaagcccgctgctgatcaatgatcagagtggggatgctaacattttgatgcccgtgccttttaggatatcttgtgatgctgtgagagtggtgattatgcttcttcattctcctaggtttgtcattggtgtctcgtcttttggaaggcaaattgttccctagttcctggtggttgctgaaaactgtatgttctcttgaaatctgccaatctggttaagagtacttgcgcaagttctgtttcatcaaatgctggatgcttcttttgtctagactatagttgttgagtattatgtgtttaggggtatattagtaatttcctatctctattgtactcatatatgccctttgggctaacacaatagcaactgttcattctccaaaaa

SEQ ID NO: 135 - protein, 188 aa, Oryza sativa
MSAVNITNVAVLDNPTAFLNPFQFEISYECLIPLDDDLEWKLIYVGSAEDENYDQQLESVLVGPVNVGTYRFVLQADPPDPSKIREEDIIGVTVLLLTCSYMGQEFMRVGYYVNNDYDDEQLREEPPAKLLIDRVQRNILADKPRVTKFPINFHPEPSTSAGQQQQEPQTASPENHTGGEGSKPAADQ

SEQ ID NO: 136 - DNA, 981 bp, Arabidopsis thaliana
acgaaccgtctctgaatctgaccgaccaccattcttctcgcgccggcgatttcactgtttacagagaaatctcgagaaccctaacctgggtctctctagatttctttgaaatttcgagaatctagggtttcaaactaaaatcgagtccttgagttttcccaatttaaatcgttatgagctctatcaatatcactaacgtcaccgtcttggacaatcctgctccgtttgtgaatccattccagttcgagatttcttacgaatgcttgacctctctcaaagacgatttggaatggaagcttatatacgtagggtcagctgaagacgaaacgtatgatcaagttttggaaagtgttcttgttggtcctgttaacgttgggaactatcgatttgtgttgcaggctgactctccagatccgttaaagattcgtgaggaagatattattggtgttactgtgttattgttgacttgctcatacatggatcaagagtttataagagttggctattatgtgaacaatgactatgatgatgaacagctcagggaagagcctcctaccaaggttttgattgataaggtccaaaggaacatactcacagacaaacctagagtaactaagttccctatcaactttcatcctgagaatgagcagactcttggtgatgggcctgcacctactgaaccatttgctgattctgttgtaaatggagaagctccggtgtttcttgagcagccacaaaagcttcaggagatagaacaatttgatgattctgatgtaaatggagaagctatagcgttgcttgatcagccacaaaatctccaggagacatgattcttgtttgactcaagcttaactggaaactggattagaactatcatctcaattctaatcgaaagatttgtatttttgtttcttttatctggaacttgaactccagttgtgttactgtttgtagaaatttaagttcttcttgcaacatccc

SEQ ID NO: 137 - protein, 218 aa, Arabidopsis thaliana
MSSINITNVTVLDNPAPFVNPFQFEISYECLTSLKDDLEWKLIYVGSAEDETYDQVLESVLVGPVNVGNYRFVLQADSPDPLKIREEDIIGVTVLLLTCSYMDQEFIRVGYYVNNDYDDEQLREEPPTKVLIDKVQRNILTDKPRVTKFPINFHPENEQTLGDGPAPTEPFADSVVNGEAPVFLEQPQKLQEIEQFDDSDVNGEAIALLDQPQNLQET

SEQ ID NO: 138 - DNA, 1055 bp, Arabidopsis thaliana
cattttcatcgtcttcttaatcaaaaaaaaaaaaaaaaaaaaactctgaagcttcttctttgattaattctctcctggggaaaaaaaaccctagctcttccttcttctctcttctcttgaattatctgctttcgaattttttgaaaagggagaaactttttcatctgggtctctctctctcccgagtttggatgaagtttattgaaacctagggtttttctccgacttggttgttgattagagataatgagtgcaatcaaaatcaccaacgtcgctgtattgcataatcctgctccttttgttagcccttttcagttcgagatttcttacgagtgtttgaattctctcaaagacgatttggaatggaagcttatctatgtaggctcagcagaagatgagacttatgatcaacttctagagagtgtgcttgtagggcctgttaatgttggcaactaccgctttgtatttcaggctgatcctccggatccatcaaagattcaggaggaagacatcatcggtgttactgtgctattgttgacatgttcttacatgggtcaagagttcttgagagttggatattacgtgaacaatgattatgaggatgagcaactcaaggaagagcctccaactaaggttttgattgataaagttcagaggaacatactttccgacaaacctagagttactaaatttcctatagattttcatccagaagaagagcagactgctgctactgccgctcctcctgaacaatctgatgaacaacaacctaatgtcaatggtgaagctcaggttttacctgatcagtcagtagaaccaaaacctgaggaatcatgatccttataccaagtctagttgaagaaaagtggatgagaattgagaactattgtctccacagatgttgcgctttgcttttctgtctttcaaaagcattatatgctgtttgttgtacttaattctagaggctttagggaagtgattcttgacatttttgtatgtttatgttttgggcaaaggttttttaaatcgaagcaaagccaacagttgccaaacaa

SEQ ID NO: 139 - DNA, 896 bp, Glycine max
ggggggggtgaggccgaggcgagttgtgaatgtgagtgtgttttctcttcaaaaccctctcccgccaacacaccgcgcttcttcttcttcttcttttttgtgttttgtgaatactgagatgagtgctgtgaacatcaccaacgtcaccgtcctggacaaccctgcttcctttctgaccccctttcagttcgagatttcctacgagtgtctcaccgctctcaaagatgatttggaatggaagctcatttatgttggatctgctgaggatgagacctatgatcaattattagagagtgtccttgttggtcctgtcaacgttggaaactatcgttttgttttacaggcagatccaccagatccatccaagattcgtgaagaagatataattggtgtcactgtgcttctgttgacctgctcctatctgggtcaggaatttattcgtgttggctattatgtgaacaatgattatgatgatgagcagctgagagaggaacctccaccaaaggttttaatcgatagggttcaaaggaacattttgtctgataaaccaagggtcacaaagttccccatcaatttccaccctgagaacaatgaaaatgaagagcaacaaccccctccatctgagcacccatcggaaactggagaagatccacttgctgtagttgatcgtgatcctccagatgagaaggattcttaacattttgtaggtatgcagacctttcaattcctaaatatccaacatctaattcgctgtatggattatgatatcatttttgtgaatctctgttcgatgatgtgaatgagatttatgagctcttttagaagtagtggcctcagagtgctaggttcaaaatgattgtaacgtatgaaaaccagtttgctttgaatgaagcttt

SEQ ID NO: 140 - DNA, 1108 bp, Hordeum vulgare
ccaaatccagccaaaacccctccccccctcctctcggttcggcgatcggcggcggcggcggcgatgagcgcggtgaacctgacgaacgtggcggtgctgaacaaccctacctctttcgtcaaccccttccagttcgagatctcgtacgagtgcctcgttgccctcgaggatgatctggagtggaagcttatatatgttggatcagctgaagatgagaactatgatcaacaacttgagagcgtgcttgttggccctgtgaatgttgggacataccgttttgttctgcaggctgatccccctgatccctcaaagatccgtgaggaggacataatcggtgttacggtgcttttgttgacctgttcatacatggggcaggagttcatcagagtaggttactacgtgaacaatgattatgatgatgagcagcttagagaagaacccccagcgaagctgctgattgaccgggtgcagagaaatattttgaccgacaaaccccgagtcaccaagttccccatcaacttccatcctgaaaccagtggaggacagcagcaagatcaaccacaatcagctgtacctgaaaaccatacaggcgaagggagcaaggccaacacagatctttgactgggtctggaaacttgggagtgccaaaattttgatctgcatgccttcgtaggtgagatcacaattcacaatatgagatggcttcctggatccggatgctgctactgaatcgtctggtctgggggacactaaactagactaccttaaccgaaactcacattggttggttacattgtgctctagtgggtaggtccgacacactgtgaaattgccgtcttttgtagtgatgactataagctgatgttcaccagtgatccggccaacggagtgtctttcccttgataaggttcacacccaagtttttggtaaaaaaaagaaaattgagagtgaaaaaaaggagagggggcccggccacttccctatttgttgttttcgccccccctggcccgtttacgcctgacgtgaaacctgggttccaattattcctttgccatccccctcccgcgggattagagggccccccgaccctcccaatttccgtaatggg

SEQ ID NO: 141 - DNA, 975 bp, Hordeum vulgare
gcacgaggacatcgtaataacaacacaaactgcatatcatctagagaaactatagttcactttaaaaagccattagagctaatttacattgtgtaagagaagctacggcggaagaaaacatgtcggtagtttcactgctaggcgtcaatgttcttcagaatccggcccggtttggtgacccatatgagtttgaaatcacgttcgaatgtttagaaacactccagaaagatctcgaatggaagttgacttatgttggatcagctacatccaatgatcacgatcaagagcttgatagcttactcgttgggcctattccagttggcgttaacaaatttattttcgtagcagaccctccggacaccaataagataccagacgccgaaattctaggtgtcaccgtcatacttctaacatgtgcttacgacggtcgagaatttgtccgtgttggatactacgtcaataacgagtatgactcagatgaactgaacacagaccctcccgcaaagcccatattagagaaagttcggcgtaatattctggccgagaagccaagggtaactcgctttgcaataaagtgggactctgatgattctgcgccaccactctatccacctgagcaaccagaagcagacttagtggccgatggtgaagaatacggtgccgaagaggctgaggacgaagacgaagaagagtctgcggatgggccagaagttccagcagaccctgacgtcatgatcgatgattctgaagccgcaggtgccatggtagagactgtcaaagcaaccgaagaagaatccgatgccggcagcgaagatttggaagctgaaagcagtgggagcgaggaagatgagattgaagaagatgaagagcgcgaggatgaacctgaagaagccatggatttggatggcgcaggtaaacgaaacgctgctatatctagcagcaacaacaccgataccacaatggctcattaattta

SEQ ID NO: 142 - DNA, 934 bp, Hordeum vulgare
  misc_feature (19)..(19): n is a, c, g, or t
ttcggcacgaggcgcaagnatctcgacccgccgcgtccccctcttgcgtcgattgacgggagaggccgggtcggcggaaggcggcggcgatgagcgcggtgaacatcaccaacgtggcggtgctggacaaccccaccgccttcctcaaccccttccagttcgagatctcctacgagtgcctcgtccccctcgacgacgatctggaatggaagctcacatatgttggatcagcggaagatgaaacctatgatcagcaacttgagagtgtgcttgttggacctgtcaatgttggaacctaccgttttgtattccaggctgacccaccggaccctttgaagatccgtgaagaagacatcatcggtgtcaccgtgctgctattgacatgctcctacgtggggcaggagttcatgagagtgggttattatgtgaacaacgactatgacgatgaacagctgagagaagagcccccagcgaagctattacttgatagggtgcagagaaacattttggccgacaagccccgtgtcaccaagttccctatcaacttccaccctgaacccggcacgagctcagaacagccgcagcaggatgcagaacagctgcagcaaccggcttcgccggaaccacagatggctccagtagaaccacagacggcgccactagaggacggcacggctgatgaaattaagcccagcgctatcgccctatgatcggagttggggtgctaatgttctgttctccgtgcctttgaggtgtgtcttgtaatgctacgaatcaagagtggtggttctgcattgtactggtctgttgttgacgtttcacaatctgttctgttgcaaatgttcggtgtgtagctattttcttgataaagagtacttctgcctgttttgttgcacatatttgtttttttactggaccagattgcttcagcctg

SEQ ID NO: 143 - DNA, 763 bp, Hordeum vulgare
cgggtcggcggaaggcggcggcgatgagcgcggtgaacatcaccaacgtggcggtgctggacaaccccaccgcctttctcaaccccttccagttcgagatctcctacgaatgcctcgtccccctcgacgacgatctggaatggaagctcacatatgttggatcagcggaagatgaaacctatgatcagcaacttgagagtgtgcttgttggacctgtcaatgttggaacctaccgttttgtattccaggctgacccaccggaccctttgaagatccgtgaagaagacatcatcggtgtcaccgtgctgctattgacatgctcctacgtggggcaggagttcatgagagtgggttattatgtgaacaacgactatgacgatgaacagctgagagaagagcccccaacgaagctattacttgatagggtgcagagaaacattttggccgacaagccccgtgtcaccaagttccctatcaacttccaccctgaacccggcacgagctcagaacagccgcagcaagatgcagaacagctgcagcaaccggcttcgccgggaccccagatggctccagtagaaccacaaacggcgccactagaggacaggcacggctgatgaaatttagcccaacgctatcgccctatgaatcggagttggggggctattggtctgttctccggccctttgagtgtgtcttggaatgctccaaatcaaagaggggggttcgccttggactggcttgttgttgcgcgttccaaatctgc

SEQ ID NO: 144 - DNA, 878 bp, Medicago truncatula
actccctcccgccaacacatttcattgccctctcttgttttcattttcgtcccctttccctctttttgaattgaataagtagaagaaagagtgttgcgaagaagattcgagaagaagaagaaggaggaggaggatgagcgcggtgaatatcacgaacgtgacggtcctcgacaatccagcttcgtttctcaatccctttcagttcgagatttcctatgagtgtttggccgctctcaaagatgatttggaatggaagctcatctatgttggatctgctgaggatgagacttatgaccagttattggagagtgtccttgttggtcctgtcaatgttggaaactatcgctttgttttacaggcagatccaccagacccatccaagattcgtgaagaagatatcattggtgttacagtgcttctactcacctgctcttatttgggtcaagagttcattcgtgttggctactatgtcaacaatgactatgacgacgagcagctcagagaggaacctccaaccaaggttttaacagatagggttcaaaggaacatcttatccgataaaccaagggtcactaagttccccatcaatttccatcctgagaacaatgaaaatgaagaacaaccccctccctccgagcaacaacctgaaactggagaagaagaagatccacttgctgcaccggataccattcccccaaatgttcccccaaatgagggggggttcttaacattttgttggtatacaggcttttcaattccccaatgtccatctttacattacctgtatggattaattaagatacttttttggaatctcttatgggataaaggtggatgatataattcatgagttctattgggagtcttttgatgactaa

SEQ ID NO: 145 - DNA, 531 bp, Medicago truncatula
cattttcgtcccctttccctctttttgaattgaataagtagaagaaagagtgttgcgaagaagattcgagaagaagaagaagaagaagaaggaggaggaggatgagcgcggtgaatatcacgaacgtgacggtcctcgacaatccagcttcgtttctcaatccctttcagttcgagatttcctatgagtgtttggccgctctcaaagatgatttggaatggaagctcatctatgttggatctgctgaggatgagacttatgaccagttattggagagtgtccttgttggtcctgtcaatgttggaaactatcgctttgttttacaggcagatccaccagacccatccaagattcgtgaagaagatatcattggtgttacagtgcttctactcacctgctcttatttgggtcaagagttcattcgtgttggctactatgtgcaacaatgactatgacgacgagcagcctcagagaggaacctccaaccaaggttttaacagataagggttcaaaggaacattcttatcttga

SEQ ID NO: 146 - DNA, 1702 bp, Physcomitrella patens
ctcctcttcttctcccatgtcgttttggtcggaaccaaactaagggctcttcgccgtctctccagcttcaccttcggttgccttggctcctctcccctccctccaacaattggcacctttccccgtcaccctacgtctctcgtctgtcgcgggcattgccttcttgtcaatcctgcctcattgccttactccatccccgcctctcctcgttccacctaccgttcgtccgaatttttagccgtgctcgtggtttcttttggtccacctctgggagagcataccacggatcgcatctcggtgcttttaagctttcagcttacattgaaggtgatgggattttcctagggagacgtgcccgtggacgagcaattatcttgattgcgagagttccattttctatgagtcaacagttggagttcagcaggcgcgggagaagggcgtggtggttaacattgtctgttgccgcgtggacttagtctttgcaggattgcacaaaagaggttttagcactttgagcttcaattttgaagcgctcagcggtgtggtggttgtttcattgcccccgagggcatgacgttttggtctctgcactccatgatcgcagtctggccttgatcagtcaccgggtgtatttttacaagcccgttttccggagtcttatgttcacattcgttttcagttctctgaaactggacttacacttatctcagtagctgcccaggaggttgctacaatgagtgccgttaatgtgacgaacgtgaccgtgctggacaatccgtctatgttccagaacccctttcaattcgagatttcttacgaatgtctggttccacttaaagacgatctcgaatggaagctgatatatgtcgggtctgccgaggatgaaaagtatgatcaagttcttgagagcgttcttgttggtccggtcaacgttggaaactacagatttgtttttcaggcagatccaccggagccatccaagatccccgaggaagatattattggagtcaccgtgctattgttgacatgctcttatgtgggacaggagttcctcagagttgggtattatgttagcaacgagtacgttgatgagcctttacgtgaagagccacccgccaaagttcttatagatagggtgcaacggaacatccttgccgacaaaccacgggtcaccaagttcccaatcgtcttcaatgccacacccccttctaaccaagtgacagataatgcatctgccaacagctatcccatggtttacaacgcgccacctccaagccaagtgaccgatgcggaactagatgacggtgtcagaccgatggaaatctcgtcagcttatccacagcaggactaggtctgcggtgagtaatattgtacatctgcatagtctcacctgtaaaacatgagggtacacaagtttgggtaaatcaggatgaagccctcatctgaacactttagttctagaaatagcttcaggatcagtacgcattagattcagagcctggtgagatttttgtgggggagggcagggctttactgtagattcatgttgactaccccaggttacatgtgaaaagatagtttcaaaactttggaggtacacttgaaagttgaatgcagggtccgtagcgtaggcgaagctctctttgatctttgttgtggtcgatttgatggcatgtgaagagtttctgagttggctttgtgc

SEQ ID NO: 147 - DNA, 1401 bp, Physcomitrella patens
tggcctctttacactccatctcctgcttactccaggtcgattgcggtcactctgcggacgccgattctgagcggcaacactgctgcggaagaggaaggctgacctccatgcatttctagtttttgaggttatgaagaagatatggcagtgaggtgattggtttgtggattctcggatttgcagtaccgcgctttggattgaggcagttgattggaaagggtgacagtctatcatcatgagtgctgtgaatgtcaccaatgttgcagtgctcgataacccttccatgttccagaacccgtttcagtttgagatatcttacgagtgtctcactgcccttcaagatgacctagagtggaagcttatatatgtcggatcagctgaggatgagaagtatgatcaagttctggagagtgtgctcgttggccccgtgaacattggaaattatcgatttgtccttcaggcggatcctccggatgtatccaggatccctgaggaagatgttattggggtcactgtgttgcttttgacatgctcctaccgaggacaggaattcatacgagtgggctattacgtgagcaatgactacgtggacgaatctcttcgcgaggagcctccagtcagagttctcattgacaaggtccagcgcaacatccttgccgacaagccacgagtcaccaagttcccgatcctttttaattcccctggggtgatagctcctgagcctcaggaggtttcagatgcattcatctctccgactactgagttcgacatgatgaacaccaatgagaaggcttcaggatcacacactccagctgttgtgttggacttcacgccaacagttgagcaggattcagatgcacgcgcttcacagccatccctaatgttcttgatgcaaaacggagctgtcagaccagttaatctatgtgcgcctcaggttttacaagaagtatgttgagaagagagtcgtgtttcaataactgtataatttggaaaggctgttgcccttgtgatgcttgcacccaggttaggtcatactcggcatccaagctcataggattgcgggaattagtgggaagttctgtgtgttgattagcaccgtgcatcccaaattagacagctttactttcttattgtaggacatgctacacggtgcgggcgaaagtgcccatccatcattacattctgcatcaatttaggatagtttagatgctggcggagagattagtgttttcattctttgaattcatagtggagcaatggtagtactcaaattgctttgtcactatcactgctgttttacagcctgtacaagtagccagtgcgattggcatggcgtctccactttcatcactgaaaactgtaaatatttggttttggtgcacacatgctaaatcttaatat

SEQ ID NO: 148 - DNA, 683 bp, Populus trichocarpa
tgttttaatttgatagaaagacgatagataatgagtgcagtgaatcttactaacgttaccgtactagataatccggcgccgtttccgtctccctttcagttcgagatctcatacgagtgcttgactcctcttaaagacgatttggaatggaaactaatctacgtggggtctgctgaggatgaaacatatgatcaactattggagagtgtacttgttgggcctgtcaatgttggaaattatcgttttgcaaacccaccagatccatcaaagattcgtgaagaagatatcattggtgtcacagtacttttgttgacatgctcttatttaggtcaggaatttgttcgagttggctactatgtgaacaatgattacgaagatgagcagcttagagaggaacctccacctaaagtgttgattgataaggtccaaagaaacatcctatctgataaacccagggttacaaagttccctatcaatttttatcctgaaaatactgaggcagcagaggaaccccctgagaatgatcaacctgctaaaactgatggaaatgaagaacaattgcctgcttctccacatcatgctttagataaagagggaccttgaattttacaatcagcatcaactactaacctcccaagtttctcattcaggtttcacatcatcagggtcattttaggcgatgc

SEQ ID NO: 149 - DNA, 685 bp, Solanum lycopersicum
cgagattaacctcgagtcagccacccgaataaggcaatcatgagcgctgtgaatgttacaaacatcaccgtgctaaataatccgtcatcgttcctttcgccactgaagtttgaaatcacttatgactgcgttaatgctctcaaagaagatttggaatggaaactcatctatgttggatctgctgaggatgacacgtatgaccaattactagaaagtgtgtttgttggtcctgtcaatgttggaggtttccgctttgtattgcaggcggaccctccagatcctgccaaaattcgtgctgaagatatacttggtgtcactgtgctccttttgacctgttcctatgtgggtcaagagtttgtacgaattggctactatgtgaacaatgattacaatgatgagaatttgagacaacaaccttcccaaatggttaaaattgacatgcttcaaaggaacatactaacagacaaacctagagtaacaaagttccctatcaattttcaccccgaaaacagcgagactggagagcaagccgctgcccctccacctgatgataatacggctgaagcagatggttatgaagagtgactaccttcaactaggaatggatcagatgagggtggggcctaactgtggaaagatgttaatcttttgcgcaactcctaaccttttagtttgacatcgttct

SEQ ID NO: 150 - DNA, 614 bp, Solanum lycopersicum
gggttcgattagggtttcggagtcggagaattttctagagagatgagcgccgtcaacattacgaacgtcgccgtactggataatccggcaccgttcctcagtccttttcagtttgagatctcttacgagtgcctcgatgctcttaaagatgatttagagtggaagctaacttatgtgggatctgctgaggatgatacttatgaccagcaactagaaagtgtttttgttggacctgtaaatgttgggaaatatcgttttgtgcttcaggctgatccccctgaaccatccaaaattcgcgaagaagatataattggtgtcacagtattgctattaacatgctcttatgtgggtcaagaatttattcgagtaggatactatgtgaacaatgattatgatgatgaacagctaaaagaagaaccgcctcagaaggttttggttgataggatccagaggaatattttggttgacaaacctcgagtcacaaaattccccattaatttccatccagaaaacaatgaagatggagaacaagctcctcctgacaatgcaacagaagaaaaggcgcttcgagaagaacccgtttcttcacccaagcaatgtaatgagcagtgtcc

SEQ ID NO: 151 - DNA, 736 bp, Triticum aestivum
gccgcgctccctcctccaaaaaggccccgccttgattcaccatcccccctccgcccggacccgcaagatctccactcctcgatcgattgatatcagacgcggccgggtcggcggaagtcggcggcggcgatgagcgcggtgaacatcaccaacgtggcggtgctggacaaccccaccgccttcctcaaccccttccagttcgagatctcctacgagtgcctcgtcccgctcgacgacgatctggaatggaagctcacatatgttggatcagctgaagacgaaacctatgatcagcaacttgagagcgtgcttgttggacctgtcaatgttgggacctaccgttttgtcttccaggctgacccaccggaccctttgaagatacgtgaagaagacatcattggtgtcaccgtgctgctgttgacatgctcctacgtgggtcaggagttcatgagggtgggttattatgtgaacaatgattacgacgaggagcagctgagagaagagcccccagcaaagctattgcttgacagggtgcagagaaacattttggctgacaagccccgtgtcaccaagttccccatcaacttccaccctgaacccggcacgagcgcagaacagccgcagcaggatgcagaacagcagcagcagccgacttcaccggaaccacagaaggctccagtagagccacagatggcgccactagagaacgtccacattgctgccgagcaatgatcag

SEQ ID NO: 152 - DNA, 1157 bp, Zea mays
gcctctccactccaaacctgcagctccgagccgtctcccgccaacccaatttcgcttcccgccgctccccatctcatcccacaccgtcaccattacaccgcccgaaaggccgctagaaatccgatcccgagccgccgcgtcttgctcgcgtcttttcccctctgctcgagcacgcgcgggcgcgcggctgctcccggcggcggcgatgagcgcggtgaacatcaccaacgtggcggtgctggacaaccccaccgccttcatcaaccccttccagttcgagatatcctacgagtgcctcgtgcccctcgacgacgatctggagtggaagcttatatatgttggatcagctgaagatgaaaactacgaccaacagcttgagagtgtgcttgttggccctgtcaatgttgggacctaccgttttgtcctccaggctgatccaccggatccctcaaagatccgtgaggaagacataattggtgtgactgtgctgctattgacttgctcttacatgggccaggagttcatgagagtaggctactacgtgaacaatgattacggcgatgagcagttgagagaggagcctccagcaaaggtgctaatcgatcgggtgcagagaaatatcctggccgacaagccccgggttaccaagttccctatcaacttccatcctgaaccaagtacaggcacggggcggcagcagcagcagcaggagcctcagacggcctcaccagagaagcacgcaggcagcggtgagggcaatggaagcaagcctgaggctgaccaatgaacacagtcgacttcaagtatttttgaagcgcgtccctacaggttgtgctgtaaggttacgaacgggattagcggttatacattgccctgggatcctgtttttagcacatggtggctgtttagaactctttgttctgtacacattgagatgcaaatgctgggtagtgggtagctgggtgtttccttggcaaagagtattttaagcctgttctcctgcaattgctttatgcttctttagagtttagagtagcggtttacctcaaaactaactggctagtcttggtcaccagctttggcctttgcggtatagcttgtaacgggaatccttctgttcacttaggcaagtggtgaaatgaaatgatttgtgattg

SEQ ID NO: 153 - DNA, 1053 bp, Zea mays
gccgccgtgcctcgctttcgtcttttcccctctgctcgagtacgagcggctactcccagcggctgcggcggcggcggcgatgagcgcggtgaacatcaccaacgtggcggtgctggataaccccaccgccttcctcaatcccttccagtttgagatctcctacgagtgcctcgtgcccctcgacgacgatctggagtggaagcttatatatgttggatcagctgaagatgaaaactacgatcaacagcttgagagcgtgcttgttggccctgtcaatgttgggacctaccgttttgtccttcaggctgacccaccggatccctcaaagatacgtgaggaagacataattggtgtgactgtgctgctattgacatgctcttacatgggccaggagttcatgagagtaggctactacgtgaacaatgattatgatgatgaacaattgagagaagagcctccagcaaaggtgctaattgacagggtgcaaagaaatatcttggccgacaagccccgagtcaccaagttccctatcaacttccatcctgaacccagtacaggcccggggcagcagcagcaggaaccccagacgacctcgccagaaaaccacacaggcaatggcgaggccaatggtagcaagcctgaggctgaccaatgaacacagttggcttcagatattttgatgcgtgccccttacaggttgtgctgtaatattacaaacgggattagtggttgtgcattgccctgggatcctgaactctgttctgtaacttgagatgcaaatgctgggtacctggatgttttcttaagcacgagtatttcagcctcgagatgtgatcaattgcatcgaaagcatgtgctccaagaacgcaagcatgaggaaatacagcaaaaaacagcagattaactgaatcctttctctgaatctttttgagaaacaactttgcacgacgattcactgctgtaccaggacgcgctacgtgaatttttctagacctttttggtgcttgcagtcacgatcatttcatttgtcatcttttcacgtctcttttgtgt

SEQ ID NO: 154 - protein, 196 aa, Arabidopsis thaliana
MSAIKITNVAVLHNPAPFVSPFQFEISYECLNSLKDDLEWKLIYVGSAEDETYDQLLESVLVGPVNVGNYRFVFQADPPDPSKIQEEDIIGVTVLLLTCSYMGQEFLRVGYYVNNDYEDEQLKEEPPTKVLIDKVQRNILSDKPRVTKFPIDFHPEEEQTAATAAPPEQSDEQQPNVNGEAQVLPDQSVEPKPEES

SEQ ID NO: 155 - protein, 192 aa, Glycine max
MSAVNITNVTVLDNPASFLTPFQFEISYECLTALKDDLEWKLIYVGSAEDETYDQLLESVLVGPVNVGNYRFVLQADPPDPSKIREEDIIGVTVLLLTCSYLGQEFIRVGYYVNNDYDDEQLREEPPPKVLIDRVQRNILSDKPRVTKFPINFHPENNENEEQQPPPSEHPSETGEDPLAVVDRDPPDEKDS

SEQ ID NO: 156 - protein, 185 aa, Hordeum vulgare
MSAVNLTNVAVLNNPTSFVNPFQFEISYECLVALEDDLEWKLIYVGSAEDENYDQQLESVLVGPVNVGTYRFVLQADPPDPSKIREEDIIGVTVLLLTCSYMGQEFIRVGYYVNNDYDDEQLREEPPAKLLIDRVQRNILTDKPRVTKFPINFHPETSGGQQQDQPQSAVPENHTGEGSKANTDL

SEQ ID NO: 157 - protein, 283 aa, Hordeum vulgare
MSVVSLLGVNVLQNPARFGDPYEFEITFECLETLQKDLEWKLTYVGSATSNDHDQELDSLLVGPIPVGVNKFIFVADPPDTNKIPDAEILGVTVILLTCAYDGREFVRVGYYVNNEYDSDELNTDPPAKPILEKVRRNILAEKPRVTRFAIKWDSDDSAPPLYPPEQPEADLVADGEEYGAEEAEDEDEEESADGPEVPADPDVMIDDSEAAGAMVETVKATEEESDAGSEDLEAESSGSEEDEIEEDEEREDEPEEAMDLDGAGKRNAAISSSNNTDTTMAH

SEQ ID NO: 158 - protein, 206 aa, Hordeum vulgare
MSAVNITNVAVLDNPTAFLNPFQFEISYECLVPLDDDLEWKLTYVGSAEDETYDQQLESVLVGPVNVGTYRFVFQADPPDPLKIREEDIIGVTVLLLTCSYVGQEFMRVGYYVNNDYDDEQLREEPPAKLLLDRVQRNILADKPRVTKFPINFHPEPGTSSEQPQQDAEQLQQPASPEPQMAPVEPQTAPLEDGTADEIKPSAIAL

SEQ ID NO: 159 - protein, 196 aa, Hordeum vulgare
MSAVNITNVAVLDNPTAFLNPFQFEISYECLVPLDDDLEWKLTYVGSAEDETYDQQLESVLVGPVNVGTYRFVFQADPPDPLKIREEDIIGVTVLLLTCSYVGQEFMRVGYYVNNDYDDEQLREEPPTKLLLDRVQRNILADKPRVTKFPINFHPEPGTSSEQPQQDAEQLQQPASPGPQMAPVEPQTAPLEDRHG

SEQ ID NO: 160 - protein, 234 aa, Medicago truncatula
MSAVNITNVTVLDNPASFLNPFQFEISYECLAALKDDLEWKLIYVGSAEDETYDQLLESVLVGPVNVGNYRFVLQADPPDPSKIREEDIIGVTVLLLTCSYLGQEFIRVGYYVNNDYDDEQLREEPPTKVLTDRVQRNILSDKPRVTKFPINFHPENNENEEQPPPSEQQPETGEEEDPLAAPDTIPPNVPPNEGGFLTFCWYTGFSIPQCPSLHYLYGLIKILFWNLLWDKGG

SEQ ID NO: 161 - protein, 115 aa, Medicago truncatula
MSAVNITNVTVLDNPASFLNPFQFEISYECLAALKDDLEWKLIYVGSAEDETYDQLLESVLVGPVNVGNYRFVLQADPPDPSKIREEDIIGVTVLLLTCSYLGQEFIRVGYYVQQ

SEQ ID NO: 162 - protein, 205 aa, Physcomitrella patens
MSAVNVTNVTVLDNPSMFQNPFQFEISYECLVPLKDDLEWKLIYVGSAEDEKYDQVLESVLVGPVNVGNYRFVFQADPPEPSKIPEEDIIGVTVLLLTCSYVGQEFLRVGYYVSNEYVDEPLREEPPAKVLIDRVQRNILADKPRVTKFPIVFNATPPSNQVTDNASANSYPMVYNAPPPSQVTDAELDDGVRPMEISSAYPQQD

SEQ ID NO: 163 - protein, 239 aa, Physcomitrella patens
MSAVNVTNVAVLDNPSMFQNPFQFEISYECLTALQDDLEWKLIYVGSAEDEKYDQVLESVLVGPVNIGNYRFVLQADPPDVSRIPEEDVIGVTVLLLTCSYRGQEFIRVGYYVSNDYVDESLREEPPVRVLIDKVQRNILADKPRVTKFPILFNSPGVIAPEPQEVSDAFISPTTEFDMMNTNEKASGSHTPAVVLDFTPTVEQDSDARASQPSLMFLMQNGAVRPVNLCAPQVLQEVC

SEQ ID NO: 164 - protein, 190 aa, Populus trichocarpa
MSAVNLTNVTVLDNPAPFPSPFQFEISYECLTPLKDDLEWKLIYVGSAEDETYDQLLESVLVGPVNVGNYRFANPPDPSKIREEDIIGVTVLLLTCSYLGQEFVRVGYYVNNDYEDEQLREEPPPKVLIDKVQRNILSDKPRVTKFPINFYPENTEAAEEPPENDQPAKTDGNEEQLPASPHHALDKEGP

SEQ ID NO: 165 - protein, 181 aa, Solanum lycopersicon
MSAVNVTNITVLNNPSSFLSPLKFEITYDCVNALKEDLEWKLIYVGSAEDDTYDQLLESVFVGPVNVGGFRFVLQADPPDPAKIRAEDILGVTVLLLTCSYVGQEFVRIGYYVNNDYNDENLRQQPSQMVKIDMLQRNILTDKPRVTKFPINFHPENSETGEQAAAPPPDDNTAEADGYEE

SEQ ID NO: 166 - protein, 191 aa, Solanum lycopersicon
MSAVNITNVAVLDNPAPFLSPFQFEISYECLDALKDDLEWKLTYVGSAEDDTYDQQLESVFVGPVNVGKYRFVLQADPPEPSKIREEDIIGVTVLLLTCSYVGQEFIRVGYYVNNDYDDEQLKEEPPQKVLVDRIQRNILVDKPRVTKFPINFHPENNEDGEQAPPDNATEEKALREEPVSSPKQCNEQCP

SEQ ID NO: 167 - protein, 200 aa, Triticum aestivum
MSAVNITNVAVLDNPTAFLNPFQFEISYECLVPLDDDLEWKLTYVGSAEDETYDQQLESVLVGPVNVGTYRFVFQADPPDPLKIREEDIIGVTVLLLTCSYVGQEFMRVGYYVNNDYDEEQLREEPPAKLLLDRVQRNILADKPRVTKFPINFHPEPGTSAEQPQQDAEQQQQPTSPEPQKAPVEPQMAPLENVHIAAEQ

SEQ ID NO: 168 - protein, 193 aa, Zea mays
MSAVNITNVAVLDNPTAFINPFQFEISYECLVPLDDDLEWKLIYVGSAEDENYDQQLESVLVGPVNVGTYRFVLQADPPDPSKIREEDIIGVTVLLLTCSYMGQEFMRVGYYVNNDYGDEQLREEPPAKVLIDRVQRNILADKPRVTKFPINFHPEPSTGTGRQQQQQEPQTASPEKHAGSGEGNGSKPEADQ

SEQ ID NO: 169 - protein, 191 aa, Zea mays
MSAVNITNVAVLDNPTAFLNPFQFEISYECLVPLDDDLEWKLIYVGSAEDENYDQQLESVLVGPVNVGTYRFVLQADPPDPSKIREEDIIGVTVLLLTCSYMGQEFMRVGYYVNNDYDDEQLREEPPAKVLIDRVQRNILADKPRVTKFPINFHPEPSTGPGQQQQEPQTTSPENHTGNGEANGSKPEADQ

SEQ ID NO: 170 - DNA, 50 bp, Artificial sequence
  primerprm09810
ggggacaagtttgtacaaaaaagcaggcttaaacaatgagcgcggtgaac

SEQ ID NO: 171 - DNA, 47 bp, Artificial sequence
  primerprm09811
ggggaccactttgtacaagaaagctgggtcatccccactctgatcat

SEQ ID NO: 172 - DNA, 59 bp, Artificial sequence
  primerprm09544
ggggacaagtttgtacaaaaaagcaggcttaaacaatgagctctatcaatatcactaac

SEQ ID NO: 173 - DNA, 50 bp, Artificial sequence
  primerprm09545
ggggaccactttgtacaagaaagctgggtagttaagcttgagtcaaacaa

SEQ ID NO: 174 - DNA, 2194 bp, Oryza sativa; identical to SEQ ID NO: 9

SEQ ID NO: 175 - DNA, 5024 bp, Solanum lycopersicum
gaagcgaatggtatcgcggataaaggataaagttgatgcttaatcgatcggtttttcgttgtcggcgatgtctaagcataaggaaagaagtttgaatgagttatacaatgtgactttcactctttctaaacccaagattactcctctattgagaggaagccatcggatgcaagggccgtttgttgaacctatctgtagcttccagacgaatacggtgagattacagggtgtcaactgaagtcaataaggaatctatcccgtgtactcagaatcagactgtcggtggaagactagtcgctggatcgtgtaacgtgtgctccactccttgctcttcttgttttcctgctagtcaaagtctcatggagtcaaaagttgatgaattatctggggaaacaggtataaatagcttaagtttctctgtcaatgatgtttcatcttctgataagactagaaaatgtgaaattagacagagtagtgaaatagacagcgcaatctgcactagttcaagtagcttatctttctctgcaaacgctgaagttaaagcaaatgcaaggacttctgatgtttcatcagttacttcagatggtgcagtgcttgtggagttaaaggatctcaaatcttttgaaggccttgatgacaacatgtcgtgtatcgttggaggctatgaagctaataaattatccagcttcagtaagatgagggaagacaaatcaagtcttcagtgttcttctacttctactgggaaaactataaataatcaaacttctgctggatgtgtacacgtgaaagttgaggctgatgatggtagtccaattgaccatagtaggcagaatgaaagcagtggggaagaaaataataaggctcctactgaggcgacctcttcaagaaatgtacatagtacgggagactgtttggaaaataaccattcatcattaaagaatgacgtgaaatctgaagcttctgatgatctacctgctgatacttgtcctgagaagaatgaccaaaagaatgttggatcacctgtgtcctctgatacaaagaatgccttacaatcacatcaaatggatgagagtgaggaatccgacgttgaggagctagatgtgaaagtttgtgatatatgtggagatgctggtcgggaggatttacttgccatatgttgtaaatgtacagatggtgcagtacatacttattgcatgcgagaaatgttacaaaaagttccagagggtgattggatgtgcgaggaatgcaaatttgatgaggaaatgagaaatcggaaagaagataaatctgtgaagtttgatggaaatggaaaaagttatcctactggtcaaaaaattgcagttggcaatacaggccttaccataaaaacggaatcggagcctcctgattttgacggtgatatagcttctgaccctaaaactcctggcaagaggcgcatggatgatactgaatattctgcagcaaagaagcaggctcttgaaccagttccggcatcacccaaaacactgagtcccaataaacttcctgccctttctcgtgaaagttcatttaaaaactcagataaggggaagttgaaatctgcaaatcagatttcttctggaggtctttctgttcatgatacgccagcttgggggtcacgactacaaacttctagaggtactttttccaagtcaaattctttcagttccctggctgcaaaacgaaaagtgctacttgtagatgaaggttttctgccgaagcagaaattggtcagagagtccactggtcttgatgtgaaagagagttctactcgatcaatgaacaaatctatgtcatttagatcgataagcactagccgcaacaatgtctctgaatcaaaagttaagatgttatcccccaagtttccctctgctcaggacaaaggacaaatgcagacaaaagaacgaaatcaatttgaaaggaagaattcttttagatcagagcgttctcccggtacttctgttccttctagaaccgatcaaagatcagcatttcgaggtgacccttcgccacttccttcctcaagtaacatccgtgatacgcgaacaggtcagcttgacagcaaacctatgtcactattgaaatcttctggtgccgttgctcgtaggacacaagatatatctgttcattcagatgaagctaagaagaaaacatctcacacatccatgtccacaggagctcctgctaccaataaaattagtagctctgatcagcgacctgaccagagtagtgcaagagatgattctttgccgaactcttatattgctgagagaccaacatctaacactggtgaaggtctgtctgatggtctgccccagccgagtgaatcaaaaaatgttggtgagaggacaaaggagagttctgggagacgcttaaaacacactggaactggtactaagtcactcttctgccagaagtgtaaaggaagcggtcacttgacagatggttgcactgttgaggtgtctgaattattttcttctgatgtttctgctgtaagaaattctagagaggccccaaatggcacgagcaatcttaaagctgcaattgaggcggctatgctaaagaagcctggagtatgctggaagaatagggttgttgatcaatctgatgatttagctgtgtcaaacacaaatgctgaaacaacagctccggatccattatgtggttcaagtagcagaagaatgttgtcatccaacgaggatggccatggggtgccattaaactctattactggctctcataaacaggaaatcggtagcttgaggcagctgtcagtgcttcctgctgaagcccttaccggagcagggaatctggtgcctattcttctgtctgatggaaaatcttcacttgttgatttacatagatattcacaagcagcaatgtcgatactttcgaagacagcatttccagagcatgaatatatatggcagggtgcttttgaggttcagaagagtggaagaactcttgacttatgtgatggaattcaggctcatttatcaagttgtgcatcacccaatgttcttgacgcagtacacaaatttcctcaaaaggtcctctttaatgaggtatcacgatcgagtacatggccaatacaatttcaggagtatggtgttaaagaagataatattgcactgttcttttttgctcaagatgttggaagctatgagagatgctacaaaattttgctggagaatatgattaggaacgacacggctctcaaagcaaatcttcaaggtgttgaacttctgatattcccatctaaccgacttcctgaaaaatctcaacggtggaatatgatgttcttcctatggggtgtctttagagtgaagaaggtgcaggcaacgactggaaagccatctcttgtaccccaagatactccaaaattaatcatgccttttccggagaatatacattgtctcggacctgtagacaatgttacaagtggtaatgttcccatggatgttgaggtaactactccaaagaagtctagctgtccattagttaatggaaatgttgattctatagcggcccaagtatgcaaaggtgactctgcacacacaaatttggagcatctggagcctagatccatgagttctgtaccggtcagccacatggatgttgccccagagaggagacagtttggcattttccaggtggttggagatgctggacgtgaatgcaaagtggaagtgccaagtaattctgcaccagctgccaattctcagccatcccgctctgttaatgaagctgcaggtcatatgcaggagaaaacatctgtgggcagcatggagaaaggcttctgtagcacaaatggtaggaaatttgagataaatctggaagacgagtataaagatgaagaggcatctgaaacgagtggaagtgcagctacggaaccgacacggaaggagctcaataatgatgtgtcgaaccacctgaaacgtccacgttcagtggacactgtgatgcaatatgctgactctggagttaatcgagcaactcgactttttaacgataatgaccaagttgaagaggcacaccatgacaaaaagttgaagactagtattggtgggtcttatggtaatagcgagcaaactagttccagtgatgattttttgtcacggatgcgtggttcctcttatggaccctaccttccggatactgggtatgatgaagttctgagtaaggcacctgttccggagtgcacagagagtgctgaaagatatttcttccctgttgatccaaatcctggtaaggctagctcgacgccttggcaaatgcatcatccagacaatgatcggcttagtgatagagtcccgaatcttgagctagcattaggtggtgagtcaaattcacagactcggggaatcccaccctttttagttgggaaagtagacaagaaaattattcagctccaaggtggtgagacacaatcgctgacccagggcatcccaccctttttagttgggaaagtagacaagaaaatcattaaggaccatggtggtgagacacatccggcaactccaggaatcccatcctttttagttgggaaagtagacaagaaagtgagtcaggaccattcttcagctaaggaagcagttggagtggaggaggtagaggatgtctctgcttctctctccctttctctttcatttcctttcccggaaaaggaacaacagaaaggttctgtttcacaaactgagcaggcaatatctgaaacaagacgcggtaatacacctctcctcttctttgggggactcggcaacaagtaggagggcgatcttgccaataatggtgtacattttagttttattcacatggatgcggtaggtttacagcatgttcataaggatacaggggctataattttgttaggtgtttgcagtcaatttctctttttagtttgcaagtgcagagtgacggcccttttgtatatataaagagaatattagccgttgtgggctctggtaccatagaaaaaattaacatttatgcgtcagttaattctg

SEQ ID NO: 176 - protein, 1475 aa, Solanum lycopersicum
MESKVDELSGETGINSLSFSVNDVSSSDKTRKCEIRQSSEIDSAICTSSSSLSFSANAEVKANARTSDVSSVTSDGAVLVELKDLKSFEGLDDNMSCIVGGYEANKLSSFSKMREDKSSLQCSSTSTGKTINNQTSAGCVHVKVEADDGSPIDHSRQNESSGEENNKAPTEATSSRNVHSTGDCLENNHSSLKNDVKSEASDDLPADTCPEKNDQKNVGSPVSSDTKNALQSHQMDESEESDVEELDVKVCDICGDAGREDLLAICCKCTDGAVHTYCMREMLQKVPEGDWMCEECKFDEEMRNRKEDKSVKFDGNGKSYPTGQKIAVGNTGLTIKTESEPPDFDGDIASDPKTPGKRRMDDTEYSAAKKQALEPVPASPKTLSPNKLPALSRESSFKNSDKGKLKSANQISSGGLSVHDTPAWGSRLQTSRGTFSKSNSFSSLAAKRKVLLVDEGFLPKQKLVRESTGLDVKESSTRSMNKSMSFRSISTSRNNVSESKVKMLSPKFPSAQDKGQMQTKERNQFERKNSFRSERSPGTSVPSRTDQRSAFRGDPSPLPSSSNIRDTRTGQLDSKPMSLLKSSGAVARRTQDISVHSDEAKKKTSHTSMSTGAPATNKISSSDQRPDQSSARDDSLPNSYIAERPTSNTGEGLSDGLPQPSESKNVGERTKESSGRRLKHTGTGTKSLFCQKCKGSGHLTDGCTVEVSELFSSDVSAVRNSREAPNGTSNLKAAIEAAMLKKPGVCWKNRVVDQSDDLAVSNTNAETTAPDPLCGSSSRRMLSSNEDGHGVPLNSITGSHKQEIGSLRQLSVLPAEALTGAGNLVPILLSDGKSSLVDLHRYSQAAMSILSKTAFPEHEYIWQGAFEVQKSGRTLDLCDGIQAHLSSCASPNVLDAVHKFPQKVLFNEVSRSSTWPIQFQEYGVKEDNIALFFFAQDVGSYERCYKILLENMIRNDTALKANLQGVELLIFPSNRLPEKSQRWNMMFFLWGVFRVKKVQATTGKPSLVPQDTPKLIMPFPENIHCLGPVDNVTSGNVPMDVEVTTPKKSSCPLVNGNVDSIAAQVCKGDSAHTNLEHLEPRSMSSVPVSHMDVAPERRQFGIFQVVGDAGRECKVEVPSNSAPAANSQPSRSVNEAAGHMQEKTSVGSMEKGFCSTNGRKFEINLEDEYKDEEASETSGSAATEPTRKELNNDVSNHLKRPRSVDTVMQYADSGVNRATRLFNDNDQVEEAHHDKKLKTSIGGSYGNSEQTSSSDDFLSRMRGSSYGPYLPDTGYDEVLSKAPVPECTESAERYFFPVDPNPGKASSTPWQMHHPDNDRLSDRVPNLELALGGESNSQTRGIPPFLVGKVDKKIIQLQGGETQSLTQGIPPFLVGKVDKKIIKDHGGETHPATPGIPSFLVGKVDKKVSQDHSSAKEAVGVEEVEDVSASLSLSLSFPFPEKEQQKGSVSQTEQAISETRRGNTPLLFFGGLGNK

SEQ ID NO: 177 - DNA, 4607 bp, Populus trichocarpa
caggttgaaaaaggattaggcaaaccttccatgagacggaaagttcgtaccagcactgagtctgggacctgtaatgtgtgctctgctccctgttcatcttgtatgcatcttaagctagcctgtatgggatcaaagggtgatgagttttctgatgaaacctgtcgtgtaactgcatcaagtcagtattctaataatgatggtgatggtttagtctcgtttaaaagtagagcacgtgacagcttacagcataccaccagtgaagcaagcaacccgctcagtgtcagttcaagtcatgattctctgtctgaaaatgcagaaagtaaagtaaacagaaagtcatctgatgctgatgcgtcagctgagtctcagatgcgtccgaagatgtcctctggtagagctgttgcagaggatcagttttctccaaaagcagagagttttccagatcagaaaactttctcaaagaacaatgtggattctaaatctgaagagggccatgatgataacatgtcatgtgttagtagagctaatgatgcaagcaaagtggttagttattataacaagaatttagacatgaaaaattgtttgcccagttcagctttagaagtggaaggatctggaaaggcaccattttctcataaatcaggttcatttgagactccttcaaatgatgttgatgcttgcagtagctcaccaaaggtacagactaagtgcctttcctctaattcaaatggtaaacatttagatgaagatccagctttacatgaccatggaaaacggtttgaatgtccaacagaacaagtcaatctgtcattgtcaaaagaagcatcagctaatattgattgtgttggcaacttggctgcacacaacattgctgataacaatgcaaatggtaaaagtaccctcaatgcagatagttccaaggtttcatgtaaaatcaattcaaaattagaattagaggcagacgaagatagtggggaccaagcagatgagggttttaaatgttctgaccaagttgaacgaaaagagaagttgaatgagtcagatgagttagcagatatgcaggagcctatgttgcaatctgcatctggggatgagagtgacgagtctgaaattctggaacatgatgtaaaagtgtgtgatatttgcggggatgcaggtcgggaggattttcttgccatatgtagtaggtgcgcagatggtgcagaacacatctattgtatgcgagagatgcttcagaaacttcctgaaggtgactggttgtgtgaagaatgcaagttggctgaggaagctgaaaatcaaaagcaagatgctgaggaaaaaaggatgaacgtagcaagtactcagagctctggcaagagacatgcagaacatatggagctggcttcagcacccaaaaggcaggcaactgaatcaagtttggcatcacccaagtcatgcagtcctagcagaatagctgcagtgtctcgggatacttcattcaagagcttagataagggaaaagtaaagatagctcatcaaacatcttttggcaatcgctccaatattgatattccggaaattgcgcgcccttctgtgaatggtccacatgttcaaactcccaagggggccttattgaagtccaagtcgttcaacaccttaaattccaaaatgaaagtgaaacttgtcgatgaagttcctcaaaagcataagggcgcgagagagagttctcttgatatgaaggagggggctgctagaatgatgaggaaaagtatgtcatttaaatctgcgagctcaggccgatccagcactaatgagttgaaagttaaaatgctttcatccaaattctctcacattcaagattcaagaggattgaaacaagtgaaagactgggatgctgttgatagaaaaaaaatgttgagattgggtcgccctccaggtagttcaatgacaagtagtgctgttgtttcaacacctaaggtcgatcaagggttcactcctcgtggtgaaagtgtcatagcatcatccacaggcaacaacagagagttgaagtccgcacaatctaatggaaaattgggtaccctatcaagatcaactagcaatgtaggttgtaaaggtgcagatacttcagttacttcagttcaagcctcgtctaaaaatggaataagcagtaattctgcggaacaaaaattgaaccaaattagccccaaggatgaaccctcatccagttcttggaatgctgccagtaatgctactgaaaatttgcaagatggcctacctcgatcacgggaatcatcaaatcaaggtgaaaaggctagggaaaattctctcagtcgcttgagacctactggtatcactgggttgaaaaatgtcccttgtcaaaagtgcaaggaaatttgtcatgctacagaaaattgcactgttgttagtcctttggcttctggtactgatgtatctgcttctagaattcctagagaggagatgagcaaaggtagaaaattgaaagctgcaattgaggctgctgctatgcttaagaagcctggaatatacagaaagaagaaagaaattgatcaatctgatgggttgtcctcatcaaatgtggatgaaagtggcgagatggcttctcaagatcagctctcagttttaaataagttgagtgaaggaacagatgaagggcaagcaaatatcggtgcttcttcctctgagttttgcaaatcgacaattattaataatgtgaagcagcttaatgagcactccaatgatgctgtatgtcctttcaaggtggggtcagattccattgccccttatttgggaacgtctgttcatgcttcagcagagaagtctgtccttacaaagatgtcagctatcccagagcatgaatatatctggcagggggtgtttgaagtgcatagagctgaaaaggttgttgacttatatgatggaattcaagcacatctatctacttgtgcatctcctaaagtccttgatgtggttagcaaattcccccagaaaattaagttggatgaagtacctcgcattagcacatggccgagacaattccttgtcactggtgccaaagaggaaaatattgctctttacttctttgcaaagaattttgaaagttatgagaactacaagagattgttagacaacatgattaaaaaggatttggccctcaaaggatcatttgaaggtgttgaattcttcatattcccatccacacagcttccagagaactcacagcgctggaacatgttatatttcttgtggggagtgttcaggggaaggagatctgattgttcagattcattcaagaagttagttatgcccagtttgaatggggtgcccagggacaaagacattcccgctgcagtcatgacttcatctgagaatctctgtgtgcctgaatgtatagttaaaaatacatctgcatgtgacagtccatgttcttctgatgtgcatcttgcagcaaatgctcctgagaaaccaagtgtttccttaaatgggaactctgatgacaaagtatttaattcacaaaccaacctagagaagcaagatggtaaagttgactccagatcgttgacaaagattcgaggaagcagtaccccatggtgcccagaagctagatgcagcagtccttccctggaagaagttggtcctcctaggtgcagtctggatgtggacccgaaaccctgtactgaggtaactcggactaattctgtctctgatgtgaaggagatacaaattcatgaaggtgcttcatgtcttggagaagatatgcccttcaagatttttggtgttggtagtcaaaattcaggctgcaggaggatttttggtgaggataaaatagttgatagaacattcagtgacaaagataatattatagttgaaagggacttgaatgaagataacgtgaatatagatgtggagactttctcagggaaaggtccaaggaaacgaccatttttgtatttgtcagatactgcacctctgatttcgagtagcatgactcaaaaggctccgtggaacaaggcagataataataatacgttggtggatggagagagcatcagtaagaagctgaagacgggttttagtgggctatatgggggtagtggttcaagagaggaaaattctttgagcggtagttttacttcacagacatgtgatttgggttccagctcctccgttgaggagaggagctatgacaaagcatctgctgaaaaggtaatcttggagggcctgggaactagtgaaaggtacttctttcccgtggattcacatcatgtcaaggatagtcggttgcctgctatcttcatgccctggaactcatcaaatgatgaggatcgagttcgtgatgggattccaaatcttgagcttgccttaggagctgagacgaaatccccaaacaagcgaatcctgcctttctttggaatggctgaaaaaaatcatatccagaacaagcctccagacaaggtaatgaacaaggaagaagaagatggtgtctctgcttccctttccctctccctctcattcccattcccagactaggaacaaactgtaaaacctgtttcaaaaactgagcaacttgtgcctgaaaggtgtcatgtgaatacttcactgctcctctt

SEQ ID NO: 178 - protein, 1498 aa, Populus trichocarpa
MRRKVRTSTESGTCNVCSAPCSSCMHLKLACMGSKGDEFSDETCRVTASSQYSNNDGDGLVSFKSRARDSLQHTTSEASNPLSVSSSHDSLSENAESKVNRKSSDADASAESQMRPKMSSGRAVAEDQFSPKAESFPDQKTFSKNNVDSKSEEGHDDNMSCVSRANDASKVVSYYNKNLDMKNCLPSSALEVEGSGKAPFSHKSGSFETPSNDVDACSSSPKVQTKCLSSNSNGKHLDEDPALHDHGKRFECPTEQVNLSLSKEASANIDCVGNLAAHNIADNNANGKSTLNADSSKVSCKINSKLELEADEDSGDQADEGFKCSDQVERKEKLNESDELADMQEPMLQSASGDESDESEILEHDVKVCDICGDAGREDFLAICSRCADGAEHIYCMREMLQKLPEGDWLCEECKLAEEAENQKQDAEEKRMNVASTQSSGKRHAEHMELASAPKRQATESSLASPKSCSPSRIAAVSRDTSFKSLDKGKVKIAHQTSFGNRSNIDIPEIARPSVNGPHVQTPKGALLKSKSFNTLNSKMKVKLVDEVPQKHKGARESSLDMKEGAARMMRKSMSFKSASSGRSSTNELKVKMLSSKFSHIQDSRGLKQVKDWDAVDRKKMLRLGRPPGSSMTSSAVVSTPKVDQGFTPRGESVIASSTGNNRELKSAQSNGKLGTLSRSTSNVGCKGADTSVTSVQASSKNGISSNSAEQKLNQISPKDEPSSSSWNAASNATENLQDGLPRSRESSNQGEKARENSLSRLRPTGITGLKNVPCQKCKEICHATENCTVVSPLASGTDVSASRIPREEMSKGRKLKAAIEAAAMLKKPGIYRKKKEIDQSDGLSSSNVDESGEMASQDQLSVLNKLSEGTDEGQANIGASSSEFCKSTIINNVKQLNEHSNDAVCPFKVGSDSIAPYLGTSVHASAEKSVLTKMSAIPEHEYIWQGVFEVHRAEKVVDLYDGIQAHLSTCASPKVLDVVSKFPQKIKLDEVPRISTWPRQFLVTGAKEENIALYFFAKNFESYENYKRLLDNMIKKDLALKGSFEGVEFFIFPSTQLPENSQRWNMLYFLWGVFRGRRSDCSDSFKKLVMPSLNGVPRDKDIPAAVMTSSENLCVPECIVKNTSACDSPCSSDVHLAANAPEKPSVSLNGNSDDKVFNSQTNLEKQDGKVDSRSLTKIRGSSTPWCPEARCSSPSLEEVGPPRCSLDVDPKPCTEVTRTNSVSDVKEIQIHEGASCLGEDMPFKIFGVGSQNSGCRRIFGEDKIVDRTFSDKDNIIVERDLNEDNVNIDVETFSGKGPRKRPFLYLSDTAPLISSSMTQKAPWNKADNNNTLVDGESISKKLKTGFSGLYGGSGSREENSLSGSFTSQTCDLGSSSSVEERSYDKASAEKVILEGLGTSERYFFPVDSHHVKDSRLPAIFMPWNSSNDEDRVRDGIPNLELALGAETKSPNKRILPFFGMAEKNHIQNKPPDKVMNKEEEDGVSASLSLSLSFPFPD

SEQ ID NO: 179 - DNA, 2270 bp, Oryza sativa
cggcacgaggtctaaagacacctgtgtggtgaaggcatcagatccactaatcccaatggataaaataaaaaatgatagcacagatggtgcatgtgaaagtccactaattttgctgaataacgataatgaaatgtcaactaaacctgaggtgctttccattccacgtgcttcaaagacttgtggatctgatttccaagatattgcgccaacaagttcctcagaagatttgcctccagaagaggtacagtatgaacagaaagttgtggaaagtgatgggaacatctcctgtaaaagtgcagcggcgattcaggcttccgaagaccttttgccggagagcccacaaggctgtctagtggcacaaaatccgtacagccctgacactaaatcgaatgacctgaacttaaagcagcaagctttggttgatcaatcttctactgttggaagttctttgggggctttagttattccagagcagtcttacatctggcaaggtacctttgaggtttcaagacctggaagctctcctgaaatgtacgatgggtttcaggctcacttatctacctgtgcatcgctgaaagtacttgaaatagtgaaacaattacctcagagaattcagttggtagaagttccacggcattcctcatggccactgcaatttaaggaagtaaagccgaatgaagataacattgctctttatttctttgctaaagatgttgaaagttatgaaagagcatatgggaaactgttggaaaacatgcttgctggagatttgtccctcacagcaaatatttgtggcattgaacttctcatttttacgtctgataagctgcctgagaggactcaacggtggaatggcttacttttcttttggggtgtcctttatgccagaaaggcaagtagttcaactgagctgcttgtcaaagggatgaatcatagtccattagaacaaattaatggacctgttaatcaacttgtctgttcccctaagatgcctcagtctttgggcatagatttgaatgagtgccctgttgatgaattgtatgatccagctgtatcagttcaaacggagatggagaatcgtggtgcatctgtaaaccatgagactttgttgaggtccaaccatgaggctgaaaggctgaatttatgtgaaatacatttcccagaaactgcagggactgggaaaattttgttaggaactcctactgcagttccctatggagttcatgttcacacaagttcaaaacgtgaatgcctcaacattaaaccagaatatccaagtgatataataggtagcgaaggaacagcgggcagggacaacatggaggaggaagagagctttaccaagaatggagttccatgctttactaagcagcatacaggtgcaaccaccagatcagtatctgatgagatattggcaaatacacaggcacgcgtatcctttcaagaagtatccccacagcattctgtcaggccaaagctttctgatgatccaagtgattcagttttaaaggactttgttttgcctgattctagttccatctacaaacggcaaaagacctctgagggaaaatactctacttgcagttttggagatggtcaactgactagcaaatgcttgtccaagatacccttgccagctgatcagcatacttcattagatgatgtgcaatatattggtagggttccagcagatccctgttctccaactaaaccaatcttggatcatgtgatccatgtcctatcttcagatgatgaagactccccagaacctcgtaataatctgaataagacatcactgaaggaagaagagggcccttctcctctactgtcactgtccctttctatggcctcaaagaagcataatcttaccggttctgatacaggagatgatggaccgctgtctctgtctcttgggctccctggtgtagtgactagcaaccaggctcttgagatgaagcagtttctgccagagaaacctggcatgaacacttcattgcttctctagatatttgagtgtacagttttgtgctgtgttttactttatggggtttagacagggtagtagtatggtatagctgttaaattatggaaacctttggtttatttcactgttctatgcatctggttgttgctagagatgctggtctggtgatggtattaggcttctgcattttgtgtgattatggtgttttagttcccaaacgaactggtatgacataaaggatagatcagaatgtgaagtgggataaatgcaagttttttccatgctt

SEQ ID NO: 180 - protein, 649 aa, Oryza sativa
MDKIKNDSTDGACESPLILLNNDNEMSTKPEVLSIPRASKTCGSDFQDIAPTSSSEDLPPEEVQYEQKVVESDGNISCKSAAAIQASEDLLPESPQGCLVAQNPYSPDTKSNDLNLKQQALVDQSSTVGSSLGALVIPEQSYIWQGTFEVSRPGSSPEMYDGFQAHLSTCASLKVLEIVKQLPQRIQLVEVPRHSSWPLQFKEVKPNEDNIALYFFAKDVESYERAYGKLLENMLAGDLSLTANICGIELLIFTSDKLPERTQRWNGLLFFWGVLYARKASSSTELLVKGMNHSPLEQINGPVNQLVCSPKMPQSLGIDLNECPVDELYDPAVSVQTEMENRGASVNHETLLRSNHEAERLNLCEIHFPETAGTGKILLGTPTAVPYGVHVHTSSKRECLNIKPEYPSDIIGSEGTAGRDNMEEEESFTKNGVPCFTKQHTGATTRSVSDEILANTQARVSFQEVSPQHSVRPKLSDDPSDSVLKDFVLPDSSSIYKRQKTSEGKYSTCSFGDGQLTSKCLSKIPLPADQHTSLDDVQYIGRVPADPCSPTKPILDHVIHVLSSDDEDSPEPRNNLNKTSLKEEEGPSPLLSLSLSMASKKHNLTGSDTGDDGPLSLSLGLPGVVTSNQALEMKQFLPEKPGMNTSLLL

SEQ ID NO: 181 - DNA, 2194 bp, Oryza sativa; identical to SEQ ID NO: 9

SEQ ID NO: 182 - DNA, 56 bp, Artificial sequence
  primer prm18894
ggggacaagtttgtacaaaaaagcaggcttaaacaatggagtcaaaagttgatgaa

SEQ ID NO: 183 - DNA, 50 bp, Artificial sequence
  primer prm18895
ggggaccactttgtacaagaaagctgggtgtacaccattattggcaagat

SEQ ID NO: 184 - DNA, 56 bp, Artificial sequence
  primer prm18896
ggggacaagtttgtacaaaaaagcaggcttaaacaatgagacggaaagttcgtacc

SEQ ID NO: 185 - DNA, 50 bp, Artificial sequence
  primer prm18897
ggggaccactttgtacaagaaagctgggttgctcagtttttgaaacaggt

SEQ ID NO: 186 - DNA, 59 bp, Artificial sequence
  primer prm18908
ggggacaagtttgtacaaaaaagcaggcttaaacaatggataaaataaaaaatgatagc

SEQ ID NO: 187 - DNA, 50 bp, Artificial sequence
  primer prm18909
ggggaccactttgtacaagaaagctgggtcagcacaaaactgtacactca

SEQ ID NO: 188 - DNA, 429 bp, Arabidopsis thaliana
atggccggaattggaccgattactcaggattgggaaccagttgtgatccgcaagagagctcctaacgctgcagctaagcgcgacgagaagactgtcaacgccgctcgtcgaagcggcgccgatattgagaccgttcgaaaattcaatgctggatcgaacaaggctgcatcaagcggcacctccttgaacacaaagaagctagatgatgatactgagaacttatctcatgatcgtgtgcccactgaattgaagaaagccatcatgcaagctagaggggagaagaagctgactcagtcccaacttgcccatctgatcaatgagaagccacaagtgatccaagaatacgagtctgggaaagcaattccgaatcaacagatcctttcaaagctggagagggcacttggtgctaaactccgtggaaagaagtag

SEQ ID NO: 189 - protein, 142 aa, Arabidopsis thaliana
MAGIGPITQDWEPVVIRKRAPNAAAKRDEKTVNAARRSGADIETVRKFNAGSNKAASSGTSLNTKKLDDDTENLSHDRVPTELKKAIMQARGEKKLTQSQLAHLINEKPQVIQEYESGKAIPNQQILSKLERALGAKLRGKK

SEQ ID NO: 190 - DNA, 429 bp, Arabidopsis thaliana
atggccggaattggaccgataactcaggattgggagccggtggtgatccgtaagaaacccgctaacgccgctgccaagcgcgacgagaaaactgtcaacgccgctcgtcgatccggcgccgatatcgagaccgtcagaaaattcaatgctggaaccaacaaggcggcatcaagcggcacatctctgaacacaaaaatgcttgatgatgacactgagaaccttactcatgaacgtgtgcctactgagctaaagaaagccattatgcaagccaggacagacaagaagctaacccagtcccaacttgctcaaatcatcaatgagaagccacaagtgattcaagagtatgagtctggcaaagctatacccaaccagcaaatcctttctaagctggagagagcgcttggagctaagcttcgtggaaagaagtga

SEQ ID NO: 191 - protein, 142 aa, Arabidopsis thaliana
MAGIGPITQDWEPVVIRKKPANAAAKRDEKTVNAARRSGADIETVRKFNAGTNKAASSGTSLNTKMLDDDTENLTHERVPTELKKAIMQARTDKKLTQSQLAQIINEKPQVIQEYESGKAIPNQQILSKLERALGAKLRGKK

SEQ ID NO: 192 - DNA, 429 bp, Medicago truncatula
atgtcaggtctaggccatatttctcaagattgggaaccagtcgttatccgcaagaaagcacccaacgccgccgccaagaaagatgagaaagccgtcaacgccgctcgccgtgccggcgccgatatcgacaccgtcaagaaacataatgctgcaacaaacaaagctgcatctagcagcacttcattgaacactaagaggctggacgaggatactgagaatctagctcatgatcgtgtaccaactgaactcaagaaggctataatgcaagctaggatggacaaaaagcttactcagtctcagcttgctcaaatcatcaatgagaagcctcaagtgatccaagagtatgagtcagggaaagccattccaaaccagcagataattagcaagttggagagagcacttggagctaaactgcgtggcaagaaatga

SEQ ID NO: 193 - protein, 142 aa, Medicago truncatula
MSGLGHISQDWEPVVIRKKAPNAAAKKDEKAVNAARRAGADIDTVKKHNAATNKAASSSTSLNTKRLDEDTENLAHDRVPTELKKAIMQARMDKKLTQSQLAQIINEKPQVIQEYESGKAIPNQQIISKLERALGAKLRGKK

SEQ ID NO: 194 - DNA, 429 bp, Triticum aestivum
atggctgggattggtcctatcaggcaggactgggagccgatagtggtgcggaagaaggcgcagaacgccgccgacaagaaggacgaaaaggccgtcaacgctgcccgccgctccggcgccgagatcgacaccaccaagaagtacaacgctggaacgaacaaggctgcatctagcggaacttccctcaacaccaagcggctcgacgacgacacggagaacctttcccatgagcgtgtttcaagtgacctgaagaaaaaccttatgcaagcaagactggataagaagatgacccaggcacaacttgctcagatgatcaatgagaagccacaggtgatccaggagtacgagtcgggcaaggcgattccgaacaatcagataattggaaagctcgagagggcacttggagctaagctgcgtagcaagaagtaa

SEQ ID NO: 195 - protein, 142 aa, Triticum aestivum
MAGIGPIRQDWEPIVVRKKAQNAADKKDEKAVNAARRSGAEIDTTKKYNAGTNKAASSGTSLNTKRLDDDTENLSHERVSSDLKKNLMQARLDKKMTQAQLAQMINEKPQVIQEYESGKAIPNNQIIGKLERALGAKLRSKK

SEQ ID NO: 196 - DNA, 429 bp, Elaeis guineensis
atggccgggattggtccgatcacccaggattgggagcccgtcgtggtccgcaagaaggccccgaacgccgctgcgaagaaggacgagaaggccgtcaacgccgcccgacgcagtggtgccgaaatcgataccgtaaagaagtctaatgccggtacaaacaaggctgcttctagcagcacaactttaaacacaaggaagcttgatgaggatacagagagtctctctcatgaacgagtgccaatggagctgaagaagaatatcatgcaggctcgaatgggtaaaaggttgactcaggcacaacttgcgcagctgatcaatgagaagccccaagtgattcaagaatatgaatctgggaaggccattccaaatcaacaaataatcaccaaactcgaaagagttcttggggtgaaactgcgaggtaaaaaatga

SEQ ID NO: 197 - protein, 142 aa, Elaeis guineensis
MAGIGPITQDWEPVVVRKKAPNAAAKKDEKAVNAARRSGAEIDTVKKSNAGTNKAASSSTTLNTRKLDEDTESLSHERVPMELKKNIMQARMGKRLTQAQLAQLINEKPQVIQEYESGKAIPNQQIITKLERVLGVKLRGKK

SEQ ID NO: 198 - DNA, 429 bp, Elaeis guineensis
atggccggagtgggacctataacgcaggactgggagccggtggtgatccgcaagaaggcccccaacgccgccaccaagaaggacgagaaggccgtcaacgccgcccgtcgcagcggcgccgagatcgagaccctcaggaagtccactgctggtatcaatagagctgcatctagcagcacatcgctgaatacaaggaagcttgatgaagaaacagagactctttctcatgaacgagtaccatcagaactgaagaagaatatcatgaaagctcgaatggacaagaaattgacccaagctcagcttgcacagctgatcaatgagaagcctcaagtgattcaagagtatgaatcagggaaggctattcctaatcaacagatcataatcaaactggaaagggttcttggagcgaaactgcgaggtaaaaagtaa

SEQ ID NO: 199 - protein, 142 aa, Elaeis guineensis
MAGVGPITQDWEPVVIRKKAPNAATKKDEKAVNAARRSGAEIETLRKSTAGINRAASSSTSLNTRKLDEETETLSHERVPSELKKNIMKARMDKKLTQAQLAQLINEKPQVIQEYESGKAIPNQQIIIKLERVLGAKLRGKK

SEQ ID NO: 200 - DNA, 429 bp, Glycine max
atgtctggtgttggccctctttctcaggattgggaacctgtcgtcctccgcaagaaggctcccaccgccgccgccaagaaggacgagaaagccgtcaacgccgcccgccgctctggcgccgaaatcgaaaccctaaaaaagtataatgctgggacaaacaaagcagcatctagcggcacttcattgaacactaagaggctggatgatgatactgagagtctagctcatgagaaggtgccaactgaacttaagaaggctataatgcaagctaggatggacaaaaagcttactcagtctcagcttgctcaactgatcaatgagaagcctcaagtgatccaggagtatgagtcagggaaggccattccaaaccagcagataattagcaagttggaaagagctcttggagctaaactgcgtggcaagaaataa

SEQ ID NO: 201 - protein, 142 aa, Glycine max
MSGVGPLSQDWEPVVLRKKAPTAAAKKDEKAVNAARRSGAEIETLKKYNAGTNKAASSGTSLNTKRLDDDTESLAHEKVPTELKKAIMQARMDKKLTQSQLAQLINEKPQVIQEYESGKAIPNQQIISKLERALGAKLRGKK

SEQ ID NO: 202 - DNA, 429 bp, Gymnadenia conopsea
atggccggaattggtccaattacgcaggacagggagcccgtcattatccgcaagaaggcccctaacgcctctgccaagaaggacgagaaggctgtcaacgctgcccggcgaagcggcgcagagatcgaaactttaaagaagtctaatgcgggcaccaacaaagcagcttcgagtggaacaacgttgaatacaagaaagcttgatgaagaaacagaaaacctttctcatgataaggtgcccaccgagttgaagaagaacatcatgcaagctcgaatggacaaaaagctaacacagtctcagcttgcacagttgatcaatgagaaaccccaggtgattcaggagtacgagtcggggaaggcaattccaaatcagcagatcgtcagcaaactcgaaagagttcttggcgtgaaactgcgggggaagaaataa

SEQ ID NO: 203 - protein, 142 aa, Gymnadenia conopsea
MAGIGPITQDREPVIIRKKAPNASAKKDEKAVNAARRSGAEIETLKKSNAGTNKAASSGTTLNTRKLDEETENLSHDKVPTELKKNIMQARMDKKLTQSQLAQLINEKPQVIQEYESGKAIPNQQIVSKLERVLGVKLRGKK

SEQ ID NO: 204 - DNA, 429 bp, Hordeum vulgare
atggctgggattggtccgctcaggcaggactgggagccgatagtggtgcggaagagggcccagaacgccgcggacaagaaggacgaaaaggccgtcaacgctgcccgccgctccggcgccgagatcgacaccaccaagaagtataacgctggaacgaacaaggctgcatctagcggaacttccctcaacaccaagcggctcgacgacgacactgagaacctttcccatgagcgtgtttcaagcgacctgaagaaaaaccttatgcaagcaaggctggataagaagatgacccaggcacaacttgctcagatgatcaatgagaagccacaggtgatccaggagtacgagtcgggcaaggcgattccgaacaatcagataattggaaagctcgagagggcacttggagctaagctgcgtagcaagaagtaa

SEQ ID NO: 205 - protein, 142 aa, Hordeum vulgare
MAGIGPLRQDWEPIVVRKRAQNAADKKDEKAVNAARRSGAEIDTTKKYNAGTNKAASSGTSLNTKRLDDDTENLSHERVSSDLKKNLMQARLDKKMTQAQLAQMINEKPQVIQEYESGKAIPNNQIIGKLERALGAKLRSKK

SEQ ID NO: 206 - DNA, 429 bp, Hordeum vulgare
atgtctcgcacgggaccgatcgctcaggactgggagccggtggtcgtgcgcaagaagctgcccaacgccgccgccaagaaggacgagaaggccgtcaacgccgcccgccgcgccggcgtcgacatcgacatcgccaagaaacataatgctgggaccaacaaagctgctcatagcaccacatcgctcaatacaaagaggcttgatgatgatacagagaatcttgctcatgagcgtgtgccgtcagacctgaagaagagcattatgcaggctagaacagacaagaagctcacacaggcacagcttgcacagctgatcaatgagaagccacaagtcatccaggagtacgagtcaggcaaagctatcccaaaccaacagatcatcggcaagctggaaagggctcttggcacaaagctgcgaggcaagaagtga

SEQ ID NO: 207 - protein, 142 aa, Hordeum vulgare
MSRTGPIAQDWEPVVVRKKLPNAAAKKDEKAVNAARRAGVDIDIAKKHNAGTNKAAHSTTSLNTKRLDDDTENLAHERVPSDLKKSIMQARTDKKLTQAQLAQLINEKPQVIQEYESGKAIPNQQIIGKLERALGTKLRGKK

SEQ ID NO: 208 - DNA, 423 bp, Linum usitatissum
atgtcaggaccattctctcaggactgggaacccgtcgtaatccgcaagaaagctcccaccgccgctctcaagaaggacgagaaggtcgttaacgctgctcgccgcgccggcgctgagatcgaatccatcaagaagtcaaatgctggtgtgaacaaggctgcttctagcagtacttccttgaacacaaggaagcttgatgaagagactgagattgttgctcatgagcgggtaccgagtgaactgaacaaggccataatgcaaggtcgaatggataagaagcttacccagtctcaacttgctcagctcatcaatgagaagcctcagataatacaagagtacgagtccggaaaagccattcctaaccagcagattataggcaagttagagagagctcttggggtgaagctacgaggcaagaagtga

SEQ ID NO: 209 - protein, 140 aa, Linum usitatissum
MSGPFSQDWEPVVIRKKAPTAALKKDEKVVNAARRAGAEIESIKKSNAGVNKAASSSTSLNTRKLDEETEIVAHERVPSELNKAIMQGRMDKKLTQSQLAQLINEKPQIIQEYESGKAIPNQQIIGKLERALGVKLRGKK

SEQ ID NO: 210 - DNA, 423 bp, Nicotiana tabacum
atgagtggaggaatagcacaagactgggagccggtggtgatccgcaagaaggcgcctaccgccgctgcacgcaaggatgagaaagccgtcaacgccgcccgtcgctccggtgctgagatcgaaaccatccgaaaatctgctgctggcacaaacaaagctgcctccagtagtacgaccttgaacaccaggaaacttgatgaagatactgagaatttggctcatcaaaaggtaccaactgaactgaagaaagccatcatgcaagctcgacaagataagaagctgacccaggctcaacttgcccagttgataaatgagaagcctcaaatcatccaggagtatgagtctggaaaggcgattccaaatcaacagataatctctaaactggagagagctcttggtgcgaaacttagaggaaagaaatga

SEQ ID NO: 211 - protein, 140 aa, Nicotiana tabacum
MSGGIAQDWEPVVIRKKAPTAAARKDEKAVNAARRSGAEIETIRKSAAGTNKAASSSTTLNTRKLDEDTENLAHQKVPTELKKAIMQARQDKKLTQAQLAQLINEKPQIIQEYESGKAIPNQQIISKLERALGAKLRGKK

SEQ ID NO: 212 - DNA, 429 bp, Oryza sativa
atggccgggattggtccgatcaggcaggactgggagccggtggtggtgcggaagaaggcgcccaccgccgccgccaagaaggatgagaaggccgtcaacgccgcccgccgctccggcgccgagatcgagaccatgaagaagtataacgctggaacaaacaaggcggcgtccagtggcacatccctcaacaccaagcggctggatgacgacaccgagagccttgcccatgagcgtgtctcaagtgacctgaagaaaaacctcatgcaagcaaggctggacaagaagatgacccaggcacagcttgcacagatgatcaatgagaagccccaggtgatccaggagtacgagtcaggtaaagctattccgaaccagcagatcatcgggaagcttgaaagggctcttggaacaaagctgcgcggcaagaaataa

SEQ ID NO: 213 - protein, 142 aa, Oryza sativa
MAGIGPIRQDWEPVVVRKKAPTAAAKKDEKAVNAARRSGAEIETMKKYNAGTNKAASSGTSLNTKRLDDDTESLAHERVSSDLKKNLMQARLDKKMTQAQLAQMINEKPQVIQEYESGKAIPNQQIIGKLERALGTKLRGKK

SEQ ID NO: 214 - DNA, 456 bp, Picea sitchensis
atggctggagtaggaccgatcagtcaggattgggaacccgttgttatccggaagaaggctcccaacgctgcagccaagaaggacgagaaggcggtcaatgctgcccgtcgaaccggaggccccatcgaaactatcaagaaatttaatgcaggatcaaacaaagcagcctcgagcagcaccaccctgaacaccaggaagcttgatgatgagacagaagttcttgcacacgaaagagtttcaacggatttgaagaaaaacataatgcaggcccgtttagataaaaagttaacacaagctcagcttgcacagcaaattaatgaaaaacctcaaattattcaagagtacgagtctgggaaagcaattcccaatcagcagatcattgcaaagctggaaagggttcttagtgtgaaactgcgtggaacttctggaacttctggaacttctggaaagaaataa

SEQ ID NO: 215 - protein, 151 aa, Picea sitchensis
MAGVGPISQDWEPVVIRKKAPNAAAKKDEKAVNAARRTGGPIETIKKFNAGSNKAASSSTTLNTRKLDDETEVLAHERVSTDLKKNIMQARLDKKLTQAQLAQQINEKPQIIQEYESGKAIPNQQIIAKLERVLSVKLRGTSGTSGTSGKK

SEQ ID NO: 216 - DNA, 429 bp, Populus tremuloides
atgtcaggaggtggaccaatctcacaggactgggaacccgtagtgatccgcaagaaagctcccaacgccgccgccaagaaggatgagaaggccgtcaacgccgcccgccgctccggtgccgagatcgaaaccatcaaaaaatcaactgctggtacgaacaaggctgcttctagcagcacttctttgaacacaaggaagctcgatgaagaaacagagaaccttgctcatgaccgagtgccaactgaactgaagaaagcaattatgcagggtagaacggacaagaaacttacccaggctcaacttgcacagttgatcaacgagaagccccagataattcaggagtatgaatccggaaaagccattcctaatcagcagattataggcaaactggagagggctcttggtgtgaagctgcggggaaagaagtga

SEQ ID NO: 217 - protein, 142 aa, Populus tremuloides
MSGGGPISQDWEPVVIRKKAPNAAAKKDEKAVNAARRSGAEIETIKKSTAGTNKAASSSTSLNTRKLDEETENLAHDRVPTELKKAIMQGRTDKKLTQAQLAQLINEKPQIIQEYESGKAIPNQQIIGKLERALGVKLRGKK

SEQ ID NO: 218 - DNA, 423 bp, Populus tremuloides
atgtcaggaccaatctcacaggactgggagccggtggtgatccgtaagaaagctcccaacgccgccgccaagaaggatgagaaggccgtcaacgccgcccgccgcgctggtgctgagatcgaaaccgtcaaaaaatcaactgctggtacaaacaaggccgcttctagcagcacttctttgaacacaaggaagctcgatgacgaaacagagaaccttactcatgaccgagtgccaactgaactgaagaaagcaattatgcaggctagaatggacaagaaacttacccaggctcaacttgcacaggtgatcaatgagaagccccagataattcaggagtatgaatctggaaaagccattcctaatcagcagattataggaaaactggagagggctcttggtgtgaagctacggggaaagaagtag

SEQ ID NO: 219 - protein, 140 aa, Populus tremuloides
MSGPISQDWEPVVIRKKAPNAAAKKDEKAVNAARRAGAEIETVKKSTAGTNKAASSSTSLNTRKLDDETENLTHDRVPTELKKAIMQARMDKKLTQAQLAQVINEKPQIIQEYESGKAIPNQQIIGKLERALGVKLRGKK

SEQ ID NO: 220 - DNA, 429 bp, Ricinus communis
atggcaggagttggaccaatctcacaggactgggaaccagtagtcatccgcaaaaaggctcccaccgccgccgctaagaaggacgagaaggtcgtcaacgctgctcgtcgcgctggtgccgagatcgaaactctaaaaaaatctaatgctggtactaataaagcagcctctagcagcacttctttaaacacaaggaagcttgatgaagaaacagagaacctaactcatgaccgagtaccgactgaattgaagaaagccataatgcaggctcggatggaaaagaaatttacccaggctcagcttgctcagatgatcaatgaaaagccccagataatccaagagtatgaatctggaaaagcaattcccaatcaacagataataggcaaactggagagggcccttggtgtgaagctgcgaggaaagaaatga

SEQ ID NO: 221 - protein, 142 aa, Ricinus communis
MAGVGPISQDWEPVVIRKKAPTAAAKKDEKVVNAARRAGAEIETLKKSNAGTNKAASSSTSLNTRKLDEETENLTHDRVPTELKKAIMQARMEKKFTQAQLAQMINEKPQIIQEYESGKAIPNQQIIGKLERALGVKLRGKK

SEQ ID NO: 222 - DNA, 420 bp, Solanum tuberosum
atgagtggaatatcgcaagactgggagccggtagtaatcaggaagaaggcgcctacctccgccgctcgcaaggatgagaaagccgttaacgccgcccgtcgctccggcgccgagatcgaaaccgttaagaagtctaatgcaggctcaaacagggctgcctctagtagtacatcattgaacactaggaaacttgatgaagacactgagaatttgtctcatgaaaaggtaccaactgaactgaagaaagctatcatgcaagcacgacaagacaagaagctgactcagtctcaacttgctcaattgataaatgagaagccacagattatccaagaatacgagtcgggaaaggcaattccaaaccaacagataatctcaaaactggagagagctcttggagcgaaacttcgaggaaagaaataa

SEQ ID NO: 223 - protein, 139 aa, Solanum tuberosum
MSGISQDWEPVVIRKKAPTSAARKDEKAVNAARRSGAEIETVKKSNAGSNRAASSSTSLNTRKLDEDTENLSHEKVPTELKKAIMQARQDKKLTQSQLAQLINEKPQIIQEYESGKAIPNQQIISKLERALGAKLRGKK

SEQ ID NO: 224 - DNA, 429 bp, Zea mays
atggccgggatcgggccgatcaggcaggactgggagccggtggttgtgcggaagaaggcacccaccgccgctgccaagaaggatgagaaggccgtcaacgccgcgcgccgctccggcgcggagatcgagaccatgaagaagttcaacgctggtatgaacaaggcggcgtccagcggcacatccctcaacaccaagcgcctcgacgacgacacagagaacctcgcccatgagcgagttccaagtgacctgaagaaaaatctcatgcaagcaaggctcgataagaagttgacccaggcacagcttgctcagatgatcaatgagaagccacaggtgatccaggagtatgagtcaggcaaggcaattcccaaccagcagatcattggcaagctcgagagggccctgggaacgaagctgcgtggcaagaaataa

SEQ ID NO: 225 - protein, 142 aa, Zea mays
MAGIGPIRQDWEPVVVRKKAPTAAAKKDEKAVNAARRSGAEIETMKKFNAGMNKAASSGTSLNTKRLDDDTENLAHERVPSDLKKNLMQARLDKKLTQAQLAQMINEKPQVIQEYESGKAIPNQQIIGKLERALGTKLRGKK

SEQ ID NO: 226 - DNA, 429 bp, Zea mays
atggccgggatcggaccgatcaggcaggactgggagccggtcgttgtgcggaagaaggcacccaccgccgccgccaagaaggatgagaaggccgtcaacgccgcgcgccgcgccggtgcggagatcgataccatgaagaagtacaacgctggtacgaacaaggcggcatccagcggtacatccctcaacaccaagcgcctcgacgacgacaccgaaaacctcgcccatgagcgagttccaagtgatctgaagaagaatctcatgcaagcaaggctcgataagaagctgacacaggcacaacttgctcagatgataaatgagaagccacaggtgattcaggagtatgaatcaggcaaggcaatccccaaccagcagatcattagcaagctcgagagggccctgggaaccaagttgcgtggcaagaaatag

SEQ ID NO: 227 - protein, 142 aa, Zea mays
MAGIGPIRQDWEPVVVRKKAPTAAAKKDEKAVNAARRAGAEIDTMKKYNAGTNKAASSGTSLNTKRLDDDTENLAHERVPSDLKKNLMQARLDKKLTQAQLAQMINEKPQVIQEYESGKAIPNQQIISKLERALGTKLRGKK

SEQ ID NO: 228 - DNA, 435 bp, Allium cepa
atgccatcccgatcaaccggagcaatcgtccagcaatgggatccggtcgtcatctcccgccgtaaaccaaaaaccgctgatctgaaagacccgaaagtggttaacagtgcgatccgtgccggtgcgcaagtcgagacgatcaaaaagttcgacgctggtcagaacaagaagaaggctgagccggtggttaatgcgagaaagctcgacgaacagacggaacctgggacgctgaaccgtgtgccaggggaggtgagggcggagatccagaaggcgaggttggcgaagaagatgagtcaggcggagctggcgaagcagatcaacgagcgagtgcaggtggtgcaggaatatgagaacggtaaggctgttttaaaccagggtgttttggctaagatggagaaggttcttggggttaaactaaggggaaagcataagtaa

SEQ ID NO: 229 - protein, 144 aa, Allium cepa
MPSRSTGAIVQQWDPVVISRRKPKTADLKDPKVVNSAIRAGAQVETIKKFDAGQNKKKAEPVVNARKLDEQTEPGTLNRVPGEVRAEIQKARLAKKMSQAELAKQINERVQVVQEYENGKAVLNQGVLAKMEKVLGVKLRGKHK

SEQ ID NO: 230 - DNA, 447 bp, Arabidopsis thaliana
atgccgagcagatacccaggagcagtaacacaagactgggaaccagtagttctccacaaatcaaaacaaaagagccaagacctacgcgatccgaaagcggttaacgcagctctgagaaacggtgtcgcggttcaaacggttaagaaattcgatgccggttcgaacaaaaaggggaaatctacggcggttccggtgattaacacgaagaagctggaagaagaaacagagcctgcggcgatggatcgtgtgaaagcagaggtgaggttgatgatacagaaagcgagattggagaagaagatgtcacaagcggatttggcgaaacagatcaatgagaggactcaggtagttcaggaatatgagaatggtaaagctgttcctaatcaggctgtgcttgcgaagatggagaaggttctaggtgttaaacttaggggtaaaattgggaaatga

SEQ ID NO: 231 - protein, 148 aa, Arabidopsis thaliana
MPSRYPGAVTQDWEPVVLHKSKQKSQDLRDPKAVNAALRNGVAVQTVKKFDAGSNKKGKSTAVPVINTKKLEEETEPAAMDRVKAEVRLMIQKARLEKKMSQADLAKQINERTQVVQEYENGKAVPNQAVLAKMEKVLGVKLRGKIGK

SEQ ID NO: 232 - DNA, 420 bp, Chlamydomonas reinhardtii
atgaacatgaactctcaagactgggacaccgttgtgcttcgcaagaagcagcctactggcgcagcgctgaaggacgaagccgctgtcaatgcggcacggcggcaaggtgcagctgtggagacgtcgcagaaatttaacgctggaaagaacaagcctggtgcggctcagactgtgagcggcaagcctgcagccaagctggagcaggagacggaggacttccatcacgagcgcgtgtcttcgaacctcaagcagcagattgtgcaggcgcgcacggcgaagaagatgacccaggcgcagctagcgcaggctatcaacgagaagccgcaggtgatccaggagtacgagcagggcaaggccatccccaacccccaggtgctctcgaagctgtcccgtgcgctcggcgtggtgctgaagaagtaa

SEQ ID NO: 233 - protein, 139 aa, Chlamydomonas reinhardtii
MNMNSQDWDTVVLRKKQPTGAALKDEAAVNAARRQGAAVETSQKFNAGKNKPGAAQTVSGKPAAKLEQETEDFHHERVSSNLKQQIVQARTAKKMTQAQLAQAINEKPQVIQEYEQGKAIPNPQVLSKLSRALGVVLKK

SEQ ID NO: 234 - DNA, 441 bp, Lycopersicon esculentum
atgccgatgcgaccaacagggggattgaaacaagattgggatccaatcgtgctgcagaagccaaagatgaaggcccaagacctgaaggatccaaaaattgtgaatcaggcattgcgagctggagcacaagttcaaacggtgaagaaaatcgacgctggtttgaataagaaggcggcgacgttggcagttaatgtaagaaagctagatgaggcggcggaaccagcggcacttgagaaattgccggtgggtgtaaggcaagcaatacagaaagcgcggattgagaagaagatgagccaagctgatctagcgaagaagatcaatgaaaggacgcaggttgttgccgagtatgagaatggtaaggcagtgcctaatcaactagtgttggggaaaatggagaacgttcttggtgttaaacttagaggtaaaattcacaagtcatga

SEQ ID NO: 235 - protein, 146 aa, Lycopersicon esculentum
MPMRPTGGLKQDWDPIVLQKPKMKAQDLKDPKIVNQALRAGAQVQTVKKIDAGLNKKAATLAVNVRKLDEAAEPAALEKLPVGVRQAIQKARIEKKMSQADLAKKINERTQVVAEYENGKAVPNQLVLGKMENVLGVKLRGKIHKS

SEQ ID NO: 236 - DNA, 468 bp, Oryza sativa
atgccgacggggaggttgagcggcaacatcacgcaggactgggagccggtggtgctgcggcggacgaagccgaaggcggcggaccttaagtcgacgagggcggtgaaccaggcgatgcggacgggggcgccggtggagacggtgcggaaggcggcagcgggcacgaacaaggcggcggcgggggcggcggcgcccgcgcggaagctggacgagtcgacggagccggcggggctggggcgcgtgggcgcggaggtgcgcggcgcgattcagaaggcccgggtggcgaaggggtggagccaggcggagctcgccaagcgcatcaacgagcgggcgcaggtggtgcaggagtacgagagcggcaaggccgtccccgtccaggccgtgctcgccaagatggagcgcgcgctcgaggtcaagctccgcggcaaggcggtcggcgcgccggccgcgcccgccggcgccaagtga

SEQ ID NO: 237 - protein, 155 aa, Oryza sativa
MPTGRLSGNITQDWEPVVLRRTKPKAADLKSTRAVNQAMRTGAPVETVRKAAAGTNKAAAGAAAPARKLDESTEPAGLGRVGAEVRGAIQKARVAKGWSQAELAKRINERAQVVQEYESGKAVPVQAVLAKMERALEVKLRGKAVGAPAAPAGAK

SEQ ID NO: 238 - DNA, 429 bp, Physcomitrella patens
atggctgatctgagacaagattgggagcctgtggtggtcaggaagaaggctccaacttcgggtgcgaagaaggacgagaaggcagtcaatgcagcacgacgagctggcggcccaattgagactatcaagaaattcaacgctgggtccaacaaggcagctaccagtgctactggtttgaatacaaagaagcttgatgacgagactgacgttcttgctcacgagaaagtgcccacagaactcaagagaaaaattatgcaggctcggttggataagaagatgacacaggctcaacttgcacagctcataaatgaaaagccacaaattgtacaagagtacgagtcagggaaagcaattcccaaccaacagatcatatcgaaattggaacgtgtgcttggtacgaagctacgaggagcaggagctaaaaagtga

SEQ ID NO: 239 - protein, 142 aa, Physcomitrella patens
MADLRQDWEPVVVRKKAPTSGAKKDEKAVNAARRAGGPIETIKKFNAGSNKAATSATGLNTKKLDDETDVLAHEKVPTELKRKIMQARLDKKMTQAQLAQLINEKPQIVQEYESGKAIPNQQIISKLERVLGTKLRGAGAKK

SEQ ID NO: 240 - DNA, 429 bp, Physcomitrella patens
atgcctgccagaacagccggccctatctcccaagattgggcccctgtcgttgtacacaagcgacccgtgaaagctgctgatgctcgtgatccgaaagctattgctgctgcaattcgagctggtgcggaagttcaaacagtcaggaagttcgactctgggacaaacaagaagaccggtccttcattaaatgctcgaaagcttgacgaggaacatgagcccgcgccactggaacgtgtttcatctgagataaagcattctatccagaaagcccgtttggacaagaaactcacccaagctcagctggcgcaactgatcaacgagcgtccacaagttgtgcaagagtatgagtccgggaaagcaataccttcgcagcaagtgctcgccaagttggagcgcgccctgggtgtgaagttgagaggaaagaagtaa

SEQ ID NO: 241 - protein, 142 aa, Physcomitrella patens
MPARTAGPISQDWAPVVVHKRPVKAADARDPKAIAAAIRAGAEVQTVRKFDSGTNKKTGPSLNARKLDEEHEPAPLERVSSEIKHSIQKARLDKKLTQAQLAQLINERPQVVQEYESGKAIPSQQVLAKLERALGVKLRGKK

SEQ ID NO: 242 - DNA, 438 bp, Picea sinensis
atgccgagccgaacgaacgggcctataacgcaagactggacgcctgttgttatccacaagcgtcttcaaaaggcgagtgaagcgcgtgatcccaaggcggttaatgctgcgatcagagcgggcgcacaggttcagagcattaagaagtttgagggcggaagcaacaagaaggctcaacctccgctcaatacccggaaattggacgaggagactgagccggctgcccttcagaaagtaccggcagagattcgacatgctatacagaaggcgcgtcttgatcagaagctgagccaggcggagctggggaagcgtataaatgagagagcgcaagtaattcaggagtatgaaagtggtaaagctatccctaatcaggccattctgtctaagttggagaaggtcctcggcgtcaaattgaggggcaaactaaattctcactaa

SEQ ID NO: 243 - protein, 145 aa, Picea sinensis
MPSRTNGPITQDWTPVVIHKRLQKASEARDPKAVNAAIRAGAQVQSIKKFEGGSNKKAQPPLNTRKLDEETEPAALQKVPAEIRHAIQKARLDQKLSQAELGKRINERAQVIQEYESGKAIPNQAILSKLEKVLGVKLRGKLNSH

SEQ ID NO: 244 - DNA, 438 bp, Retama raetam
atgccaactcgagcaacaggaaccattacccaagactgggaaacagtagtcctccacaaatcaaagcccaaggcgcaggaccttcgcaacccgaaagccataagccaagccctccgagccggcgcagaggtccaaacaatcaaaaaattcgacgccggttcaaacgagaaaaccgccggtccggtcgtctatgcgaggaagctggatgaagcggctgaaccggcagcgttggagagagttgcgggcgaggtgaggcacgcgatacagaaggcgcgtttggaaaagaagatgagtcaggctgaggtggcaaaacagattaatgaaaggcctcaggtggttcaggaatatgagaatgggaaagcggttccgaaccaggccgtgttggctaagatggagagggtgcttggtgttaagcttaggggcaaaattggtaaatga

SEQ ID NO: 245 - protein, 145 aa, Retama raetam
MPTRATGTITQDWETVVLHKSKPKAQDLRNPKAISQALRAGAEVQTIKKFDAGSNEKTAGPVVYARKLDEAAEPAALERVAGEVRHAIQKARLEKKMSQAEVAKQINERPQVVQEYENGKAVPNQAVLAKMERVLGVKLRGKIGK

SEQ ID NO: 246 - DNA, 471 bp, Triticum aestivum
atgccgacgggcaggatgagcggcaacatcacgcaggactgggagccggtggtgctgcggcgggcgaagcccaaggcggccgacctcaagtccgccaaggcggtgaaccaggcgctgcggacgggcgcgccggtggagacggtgcgcaaggcggcggcggggacgaacaagaatgcctccgccgcggccgtggcggcgcccgcgcggaagctggacgagatgacggagcctgcggggctggggcgcgtgggcggcgacgtgcgcgcggccatccagaaggcgcgcgtggcgaaaggatggagccaggcggagctggccaagcgcatcaacgagcgggcgcaggtggtgcaggagtacgagagcggcaaggccgtccccgtccaggccgtgctcgccaagatggagcgcgccctcgaggtcaagctccgcggcaaggcggttggggcgcccgcgcccgccgggacaaagtga

SEQ ID NO: 247 - protein, 156 aa, Triticum aestivum
MPTGRMSGNITQDWEPVVLRRAKPKAADLKSAKAVNQALRTGAPVETVRKAAAGTNKNASAAAVAAPARKLDEMTEPAGLGRVGGDVRAAIQKARVAKGWSQAELAKRINERAQVVQEYESGKAVPVQAVLAKMERALEVKLRGKAVGAPAPAGTK

SEQ ID NO: 248 - DNA, 462 bp, Zea mays
atgccaactggtaggctgagcggcaacatcacccaggactgggagccggtggttctgcgccgtacgaagccgaaggcggccgacctcaagtcgtcgaaggcggtgaaccaggcgctgcgatcgggcgcggccgtggagacggtgcgcaagtcagcagcgggcatgaacaagcactccgctgcggtggcgcccgcgcgtaagctggacgagacgacggagcccgctgcggtggagcgggtggctgtggaggtgcgcgcggccattcagaaggcgcgcgtggccaagggatggagccaggcggagctggcgaagcacatcaacgagcgcgcgcaggtggtgcaggagtacgagagcagcaaggcggcgccggcccaggccgtgcttgccaagatggagcgcgctctcgaggtcaagctccgcgggaagggcgtcggcgcgccactggcggccgtcgggaagtga

SEQ ID NO: 249 - protein, 153 aa, Zea mays
MPTGRLSGNITQDWEPVVLRRTKPKAADLKSSKAVNQALRSGAAVETVRKSAAGMNKHSAAVAPARKLDETTEPAAVERVAVEVRAAIQKARVAKGWSQAELAKHINERAQVVQEYESSKAAPAQAVLAKMERALEVKLRGKGVGAPLAAVGK

SEQ ID NO: 250 - protein, 71 aa, Artificial sequence
  IPR0013729 N-terminal multibridging domain (PFAM entry PF08523 MBF1)of SEQ ID NO 2
QDWEPVVIRKRAPNAAAKRDEKTVNAARRSGADIETVRKFNAGSNKAASSGTSLNTKKLDDDTENLSHDRV

SEQ ID NO: 251 - protein, 55 aa, Artificial sequence
  IPR001387 HELIX-TURN-HELIX TYPE 3 DOMAIN (PFAM ENTRY PF01381 HTH_3)OF SEQ ID NO 2
IMQARGEKKLTQSQLAHLINEKPQVIQEYESGKAIPNQQILSKLERALGAKLRGK

SEQ ID NO: 252 - protein, 144 aa, Artificial sequence
  group I MBF1 consensus sequence
MAGIGPITQDWEPVVIRKKAPXAAAKKDEKAVNAARRSGAEIETVKKFNAGTNKXXAASSGTSLNTRKLDEDTEXLAHERVPTELKKAIMQARLDKKLTQAQLAQLINEKPQVIQEYESGKAIPNQQIISKLERALGVKLRGKK

SEQ ID NO: 253 - DNA, 1130 bp, Oryza sativa
catgcggctaatgtagatgctcactgcgctagtagtaaggtactccagtacattatggaatatacaaagctgtaatactcgtatcagcaagagagaggcacacaagttgtagcagtagcacaggattagaaaaacgggacgacaaatagtaatggaaaaacaaaaaaaaacaaggaaacacatggcaatataaatggagaaatcacaagaggaacagaatccgggcaatacgctgcgaaagtactcgtacgtaaaaaaaagaggcgcattcatgtgtggacagcgtgcagcagaagcagggatttgaaaccactcaaatccaccactgcaaaccttcaaacgaggccatggtttgaagcatagaaagcacaggtaagaagcacaacgccctcgctctccaccctcccacccaatcgcgacgcacctcgcggatcggtgacgtggcctcgccccccaaaaatatcccgcggcgtgaagctgacaccccgggcccacccacctgtcacgttggcacatgttggttatggttcccggccgcaccaaaatatcaacgcggcgcggcccaaaatttccaaaatcccgcccaagcccctggcgcgtgccgctcttccacccaggtccctctcgtaatccataatggcgtgtgtaccctcggctggttgtacgtgggcgggttaccctgggggtgtgggtggatgacgggtgggcccggaggaggtccggccccgcgcgtcatcgcggggcggggtgtagcgggtgcgaaaaggaggcgatcggtacgaaaattcaaattaggaggtggggggcggggcccttggagaataagcggaatcgcagatatgcccctgacttggcttggctcctcttcttcttatcccttgtcctcgcaaccccgcttccttctctcctctcctcttctcttctcttctctggtggtgtgggtgtgtccctgtctcccctctccttcctcctctcctttcccctcctctcttcccccctctcacaagagagagagcgccagactctccccaggtgaggtgagaccagtctttttgctcgattcgacgcgcctttcacgccgcctcgcgcggatctgaccgcttccctcggccttctcgcaggattcagcc

SEQ ID NO: 254 - DNA, 2194 bp, Oryza sativa; identical to SEQ ID NO: 9

SEQ ID NO: 255 - DNA, 51 bp, Artificial sequence
  primer prm09335
ggggacaagtttgtacaaaaaagcaggcttaaacaatggccggaattggac

SEQ ID NO: 256 - DNA, 52 bp, Artificial sequence
  primer prm09336
ggggaccactttgtacaagaaagctgggttgttgttacctttaagagctttg

SEQ ID NO: 257 - DNA, 50 bp, Artificial sequence
  primer prm09337
ggggaccactttgtacaagaaagctgggtagaacttggctcacttctttc

SEQ ID NO: 258 - DNA, 52 bp, Artificial sequence
  primer prm10242
ggggacaagtttgtacaaaaaagcaggcttaaacaatggctgggattggtcc

SEQ ID NO: 259 - DNA, 50 bp, Artificial sequence
  primer prm10243
ggggaccactttgtacaagaaagctgggtgtaaggcaaatagacagggct

SEQ ID NO: 260 - DNA, 56 bp, Artificial sequence
  primer prm10244
ggggacaagtttgtacaaaaaagcaggcttaaacaatgtcaggtctaggccatatt

SEQ ID NO: 261 - DNA, 50 bp, Artificial sequence
  primer prm10245
ggggaccactttgtacaagaaagctgggtattaggtcttcatttcttgcc

SEQ ID NO: 262 - protein, 12 aa, Artificial sequence
  MOTIF I
DLEWKLXYVGSA

SEQ ID NO: 263 - protein, 24 aa, Artificial sequence
  MOTIF II
XPXXXXIXXXXXXGVTVXLLTCXY

SEQ ID NO: 264 - protein, 12 aa, Artificial sequence
  MOTIF III
XEFXRXGYYVXX

SEQ ID NO: 265 - protein, 17 aa, Artificial sequence
  MOTIF IV
XXRNILXXKPRVTXFXI

(19) United States
(12) Patent Application Publication
(10) Pub. No.: US 2011/0247098 A1
(43) Pub. Date: Oct. 6, 2011

(54) Plants Having Enhanced Abiotic Stress Tolerance and/or Enhanced Yield-Related Traits and a Method for Making the Same

(75) Inventors: Yves Hatzfeld, Lille (FR); Christophe Reuzeau, Tocan Saint Apre (FR); Valerie Frankard, Zwijnaarde (BE); Ana Isabel Sanz Molinero, Gentbrugge (BE)

(73) Assignee: BASF Plant Science GmbH, Ludwigshafen (DE)

(21) Appl. No.: 13/128,733

(22) PCT Filed: Nov. 10, 2009
(86) PCT No.: PCT/EP2009/064883

§ 371 Date: Jun. 9, 2011

Related U.S. Application Data
(60) Provisional application No. 61/115,155, filed on Nov. 17, 2008.
(60) Provisional application No. 61/115,572, filed on Nov. 18, 2008.
(60) Provisional application No. 61/116,322, filed on Nov. 20, 2008.
(60) Provisional application No. 61/116,651, filed on Nov. 21, 2008.
(60) Provisional application No. 61/119,491, filed on Dec. 3, 2008.
(60) Provisional application No. 61/119,759, filed on Dec. 4, 2008.
(60) Provisional application No. 61/119,739, filed on Dec. 4, 2008.

(30) Foreign Application Priority Data
Nov. 12, 2008 (EP) 08168973.9
Nov. 17, 2008 (EP) 08169265.9
Nov. 19, 2008 (EP) 08169417.6
Nov. 21, 2008 (EP) 08169612.2
Nov. 24, 2008 (EP) 08169820.1
Nov. 26, 2008 (EP) 08169951.4
Nov. 28, 2008 (EP) 08170295.3

(51) Int. Cl. A01H 1/06; A01H 5/00; A01H 5/10; C12N 15/82; C12N 5/10; C07H 21/04; C12N 9/14

(52) U.S. Cl. 800/290; 800/278; 800/298; 800/320; 800/320.2; 800/320.1; 800/320.3; 435/320.1; 435/419; 435/412; 536/23.2; 435/195

(57) ABSTRACT

The present invention relates generally to the field of molecular biology and concerns a method for enhancing abiotic stress tolerance in plants by modulating expression in a plant of a nucleic acid encoding a cytochrome c oxidase (COX) VIIa subunit polypeptide (COX VIIa subunit). The present invention also concerns plants having modulated expression of a nucleic acid encoding a COX VIIa subunit, which plants have enhanced abiotic stress tolerance relative to corresponding wild type plants or other control plants. The invention also provides constructs useful in the methods of the invention. Furthermore, the present invention relates generally to the field of molecular biology and concerns a method for improving various plant growth characteristics by modulating expression in a plant of a nucleic acid encoding a YLD-ZnF polypeptide. The present invention also concerns plants having modulated expression of a nucleic acid encoding a YLD-ZnF polypeptide, which plants have improved growth characteristics relative to corresponding wild type plants or other control plants. The invention also provides constructs useful in the methods of the invention. Furthermore, the present invention relates generally to the field of molecular biology and concerns a method for enhancing abiotic stress tolerance in plants by modulating expression in a plant of a nucleic acid encoding a PKT (protein kinase with TPR repeat). The present invention also concerns plants having modulated expression of a nucleic acid encoding a PKT, which plants have enhanced abiotic stress tolerance relative to corresponding wild type plants or other control plants. The invention also provides constructs useful in the methods of the invention. Furthermore, the present invention relates generally to the field of molecular biology and concerns a method for improving various plant growth characteristics by modulating expression in a plant of a nucleic acid encoding a NOA (Nitric Oxide Associated) polypeptide. The present invention also concerns plants having modulated expression of a nucleic acid encoding a NOA polypeptide, which plants have improved growth characteristics relative to corresponding wild type plants or other control plants. The invention also provides constructs useful in the methods of the invention. Furthermore, the present invention relates generally to the field of molecular biology and concerns a method for improving various yield-related traits in plants by modulating expression in a plant of a nucleic acid encoding an Anti-silencing factor 1 (ASF1)-like polypeptide. The present invention also concerns plants having modulated expression of a nucleic acid encoding an ASF1-like polypeptide, which plants have enhanced yield-related traits relative to corresponding wild type plants or other control plants. The invention also provides constructs useful in the methods of the invention. Furthermore, the present invention relates generally to the field of molecular biology and concerns a method for enhancing abiotic stress tolerance in plants by modulating expression in a plant of a nucleic acid encoding a plant homeodomain finger (PHDF). The present invention also concerns plants having modulated expression of a nucleic acid encoding a PHDF, which plants have enhanced abiotic stress tolerance relative to corresponding wild type plants or other control plants. The invention also provides constructs useful in the methods of the invention. Furthermore, the present invention relates generally to the field of molecular biology and concerns a method for increasing various plant yield-related traits by increasing expression in a plant of a nucleic acid sequence encoding a group multi-protein bridging factor 1 (MBF1) polypeptide. The present invention also concerns plants having increased expression of a nucleic acid sequence encoding a group I MBF1 polypeptide, which plants have increased yield-related traits relative to control plants. The invention additionally relates to nucleic acid sequences, nucleic acid constructs, vectors and plants containing said nucleic acid sequences.

The present invention relates generally to the field of molecular biology and concerns a method for enhancing abiotic stress tolerance in plants by modulating expression in a plant of a nucleic acid encoding a cytochrome c oxidase (COX) VIIa subunit polypeptide (COX VIIa subunit). The present invention also concerns plants having modulated expression of a nucleic acid encoding a COX VIIa subunit, which plants have enhanced abiotic stress tolerance relative to corresponding wild type plants or other control plants. The invention also provides constructs useful in the methods of the invention.

Furthermore, the present invention relates generally to the field of molecular biology and concerns a method for improving various plant growth characteristics by modulating expression in a plant of a nucleic acid encoding a YLD-ZnF polypeptide. The present invention also concerns plants having modulated expression of a nucleic acid encoding a YLD-ZnF polypeptide, which plants have improved growth characteristics relative to corresponding wild type plants or other control plants. The invention also provides constructs useful in the methods of the invention.

Furthermore, the present invention relates generally to the field of molecular biology and concerns a method for enhancing abiotic stress tolerance in plants by modulating expression in a plant of a nucleic acid encoding a PKT (protein kinase with TPR repeat). The present invention also concerns plants having modulated expression of a nucleic acid encoding a PKT, which plants have enhanced abiotic stress tolerance relative to corresponding wild type plants or other control plants. The invention also provides constructs useful in the methods of the invention.

Furthermore, the present invention relates generally to the field of molecular biology and concerns a method for improving various plant growth characteristics by modulating expression in a plant of a nucleic acid encoding a NOA (Nitric Oxide Associated) polypeptide. The present invention also concerns plants having modulated expression of a nucleic acid encoding a NOA polypeptide, which plants have improved growth characteristics relative to corresponding wild type plants or other control plants. The invention also provides constructs useful in the methods of the invention.

Furthermore, the present invention relates generally to the field of molecular biology and concerns a method for improving various yield-related traits in plants by modulating expression in a plant of a nucleic acid encoding an Anti-silencing factor 1 (ASF1)-like polypeptide. The present invention also concerns plants having modulated expression of a nucleic acid encoding an ASF1-like polypeptide, which plants have enhanced yield-related traits relative to corresponding wild type plants or other control plants. The invention also provides constructs useful in the methods of the invention.

Furthermore, the present invention relates generally to the field of molecular biology and concerns a method for enhancing abiotic stress tolerance in plants by modulating expression in a plant of a nucleic acid encoding a plant homeodomain finger (PHDF). The present invention also concerns plants having modulated expression of a nucleic acid encoding a PHDF, which plants have enhanced abiotic stress tolerance relative to corresponding wild type plants or other control plants. The invention also provides constructs useful in the methods of the invention.

Furthermore, the present invention relates generally to the field of molecular biology and concerns a method for increasing various plant yield-related traits by increasing expression in a plant of a nucleic acid sequence encoding a group I multiprotein bridging factor 1 (MBF1) polypeptide. The present invention also concerns plants having increased expression of a nucleic acid sequence encoding a group I MBF1 polypeptide, which plants have increased yield-related traits relative to control plants. The invention additionally relates to nucleic acid sequences, nucleic acid constructs, vectors and plants containing said nucleic acid sequences.

The ever-increasing world population and the dwindling supply of arable land available for agriculture fuels research towards increasing the efficiency of agriculture. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic components that may not always result in the desirable trait being passed on from parent plants. Advances in molecular biology have allowed mankind to modify the germplasm of animals and plants. Genetic engineering of plants entails the isolation and manipulation of genetic material (typically in the form of DNA or RNA) and the subsequent introduction of that genetic material into a plant. Such technology has the capacity to deliver crops or plants having various improved economic, agronomic or horticultural traits.

A trait of particular economic interest is increased yield. Yield is normally defined as the measurable produce of economic value from a crop. This may be defined in terms of quantity and/or quality. Yield is directly dependent on several factors, for example, the number and size of the organs, plant architecture (for example, the number of branches), seed production, leaf senescence and more. Root development, nutrient uptake, stress tolerance and early vigour may also be important factors in determining yield. Optimizing the abovementioned factors may therefore contribute to increasing crop yield.

Seed yield is a particularly important trait, since the seeds of many plants are important for human and animal nutrition. Crops such as corn, rice, wheat, canola and soybean account for over half the total human caloric intake, whether through direct consumption of the seeds themselves or through consumption of meat products raised on processed seeds. They are also a source of sugars, oils and many kinds of metabolites used in industrial processes. Seeds contain an embryo (the source of new shoots and roots) and an endosperm (the source of nutrients for embryo growth during germination and during early growth of seedlings). The development of a seed involves many genes, and requires the transfer of metabolites from the roots, leaves and stems into the growing seed. The endosperm, in particular, assimilates the metabolic precursors of carbohydrates, oils and proteins and synthesizes them into storage macromolecules to fill out the grain.

Plant biomass is yield for forage crops like alfalfa, silage corn and hay. Many proxies for yield have been used in grain crops. Chief amongst these are estimates of plant size. Plant size can be measured in many ways depending on species and developmental stage, but include total plant dry weight, above-ground dry weight, above-ground fresh weight, leaf area, stem volume, plant height, rosette diameter, leaf length, root length, root mass, tiller number and leaf number. Many species maintain a conservative ratio between the size of different parts of the plant at a given developmental stage. These allometric relationships are used to extrapolate from one of these measures of size to another (e.g. Tittonell et al 2005 Agric Ecosys & Environ 105: 213). Plant size at an early developmental stage will typically correlate with plant size later in development. A larger plant with a greater leaf area can typically absorb more light and carbon dioxide than a smaller plant and therefore will likely gain a greater weight during the same period (Fasoula & Tollenaar 2005 Maydica 50:39). This is in addition to the potential continuation of the micro-environmental or genetic advantage that the plant had to achieve the larger size initially. There is a strong genetic component to plant size and growth rate (e.g. ter Steege et al 2005 Plant Physiology 139:1078), and so for a range of diverse genotypes plant size under one environmental condition is likely to correlate with size under another (Hittalmani et al 2003 Theoretical Applied Genetics 107:679). In this way a standard environment is used as a proxy for the diverse and dynamic environments encountered at different locations and times by crops in the field.

Another important trait for many crops is early vigour. Improving early vigour is an important objective of modern rice breeding programs in both temperate and tropical rice cultivars. Long roots are important for proper soil anchorage in water-seeded rice. Where rice is sown directly into flooded fields, and where plants must emerge rapidly through water, longer shoots are associated with vigour. Where drill-seeding is practiced, longer mesocotyls and coleoptiles are important for good seedling emergence. The ability to engineer early vigour into plants would be of great importance in agriculture. For example, poor early vigour has been a limitation to the introduction of maize (Zea mays L.) hybrids based on Corn Belt germplasm in the European Atlantic.

Harvest index, the ratio of seed yield to aboveground dry weight, is relatively stable under many environmental conditions and so a robust correlation between plant size and grain yield can often be obtained (e.g. Rebetzke et al 2002 Crop Science 42:739). These processes are intrinsically linked because the majority of grain biomass is dependent on current or stored photosynthetic productivity by the leaves and stem of the plant (Gardener et al 1985 Physiology of Crop Plants. Iowa State University Press, pp 68-73). Therefore, selecting for plant size, even at early stages of development, has been used as an indicator for future potential yield (e.g. Tittonell et al 2005 Agric Ecosys & Environ 105: 213). When testing for the impact of genetic differences on stress tolerance, the ability to standardize soil properties, temperature, water and nutrient availability and light intensity is an intrinsic advantage of greenhouse or plant growth chamber environments compared to the field. However, artificial limitations on yield due to poor pollination due to the absence of wind or insects, or insufficient space for mature root or canopy growth, can restrict the use of these controlled environments for testing yield differences. Therefore, measurements of plant size in early development, under standardized conditions in a growth chamber or greenhouse, are standard practices to provide indication of potential genetic yield advantages.

A further important trait is that of improved abiotic stress tolerance. Abiotic stress is a primary cause of crop loss worldwide, reducing average yields for most major crop plants by more than 50% (Wang et al., Planta (2003) 218: 1-14). Abiotic stresses may be caused by drought, salinity, extremes of temperature, chemical toxicity and oxidative stress. The ability to improve plant tolerance to abiotic stress would be of great economic advantage to farmers worldwide and would allow for the cultivation of crops during adverse conditions and in territories where cultivation of crops may not otherwise be possible.

Crop yield may therefore be increased by optimising one of the above-mentioned factors.

Depending on the end use, the modification of certain yield traits may be favoured over others. For example for applications such as forage or wood production, or bio-fuel resource, an increase in the vegetative parts of a plant may be desirable, and for applications such as flour, starch or oil production, an increase in seed parameters may be particularly desirable. Even amongst the seed parameters, some may be favoured over others, depending on the application. Various mechanisms may contribute to increasing seed yield, whether that is in the form of increased seed size or increased seed number.

One approach to increasing yield (seed yield and/or biomass) in plants may be through modification of the inherent growth mechanisms of a plant, such as the cell cycle or various signalling pathways involved in plant growth or in defense mechanisms.

It has now been found that tolerance to various abiotic stresses may be enhanced in plants by modulating expression in a plant of a nucleic acid encoding a COX VIIa subunit.

It has now been found that various yield-related traits may be enhanced in plants by modulating expression in a plant of a nucleic acid encoding a YLD-ZnF polypeptide.

It has now been found that tolerance to various abiotic stresses may be enhanced in plants by modulating expression in a plant of a nucleic acid encoding a PKT.

It has now been found that various growth characteristics may be improved in plants by modulating expression in a plant of a nucleic acid encoding a NOA (Nitric Oxide Associated) in a plant.

It has now been found that various yield-related traits may be enhanced in plants by modulating expression in a plant of a nucleic acid encoding an ASF1-like polypeptide.

It has now been found that tolerance to various abiotic stresses may be enhanced in plants by modulating expression in a plant of a nucleic acid encoding a PHDF polypeptide.

It has now been found that various yield-related traits may be increased in plants relative to control plants, by increasing expression in a plant of a nucleic acid sequence encoding a multiprotein bridging factor 1 (MBF1) polypeptide. The increased yield-related traits comprise one or more of: increased aboveground biomass, increased early vigor, increased seed yield per plant, increased seed fill rate, increased number of filled seeds, or increased number of primary panicles.

### Background

### 1. NOA Polypeptides

In both animals and plants, nitric oxide (NO) plays a role as signalling molecule. In plants, nitric oxide plays a role in various physiological and developmental processes, such as hormone responses, abiotic stress response, respiration, cell death, leaf expansion, root development, seed germination, fruit maturation, senescence and disease resistance. Synthesis of nitric oxide plants is believed to occur via two routes: a reduction of nitrite to nitric oxide by nitrite reductase, by a plasma membrane-bound nitrite:NO reductase, by a mitochondrial electron transport-dependent reductase or simply in a non-enzymatically catalysed reaction in acidic reducing environment. The second route encompasses oxidation of arginine to citrulline by nitric oxide synthase. An Arabidopsis mutant (Atnos1) impaired for NO production showed yellow first true leaves, reduced growth of vegetative biomass and reduced fertility (Guo et al., Science 302, 100-103, 2003). Overexpression of Atnos1 in the mutant resulted in only a partial rescue of the mutant phenotype: the plants were still dwarfed compare to wild type plants and also stomatal functioning remained impaired. AtNOS1 was later shown not to be a nitric oxide synthase, but rather a GTPase (Flores-Pérez et al., Plant Cell 20, 1303-1315, 2008; Moreau et al., J. Biol. Chem. 2008, M804838200 (in press)).

2. ASF1-like Polypeptides

Chromosome assembly begins when eight histone subunits are brought together and a double strand of DNA loops around them twice—more precisely, one and two-thirds—like thread around a spool. The result is a nucleosome. The continuous DNA strand connects the nucleosomes like beads on a string, and this DNA-protein beaded string is rolled up into a cylindrical rope-like structure, chromatin, which is further folded and looped into the compact mass of the chromosome. The main role of Asf1 is as a histone chaperone, helping to deposit histone proteins on DNA strands to form nucleosomes, the protein-DNA units that when linked together make up chromatin.

Asf1 was first identified in Saccharomyces cerevisiae, and has since been identified in many other eukaryotes. All eukaryotes have at least one version of the gene, some, including humans, have two. The first 155 amino-acid residues of Asf1, counting from the exposed amino-group end of the string (the N-terminal), are highly conserved in virtually all organisms. The rest of the sequence (the C-terminal) varies widely among organisms, and in at least one, the parasite Leishmania major, it is missing altogether.

### 3. PHDF Polypeptides

The PHD finger, a Cys₄-His-Cys₃ zinc finger, is found in many regulatory proteins from plants to animals and which are frequently associated with chromatin-mediated transcriptional regulation. The PHD finger has been shown to activate transcription in yeast, plant and animal cells (Halbach et al., Nucleic Acids Res. 2000 September 15; 28(18): 3542-3550).

4. group I MBF1 Polypeptides

Transcriptional coactivators play a crucial role in eukaryotic gene expression by communicating between transcription factors and/or other regulatory components and the basal transcription machinery. They are divided into two classes: transcriptional coactivators that recruit or possess enzymatic activities that modify chromatin structure (e.g. acetylation of histone) and transcriptional coactivators that recruit the general transcriptional machinery to a promoter where a transcription factor(s) is bound. Multiprotein bridging factor 1 (MBF1) is a highly conserved transcriptional coactivator involved in the regulation of diverse processes in different organism. The model plant Arabidopsis thaliana contains three different genes encoding MBF1.

Functional assays demonstrate that all three Arabidopsis genes can complement MBF1 deficiency in yeast (Tsuda et al., 2004). MBF1a (At2g42680) and MBF1b (At3g58680) are developmentally regulated (Tsuda K, Yamazaki K (2004) Biochim Biophys Acta 1680: 1-10), and both belong to the plant MBF1 group I. In contrast, the steady-state level of transcripts encoding MBF1c (At3g24500) is specifically elevated in Arabidopsis in response to pathogen infection, salinity, drought, heat, hydrogen peroxide, and application of the plant hormones abscisic acid or salicylic acid (Tsuda, Yamazaki (2004) supra). MBF1c belongs to the plant MBF1 group II.

Transgenic Arabidopsis plants overexpressing MBF1c using a 35S CaMV constitutive promoter appeared similar in their growth and development to wild-type plants. However, transgenic plants expressing MBF1c were 20% larger than control plants and produced more seeds (Suzuki et al. (2005) Plant Physiol 139(3): 1313-1322).

US patent application US2007214517 describes nucleic acid sequences encoding class I (referenced as SEQ ID 40130) and class II MBF1 polypeptides, and constructs comprising these. International application WO 2008/064341 “Nucleotide sequences and corresponding polypeptides conferring enhanced heat tolerance in plants” describes nucleic acid sequences encoding class I and class II MBF1 polypeptides, and methods and materials for modulating heat tolerance levels in plants.

### SUMMARY

### 1. COX VIIa Subunit Polypeptides

Surprisingly, it has now been found that modulating expression of a nucleic acid encoding a COX VIIa subunit polypeptide gives plants having enhanced tolerance to various abiotic stresses relative to control plants.

According to one embodiment, there is provided a method for enhancing tolerance in plants to various abiotic stresses, relative to tolerance in control plants, comprising modulating expression of a nucleic acid encoding a COX VIIa subunit polypeptide in a plant.

### 2. YLD-ZnF Polypeptides

Surprisingly, it has now been found that modulating expression of a nucleic acid encoding a YLD-ZnF polypeptide gives plants having enhanced yield-related traits, in particular increased yield, relative to control plants.

According to one embodiment, there is provided a method for improving yield related traits of a plant relative to control plants, comprising modulating expression of a nucleic acid encoding a YLD-ZnF polypeptide in a plant.

### 3. PKT Polypeptides

Surprisingly, it has now been found that modulating expression of a nucleic acid encoding a PKT polypeptide gives plants having enhanced tolerance to various abiotic stresses relative to control plants.

According to one embodiment, there is provided a method for enhancing tolerance in plants to various abiotic stresses, relative to tolerance in control plants, comprising modulating expression of a nucleic acid encoding a PKT polypeptide in a plant.

### 4. NOA Polypeptides

Surprisingly, it has now been found that modulating expression of a nucleic acid encoding a NOA polypeptide gives plants having enhanced yield-related traits, in particular increased yield, relative to control plants.

According to one embodiment, there is provided a method for improving yield related traits of a plant relative to control plants, comprising modulating expression of a nucleic acid encoding a NOA polypeptide in a plant.

5. ASF1-like Polypeptides

Surprisingly, it has now been found that modulating expression of a nucleic acid encoding an ASF1-like polypeptide gives plants having enhanced yield-related traits relative to control plants.

According to one embodiment, there is provided a method for enhancing yield-related traits in plants relative to control plants, comprising modulating expression of a nucleic acid encoding an ASF1-like polypeptide in a plant.

### 6. PHDF Polypeptides

Surprisingly, it has now been found that modulating expression of a nucleic acid encoding a PHDF polypeptide gives plants having enhanced tolerance to various abiotic stresses relative to control plants.

According to one embodiment, there is provided a method for enhancing tolerance in plants to various abiotic stresses, relative to tolerance in control plants, comprising modulating expression of a nucleic acid encoding a PHDF polypeptide in a plant.

### 7. Group I MBF1 Polypeptides

Surprisingly, it has now been found that increasing expression in a plant of a nucleic acid sequence encoding a group I MBF1 polypeptide as defined herein, gives plants having increased yield-related traits relative to control plants.

According to one embodiment, there is provided a method for increasing yield-related traits in plants relative to control plants, comprising increasing expression in a plant of a nucleic acid sequence encoding a group I MBF1 polypeptide as defined herein. The increased yield-related traits comprise one or more of: increased aboveground biomass, increased early vigor, increased seed yield per plant, increased seed fill rate, increased number of filled seeds, or increased number of primary panicles.

### DEFINITIONS

### Polypeptide(s)/Protein(s)

The terms “polypeptide” and “protein” are used interchangeably herein and refer to amino acids in a polymeric form of any length, linked together by peptide bonds.

### Polynucleotide(s)/Nucleic Acid(s)/Nucleic Acid Sequence(s)/Nucleotide Sequence(s)

The terms “polynucleotide(s)”, “nucleic acid sequence(s)”, “nucleotide sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length.

### Control Plant(s)

The choice of suitable control plants is a routine part of an experimental setup and may include corresponding wild type plants or corresponding plants without the gene of interest. The control plant is typically of the same plant species or even of the same variety as the plant to be assessed. The control plant may also be a nullizygote of the plant to be assessed. Nullizygotes are individuals missing the transgene by segregation.

A “control plant” as used herein refers not only to whole plants, but also to plant parts, including seeds and seed parts.

### Homologue(s)

“Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

A deletion refers to removal of one or more amino acids from a protein.

An insertion refers to one or more amino acid residues being introduced into a predetermined site in a protein. Insertions may comprise N-terminal and/or C-terminal fusions as well as intra-sequence insertions of single or multiple amino acids. Generally, insertions within the amino acid sequence will be smaller than N- or C-terminal fusions, of the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag•100 epitope, c-myc epitope, FLAG®-epitope, lacZ, CMP (calmodulin-binding peptide), HA epitope, protein C epitope and VSV epitope.

A substitution refers to replacement of amino acids of the protein with other amino acids having similar properties (such as similar hydrophobicity, hydrophilicity, antigenicity, propensity to form or break α-helical structures or β-sheet structures). Amino acid substitutions are typically of single residues, but may be clustered depending upon functional constraints placed upon the polypeptide; insertions will usually be of the order of about 1 to 10 amino acid residues. The amino acid substitutions are preferably conservative amino acid substitutions. Conservative substitution tables are well known in the art (see for example Creighton (1984) Proteins. W.H. Freeman and Company (Eds) and Table 1 below).

TABLE 1
Examples of conserved amino acid substitutions

| | Conservative | | Conservative |
|---|---|---|---|
| Residue | Substitutions | Residue | Substitutions |
| Ala | Ser | Leu | Ile; Val |
| Arg | Lys | Lys | Arg; Gln |
| Asn | Gln; His | Met | Leu; Ile |
| Asp | Glu | Phe | Met; Leu; Tyr |
| Gln | Asn | Ser | Thr; Gly |
| Cys | Ser | Thr | Ser; Val |
| Glu | Asp | Trp | Tyr |
| Gly | Pro | Tyr | Trp; Phe |
| His | Asn; Gln | Val | Ile; Leu |
| Ile | Leu, Val | | |

Amino acid substitutions, deletions and/or insertions may readily be made using peptide synthetic techniques well known in the art, such as solid phase peptide synthesis and the like, or by recombinant DNA manipulation. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols.

### Derivatives

“Derivatives” include peptides, oligopeptides, polypeptides which may, compared to the amino acid sequence of the naturally-occurring form of the protein, such as the protein of interest, comprise substitutions of amino acids with non-naturally occurring amino acid residues, or additions of non-naturally occurring amino acid residues. “Derivatives” of a protein also encompass peptides, oligopeptides, polypeptides which comprise naturally occurring altered (glycosylated, acylated, prenylated, phosphorylated, myristoylated, sulphated etc.) or non-naturally altered amino acid residues compared to the amino acid sequence of a naturally-occurring form of the polypeptide. A derivative may also comprise one or more non-amino acid substituents or additions compared to the amino acid sequence from which it is derived, for example a reporter molecule or other ligand, covalently or non-covalently bound to the amino acid sequence, such as a reporter molecule which is bound to facilitate its detection, and non-naturally occurring amino acid residues relative to the amino acid sequence of a naturally-occurring protein. Furthermore, “derivatives” also include fusions of the naturally-occurring form of the protein with tagging peptides such as FLAG, HIS6 or thioredoxin (for a review of tagging peptides, see Terpe, Appl. Microbiol. Biotechnol. 60, 523-533, 2003).

### Orthologue(s)/Paralogue(s)

Orthologues and paralogues encompass evolutionary concepts used to describe the ancestral relationships of genes. Paralogues are genes within the same species that have originated through duplication of an ancestral gene; orthologues are genes from different organisms that have originated through speciation, and are also derived from a common ancestral gene.

### Domain

The term “domain” refers to a set of amino acids conserved at specific positions along an alignment of sequences of evolutionarily related proteins. While amino acids at other positions can vary between homologues, amino acids that are highly conserved at specific positions indicate amino acids that are likely essential in the structure, stability or function of a protein. Identified by their high degree of conservation in aligned sequences of a family of protein homologues, they can be used as identifiers to determine if any polypeptide in question belongs to a previously identified polypeptide family.

### Motif/Consensus Sequence/Signature

The term “motif” or “consensus sequence” or “signature” refers to a short conserved region in the sequence of evolutionarily related proteins. Motifs are frequently highly conserved parts of domains, but may also include only part of the domain, or be located outside of conserved domain (if all of the amino acids of the motif fall outside of a defined domain).

### Hybridisation

The term “hybridisation” as defined herein is a process wherein substantially homologous complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

The term “stringency” refers to the conditions under which a hybridisation takes place. The stringency of hybridisation is influenced by conditions such as temperature, salt concentration, ionic strength and hybridisation buffer composition. Generally, low stringency conditions are selected to be about 30° C. lower than the thermal melting point (Tₘ) for the specific sequence at a defined ionic strength and pH. Medium stringency conditions are when the temperature is 20° C. below Tₘ, and high stringency conditions are when the temperature is 10° C. below Tₘ. High stringency hybridisation conditions are typically used for isolating hybridising sequences that have high sequence similarity to the target nucleic acid sequence. However, nucleic acids may deviate in sequence and still encode a substantially identical polypeptide, due to the degeneracy of the genetic code. Therefore medium stringency hybridisation conditions may sometimes be needed to identify such nucleic acid molecules.

The Tm is the temperature under defined ionic strength and pH, at which 50% of the target sequence hybridises to a perfectly matched probe. The Tₘ is dependent upon the solution conditions and the base composition and length of the probe. For example, longer sequences hybridise specifically at higher temperatures. The maximum rate of hybridisation is obtained from about 16° C. up to 32° C. below Tₘ. The presence of monovalent cations in the hybridisation solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tm decreases about 1° C. per % base mismatch. The Tm may be calculated using the following equations, depending on the types of hybrids:

1) DNA-DNA hybrids (Meinkoth and Wahl, Anal. Biochem., 138: 267-284, 1984):

Tₘ=81.5° C.+16.6×log₁₀ [Na⁺]^{a}+0.41×%[G/C^{b}]−500×[L^{c}]⁻¹−0.61×% formamide

2) DNA-RNA or RNA-RNA hybrids:

Tₘ=79.8+18.5(log₁₀ [Na⁺]^{a})+0.58(%G/C^{b})+11.8(%G/C^{b})²−820/L^{c}

3) oligo-DNA or oligo-RNAs hybrids:
For <20 nucleotides: Tₘ=2 (Iₙ)
For 20-35 nucleotides: Tₘ=22+1.46 (Iₙ)
^{a} or for other monovalent cation, but only accurate in the 0.01-0.4 M range.
^{b} only accurate for % GC in the 30% to 75% range.
^{c} L=length of duplex in base pairs.
^{d} oligo, oligonucleotide; Iₙ, =effective length of primer=2×(no. of G/C)+(no. of A/T).

Non-specific binding may be controlled using any one of a number of known techniques such as, for example, blocking the membrane with protein containing solutions, additions of heterologous RNA, DNA, and SDS to the hybridisation buffer, and treatment with Rnase. For non-homologous probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1×SSC or at 42° C. in 1×SSC and 50% formamide, followed by washing at 65° C. in 0.3×SSC. Examples of medium stringency hybridisation conditions for DNA hybrids longer than 50 nucleotides encompass hybridisation at 50° C. in 4×SSC or at 40° C. in 6×SSC and 50% formamide, followed by washing at 50° C. in 2×SSC. The length of the hybrid is the anticipated length for the hybridising nucleic acid. When nucleic acids of known sequence are hybridised, the hybrid length may be determined by aligning the sequences and identifying the conserved regions described herein. 1×SSC is 0.15M NaCl and 15 mM sodium citrate; the hybridisation solution and wash solutions may additionally include 5×Denhardt's reagent, 0.5-1.0% SDS, 100 μg/ml denatured, fragmented salmon sperm DNA, 0.5% sodium pyrophosphate.

For the purposes of defining the level of stringency, reference can be made to Sambrook et al. (2001) Molecular Cloning: a laboratory manual, 3rd Edition, Cold Spring Harbor Laboratory Press, CSH, New York or to Current Protocols in Molecular Biology, John Wiley & Sons, N.Y. (1989 and yearly updates).

### Splice Variant

The term “splice variant” as used herein encompasses variants of a nucleic acid sequence in which selected introns and/or exons have been excised, replaced, displaced or added, or in which introns have been shortened or lengthened. Such variants will be ones in which the biological activity of the protein is substantially retained; this may be achieved by selectively retaining functional segments of the protein. Such splice variants may be found in nature or may be manmade. Methods for predicting and isolating such splice variants are well known in the art (see for example Foissac and Schiex (2005) BMC Bioinformatics 6: 25).

### Allelic Variant

Alleles or allelic variants are alternative forms of a given gene, located at the same chromosomal position. Allelic variants encompass Single Nucleotide Polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms.

### Gene Shuffling/Directed Evolution

Gene shuffling or directed evolution consists of iterations of DNA shuffling followed by appropriate screening and/or selection to generate variants of nucleic acids or portions thereof encoding proteins having a modified biological activity (Castle et al., (2004) Science 304(5674): 1151-4; U.S. Pat. Nos. 5,811,238 and 6,395,547).

### Regulatory Element/Control Sequence/Promoter

The terms “regulatory element”, “control sequence” and “promoter” are all used interchangeably herein and are to be taken in a broad context to refer to regulatory nucleic acid sequences capable of effecting expression of the sequences to which they are ligated. The term “promoter” typically refers to a nucleic acid control sequence located upstream from the transcriptional start of a gene and which is involved in recognising and binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative that confers, activates or enhances expression of a nucleic acid molecule in a cell, tissue or organ.

A “plant promoter” comprises regulatory elements, which mediate the expression of a coding sequence segment in plant cells. Accordingly, a plant promoter need not be of plant origin, but may originate from viruses or micro-organisms, for example from viruses which attack plant cells. The “plant promoter” can also originate from a plant cell, e.g. from the plant which is transformed with the nucleic acid sequence to be expressed in the inventive process and described herein. This also applies to other “plant” regulatory signals, such as “plant” terminators. The promoters upstream of the nucleotide sequences useful in the methods of the present invention can be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. It is furthermore possible that the activity of the promoters is increased by modification of their sequence, or that they are replaced completely by more active promoters, even promoters from heterologous organisms. For expression in plants, the nucleic acid molecule must, as described above, be linked operably to or comprise a suitable promoter which expresses the gene at the right point in time and with the required spatial expression pattern.

For the identification of functionally equivalent promoters, the promoter strength and/or expression pattern of a candidate promoter may be analysed for example by operably linking the promoter to a reporter gene and assaying the expression level and pattern of the reporter gene in various tissues of the plant. Suitable well-known reporter genes include for example beta-glucuronidase or beta-galactosidase. The promoter activity is assayed by measuring the enzymatic activity of the beta-glucuronidase or beta-galactosidase. The promoter strength and/or expression pattern may then be compared to that of a reference promoter (such as the one used in the methods of the present invention). Alternatively, promoter strength may be assayed by quantifying mRNA levels or by comparing mRNA levels of the nucleic acid used in the methods of the present invention, with mRNA levels of housekeeping genes such as 18S rRNA, using methods known in the art, such as Northern blotting with densitometric analysis of autoradiograms, quantitative real-time PCR or RT-PCR (Heid et al., 1996 Genome Methods 6: 986-994). Generally by “weak promoter” is intended a promoter that drives expression of a coding sequence at a low level. By “low level” is intended at levels of about 1/10,000 transcripts to about 1/100,000 transcripts, to about 1/500,0000 transcripts per cell. Conversely, a “strong promoter” drives expression of a coding sequence at high level, or at about 1/10 transcripts to about 1/100 transcripts to about 1/1000 transcripts per cell. Generally, by “medium strength promoter” is intended a promoter that drives expression of a coding sequence at a lower level than a strong promoter, in particular at a level that is in all instances below that obtained when under the control of a 35S CaMV promoter.

### Operably Linked

The term “operably linked” as used herein refers to a functional linkage between the promoter sequence and the gene of interest, such that the promoter sequence is able to initiate transcription of the gene of interest.

### Constitutive Promoter

A “constitutive promoter” refers to a promoter that is transcriptionally active during most, but not necessarily all, phases of growth and development and under most environmental conditions, in at least one cell, tissue or organ. Table 2a below gives examples of constitutive promoters.

TABLE 2a
Examples of constitutive promoters

| Gene Source | Reference |
|---|---|
| Actin | McElroy et al, Plant Cell, 2: 163-171, 1990 |
| HMGP | WO 2004/070039 |
| CAMV 35S | Odell et al, Nature, 313: 810-812, 1985 |
| CaMV 19S | Nilsson et al., Physiol. Plant. 100: 456-462, 1997 |
| GOS2 | de Pater et al, Plant J November; 2(6): 837-44, 1992, |
| | WO 2004/065596 |
| Ubiquitin | Christensen et al, Plant Mol. Biol. 18: 675-689, 1992 |
| Rice cyclophilin | Buchholz et al, Plant Mol Biol. 25(5): 837-43, 1994 |
| Maize H3 histone | Lepetit et al, Mol. Gen. Genet. 231: 276-285, 1992 |
| Alfalfa H3 histone | Wu et al. Plant Mol. Biol. 11:641-649, 1988 |
| Actin 2 | An et al, Plant J. 10(1); 107-121, 1996 |
| 34S FMV | Sanger et al., Plant. Mol. Biol., 14, 1990: 433-443 |
| Rubisco small | US 4,962,028 |
| subunit | |
| OCS | Leisner (1988) Proc Natl Acad Sci USA 85(5): 2553 |
| SAD1 | Jain et al., Crop Science, 39(6), 1999: 1696 |
| SAD2 | Jain et al., Crop Science, 39(6), 1999: 1696 |
| nos | Shaw et al. (1984) Nucleic Acids Res. 12(20): |
| | 7831-7846 |
| V-ATPase | WO 01/14572 |
| Super promoter | WO 95/14098 |
| G-box proteins | WO 94/12015 |

### Ubiquitous Promoter

A ubiquitous promoter is active in substantially all tissues or cells of an organism.

### Developmentally-Regulated Promoter

A developmentally-regulated promoter is active during certain developmental stages or in parts of the plant that undergo developmental changes.

### Inducible Promoter

An inducible promoter has induced or increased transcription initiation in response to a chemical (for a review see Gatz 1997, Annu. Rev. Plant Physiol. Plant Mol. Biol., 48:89-108), environmental or physical stimulus, or may be “stress-inducible”, i.e. activated when a plant is exposed to various stress conditions, or a “pathogen-inducible” i.e. activated when a plant is exposed to exposure to various pathogens.

### Organ-Specific/Tissue-Specific Promoter

An organ-specific or tissue-specific promoter is one that is capable of preferentially initiating transcription in certain organs or tissues, such as the leaves, roots, seed tissue etc. For example, a “root-specific promoter” is a promoter that is transcriptionally active predominantly in plant roots, substantially to the exclusion of any other parts of a plant, whilst still allowing for any leaky expression in these other plant parts. Promoters able to initiate transcription in certain cells only are referred to herein as “cell-specific”.

Examples of root-specific promoters are listed in Table 2b below:

TABLE 2b
Examples of root-specific promoters

| Gene Source | Reference |
|---|---|
| RCc3 | Plant Mol Biol. 1995 January; 27(2): 237-48 |
| Arabidopsis PHT1 | Kovama et al., 2005; Mudge et al. |
| | (2002, Plant J. 31: 341) |
| Medicago phosphate | Xiao et al., 2006 |
| transporter | |
| Arabidopsis Pyk10 | Nitz et al. (2001) Plant Sci 161(2): 337-346 |
| root-expressible genes | Tingey et al., EMBO J. 6: 1, 1987. |
| tobacco auxin- | Van der Zaal et al., Plant Mol. Biol. |
| inducible gene | 16, 983, 1991. |
| β-tubulin | Oppenheimer, et al., Gene 63: 87, 1988. |
| tobacco root- | Conkling, et al., Plant Physiol. 93: 1203, 1990. |
| specific genes | |
| B. napus G1-3b gene | U. S. Pat. No. 5,401,836 |
| SbPRP1 | Suzuki et al., Plant Mol. Biol. 21: 109-119, 1993. |
| LRX1 | Baumberger et al. 2001, Genes & Dev. 15: 1128 |
| BTG-26 | US 20050044585 |
| Brassica napus | |
| LeAMT1 (tomato) | Lauter et al. (1996, PNAS 3: 8139) |
| The LeNRT1-1 | Lauter et al. (1996, PNAS 3: 8139) |
| (tomato) | |
| class I patatin | Liu et al., Plant Mol. Biol. 153: 386-395, 1991. |
| gene (potato) | |
| KDC1 | Downey et al. (2000, J. Biol. Chem. 275: 39420) |
| (Daucus carota) | |
| TobRB7 gene | W Song (1997) PhD Thesis, North Carolina |
| | State University, Raleigh, NC USA |
| OsRAB5a (rice) | Wang et al. 2002, Plant Sci. 163: 273 |
| ALF5 (Arabidopsis) | Diener et al. (2001, Plant Cell 13: 1625) |
| NRT2; 1Np (N. | Quesada et al. (1997, Plant Mol. Biol. 34: 265) |
| plumbaginifolia) | |

A seed-specific promoter is transcriptionally active predominantly in seed tissue, but not necessarily exclusively in seed tissue (in cases of leaky expression). The seed-specific promoter may be active during seed development and/or during germination. The seed specific promoter may be endosperm/aleurone/embryo specific. Examples of seed-specific promoters (endosperm/aleurone/embryo specific) are shown in Table 2c to Table 2f below. Further examples of seed-specific promoters are given in Qing Qu and Takaiwa (Plant Biotechnol. J. 2, 113-125, 2004), which disclosure is incorporated by reference herein as if fully set forth.

TABLE 2c
Examples of seed-specific promoters

| Gene source | Reference |
|---|---|
| seed-specific | Simon et al., Plant Mol. Biol. 5: 191, 1985; |
| genes | Scofield et al., J. Biol. Chem. 262: 12202, 1987.; |
| | Baszczynski et al., Plant Mol. Biol. 14: 633, 1990. |
| Brazil Nut albumin | Pearson et al., Plant Mol. Biol. 18: 235-245, 1992. |
| legumin | Ellis et al., Plant Mol. Biol. 10: 203-214, 1988. |
| glutelin (rice) | Takaiwa et al., Mol. Gen. Genet. 208: 15-22, 1986; |
| | Takaiwa et al., FEBS Letts. 221: 43-47, 1987. |
| zein | Matzke et al Plant Mol Biol, 14(3):323-32 1990 |
| napA | Stalberg et al, Planta 199: 515-519, 1996. |
| wheat LMW and | Mol Gen Genet 216: 81-90, 1989; NAR 17: |
| HMW glutenin-1 | 461-2, 1989 |
| wheat SPA | Albani et al, Plant Cell, 9: 171-184, 1997 |
| wheat α, β, γ-gliadins | EMBO J. 3: 1409-15, 1984 |
| barley Itr1 promoter | Diaz et al. (1995) Mol Gen Genet 248(5): 592-8 |
| barley B1, C, D, | Theor Appl Gen 98: 1253-62, 1999; Plant J 4: 343-55, |
| hordein | 1993; Mol Gen Genet 250: 750-60, 1996 |
| barley DOF | Mena et al, The Plant Journal, 116(1): 53-62, 1998 |
| blz2 | EP99106056.7 |
| synthetic promoter | Vicente-Carbajosa et al., Plant J. 13: 629-640, 1998. |
| rice prolamin NRP33 | Wu et al, Plant Cell Physiology 39(8) 885-889, 1998 |
| rice a-globulin Glb-1 | Wu et al, Plant Cell Physiology 39(8) 885-889, 1998 |
| rice OSH1 | Sato et al, Proc. Natl. Acad. Sci. USA, |
| | 93: 8117-8122, 1996 |
| rice α-globulin | Nakase et al. Plant Mol. Biol. 33: 513-522, 1997 |
| REB/OHP-1 | |
| rice ADP-glucose | Trans Res 6: 157-68, 1997 |
| pyrophosphorylase | |
| maize ESR gene | Plant J 12: 235-46, 1997 |
| family | |
| sorghum α-kafirin | DeRose et al., Plant Mol. Biol 32: 1029-35, 1996 |
| KNOX | Postma-Haarsma et al, Plant Mol. Biol. 39: |
| | 257-71, 1999 |
| rice oleosin | Wu et al, J. Biochem. 123: 386, 1998 |
| sunflower oleosin | Cummins et al., Plant Mol. Biol. 19: 873-876, 1992 |
| PRO0117, putative | WO 2004/070039 |
| rice 40S | |
| ribosomal protein | |
| PRO0136, rice | unpublished |
| alanine | |
| aminotransferase | |
| PRO0147, trypsin | unpublished |
| inhibitor | |
| ITR1 (barley) | |
| PRO0151, rice | WO 2004/070039 |
| WSI18 | |
| PRO0175, rice | WO 2004/070039 |
| RAB21 | |
| PRO005 | WO 2004/070039 |
| PRO0095 | WO 2004/070039 |
| α-amylase | Lanahan et al, Plant Cell 4: 203-211, 1992; |
| (Amy32b) | Skriver et al, Proc Natl Acad Sci USA |
| | 88: 7266-7270, 1991 |
| cathepsin β-like | Cejudo et al, Plant Mol Biol 20: 849-856, 1992 |
| gene | |
| Barley Ltp2 | Kalla et al., Plant J. 6: 849-60, 1994 |
| Chi26 | Leah et al., Plant J. 4: 579-89, 1994 |
| Maize B-Peru | Selinger et al., Genetics 149; 1125-38,1998 |

TABLE 2d
examples of endosperm-specific promoters

| Gene source | Reference |
|---|---|
| glutelin (rice) | Takaiwa et al. (1986) Mol Gen Genet 208: 15-22; |
| | Takaiwa et al. (1987) FEBS Letts. 221: 43-47 |
| zein | Matzke et al., (1990) Plant Mol Biol 14(3): 323-32 |
| wheat LMW and | Colot et al. (1989) Mol Gen Genet 216: 81-90, |
| HMW glutenin-1 | Anderson et al. (1989) NAR 17: 461-2 |
| wheat SPA | Albani et al. (1997) Plant Cell 9: 171-184 |
| wheat gliadins | Rafalski et al. (1984) EMBO 3: 1409-15 |
| barley Itr1 promoter | Diaz et al. (1995) Mol Gen Genet 248(5): 592-8 |
| barley B1, C, D, | Cho et al. (1999) Theor Appl Genet 98:1253-62; |
| hordein | Muller et al. (1993) Plant J 4: 343-55; |
| | Sorenson et al. (1996) Mol Gen Genet 250: 750-60 |
| barley DOF | Mena et al, (1998) Plant J 116(1): 53-62 |
| blz2 | Onate et al. (1999) J Biol Chem 274(14): 9175-82 |
| synthetic promoter | Vicente-Carbajosa et al. (1998) Plant J 13: 629- |
| | 640 |
| rice prolamin NRP33 | Wu et al, (1998) Plant Cell Physiol 39(8) 885-889 |
| rice globulin Glb-1 | Wu et al. (1998) Plant Cell Physiol 39(8) 885-889 |
| rice globulin | Nakase et al. (1997) Plant Molec Biol 33: 513-522 |
| REB/OHP-1 | |
| rice ADP-glucose | Russell et al. (1997) Trans Res 6: 157-68 |
| pyrophosphorylase | |
| maize ESR gene | Opsahl-Ferstad et al. (1997) Plant J 12: 235-46 |
| family | |
| sorghum kafirin | DeRose et al. (1996) Plant Mol Biol 32: 1029-35 |

TABLE 2e
Examples of embryo specific promoters:

| Gene source | Reference |
|---|---|
| rice OSH1 | Sato et al, Proc. Natl. Acad. Sci. USA, 93: 8117-8122, 1996 |
| KNOX | Postma-Haarsma et al, Plant Mol. Biol. 39: 257-71, 1999 |
| PRO0151 | WO 2004/070039 |
| PRO0175 | WO 2004/070039 |
| PRO005 | WO 2004/070039 |
| PRO0095 | WO 2004/070039 |

TABLE 2f
Examples of aleurone-specific promoters:

| Gene source | Reference |
|---|---|
| α-amylase (Amy32b) | Lanahan et al, Plant Cell 4: 203-211, 1992; |
| | Skriver et al, Proc Natl Acad Sci USA |
| | 88: 7266-7270, 1991 |
| cathepsin β-like gene | Cejudo et al, Plant Mol Biol 20: 849-856, 1992 |
| Barley Ltp2 | Kalla et al., Plant J. 6: 849-60, 1994 |
| Chi26 | Leah et al., Plant J. 4: 579-89, 1994 |
| Maize B-Peru | Selinger et al., Genetics 149; 1125-38, 1998 |

A green tissue-specific promoter as defined herein is a promoter that is transcriptionally active predominantly in green tissue, substantially to the exclusion of any other parts of a plant, whilst still allowing for any leaky expression in these other plant parts.

Examples of green tissue-specific promoters which may be used to perform the methods of the invention are shown in Table 2g below.

TABLE 2g
Examples of green tissue-specific promoters

| Gene | Expression | Reference |
|---|---|---|
| Maize Orthophosphate dikinase | Leaf specific | Fukavama et al., 2001 |
| Maize Phosphoenolpyruvate | Leaf specific | Kausch et al., 2001 |
| carboxylase | | |
| Rice Phosphoenolpyruvate | Leaf specific | Liu et al., 2003 |
| carboxylase | | |
| Rice small subunit Rubisco | Leaf specific | Nomura et al., 2000 |
| rice beta expansin EXBP9 | Shoot specific | WO 2004/070039 |
| Pigeonpea small subunit Rubisco | Leaf specific | Panguluri et al., 2005 |
| Pea RBCS3A | Leaf specific | |

Another example of a tissue-specific promoter is a meristem-specific promoter, which is transcriptionally active predominantly in meristematic tissue, substantially to the exclusion of any other parts of a plant, whilst still allowing for any leaky expression in these other plant parts. Examples of green meristem-specific promoters which may be used to perform the methods of the invention are shown in Table 2h below.

TABLE 2h
Examples of meristem-specific promoters

| Gene source | Expression pattern | Reference |
|---|---|---|
| rice OSH1 | Shoot apical meristem, | Sato et al. (1996) Proc. |
| | from embryo globular | Natl. Acad. Sci. USA, |
| | stage to seedling stage | 93: 8117-8122 |
| Rice | Meristem specific | BAD87835.1 |
| metallothionein | | |
| WAK1 & | Shoot and root apical | Wagner & Kohorn (2001) |
| WAK2 | meristems, and in | Plant Cell |
| | expanding leaves and | 13(2): 303-318 |
| | sepals | |

### Terminator

The term “terminator” encompasses a control sequence which is a DNA sequence at the end of a transcriptional unit which signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another plant gene, or less preferably from any other eukaryotic gene.

### Modulation

The term “modulation” means in relation to expression or gene expression, a process in which the expression level is changed by said gene expression in comparison to the control plant, the expression level may be increased or decreased. The original, unmodulated expression may be of any kind of expression of a structural RNA (rRNA, tRNA) or mRNA with subsequent translation. The term “modulating the activity” shall mean any change of the expression of the inventive nucleic acid sequences or encoded proteins, which leads to increased yield and/or increased growth of the plants.

### Expression

The term “expression” or “gene expression” means the transcription of a specific gene or specific genes or specific genetic construct. The term “expression” or “gene expression” in particular means the transcription of a gene or genes or genetic construct into structural RNA (rRNA, tRNA) or mRNA with or without subsequent translation of the latter into a protein. The process includes transcription of DNA and processing of the resulting mRNA product.

Increased Expression/Overexpression

The term “increased expression” or “overexpression” as used herein means any form of expression that is additional to the original wild-type expression level.

Methods for increasing expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of a nucleic acid encoding the polypeptide of interest. For example, endogenous promoters may be altered in vivo by mutation, deletion, and/or substitution (see, Kmiec, U.S. Pat. No. 5,565,350; Zarling et al., WO9322443), or isolated promoters may be introduced into a plant cell in the proper orientation and distance from a gene of the present invention so as to control the expression of the gene.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene.

An intron sequence may also be added to the 5′ untranslated region (UTR) or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg (1988) Mol. Cell biol. 8: 4395-4405; Callis et al. (1987) Genes Dev 1:1183-1200). Such intron enhancement of gene expression is typically greatest when placed near the 5′ end of the transcription unit. Use of the maize introns Adh1-S intron 1, 2, and 6, the Bronze-1 intron are known in the art. For general information see: The Maize Handbook, Chapter 116, Freeling and Walbot, Eds., Springer, N.Y. (1994).

### Endogenous Gene

Reference herein to an “endogenous” gene not only refers to the gene in question as found in a plant in its natural form (i.e., without there being any human intervention), but also refers to that same gene (or a substantially homologous nucleic acid/gene) in an isolated form subsequently (re)introduced into a plant (a transgene). For example, a transgenic plant containing such a transgene may encounter a substantial reduction of the transgene expression and/or substantial reduction of expression of the endogenous gene. The isolated gene may be isolated from an organism or may be manmade, for example by chemical synthesis.

### Decreased Expression

Reference herein to “decreased expression” or “reduction or substantial elimination” of expression is taken to mean a decrease in endogenous gene expression and/or polypeptide levels and/or polypeptide activity relative to control plants. The reduction or substantial elimination is in increasing order of preference at least 10%, 20%, 30%, 40% or 50%, 60%, 70%, 80%, 85%, 90%, or 95%, 96%, 97%, 98%, 99% or more reduced compared to that of control plants. Methods for decreasing expression are known in the art and the skilled person would readily be able to adapt the known methods for silencing so as to achieve reduction of expression of an endogenous gene in a whole plant or in parts thereof through the use of an appropriate promoter, for example.

For the reduction or substantial elimination of expression an endogenous gene in a plant, a sufficient length of substantially contiguous nucleotides of a nucleic acid sequence is required. In order to perform gene silencing, this may be as little as 20, 19, 18, 17, 16, 15, 14, 13, 12, 11, 10 or fewer nucleotides, alternatively this may be as much as the entire gene (including the 5′ and/or 3′ UTR, either in part or in whole). The stretch of substantially contiguous nucleotides may be derived from the nucleic acid encoding the protein of interest (target gene), or from any nucleic acid capable of encoding an orthologue, paralogue or homologue of the protein of interest. Preferably, the stretch of substantially contiguous nucleotides is capable of forming hydrogen bonds with the target gene (either sense or antisense strand), more preferably, the stretch of substantially contiguous nucleotides has, in increasing order of preference, 50%, 60%, 70%, 80%, 85%, or antisense strand). A nucleic acid sequence encoding a (functional) polypeptide is not a requirement for the various methods discussed herein for the reduction or substantial elimination of expression of an endogenous gene.

Examples of various methods for the reduction or substantial elimination of expression in a plant of an endogenous gene, or for lowering levels and/or activity of a protein, are known to the skilled in the art. A skilled person would readily be able to adapt the known methods for silencing, so as to achieve reduction of expression of an endogenous gene in a whole plant or in parts thereof through the use of an appropriate promoter, for example.

This reduction or substantial elimination of expression may be achieved using routine tools and techniques. A preferred method for the reduction or substantial elimination of endogenous gene expression is by introducing and expressing in a plant a genetic construct into which the nucleic acid (in this case a stretch of substantially contiguous nucleotides derived from the gene of interest, or from any nucleic acid capable of encoding an orthologue, paralogue or homologue of any one of the protein of interest) is cloned as an inverted repeat (in part or completely), separated by a spacer (non-coding DNA).

In such a preferred method, expression of the endogenous gene is reduced or substantially eliminated through RNA-mediated silencing using an inverted repeat of a nucleic acid or a part thereof (in this case a stretch of substantially contiguous nucleotides derived from the gene of interest, or from any nucleic acid capable of encoding an orthologue, paralogue or homologue of the protein of interest), preferably capable of forming a hairpin structure. The inverted repeat is cloned in an expression vector comprising control sequences. A non-coding DNA nucleic acid sequence (a spacer, for example a matrix attachment region fragment (MAR), an intron, a polylinker, etc.) is located between the two inverted nucleic acids forming the inverted repeat. After transcription of the inverted repeat, a chimeric RNA with a self-complementary structure is formed (partial or complete). This double-stranded RNA structure is referred to as the hairpin RNA (hpRNA). The hpRNA is processed by the plant into siRNAs that are incorporated into an RNA-induced silencing complex (RISC). The RISC further cleaves the mRNA transcripts, thereby substantially reducing the number of mRNA transcripts to be translated into polypeptides. For further general details see for example, Grierson et al. (1998) WO 98/53083; Waterhouse et al. (1999) WO 99/53050).

Performance of the methods of the invention does not rely on introducing and expressing in a plant a genetic construct into which the nucleic acid is cloned as an inverted repeat, but any one or more of several well-known “gene silencing” methods may be used to achieve the same effects.

One such method for the reduction of endogenous gene expression is RNA-mediated silencing of gene expression (downregulation). Silencing in this case is triggered in a plant by a double stranded RNA sequence (dsRNA) that is substantially similar to the target endogenous gene. This dsRNA is further processed by the plant into about 20 to about 26 nucleotides called short interfering RNAs (siRNAs). The siRNAs are incorporated into an RNA-induced silencing complex (RISC) that cleaves the mRNA transcript of the endogenous target gene, thereby substantially reducing the number of mRNA transcripts to be translated into a polypeptide. Preferably, the double stranded RNA sequence corresponds to a target gene.

Another example of an RNA silencing method involves the introduction of nucleic acid sequences or parts thereof (in this case a stretch of substantially contiguous nucleotides derived from the gene of interest, or from any nucleic acid capable of encoding an orthologue, paralogue or homologue of the protein of interest) in a sense orientation into a plant. “Sense orientation” refers to a DNA sequence that is homologous to an mRNA transcript thereof. Introduced into a plant would therefore be at least one copy of the nucleic acid sequence. The additional nucleic acid sequence will reduce expression of the endogenous gene, giving rise to a phenomenon known as co-suppression. The reduction of gene expression will be more pronounced if several additional copies of a nucleic acid sequence are introduced into the plant, as there is a positive correlation between high transcript levels and the triggering of co-suppression.

Another example of an RNA silencing method involves the use of antisense nucleic acid sequences. An “antisense” nucleic acid sequence comprises a nucleotide sequence that is complementary to a “sense” nucleic acid sequence encoding a protein, i.e. complementary to the coding strand of a double-stranded cDNA molecule or complementary to an mRNA transcript sequence. The antisense nucleic acid sequence is preferably complementary to the endogenous gene to be silenced. The complementarity may be located in the “coding region” and/or in the “non-coding region” of a gene. The term “coding region” refers to a region of the nucleotide sequence comprising codons that are translated into amino acid residues. The term “non-coding region” refers to 5′ and 3′ sequences that flank the coding region that are transcribed but not translated into amino acids (also referred to as 5′ and 3′ untranslated regions).

Antisense nucleic acid sequences can be designed according to the rules of Watson and Crick base pairing. The antisense nucleic acid sequence may be complementary to the entire nucleic acid sequence (in this case a stretch of substantially contiguous nucleotides derived from the gene of interest, or from any nucleic acid capable of encoding an orthologue, paralogue or homologue of the protein of interest), but may also be an oligonucleotide that is antisense to only a part of the nucleic acid sequence (including the mRNA 5′ and 3′ UTR). For example, the antisense oligonucleotide sequence may be complementary to the region surrounding the translation start site of an mRNA transcript encoding a polypeptide. The length of a suitable antisense oligonucleotide sequence is known in the art and may start from about 50, 45, 40, 35, 30, 25, 20, 15 or 10 nucleotides in length or less. An antisense nucleic acid sequence according to the invention may be constructed using chemical synthesis and enzymatic ligation reactions using methods known in the art. For example, an antisense nucleic acid sequence (e.g., an antisense oligonucleotide sequence) may be chemically synthesized using naturally occurring nucleotides or variously modified nucleotides designed to increase the biological stability of the molecules or to increase the physical stability of the duplex formed between the antisense and sense nucleic acid sequences, e.g., phosphorothioate derivatives and acridine substituted nucleotides may be used. Examples of modified nucleotides that may be used to generate the antisense nucleic acid sequences are well known in the art. Known nucleotide modifications include methylation, cyclization and ‘caps’ and substitution of one or more of the naturally occurring nucleotides with an analogue such as inosine. Other modifications of nucleotides are well known in the art.

The antisense nucleic acid sequence can be produced biologically using an expression vector into which a nucleic acid sequence has been subcloned in an antisense orientation (i.e., RNA transcribed from the inserted nucleic acid will be of an antisense orientation to a target nucleic acid of interest). Preferably, production of antisense nucleic acid sequences in plants occurs by means of a stably integrated nucleic acid construct comprising a promoter, an operably linked antisense oligonucleotide, and a terminator.

The nucleic acid molecules used for silencing in the methods of the invention (whether introduced into a plant or generated in situ) hybridize with or bind to mRNA transcripts and/or genomic DNA encoding a polypeptide to thereby inhibit expression of the protein, e.g., by inhibiting transcription and/or translation. The hybridization can be by conventional nucleotide complementarity to form a stable duplex, or, for example, in the case of an antisense nucleic acid sequence which binds to DNA duplexes, through specific interactions in the major groove of the double helix. Antisense nucleic acid sequences may be introduced into a plant by transformation or direct injection at a specific tissue site. Alternatively, antisense nucleic acid sequences can be modified to target selected cells and then administered systemically. For example, for systemic administration, antisense nucleic acid sequences can be modified such that they specifically bind to receptors or antigens expressed on a selected cell surface, e.g., by linking the antisense nucleic acid sequence to peptides or antibodies which bind to cell surface receptors or antigens. The antisense nucleic acid sequences can also be delivered to cells using the vectors described herein.

According to a further aspect, the antisense nucleic acid sequence is an a-anomeric nucleic acid sequence. An a-anomeric nucleic acid sequence forms specific double-stranded hybrids with complementary RNA in which, contrary to the usual b-units, the strands run parallel to each other (Gaultier et al. (1987) Nucl Ac Res 15: 6625-6641). The antisense nucleic acid sequence may also comprise a 2′-o-methylribonucleotide (Inoue et al. (1987) Nucl Ac Res 15, 6131-6148) or a chimeric RNA-DNA analogue (Inoue et al. (1987) FEBS Lett. 215, 327-330).

The reduction or substantial elimination of endogenous gene expression may also be performed using ribozymes. Ribozymes are catalytic RNA molecules with ribonuclease activity that are capable of cleaving a single-stranded nucleic acid sequence, such as an mRNA, to which they have a complementary region. Thus, ribozymes (e.g., hammerhead ribozymes (described in Haselhoff and Gerlach (1988) Nature 334, 585-591) can be used to catalytically cleave mRNA transcripts encoding a polypeptide, thereby substantially reducing the number of mRNA transcripts to be translated into a polypeptide. A ribozyme having specificity for a nucleic acid sequence can be designed (see for example: Cech et al. U.S. Pat. No. 4,987,071; and Cech et al. U.S. Pat. No. 5,116,742). Alternatively, mRNA transcripts corresponding to a nucleic acid sequence can be used to select a catalytic RNA having a specific ribonuclease activity from a pool of RNA molecules (Bartel and Szostak (1993) Science 261, 1411-1418). The use of ribozymes for gene silencing in plants is known in the art (e.g., Atkins et al. (1994) WO 94/00012; Lenne et al. (1995) WO 95/03404; Lutziger et al. (2000) WO 00/00619; Prinsen et al. (1997) WO 97/13865 and Scott et al. (1997) WO 97/38116).

Gene silencing may also be achieved by insertion mutagenesis (for example, T-DNA insertion or transposon insertion) or by strategies as described by, among others, Angell and Baulcombe ((1999) Plant J 20(3): 357-62), (Amplicon VIGS WO 98/36083), or Baulcombe (WO 99/15682).

Gene silencing may also occur if there is a mutation on an endogenous gene and/or a mutation on an isolated gene/nucleic acid subsequently introduced into a plant. The reduction or substantial elimination may be caused by a non-functional polypeptide. For example, the polypeptide may bind to various interacting proteins; one or more mutation(s) and/or truncation(s) may therefore provide for a polypeptide that is still able to bind interacting proteins (such as receptor proteins) but that cannot exhibit its normal function (such as signalling ligand).

A further approach to gene silencing is by targeting nucleic acid sequences complementary to the regulatory region of the gene (e.g., the promoter and/or enhancers) to form triple helical structures that prevent transcription of the gene in target cells. See Helene, C., Anticancer Drug Res. 6, 569-84, 1991; Helene et al., Ann. N.Y. Acad. Sci. 660, 27-36 1992; and Maher, L. J. Bioassays 14, 807-15, 1992.

Other methods, such as the use of antibodies directed to an endogenous polypeptide for inhibiting its function in planta, or interference in the signalling pathway in which a polypeptide is involved, will be well known to the skilled man. In particular, it can be envisaged that manmade molecules may be useful for inhibiting the biological function of a target polypeptide, or for interfering with the signalling pathway in which the target polypeptide is involved.

Alternatively, a screening program may be set up to identify in a plant population natural variants of a gene, which variants encode polypeptides with reduced activity. Such natural variants may also be used for example, to perform homologous recombination.

Artificial and/or natural microRNAs (miRNAs) may be used to knock out gene expression and/or mRNA translation. Endogenous miRNAs are single stranded small RNAs of typically 19-24 nucleotides long. They function primarily to regulate gene expression and/or mRNA translation. Most plant microRNAs (miRNAs) have perfect or near-perfect complementarity with their target sequences. However, there are natural targets with up to five mismatches. They are processed from longer non-coding RNAs with characteristic fold-back structures by double-strand specific RNases of the Dicer family. Upon processing, they are incorporated in the RNA-induced silencing complex (RISC) by binding to its main component, an Argonaute protein. MiRNAs serve as the specificity components of RISC, since they base-pair to target nucleic acids, mostly mRNAs, in the cytoplasm. Subsequent regulatory events include target mRNA cleavage and destruction and/or translational inhibition. Effects of miRNA overexpression are thus often reflected in decreased mRNA levels of target genes.

Artificial microRNAs (amiRNAs), which are typically 21 nucleotides in length, can be genetically engineered specifically to negatively regulate gene expression of single or multiple genes of interest. Determinants of plant microRNA target selection are well known in the art. Empirical parameters for target recognition have been defined and can be used to aid in the design of specific amiRNAs, (Schwab et al., Dev. Cell 8, 517-527, 2005). Convenient tools for design and generation of amiRNAs and their precursors are also available to the public (Schwab et al., Plant Cell 18, 1121-1133, 2006).

For optimal performance, the gene silencing techniques used for reducing expression in a plant of an endogenous gene requires the use of nucleic acid sequences from monocotyledonous plants for transformation of monocotyledonous plants, and from dicotyledonous plants for transformation of dicotyledonous plants. Preferably, a nucleic acid sequence from any given plant species is introduced into that same species. For example, a nucleic acid sequence from rice is transformed into a rice plant. However, it is not an absolute requirement that the nucleic acid sequence to be introduced originates from the same plant species as the plant in which it will be introduced. It is sufficient that there is substantial homology between the endogenous target gene and the nucleic acid to be introduced.

Described above are examples of various methods for the reduction or substantial elimination of expression in a plant of an endogenous gene. A person skilled in the art would readily be able to adapt the aforementioned methods for silencing so as to achieve reduction of expression of an endogenous gene in a whole plant or in parts thereof through the use of an appropriate promoter, for example.

### Selectable Marker (Gene)/Reporter Gene

“Selectable marker”, “selectable marker gene” or “reporter gene” includes any gene that confers a phenotype on a cell in which it is expressed to facilitate the identification and/or selection of cells that are transfected or transformed with a nucleic acid construct of the invention. These marker genes enable the identification of a successful transfer of the nucleic acid molecules via a series of different principles. Suitable markers may be selected from markers that confer antibiotic or herbicide resistance, that introduce a new metabolic trait or that allow visual selection. Examples of selectable marker genes include genes conferring resistance to antibiotics (such as nptII that phosphorylates neomycin and kanamycin, or hpt, phosphorylating hygromycin, or genes conferring resistance to, for example, bleomycin, streptomycin, tetracyclin, chloramphenicol, ampicillin, gentamycin, geneticin (G418), spectinomycin or blasticidin), to herbicides (for example bar which provides resistance to Basta®; aroA or gox providing resistance against glyphosate, or the genes conferring resistance to, for example, imidazolinone, phosphinothricin or sulfonylurea), or genes that provide a metabolic trait (such as manA that allows plants to use mannose as sole carbon source or xylose isomerase for the utilisation of xylose, or antinutritive markers such as the resistance to 2-deoxyglucose). Expression of visual marker genes results in the formation of colour (for example β-glucuronidase, GUS or β-galactosidase with its coloured substrates, for example X-Gal), luminescence (such as the luciferin/luceferase system) or fluorescence (Green Fluorescent Protein, GFP, and derivatives thereof). This list represents only a small number of possible markers. The skilled worker is familiar with such markers. Different markers are preferred, depending on the organism and the selection method.

It is known that upon stable or transient integration of nucleic acids into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can for example be used in mutants in which these genes are not functional by, for example, deletion by conventional methods. Furthermore, nucleic acid molecules encoding a selectable marker can be introduced into a host cell on the same vector that comprises the sequence encoding the polypeptides of the invention or used in the methods of the invention, or else in a separate vector. Cells which have been stably transfected with the introduced nucleic acid can be identified for example by selection (for example, cells which have integrated the selectable marker survive whereas the other cells die). The marker genes may be removed or excised from the transgenic cell once they are no longer needed. Techniques for marker gene removal are known in the art, useful techniques are described above in the definitions section.

Since the marker genes, particularly genes for resistance to antibiotics and herbicides, are no longer required or are undesired in the transgenic host cell once the nucleic acids have been introduced successfully, the process according to the invention for introducing the nucleic acids advantageously employs techniques which enable the removal or excision of these marker genes. One such a method is what is known as co-transformation. The co-transformation method employs two vectors simultaneously for the transformation, one vector bearing the nucleic acid according to the invention and a second bearing the marker gene(s). A large proportion of transformants receives or, in the case of plants, comprises (up to 40% or more of the transformants), both vectors. In case of transformation with Agrobacteria, the transformants usually receive only a part of the vector, i.e. the sequence flanked by the T-DNA, which usually represents the expression cassette. The marker genes can subsequently be removed from the transformed plant by performing crosses. In another method, marker genes integrated into a transposon are used for the transformation together with desired nucleic acid (known as the Ac/Ds technology). The transformants can be crossed with a transposase source or the transformants are transformed with a nucleic acid construct conferring expression of a transposase, transiently or stable. In some cases (approx. 10%), the transposon jumps out of the genome of the host cell once transformation has taken place successfully and is lost. In a further number of cases, the transposon jumps to a different location. In these cases the marker gene must be eliminated by performing crosses. In microbiology, techniques were developed which make possible, or facilitate, the detection of such events. A further advantageous method relies on what is known as recombination systems; whose advantage is that elimination by crossing can be dispensed with. The best-known system of this type is what is known as the Cre/lox system. Cre1 is a recombinase that removes the sequences located between the loxP sequences. If the marker gene is integrated between the loxP sequences, it is removed once transformation has taken place successfully, by expression of the recombinase. Further recombination systems are the HIN/HIX, FLP/FRT and REP/STB system (Tribble et al., J. Biol. Chem., 275, 2000: 22255-22267; Velmurugan et al., J. Cell Biol., 149, 2000: 553-566). A site-specific integration into the plant genome of the nucleic acid sequences according to the invention is possible. Naturally, these methods can also be applied to microorganisms such as yeast, fungi or bacteria.

### Transgenic/Transgene/Recombinant

For the purposes of the invention, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention, all those constructions brought about by recombinant methods in which either
(a) the nucleic acid sequences encoding proteins useful in the methods of the invention, or
(b) genetic control sequence(s) which is operably linked with the nucleic acid sequence according to the invention, for example a promoter, or
(c) a) and b)
   are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods of the present invention, as defined above—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815.

A transgenic plant for the purposes of the invention is thus understood as meaning, as above, that the nucleic acids used in the method of the invention are not at their natural locus in the genome of said plant, it being possible for the nucleic acids to be expressed homologously or heterologously. However, as mentioned, transgenic also means that, while the nucleic acids according to the invention or used in the inventive method are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, preferably, heterologous expression of the nucleic acids takes place. Preferred transgenic plants are mentioned herein.

### Transformation

The term “introduction” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., (1982) Nature 296, 72-74; Negrutiu I et al. (1987) Plant Mol Biol 8: 363-373); electroporation of protoplasts (Shillito R. D. et al. (1985) Bio/Technol 3, 1099-1102); microinjection into plant material (Crossway A et al., (1986) Mol. Gen Genet 202: 179-185); DNA or RNA-coated particle bombardment (Klein T M et al., (1987) Nature 327: 70) infection with (non-integrative) viruses and the like. Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least on the flower primordia. The plant is subsequently grown on until the seeds of the treated plant are obtained (Clough and Bent, Plant J. (1998) 16, 735-743). Methods for Agrobacterium-mediated transformation of rice include well known methods for rice transformation, such as those described in any of the following: European patent application EP 1198985 A1, Aldemita and Hodges (Planta 199: 612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14(6): 745-50, 1996) or Frame et al. (Plant Physiol 129(1): 13-22, 2002), which disclosures are incorporated by reference herein as if fully set forth. Said methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). The nucleic acids or the construct to be expressed is preferably cloned into a vector, which is suitable for transforming Agrobacterium tumefaciens, for example pBin19 (Bevan et al., Nucl. Acids Res. 12 (1984) 8711). Agrobacteria transformed by such a vector can then be used in known manner for the transformation of plants, such as plants used as a model, like Arabidopsis (Arabidopsis thaliana is within the scope of the present invention not considered as a crop plant), or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Hofgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

In addition to the transformation of somatic cells, which then have to be regenerated into intact plants, it is also possible to transform the cells of plant meristems and in particular those cells which develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic [Feldman, K A and Marks M D (1987). Mol Gen Genet 208:274-289; Feldmann K (1992). In: C Koncz, N-H Chua and J Shell, eds, Methods in Arabidopsis Research. Word Scientific, Singapore, pp. 274-289]. Alternative methods are based on the repeated removal of the inflorescences and incubation of the excision site in the center of the rosette with transformed agrobacteria, whereby transformed seeds can likewise be obtained at a later point in time (Chang (1994). Plant J. 5: 551-558; Katavic (1994). Mol Gen Genet, 245: 363-370). However, an especially effective method is the vacuum infiltration method with its modifications such as the “floral dip” method. In the case of vacuum infiltration of Arabidopsis, intact plants under reduced pressure are treated with an agrobacterial suspension [Bechthold, N (1993). C R Acad Sci Paris Life Sci, 316: 1194-1199], while in the case of the “floral dip” method the developing floral tissue is incubated briefly with a surfactant-treated agrobacterial suspension [Clough, SJ and Bent AF (1998) The Plant J. 16, 735-743]. A certain proportion of transgenic seeds are harvested in both cases, and these seeds can be distinguished from non-transgenic seeds by growing under the above-described selective conditions. In addition the stable transformation of plastids is of advantages because plastids are inherited maternally is most crops reducing or eliminating the risk of transgene flow through pollen. The transformation of the chloroplast genome is generally achieved by a process which has been schematically displayed in Klaus et al., 2004 [Nature Biotechnology 22 (2), 225-229]. Briefly the sequences to be transformed are cloned together with a selectable marker gene between flanking sequences homologous to the chloroplast genome. These homologous flanking sequences direct site specific integration into the plastome. Plastidal transformation has been described for many different plant species and an overview is given in Bock (2001) Transgenic plastids in basic research and plant biotechnology. J Mol Biol. 2001 Sep. 21; 312 (3):425-38 or Maliga, P (2003) Progress towards commercialization of plastid transformation technology. Trends Biotechnol. 21, 20-28. Further biotechnological progress has recently been reported in form of marker free plastid transformants, which can be produced by a transient co-integrated maker gene (Klaus et al., 2004, Nature Biotechnology 22(2), 225-229).

### T-DNA Activation Tagging

T-DNA activation tagging (Hayashi et al. Science (1992) 1350-1353), involves insertion of T-DNA, usually containing a promoter (may also be a translation enhancer or an intron), in the genomic region of the gene of interest or 10 kb up- or downstream of the coding region of a gene in a configuration such that the promoter directs expression of the targeted gene. Typically, regulation of expression of the targeted gene by its natural promoter is disrupted and the gene falls under the control of the newly introduced promoter. The promoter is typically embedded in a T-DNA. This T-DNA is randomly inserted into the plant genome, for example, through Agrobacterium infection and leads to modified expression of genes near the inserted T-DNA. The resulting transgenic plants show dominant phenotypes due to modified expression of genes close to the introduced promoter.

### TILLING

The term “TILLING” is an abbreviation of “Targeted Induced Local Lesions In Genomes” and refers to a mutagenesis technology useful to generate and/or identify nucleic acids encoding proteins with modified expression and/or activity. TILLING also allows selection of plants carrying such mutant variants. These mutant variants may exhibit modified expression, either in strength or in location or in timing (if the mutations affect the promoter for example). These mutant variants may exhibit higher activity than that exhibited by the gene in its natural form. TILLING combines high-density mutagenesis with high-throughput screening methods. The steps typically followed in TILLING are: (a) EMS mutagenesis (Redei G P and Koncz C (1992) In Methods in Arabidopsis Research, Koncz C, Chua N H, Schell J, eds. Singapore, World Scientific Publishing Co, pp. 16-82; Feldmann et al., (1994) In Meyerowitz E M, Somerville C R, eds, Arabidopsis. Cold Spring Harbor Laboratory Press, Cold Spring Harbor, N.Y., pp 137-172; Lightner J and Caspar T (1998) In J Martinez-Zapater, J Salinas, eds, Methods on Molecular Biology, Vol. 82. Humana Press, Totowa, N.J., pp 91-104); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (McCallum et al., (2000) Nat Biotechnol 18: 455-457; reviewed by Stemple (2004) Nat Rev Genet 5(2): 145-50).

### Homologous Recombination

Homologous recombination allows introduction in a genome of a selected nucleic acid at a defined selected position. Homologous recombination is a standard technology used routinely in biological sciences for lower organisms such as yeast or the moss Physcomitrella. Methods for performing homologous recombination in plants have been described not only for model plants (Offring a et al. (1990) EMBO J 9(10): 3077-84) but also for crop plants, for example rice (Terada et al. (2002) Nat Biotech 20(10): 1030-4; lida and Terada (2004) Curr Opin Biotech 15(2): 132-8), and approaches exist that are generally applicable regardless of the target organism (Miller et al, Nature Biotechnol. 25, 778-785, 2007).

### Yield

The term “yield” in general means a measurable produce of economic value, typically related to a specified crop, to an area, and to a period of time. Individual plant parts directly contribute to yield based on their number, size and/or weight, or the actual yield is the yield per square meter for a crop and year, which is determined by dividing total production (includes both harvested and appraised production) by planted square meters. The term “yield” of a plant may relate to vegetative biomass (root and/or shoot biomass), to reproductive organs, and/or to propagules (such as seeds) of that plant.

### Early Vigour

“Early vigour” refers to active healthy well-balanced growth especially during early stages of plant growth, and may result from increased plant fitness due to, for example, the plants being better adapted to their environment (i.e. optimizing the use of energy resources and partitioning between shoot and root). Plants having early vigour also show increased seedling survival and a better establishment of the crop, which often results in highly uniform fields (with the crop growing in uniform manner, i.e. with the majority of plants reaching the various stages of development at substantially the same time), and often better and higher yield. Therefore, early vigour may be determined by measuring various factors, such as thousand kernel weight, percentage germination, percentage emergence, seedling growth, seedling height, root length, root and shoot biomass and many more.

### Increase/Improve/Enhance

The terms “increase”, “improve” or “enhance” are interchangeable and shall mean in the sense of the application at least a 3%, 4%, 5%, 6%, 7%, 8%, 9% or 10%, preferably at least 15% or 20%, more preferably 25%, 30%, 35% or 40% more yield and/or growth in comparison to control plants as defined herein.

### Seed Yield

Increased seed yield may manifest itself as one or more of the following: a) an increase in seed biomass (total seed weight) which may be on an individual seed basis and/or per plant and/or per square meter; b) increased number of flowers per plant; c) increased number of (filled) seeds; d) increased seed filling rate (which is expressed as the ratio between the number of filled seeds divided by the total number of seeds); e) increased harvest index, which is expressed as a ratio of the yield of harvestable parts, such as seeds, divided by the total biomass; and f) increased thousand kernel weight (TKW), and g) increased number of primary panicles, which is extrapolated from the number of filled seeds counted and their total weight. An increased TKW may result from an increased seed size and/or seed weight, and may also result from an increase in embryo and/or endosperm size.

An increase in seed yield may also be manifested as an increase in seed size and/or seed volume. Furthermore, an increase in seed yield may also manifest itself as an increase in seed area and/or seed length and/or seed width and/or seed perimeter. Increased seed yield may also result in modified architecture, or may occur because of modified architecture.

### Greenness Index

The “greenness index” as used herein is calculated from digital images of plants. For each pixel belonging to the plant object on the image, the ratio of the green value versus the red value (in the RGB model for encoding color) is calculated. The greenness index is expressed as the percentage of pixels for which the green-to-red ratio exceeds a given threshold. Under normal growth conditions, under salt stress growth conditions, and under reduced nutrient availability growth conditions, the greenness index of plants is measured in the last imaging before flowering. In contrast, under drought stress growth conditions, the greenness index of plants is measured in the first imaging after drought.

### Plant

The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

Plants that are particularly useful in the methods of the invention include all plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticale sp., Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

### DETAILED DESCRIPTION OF THE INVENTION

Surprisingly, it has now been found that modulating expression in a plant of a nucleic acid encoding a COX VIIa subunit polypeptide gives plants having enhanced abiotic stress tolerance relative to control plants. According to a first embodiment, the present invention provides a method for enhancing tolerance to various abiotic stresses in plants relative to control plants, comprising modulating expression in a plant of a nucleic acid encoding a COX VIIa subunit polypeptide and optionally selecting for plants having enhanced tolerance to abiotic stress.

Furthermore surprisingly, it has now been found that modulating expression in a plant of a nucleic acid encoding a YLD-ZnF polypeptide gives plants having enhanced yield-related traits relative to control plants. According to a first embodiment, the present invention provides a method for enhancing yield-related traits in plants relative to control plants, comprising modulating expression in a plant of a nucleic acid encoding a YLD-ZnF polypeptide and optionally selecting for plants having enhanced yield-related traits.

Furthermore, it has now surprisingly been found that modulating expression in a plant of a nucleic acid encoding a PKT polypeptide gives plants having enhanced abiotic stress tolerance relative to control plants. According to a first embodiment, the present invention provides a method for enhancing tolerance to various abiotic stresses in plants relative to control plants, comprising modulating expression in a plant of a nucleic acid encoding a PKT polypeptide and optionally selecting for plants having enhanced tolerance to abiotic stress.

Furthermore, it has now surprisingly been found that modulating expression in a plant of a nucleic acid encoding a NOA polypeptide gives plants having enhanced yield-related traits relative to control plants. According to a first embodiment, the present invention provides a method for enhancing yield-related traits in plants relative to control plants, comprising modulating expression in a plant of a nucleic acid encoding a NOA polypeptide and optionally selecting for plants having enhanced yield-related traits.

Furthermore, it has now surprisingly been found that modulating expression in a plant of a nucleic acid encoding an ASF1-like polypeptide gives plants having enhanced yield-related traits relative to control plants. According to a first embodiment, the present invention provides a method for enhancing yield-related traits in plants relative to control plants, comprising modulating expression in a plant of a nucleic acid encoding an ASF1-like polypeptide.

Furthermore, it has now surprisingly been found that modulating expression in a plant of a nucleic acid encoding a PHDF polypeptide gives plants having enhanced abiotic stress tolerance relative to control plants. According to a first embodiment, the present invention provides a method for enhancing tolerance to various abiotic stresses in plants relative to control plants, comprising modulating expression in a plant of a nucleic acid encoding a PHDF polypeptide and optionally selecting for plants having enhanced tolerance to abiotic stress.

Furthermore, it has now surprisingly been found that increasing expression in a plant of a nucleic acid sequence encoding a group I MBF1 polypeptide as defined herein, gives plants having increased yield-related traits relative to control plants. According to a first embodiment, the present invention provides a method for increasing yield-related traits in plants relative to control plants, comprising increasing expression in a plant of a nucleic acid sequence encoding a group I MBF1 polypeptide.

A preferred method for modulating (preferably, increasing) expression of a nucleic acid encoding a COX VIIa subunit polypeptide, or a YLD-ZnF polypeptide, or a PKT polypeptide, or a NOA polypeptide, or an ASF1-like polypeptide, or a PHDF polypeptide, or a group I MBF1 polypeptide, is by introducing and expressing in a plant a nucleic acid encoding a COX VIIa subunit polypeptide, or a YLD-ZnF polypeptide, or a PKT polypeptide, or a NOA polypeptide, or an ASF1-like polypeptide, or a PHDF polypeptide, or a group I MBF1 polypeptide.

Concerning COX VIIa subunit polypeptides, any reference hereinafter to a “protein useful in the methods of the invention” is taken to mean a COX VIIa subunit polypeptide as defined herein. Any reference hereinafter to a “nucleic acid useful in the methods of the invention” is taken to mean a nucleic acid capable of encoding such a COX VIIa subunit polypeptide. The nucleic acid to be introduced into a plant (and therefore useful in performing the methods of the invention) is any nucleic acid encoding the type of protein which will now be described, hereinafter also named “COX VIIa subunit nucleic acid” or “COX VIIa subunit gene”.

Concerning YLD-ZnF polypeptides, any reference hereinafter to a “protein useful in the methods of the invention” is taken to mean a YLD-ZnF polypeptide as defined herein. Any reference hereinafter to a “nucleic acid useful in the methods of the invention” is taken to mean a nucleic acid capable of encoding such a YLD-ZnF polypeptide. The nucleic acid to be introduced into a plant (and therefore useful in performing the methods of the invention) is any nucleic acid encoding the type of protein which will now be described, hereinafter also named “YLD-ZnF nucleic acid” or “YLD-ZnF gene”.

Concerning PKT polypeptides, any reference hereinafter to a “protein useful in the methods of the invention” is taken to mean a PKT polypeptide as defined herein. Any reference hereinafter to a “nucleic acid useful in the methods of the invention” is taken to mean a nucleic acid capable of encoding such a PKT polypeptide. The nucleic acid to be introduced into a plant (and therefore useful in performing the methods of the invention) is any nucleic acid encoding the type of protein which will now be described, hereinafter also named “PKT nucleic acid” or “PKT gene”.

Concerning NOA polypeptides, any reference hereinafter to a “protein useful in the methods of the invention” is taken to mean a NOA polypeptide as defined herein. Any reference hereinafter to a “nucleic acid useful in the methods of the invention” is taken to mean a nucleic acid capable of encoding such a NOA polypeptide. The nucleic acid to be introduced into a plant (and therefore useful in performing the methods of the invention) is any nucleic acid encoding the type of protein which will now be described, hereinafter also named “NOA nucleic acid” or “NOA gene”.

Concerning ASF1-like polypeptides, any reference hereinafter to a “protein useful in the methods of the invention” is taken to mean an ASF1-like polypeptide as defined herein. Any reference hereinafter to a “nucleic acid useful in the methods of the invention” is taken to mean a nucleic acid capable of encoding such an ASF1-like polypeptide. The nucleic acid to be introduced into a plant (and therefore useful in performing the methods of the invention) is any nucleic acid encoding the type of protein which will now be described, hereinafter also named “ASF1-like nucleic acid” or “ASF1-like gene”.

Concerning PHDF polypeptides, any reference hereinafter to a “protein useful in the methods of the invention” is taken to mean a PHDF polypeptide as defined herein. Any reference hereinafter to a “nucleic acid useful in the methods of the invention” is taken to mean a nucleic acid capable of encoding such a PHDF polypeptide. The nucleic acid to be introduced into a plant (and therefore useful in performing the methods of the invention) is any nucleic acid encoding the type of protein which will now be described, hereinafter also named “PHDF nucleic acid” or “PHDF gene”.

Concerning a group I MBF1 polypeptides, any reference hereinafter to a “protein useful in the methods of the invention” is taken to mean a group I MBF1 polypeptide as defined herein. Any reference hereinafter to a “nucleic acid sequence useful in the methods of the invention” is taken to mean a nucleic acid sequence capable of encoding such a group I MBF1 polypeptide. The nucleic acid sequence to be introduced into a plant (and therefore useful in performing the methods of the invention) is any nucleic acid sequence encoding the type of polypeptide, which will now be described, hereinafter also named “group I MBF1 nucleic acid sequence” or “group I MBF1 gene”.

A “COX VIIa subunit polypeptide” as defined herein refers to any polypeptide comprising a COX VIIa subunit or COX VIIa subunit activity.

Examples of such COX VIIa subunit polypeptides include orthologues and paralogues of the sequences represented by any of SEQ ID NO: 2, SEQ ID NO: 4, SEQ ID NO: 6 and SEQ ID NO: 8.

COX VIIa subunit polypeptides and orthologues and paralogues thereof typically have in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by any of SEQ ID NO: 2, SEQ ID NO: 4, SEQ ID NO: 6 and SEQ ID NO: 8.

The overall sequence identity is determined using a global alignment algorithm, such as the Needleman Wunsch algorithm in the program GAP (GCG Wisconsin Package, Accelrys), preferably with default parameters and preferably with sequences of mature proteins (i.e. without taking into account secretion signals or transit peptides). Compared to overall sequence identity, the sequence identity will generally be higher when only conserved domains or motifs are considered.

Preferably, the polypeptide sequence which when used in the construction of a phylogenetic tree, clusters with the group of COX VIIa subunit polypeptides comprising the amino acid sequences represented by SEQ ID NO: 2, SEQ ID NO: 4, SEQ ID NO: 6 and SEQ ID NO: 8. rather than with any other group. Tools and techniques for the construction and analysis of phylogenetic trees are well known in the art.

A “YLD-ZnF polypeptide” as defined herein refers to any polypeptide comprising zf-DNL domain (Pfam entry PF05180) and having motif 1 and/or motif 2:

### Motif 1 (SEQ ID NO: 20):

FTC(K/N)(V/S)C(E/D/G)(T/Q/E)R(S/T)

### Motif 2 (SEQ ID NO: 21):

(C/S/N)(R/K/P)(E/D/H)(S/A)Y(E/D/T)(K/N/D)G(V/T/L)
V(V/I/F)(A/V)(R/Q)C(G/C/A)GC(N/D/L)(N/V/K)(L/F/H)
H(L/K)(I/M/L)(A/V)D(H/R/N)(L/R)(G/N)(W/L)(F/I)
(G/H/V)

Preferably, Motif 1 is

FTCKVC(E/D)TRS

Preferably, Motif 2 is

(C/S)(R/K)(E/D)SY(E/D)(K/N)GVV(V/I)(A/V)RCGGC
(N/D)NLHL(I/M)AD(H/R)(L/R)GWFG

Further preferably, the YLD-ZnF polypeptide useful in the methods of this invention also comprises Motif 3 and/or Motif 4:

### Motif 3 (SEQ ID NO: 22):

K(R/K)G(S/D)XD(T/S)(L/F/I)(N/S)

Wherein X in position 5 can be any amino acid, but preferably one of G, I, M, A, T

### Motif 4 (SEQ ID NO: 23):

T(L/F)(E/D)D(L/I)(A/T/V)G

Alternatively, the homologue of a YLD-ZnF protein has in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 19, provided that the homologous protein comprises the conserved motifs as outlined above. The overall sequence identity is determined using a global alignment algorithm, such as the Needleman Wunsch algorithm in the program GAP (GCG Wisconsin Package, Accelrys), preferably with default parameters and preferably with sequences of mature proteins (i.e. without taking into account secretion signals or transit peptides). Compared to overall sequence identity, the sequence identity will generally be higher when only conserved domains or motifs are considered.

Preferably, the polypeptide sequence which when used in the construction of a phylogenetic tree, such as the one depicted in FIG. 4, clusters with the group of YLD-ZnF polypeptides comprising the amino acid sequence represented by SEQ ID NO: 19 (TA25762) rather than with any other group.

A “PKT polypeptide” as defined herein refers to any polypeptide comprising a protein kinase (PK) domain and one or more tetratricopeptide repeats (TPR).

Examples of such PKT polypeptides include orthologues and paralogues of the sequences represented by any of SEQ ID NO: 52 and SEQ ID NO: 54.

PKT polypeptides and orthologues and paralogues thereof typically have in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by any of SEQ ID NO: 52 and SEQ ID NO: 54.

The overall sequence identity is determined using a global alignment algorithm, such as the Needleman Wunsch algorithm in the program GAP (GCG Wisconsin Package, Accelrys), preferably with default parameters and preferably with sequences of mature proteins (i.e. without taking into account secretion signals or transit peptides). Compared to overall sequence identity, the sequence identity will generally be higher when only conserved domains or motifs are considered.

Preferably, the polypeptide sequence which when used in the construction of a phylogenetic tree, clusters with the group of PKT polypeptides comprising the amino acid sequences represented by SEQ ID NO: 52 and SEQ ID NO: 54. rather than with any other group. Tools and techniques for the construction and analysis of phylogenetic trees are well known in the art.

TPR repeats are well known in the art as being a degenerate 34 amino acid sequence present in tandem arrays of 3-16 motifs, which form scaffolds to mediate protein-protein interactions and often the assembly of multiprotein complexes.

A “NOA polypeptide” as defined herein refers to a polypeptide belonging to the family of circularly permutated GTPase family, comprising a GTP-Binding Protein-Related domain (HMMPanther accession PTHR11089). Preferably the NOA polypeptide comprises at least one of the following motifs (multilevel consensus sequences identified by MEME 3.5.0):

### Motif 5 (Starting at Position 318 in SEQ ID NO: 59):

LTEAPVPGTTLGIIRIXGVLGGGAKMYDTPGLLHPYQLTMRLNREEQKLV
PIQSA PLQV AF PAKKLLFTPGVH HH MSS T DLP MA
S YD R AV

as a regular expression (SEQ ID NO: 60):

(L/P)(T/I)(E/Q)(A/S)(P/A)VPGTTLG(I/P)(I/L)(R/Q)
(I/V)X(G/A)(V/F)L(G/P/S)(G/A)(G/K)(A/K)(K/L)
(M/L/Y)(Y/F/D)(D/T)(T/P)(P/G)(G/V)(L/H)LH(P/H)
(Y/H/R)Q(L/M)(T/S/A)(M/S/V)RL(N/T)R(E/D)(E/L)(Q/P)
K(L/M)(V/A)

wherein X in position 17 can be any amino acid.

### Motif 6 (Starting at Position 449 in SEQ ID NO: 59):

LLQPPIGEERVXELGKWXEREVKVSGESWDRSSVDIAIAGLGWFSVGLKG
RTP G P W L LQI D VNA VSVS IALEP
I P G

as a regular expression (SEQ ID NO: 61):

(L/R)(L/T)(Q/P)PP(I/G)G(E/P)ERVX(E/W)LG(K/L)WXERE
(V/L/I)(K/Q)(V/I)SGE(S/D)WD(R/V)(S/N/P)(S/A)VD
(I/V)(A/S)(I/V)(A/S)GLGW(F/I)(S/A/G)(V/L)(G/E)
(L/P)KG

wherein X in positions 12 and 18 can be any amino acid.

### Motif 7 (Starting at Position 194 in SEQ ID NO: 59):

KLVDIVDFNGSFLARVRDLAGANPIILVITKVDLLPRDTDLNCVGDWVVE
V FV V KG I

as a regular expression (SEQ ID NO: 62):

KLVD(I/V)VDFNGSFLARVRD(L/F)(A/V)GANPIILV(I/V)TKV
DLLP(R/K)(D/G)TDLNC(V/I)GDWVVE

### Motif 8 (Starting at Position 130 in SEQ ID NO: 59):

```
TYELKKKHHQLRTVLCGRCQLLSHGHMITAVGGHGGYPGGKQFVSAEELR
            R R K K N S IT DQ
                                      R
```

as a regular expression (SEQ ID NO: 63):

TYELKK(K/R)H(H/R)QL(R/K)TVLCGRC(Q/K/R)LLSHGHMITA
VGG(H/N)GGY(P/S)GGKQF(V/I)(S/T)A(E/D)(E/Q)LR

### Motif 9:

```
KMYDTPGLLHPYQLSMRLNREEQKMVEIRKELKPRTYRIKAGQSVHIGGL
  LF HLMTS TGD M L LPS RVQ SF V V TI
                              T R V R L
```

as a regular expression (SEQ ID NO: 64):

K(M/L)(Y/F)DTPGLLHP(Y/H)(Q/L)(L/M)(S/T)(M/S/T)RL
(N/T)(R/G)(E/D)E(Q/M/R)K(M/L)V(E/L)(I/P/V)(R/S)K
(E/R)(L/V)(K/Q/R)PR(T/S)(Y/F)R(I/V/L)K(A/V)GQ
(S/T)(V/I)HIGGL

### Motif 10:

RLQPPIGEERVAELGKWEEREVKVSGTSWDVSSVDIAIAGLGWFGVGLKG
Q T P MEQF VRK IE E AD NTM VSVS ISL C
A F N VA

as a regular expression (SEQ ID NO: 65):

(R/Q)L(Q/T)PPIG(E/P)ER(V/M/A)(A/E)(E/Q)(L/F)GKW
(E/V)(E/R)(R/K)E(V/I/F)(K/E)V(S/E)G(T/A/N)(S/D)W
DV(S/N)(S/T)(V/M)D(I/V)(A/S)(I/V)(A/S)GLGW(F/I/V)
(G/S/A)(V/L)G(L/C)KG

Further preferably, the NOA polypeptide comprises also one or more of the following motifs:

### Motif 11 (SEQ ID NO: 66):

CYGCGA

### Motif 12 (SEQ ID NO: 67):

KLVD(V/I)VDF(NS)GSFL

### Motif 13 (SEQ ID NO: 68):

VYILG(S/A)ANVGKSAFI

### Motif 14 (SEQ ID NO: 69):

YDTPGVHLHHR

### Motif 15 (SEQ ID NO: 70):

D(V/L/I)AISGLGW(I/L/V/M)

Alternatively, the NOA protein has in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 59, provided that the homologous protein comprises the conserved motifs as outlined above. The overall sequence identity is determined using a global alignment algorithm, such as the Needleman Wunsch algorithm in the program GAP (GCG Wisconsin Package, Accelrys), preferably with default parameters and preferably with sequences of mature proteins (i.e. without taking into account secretion signals or transit peptides). Compared to overall sequence identity, the sequence identity will generally be higher when only conserved domains or motifs are considered. Preferably the motifs in a NOA polypeptide have, in increasing order of preference, at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the motifs represented by SEQ ID NO: 60 to SEQ ID NO: 65 (Motifs 5 to 10).

Preferably, the polypeptide sequence which when used in the construction of a phylogenetic tree, such as the one depicted in FIG. 9, clusters with the group of NOA-like or NOA polypeptides, preferably with the NOA polypeptides comprising the amino acid sequence represented by SEQ ID NO: 59 (AT3G47450) rather than with any other group.

An “ASF1-like polypeptide” as defined herein refers to any polypeptide comprising the following motifs:

MOTIF I:
DLEWKL I/T YVGSA,
MOTIF II:
S/P P D/E P/V/T S/L/A/N K/R I R/P/Q E/A/D E/A D/E
I/V I/L GVTV L/I LLTC S/A Y,
MOTIF III:
Q/R EF V/I/L/M R V/I GYYV N/S/Q N/Q,
MOTIF IV:
V/I/L Q/R RNIL A/T/S/V D/E KPRVT K/R F P/A I,

or a motif having in increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more sequence identity to any one or more of Motifs I to IV.

Alternatively or additionally, the ASF1-like polypeptide has in increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more overall sequence identity to the amino acid represented by SEQ ID NO: 135 or SEQ ID NO: 137.

Preferably, the ASF1-like polypeptide has in increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the N-terminal region of the amino acid represented by SEQ ID NO: 135 or SEQ ID NO: 137. A person skilled in the art would be well aware of what would constitute an N-terminal region of a polypeptide.

The overall sequence identity is determined using a global alignment algorithm, such as the Needleman Wunsch algorithm in the program GAP (GCG Wisconsin Package, Accelrys), preferably with default parameters and preferably with sequences of mature proteins (i.e. without taking into account secretion signals or transit peptides). Compared to overall sequence identity, the sequence identity will generally be higher when only conserved domains or motifs are considered.

Preferably, the polypeptide sequence which when used in the construction of a phylogenetic tree, such as the one depicted in FIG. 11, clusters with the group of ASF1-like polypeptides comprising the amino acid sequence represented by SEQ ID NO: 135 or SEQ ID NO: 137 rather than with any other group.

A “PHDF polypeptide” as defined herein refers to any polypeptide comprising a Cys₄-His-Cys₃ zinc finger.

Examples of such PHDF polypeptides include orthologues and paralogues of the sequences represented by any of SEQ ID NO: 176 and SEQ ID NO: 178.

PHDF polypeptides and orthologues and paralogues thereof typically have in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by any of SEQ ID NO: 176 and SEQ ID NO: 178.

The overall sequence identity is determined using a global alignment algorithm, such as the Needleman Wunsch algorithm in the program GAP (GCG Wisconsin Package, Accelrys), preferably with default parameters and preferably with sequences of mature proteins (i.e. without taking into account secretion signals or transit peptides). Compared to overall sequence identity, the sequence identity will generally be higher when only conserved domains or motifs are considered.

Preferably, the polypeptide sequence which when used in the construction of a phylogenetic tree, clusters with the group of PHDF polypeptides comprising the amino acid sequences represented by SEQ ID NO: 176 and SEQ ID NO: 178 rather than with any other group. Tools and techniques for the construction and analysis of phylogenetic trees are well known in the art.

A “group I MBF1 polypeptide” as defined herein refers to any polypeptide comprising (i) in increasing order of preference at least 70%, 75%, 80%, 85%, 90%, 95%, 98%, 99% or more amino acid sequence identity to an N-terminal multibridging domain with an InterPro entry IPR0013729 (PFAM entry PF08523 MBF1) as represented by SEQ ID NO: 250; and (ii) in increasing order of preference at least 70%, 75%, 80%, 85%, 90%, 95%, 98%, 99% or more amino acid sequence identity to a helix-turn-helix 3 domain with an InterPro entry IPR001387 (PFAM ENTRY PF01381 HTH_{—}3).

Alternatively or additionally, a “group I MBF1 polypeptide” as defined herein refers to any polypeptide sequence having in increasing order of preference at least 50%, 55%, 60%, 65%, 70%, 75%, 80%, 85%, 90%, 95%, 98%, 99% or more amino acid sequence identity to a polypeptide as represented by SEQ ID NO: 189, or as represented by SEQ ID NO: 191, or as represented by SEQ ID NO: 193, or as represented by SEQ ID NO: 195.

Alternatively or additionally, a “group I MBF1 polypeptide” as defined herein refers to any polypeptide having in increasing order of preference at least 50%, 55%, 60%, 65%, 70%, 75%, 80%, 85%, 90%, 95%, 98%, 99% or more amino acid sequence identity to any of the polypeptide sequences given in Table A7 herein.

Alternatively or additionally, a “group I MBF1 polypeptide” as defined herein refers to any polypeptide sequence which when used in the construction of an MBF1 phylogenetic tree, such as the one depicted in FIG. 15, clusters with the group I MBF1 polypeptides comprising the polypeptide sequences as represented by SEQ ID NO: 189, SEQ ID NO: 191, SEQ ID NO: 193, and SEQ ID NO: 195, rather than with any other group.

Alternatively or additionally, a “group I MBF1 polypeptide” as defined herein refers to any polypeptide sequence that functionally complements (i.e. restoring growth) a yeast strain deficient for MBF1 activity, as described in Tsuda et al. (2004) Plant Cell Physiol 45: 225-231.

The terms “domain”, “signature” and “motif” are defined in the “definitions” section herein. Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hulo et al., Nucl. Acids. Res. 32:D134-D137, (2004)), or Pfam (Bateman et al., Nucleic Acids Research 30(1): 276-280 (2002)). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788 (2003)). Domains or motifs may also be identified using routine techniques, such as by sequence alignment.

Concerning group I MBF1 polypeptides, an alignment of the polypeptides of Table A7 herein is shown in FIG. 17. Such alignments are useful for identifying the most conserved domains or motifs between group I MBF1 polypeptides as defined herein. Two such domains are (1) an N-terminal multibridging factor 1 (MBF1) domain with an InterPro entry IPR013729 (and PFAM entry PF08523 MBF1); and (2) a helix-turn-helix type 3 domain with an InterPro entry IPR001387 (and PFAM entry PF01381 HTH_{—}3). Both domains are marked with X's below the consensus sequence.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters. For local alignments, the Smith-Waterman algorithm is particularly useful (Smith T F, Waterman M S (1981) J. Mol. Biol 147(1); 195-7). In some instances, the default parameters may be adjusted to modify the stringency of the search. For example using BLAST, the statistical significance threshold (called “expect” value) for reporting matches against database sequences may be increased to show less stringent matches. This way, short nearly exact matches may be identified.

Concerning group I MBF1 polypeptides, Example 3 herein describes in Table B3 the percentage identity between a group I MBF1 polypeptide as represented by SEQ ID NO: 189 and a group I MBF1 polypeptides listed in Table A7, which can be as low as 74% amino acid sequence identity.

The task of protein subcellular localisation prediction is important and well studied. Knowing a protein's localisation helps elucidate its function. Experimental methods for protein localization range from immunolocalization to tagging of proteins using green fluorescent protein (GFP) or beta-glucuronidase (GUS). Such methods are accurate although labor-intensive compared with computational methods. Recently much progress has been made in computational prediction of protein localisation from sequence data. Among algorithms well known to a person skilled in the art are available at the ExPASy Proteomics tools hosted by the Swiss Institute for Bioinformatics, for example, PSort, TargetP, ChloroP, LocTree, Predotar, LipoP, MITOPROT, PATS, PTS1, SignalP, TMHMM, and others.

Furthermore, COX VIIa subunit polypeptides (at least in their native form) typically have, COX VIIa subunit activity. In addition, COX VIIa subunit polypeptides, when expressed in plants, in particular in rice plants, confer enhanced tolerance to abiotic stresses to those plants.

Furthermore, as YLD-ZnF polypeptides (at least in their native form) typically have a zf-DNL domain (Pfam entry PF05180); they may be involved in protein import into mitochondria. Tools and techniques for measuring protein import into mitochondria are known in the art (see for example Burri et al., J. Biol. Chem. 279, 50243-50249, 2004).

In addition, YLD-ZnF polypeptides, when expressed in rice according to the methods of the present invention as outlined in Examples 8 and 9, give plants having increased yield related traits, in particular increased seed yield or increased early vigour.

Furthermore, PKT polypeptides (at least in their native form) typically have kinase activity. Methods and materials for measuring kinase activity are well known in the art. In addition, PKT polypeptides, when expressed in plants, in particular in rice plants, confer enhanced tolerance to abiotic stresses to those plants.

Furthermore, NOA polypeptides (at least in their native form) typically have GTPase activity. Tools and techniques for measuring GTPase activity are well known in the art (Moreau et al., 2008). Further details are provided in Example 7.

In addition, NOA polypeptides, when expressed in rice according to the methods of the present invention as outlined in Examples 8 and 9, give plants having increased yield related traits, in particular increased seed yield.

In addition, ASF1-like polypeptides, when expressed in rice according to the methods of the present invention as outlined in the Examples section herein, give plants having increased yield-related traits, such as the ones described herein.

PHDF polypeptides, when expressed in plants, in particular in rice plants, confer enhanced tolerance to abiotic stresses to those plants.

Concerning COX VIIa subunit polypeptides, the present invention may be performed, for example, by transforming plants with the nucleic acid sequence represented by any of SEQ ID NO: 1 encoding the polypeptide sequence of SEQ ID NO: 2, SEQ ID NO: 3 encoding the polypeptide sequence of SEQ ID NO: 4, SEQ ID NO: 5 encoding the polypeptide sequence of SEQ ID NO: 6, or SEQ ID NO: 7 encoding the polypeptide sequence of SEQ ID NO: 8. However, performance of the invention is not restricted to these sequences; the methods of the invention may advantageously be performed using any COX VIIa subunit-encoding nucleic acid or COX VIIa subunit polypeptide as defined herein.

Examples of nucleic acids encoding COX VIIa subunit polypeptides are given in Table A1 of the Examples section herein. Such nucleic acids are useful in performing the methods of the invention. Orthologues and paralogues of the amino acid sequences given in Table A1 may be readily obtained using routine tools and techniques, such as a reciprocal blast search. Typically, this involves a first BLAST involving BLASTing a query sequence (for example using any of the sequences listed in Table A1 of the Examples section) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 1 or SEQ ID NO: 2, the second BLAST would therefore be against Physcomitrella sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

Concerning YLD-ZnF polypeptides, the present invention is illustrated by transforming plants with the nucleic acid sequence represented by SEQ ID NO: 18, encoding the polypeptide sequence of SEQ ID NO: 19. However, performance of the invention is not restricted to these sequences; the methods of the invention may advantageously be performed using any YLD-ZnF-encoding nucleic acid or YLD-ZnF polypeptide as defined herein.

Examples of nucleic acids encoding YLD-ZnF polypeptides are given in Table A2 of the Examples section herein. Such nucleic acids are useful in performing the methods of the invention. The amino acid sequences given in Table A2 of the Examples section are example sequences of orthologues and paralogues of the YLD-ZnF polypeptide represented by SEQ ID NO: 19, the terms “orthologues” and “paralogues” being as defined herein. Further orthologues and paralogues may readily be identified by performing a so-called reciprocal blast search. Typically, this involves a first BLAST involving BLASTing a query sequence (for example using any of the sequences listed in Table A2 of the Examples section) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 18 or SEQ ID NO: 19, the second BLAST would therefore be against Medicago truncatula sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

Concerning PKT polypeptides, the present invention may be performed, for example, by transforming plants with the nucleic acid sequence represented by any of SEQ ID NO: 51 encoding the polypeptide sequence of SEQ ID NO: 52, or SEQ ID NO: 53 encoding the polypeptide sequence of SEQ ID NO: 54. However, performance of the invention is not restricted to these sequences; the methods of the invention may advantageously be performed using any PKT-encoding nucleic acid or PKT polypeptide as defined herein.

Examples of nucleic acids encoding PKT polypeptides are given in Table A3 of the Examples section herein. Such nucleic acids are useful in performing the methods of the invention. Orthologues and paralogues of the amino acid sequences given in Table A3 may be readily obtained using routine tools and techniques, such as a reciprocal blast search. Typically, this involves a first BLAST involving BLASTing a query sequence (for example using any of the sequences listed in Table A3 of the Examples section) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 51 or SEQ ID NO: 52, the second BLAST would therefore be against Populus sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

Concerning NOA polypeptides, the present invention is illustrated by transforming plants with the nucleic acid sequence represented by SEQ ID NO: 58, encoding the polypeptide sequence of SEQ ID NO: 59. However, performance of the invention is not restricted to these sequences; the methods of the invention may advantageously be performed using any NOA-encoding nucleic acid or a NOA polypeptide as defined herein.

Examples of nucleic acids encoding NOA polypeptides are given in Table A4 of the Examples section herein. Such nucleic acids are useful in performing the methods of the invention. The amino acid sequences given in Table A4 of the Examples section are example sequences of orthologues and paralogues of the NOA polypeptide represented by SEQ ID NO: 59, the terms “orthologues” and “paralogues” being as defined herein. Further orthologues and paralogues may readily be identified by performing a so-called reciprocal blast search. Typically, this involves a first BLAST involving BLASTing a query sequence (for example using any of the sequences listed in Table A4 of the Examples section) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 58 or SEQ ID NO: 59, the second BLAST would therefore be against Arabidopsis thaliana sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

The present invention is illustrated by transforming plants with the nucleic acid sequence represented by SEQ ID NO: 134 or SEQ ID NO: 136, respectively encoding the polypeptide sequence of SEQ ID NO: 135 or SEQ ID NO: 137. However, performance of the invention is not restricted to these sequences; the methods of the invention may advantageously be performed using any ASF1-like-encoding nucleic acid or ASF1-like polypeptide as defined herein.

Examples of nucleic acids encoding ASF1-like polypeptides are given in Table A5 of Example 1 herein. Such nucleic acids are useful in performing the methods of the invention. The amino acid sequences given in Table A5 of Example 1 are example sequences of orthologues and paralogues of the ASF1-like polypeptide represented by SEQ ID NO: 135 or SEQ ID NO: 137, the terms “orthologues” and “paralogues” being as defined herein. Further orthologues and paralogues may readily be identified by performing a so-called reciprocal blast search. Typically, this involves a first BLAST involving BLASTing a query sequence (for example using any of the sequences listed in Table A5 of Example 1) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 134 or SEQ ID NO: 136, the second BLAST would therefore be against rice sequences; where the query sequence is SEQ ID NO: 135 or SEQ ID NO: 137, the second BLAST would therefore be against Arabidopsis sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

The present invention may be performed, for example, by transforming plants with the nucleic acid sequence represented by any of SEQ ID NO: 175 encoding the polypeptide sequence of SEQ ID NO: 176, or SEQ ID NO: 177 encoding the polypeptide sequence of SEQ ID NO: 178. However, performance of the invention is not restricted to these sequences; the methods of the invention may advantageously be performed using any PHDF-encoding nucleic acid or PHDF polypeptide as defined herein.

Examples of nucleic acids encoding PHDF polypeptides are given in Table A6 of the Examples section herein. Such nucleic acids are useful in performing the methods of the invention. Orthologues and paralogues of the amino acid sequences given in Table A6 may be readily obtained using routine tools and techniques, such as a reciprocal blast search. Typically, this involves a first BLAST involving BLASTing a query sequence (for example using any of the sequences listed in Table A6 of the Examples section) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 175 or SEQ ID NO: 176, the second BLAST would therefore be against Solanum lycopersicum sequences; where the query sequence is SEQ ID NO: 177 or SEQ ID NO: 178, the second BLAST would therefore be against Populus trichocarpa sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

The present invention is illustrated by transforming plants with the nucleic acid sequence represented by SEQ ID NO: 188, or as represented by SEQ ID NO: 190, or as represented by SEQ ID NO: 192, or as represented by SEQ ID NO: 194, encoding a group I MBF1 polypeptide sequence of respectively SEQ ID NO: 189, SEQ ID NO: 191, SEQ ID NO: 193, and SEQ ID NO: 195. However, performance of the invention is not restricted to these sequences; the methods of the invention may advantageously be performed using any nucleic acid sequence encoding a group I MBF1 polypeptide as defined herein.

Examples of nucleic acid sequences encoding group I MBF1 polypeptides are given in Table A7 of Example 1 herein. Such nucleic acid sequences are useful in performing the methods of the invention. The polypeptide sequences given in Table A7 of Example 1 are example sequences of orthologues and paralogues of a group I MBF1 polypeptide represented by SEQ ID NO: 189, or by SEQ ID NO: 191, or by SEQ ID NO: 193, or by SEQ ID NO: 195, the terms “orthologues” and “paralogues” being as defined herein. Further orthologues and paralogues may readily be identified by performing a so-called reciprocal blast search. Typically, this involves a first BLAST involving BLASTing a query sequence (for example using any of the sequences listed in Table A7 of Example 1) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 188 or SEQ ID NO: 189, the second BLAST would therefore be against Arabidopsis thaliana sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbour joining tree, to help visualize clustering of related genes and to identify orthologues and paralogues.

Nucleic acid variants may also be useful in practising the methods of the invention. Examples of such variants include nucleic acids encoding homologues and derivatives of any one of the amino acid sequences given in Table A1 to A7 of the Examples section, the terms “homologue” and “derivative” being as defined herein. Also useful in the methods of the invention are nucleic acids encoding homologues and derivatives of orthologues or paralogues of any one of the amino acid sequences given in Table A1 to A7 of the Examples section. Homologues and derivatives useful in the methods of the present invention have substantially the same biological and functional activity as the unmodified protein from which they are derived. Nucleic acid variants also include variants in which the codon usage is optimised for a particular species, or in which miRNA target sites are removed or added, depending of the purpose.

Further nucleic acid variants useful in practising the methods of the invention include portions of nucleic acids encoding COX VIIa subunit polypeptides, or YLD-ZnF polypeptides, or PKT polypeptides, or NOA polypeptides, or ASF1-like polypeptides, or PHDF polypeptides, or group I MBF1 polypeptides, nucleic acids hybridising to nucleic acids encoding COX VIIa subunit polypeptides, or YLD-ZnF polypeptides, or PKT polypeptides, or NOA polypeptides, or ASF1-like polypeptides, or PHDF polypeptides, or group I MBF1 polypeptides, splice variants of nucleic acids encoding COX VIIa subunit polypeptides, or YLD-ZnF polypeptides, or PKT polypeptides, or NOA polypeptides, or ASF1-like polypeptides, or PHDF polypeptides, or group I MBF1 polypeptides, allelic variants of nucleic acids encoding COX VIIa subunit polypeptides, or YLD-ZnF polypeptides, or PKT polypeptides, or NOA polypeptides, or ASF1-like polypeptides, or PHDF polypeptides, or group I MBF1 polypeptides, and variants of nucleic acids encoding COX VIIa subunit polypeptides, or YLD-ZnF polypeptides, or PKT polypeptides, or NOA polypeptides, or ASF1-like polypeptides, or PHDF polypeptides, or group I MBF1 polypeptides, obtained by gene shuffling. The terms hybridising sequence, splice variant, allelic variant and gene shuffling are as described herein.

Nucleic acids encoding COX VIIa subunit polypeptides, or YLD-ZnF polypeptides, or PKT polypeptides, or NOA polypeptides, or ASF1-like polypeptides, or PHDF polypeptides, or group I MBF1 polypeptides, need not be full-length nucleic acids, since performance of the methods of the invention does not rely on the use of full-length nucleic acid sequences. According to the present invention, there is provided a method for enhancing abiotic stress tolerance in plants, comprising introducing and expressing in a plant a portion of any one of the nucleic acid sequences given in Table A1 to A7 of the Examples section, or a portion of a nucleic acid encoding an orthologue, paralogue or homologue of any of the amino acid sequences given in Table A1 to A7 of the Examples section.

A portion of a nucleic acid may be prepared, for example, by making one or more deletions to the nucleic acid. The portions may be used in isolated form or they may be fused to other coding (or non-coding) sequences in order to, for example, produce a protein that combines several activities. When fused to other coding sequences, the resultant polypeptide produced upon translation may be bigger than that predicted for the protein portion.

Concerning COX VIIa subunit polypeptides, portions useful in the methods of the invention, encode a COX VIIa subunit polypeptide as defined herein, and have substantially the same biological activity as the amino acid sequences given in Table A1 of the Examples section. Preferably, the portion is a portion of any one of the nucleic acids given in Table A1 of the Examples section, or is a portion of a nucleic acid encoding an orthologue or paralogue of any one of the amino acid sequences given in Table A1 of the Examples section. Preferably the portion is at least 400, 450, 500, 550, 600, 650, 700, 750, 800, 850, 900, 950, 1000 consecutive nucleotides in length, the consecutive nucleotides being of any one of the nucleic acid sequences given in Table A1 of the Examples section, or of a nucleic acid encoding an orthologue or paralogue of any one of the amino acid sequences given in Table A1 of the Examples section. Most preferably the portion is a portion of the nucleic acid of SEQ ID NO: 1, SEQ ID NO: 3, SEQ ID NO: 5 or SEQ ID NO: 7. Preferably, the portion encodes a fragment of an amino acid sequence which, when used in the construction of a phylogenetic tree, clusters with the group of COX VIIa subunit polypeptides comprising the amino acid sequence represented by SEQ ID NO: 2, SEQ ID NO: 4, SEQ ID NO: 6 or SEQ ID NO: 8, rather than with any other group.

Concerning YLD-ZnF polypeptides, portions useful in the methods of the invention, encode a YLD-ZnF polypeptide as defined herein, and have substantially the same biological activity as the amino acid sequences given in Table A2 of the Examples section. Preferably, the portion is a portion of any one of the nucleic acids given in Table A2 of the Examples section, or is a portion of a nucleic acid encoding an orthologue or paralogue of any one of the amino acid sequences given in Table A2 of the Examples section. Preferably the portion is at least 300, 350, 400, 450, 500, 550, 600, 650, 700, 750, 800, 850, 900, 950, 1000 consecutive nucleotides in length, the consecutive nucleotides being of any one of the nucleic acid sequences given in Table A2 of the Examples section, or of a nucleic acid encoding an orthologue or paralogue of any one of the amino acid sequences given in Table A2 of the Examples section. Most preferably the portion is a portion of the nucleic acid of SEQ ID NO: 18. Preferably, the portion encodes a fragment of an amino acid sequence which when used in the construction of a phylogenetic tree, such as the one depicted in FIG. 4, clusters with the group of YLD-ZnF polypeptides comprising the amino acid sequence represented by SEQ ID NO: 19 (TA25762) rather than with any other group.

Concerning PKT polypeptides, portions useful in the methods of the invention, encode a PKT polypeptide as defined herein, and have substantially the same biological activity as the amino acid sequences given in Table A3 of the Examples section. Preferably, the portion is a portion of any one of the nucleic acids given in Table A3 of the Examples section, or is a portion of a nucleic acid encoding an orthologue or paralogue of any one of the amino acid sequences given in Table A3 of the Examples section. Preferably the portion is at least 1000, 1250, 1500, 2,000, 2170 consecutive nucleotides in length, the consecutive nucleotides being of any one of the nucleic acid sequences given in Table A3 of the Examples section, or of a nucleic acid encoding an orthologue or paralogue of any one of the amino acid sequences given in Table A3 of the Examples section. Most preferably the portion is a portion of the nucleic acid of SEQ ID NO: 51 or SEQ ID NO: 53. Preferably, the portion encodes a fragment of an amino acid sequence which, when used in the construction of a phylogenetic tree, clusters with the group of PKT polypeptides comprising the amino acid sequence represented by SEQ ID NO: 52 or SEQ ID NO: 54, rather than with any other group.

Concerning NOA polypeptides, portions useful in the methods of the invention, encode a NOA polypeptide as defined herein, and have substantially the same biological activity as the amino acid sequences given in Table A4 of the Examples section. Preferably, the portion is a portion of any one of the nucleic acids given in Table A4 of the Examples section, or is a portion of a nucleic acid encoding an orthologue or paralogue of any one of the amino acid sequences given in Table A4 of the Examples section. Preferably the portion is at least 500, 550, 600, 650, 700, 750, 800, 850, 900, 950, 1000, 1150, 1200, 1250, 1300, 1350, 1400, 1450, 1500, 1550, 1600, 1650, 1700, 1750, 1800, 1850, 1900, 1950, 2000, 2050, 2100, 2150, 2200 consecutive nucleotides in length, the consecutive nucleotides being of any one of the nucleic acid sequences given in Table A4 of the Examples section, or of a nucleic acid encoding an orthologue or paralogue of any one of the amino acid sequences given in Table A4 of the Examples section. Most preferably the portion is a portion of the nucleic acid of SEQ ID NO: 58. Preferably, the portion encodes a fragment of an amino acid sequence which, when used in the construction of a phylogenetic tree, such as the one depicted in FIG. 9, clusters with the group of NOA-like or NOA polypeptides, preferably with the NOA polypeptides comprising the amino acid sequence represented by SEQ ID NO: 59 (AT3G47450) rather than with any other group.

Concerning ASF1-like polypeptides, portions useful in the methods of the invention, encode an ASF1-like polypeptide as defined herein, and have substantially the same biological activity as the amino acid sequences given in Table A5 of Example 1. Preferably, the portion is a portion of any one of the nucleic acids given in Table A5 of Example 1, or is a portion of a nucleic acid encoding an orthologue or paralogue of any one of the amino acid sequences given in Table A5 of Example 1. Preferably the portion is at least 850, 900, 950, 1000, 1050, 1100, 1150, 1200, 1250, 1300, 1350, 1400, 1450, 1500, 1550, 1600, 1650, 1700 consecutive nucleotides in length, the consecutive nucleotides being of any one of the nucleic acid sequences given in Table A5 of Example 1, or of a nucleic acid encoding an orthologue or paralogue of any one of the amino acid sequences given in Table A5 of Example 1. Most preferably the portion is a portion of the nucleic acid of SEQ ID NO: 134 or SEQ ID NO: 136. Preferably, the portion encodes a fragment of an amino acid sequence which, when used in the construction of a phylogenetic tree, such as the one depicted in FIG. 11, clusters with the group of ASF1-like polypeptides comprising the amino acid sequence represented by SEQ ID NO: 135 or SEQ ID NO: 137 rather than with any other group.

Concerning PHDF polypeptides, portions useful in the methods of the invention, encode a PHDF polypeptide as defined herein, and have substantially the same biological activity as the amino acid sequences given in Table A6 of the Examples section. Preferably, the portion is a portion of any one of the nucleic acids given in Table A6 of the Examples section, or is a portion of a nucleic acid encoding an orthologue or paralogue of any one of the amino acid sequences given in Table A6 of the Examples section. Preferably the portion is at least 2500, 2750, 3000, 3250, 3500, 3750, 4000, 4250, 4500, 4750, 5000 or more consecutive nucleotides in length, the consecutive nucleotides being of any one of the nucleic acid sequences given in Table A6 of the Examples section, or of a nucleic acid encoding an orthologue or paralogue of any one of the amino acid sequences given in Table A6 of the Examples section. Most preferably the portion is a portion of the nucleic acid of SEQ ID NO: 175 or SEQ ID NO: 177. Preferably, the portion encodes a fragment of an amino acid sequence which, when used in the construction of a phylogenetic tree, clusters with the group of PHDF polypeptides comprising the amino acid sequence represented by SEQ ID NO: 176 or SEQ ID NO: 178, rather than with any other group.

Concerning group I MBF1 polypeptides, portions useful in the methods of the invention, encode a group I MBF1 polypeptide as defined herein, and have substantially the same biological activity as the polypeptide sequences given in Table A7 of Example 1. Preferably, the portion is a portion of any one of the nucleic acid sequences given in Table A7 of Example 1, or is a portion of a nucleic acid sequence encoding an orthologue or paralogue of any one of the polypeptide sequences given in Table A7 of Example 1. Preferably the portion is, in increasing order of preference at least 250, 300, 350, 375, 400, 425 or more consecutive nucleotides in length, the consecutive nucleotides being of any one of the nucleic acid sequences given in Table A7 of Example 1, or of a nucleic acid sequence encoding an orthologue or paralogue of any one of the polypeptide sequences given in Table A7 of Example 1. Preferably, the portion is a portion of a nucleic sequence encoding a polypeptide sequence comprising (i) in increasing order of preference at least 70%, 75%, 80%, 85%, 90%, 95%, 98%, 99% or more amino acid sequence identity to an N-terminal multibridging domain with an InterPro entry IPR0013729 (PFAM entry PF08523 MBF1) as represented by SEQ ID NO: 250; and (ii) in increasing order of preference at least 70%, 75%, 80%, 85%, 90%, 95%, 98%, 99% or more amino acid sequence identity to a helix-turn-helix 3 domain with an InterPro entry IPR001387 (PFAM ENTRY PF01381 HTH_{—}3). More preferably, the portion is a portion of a nucleic sequence encoding a polypeptide sequence having in increasing order of preference at least 70%, 75%, 80%, 85%, 90%, 95%, 98%, 99% or more amino acid sequence identity to a group I MBF1 polypeptide as represented by SEQ ID NO: 189 or to any of the polypeptide sequences given in Table A7 herein. Most preferably, the portion is a portion of the nucleic acid sequence of SEQ ID NO: 188, or of SEQ ID NO: 190, or of SEQ ID NO: 192, or of SEQ ID NO: 194.

Another nucleic acid variant useful in the methods of the invention is a nucleic acid capable of hybridising, under reduced stringency conditions, preferably under stringent conditions, with a nucleic acid encoding a COX VIIa subunit polypeptide, or a YLD-ZnF polypeptide, or a PKT polypeptide, or a NOA polypeptide, or an ASF1-like polypeptide, or a PHDF polypeptide, or a group I MBF1 polypeptide, as defined herein, or with a portion as defined herein.

According to the present invention, there is provided a method for enhancing abiotic stress tolerance and/or enhancing yield-related traits in plants, comprising introducing and expressing in a plant a nucleic acid capable of hybridizing to any one of the nucleic acids given in Table A1 to A7 of the Examples Section, or comprising introducing and expressing in a plant a nucleic acid capable of hybridising to a nucleic acid encoding an orthologue, paralogue or homologue of any of the nucleic acid sequences given in Table A1 to A7 of the Examples Section.

Concerning COX VIIa subunit polypeptides, hybridising sequences useful in the methods of the invention encode a COX VIIa subunit polypeptide as defined herein, having substantially the same biological activity as the amino acid sequences given in Table A1 of the Examples section. Preferably, the hybridising sequence is capable of hybridising to the complement of any one of the nucleic acids given in Table A1, or to a portion of any of these sequences, a portion being as defined above, or the hybridising sequence is capable of hybridising to the complement of a nucleic acid encoding an orthologue or paralogue of any one of the amino acid sequences given in Table A1. Most preferably, the hybridising sequence is capable of hybridising to the complement of a nucleic acid as represented by SEQ ID NO: 1 or to a portion thereof.

Preferably, the hybridising sequence encodes a polypeptide with an amino acid sequence which, when full-length and used in the construction of a phylogenetic tree, clusters with the group of COX VIIa subunit polypeptides comprising the amino acid sequence represented by SEQ ID NO: 2, SEQ ID NO: 4, SEQ ID NO: 6 or SEQ ID NO: 8 rather than with any other group.

Concerning YLD-ZnF polypeptides, hybridising sequences useful in the methods of the invention encode a YLD-ZnF polypeptide as defined herein, having substantially the same biological activity as the amino acid sequences given in Table A2 of the Examples section. Preferably, the hybridising sequence is capable of hybridising to the complement of any one of the nucleic acids given in Table A2 of the Examples section, or to a portion of any of these sequences, a portion being as defined above, or the hybridising sequence is capable of hybridising to the complement of a nucleic acid encoding an orthologue or paralogue of any one of the amino acid sequences given in Table A2 of the Examples section. Most preferably, the hybridising sequence is capable of hybridising to the complement of a nucleic acid as represented by SEQ ID NO: 18 or to a portion thereof.

Preferably, the hybridising sequence encodes a polypeptide with an amino acid sequence which, when full-length and used in the construction of a phylogenetic tree, such as the one depicted in FIG. 4, clusters with the group of YLD-ZnF polypeptides comprising the amino acid sequence represented by SEQ ID NO: 19 (TA25762) rather than with any other group.

Concerning PKT polypeptides, hybridising sequences useful in the methods of the invention encode a PKT polypeptide as defined herein, having substantially the same biological activity as the amino acid sequences given in Table A3 of the Examples section. Preferably, the hybridising sequence is capable of hybridising to the complement of any one of the nucleic acids given in Table A3, or to a portion of any of these sequences, a portion being as defined above, or the hybridising sequence is capable of hybridising to the complement of a nucleic acid encoding an orthologue or paralogue of any one of the amino acid sequences given in Table A3. Most preferably, the hybridising sequence is capable of hybridising to the complement of a nucleic acid as represented by SEQ ID NO: 51 or SEQ ID NO: 53 or to a portion thereof.

Preferably, the hybridising sequence encodes a polypeptide with an amino acid sequence which, when full-length and used in the construction of a phylogenetic tree, clusters with the group of PKT polypeptides comprising the amino acid sequence represented by SEQ ID NO: 52 or SEQ ID NO: 54 rather than with any other group.

Concerning NOA polypeptides, hybridising sequences useful in the methods of the invention encode a NOA polypeptide as defined herein, having substantially the same biological activity as the amino acid sequences given in Table A4 of the Examples section. Preferably, the hybridising sequence is capable of hybridising to the complement of any one of the nucleic acids given in Table A4 of the Examples section, or to a portion of any of these sequences, a portion being as defined above, or the hybridising sequence is capable of hybridising to the complement of a nucleic acid encoding an orthologue or paralogue of any one of the amino acid sequences given in Table A4 of the Examples section. Most preferably, the hybridising sequence is capable of hybridising to the complement of a nucleic acid as represented by SEQ ID NO: 58 or to a portion thereof.

Preferably, the hybridising sequence encodes a polypeptide with an amino acid sequence which, when full-length and used in the construction of a phylogenetic tree, such as the one depicted in FIG. 9, clusters with the group of NOA-like or NOA polypeptides, preferably with the NOA polypeptides comprising the amino acid sequence represented by SEQ ID NO: 59 (AT3G47450) rather than with any other group.

Concerning ASF1-like polypeptides, hybridising sequences useful in the methods of the invention encode an ASF1-like polypeptide as defined herein, having substantially the same biological activity as the amino acid sequences given in Table A5 of Example 1. Preferably, the hybridising sequence is capable of hybridising to the complement of any one of the nucleic acids given in Table A5 of Example 1, or to a portion of any of these sequences, a portion being as defined above, or the hybridising sequence is capable of hybridising to the complement of a nucleic acid encoding an orthologue or paralogue of any one of the amino acid sequences given in Table A5 of Example 1. Most preferably, the hybridising sequence is capable of hybridising to the complement of a nucleic acid as represented by SEQ ID NO: 134 or SEQ ID NO: 136 or to a portion of either.

Preferably, the hybridising sequence encodes a polypeptide with an amino acid sequence which, when full-length and used in the construction of a phylogenetic tree, such as the one depicted in FIG. 11, clusters with the group of ASF1-like polypeptides comprising the amino acid sequence represented by SEQ ID NO: 135 or SEQ ID NO: 137 rather than with any other group.

Concerning PHDF polypeptides, hybridising sequences useful in the methods of the invention encode a PHDF polypeptide as defined herein, having substantially the same biological activity as the amino acid sequences given in Table A6 of the Examples section. Preferably, the hybridising sequence is capable of hybridising to the complement of any one of the nucleic acids given in Table A6, or to a portion of any of these sequences, a portion being as defined above, or the hybridising sequence is capable of hybridising to the complement of a nucleic acid encoding an orthologue or paralogue of any one of the amino acid sequences given in Table A6. Most preferably, the hybridising sequence is capable of hybridising to the complement of a nucleic acid as represented by SEQ ID NO: 175 or SEQ ID NO: 177 or to a portion thereof.

Preferably, the hybridising sequence encodes a polypeptide with an amino acid sequence which, when full-length and used in the construction of a phylogenetic tree, clusters with the group of PHDF polypeptides comprising the amino acid sequence represented by SEQ ID NO: 176 or SEQ ID NO: 178 rather than with any other group.

Concerning group I MBF1 polypeptides, hybridising sequences useful in the methods of the invention encode a group I MBF1 polypeptide as defined herein, and have substantially the same biological activity as the polypeptide sequences given in Table A7 of Example 1. Preferably, the hybridising sequence is capable of hybridising to any one of the nucleic acid sequences given in Table A7 of Example 1, or to a complement thereof, or to a portion of any of these sequences, a portion being as defined above, or wherein the hybridising sequence is capable of hybridising to a nucleic acid sequence encoding an orthologue or paralogue of any one of the polypeptide sequences given in Table A7 of Example 1, or to a complement thereof. Preferably, the hybridising sequence is capable of hybridising to a nucleic acid sequence encoding a polypeptide sequence comprising (i) in increasing order of preference at least 70%, 75%, 80%, 85%, 90%, 95%, 98%, 99% or more amino acid sequence identity to an N-terminal multibridging domain with an InterPro entry IPR0013729 (PFAM entry PF08523 MBF1) as represented by SEQ ID NO: 250; and (ii) in increasing order of preference at least 70%, 75%, 80%, 85%, 90%, 95%, 98%, 99% or more amino acid sequence identity to a helix-turn-helix 3 domain with an InterPro entry IPR001387 (PFAM ENTRY PF01381 HTH_{—}3). More preferably, the hybridising sequence is capable of hybridising to a nucleic acid sequence encoding a polypeptide sequence having in increasing order of preference at least 70%, 75%, 80%, 85%, 90%, 95%, 98%, 99% or more amino acid sequence identity to a group I MBF1 polypeptide as represented by SEQ ID NO: 189 or to any of the polypeptide sequences given in Table A7 herein. Most preferably, the hybridising sequence is capable of hybridising to a nucleic acid sequence as represented by SEQ ID NO: 188, or of SEQ ID NO: 190, or of SEQ ID NO: 192, or of SEQ ID NO: 194 or to a portion thereof.

Another nucleic acid variant useful in the methods of the invention is a splice variant encoding a COX VIIa subunit polypeptide, or a YLD-ZnF polypeptide, or a PKT polypeptide, or a NOA polypeptide, or an ASF1-like polypeptide, or a PHDF polypeptide, or a group I MBF1 polypeptide, as defined hereinabove, a splice variant being as defined herein.

According to the present invention, there is provided a method for enhancing abiotic stress tolerance and/or enhancing yield-related traits in plants, comprising introducing and expressing in a plant a splice variant of any one of the nucleic acid sequences given in Table A1 to A7 of the Examples Section, or a splice variant of a nucleic acid encoding an orthologue, paralogue or homologue of any of the amino acid sequences given in Table A1 to A7 of the Examples Section.

Concerning COX VIIa subunit polypeptides, preferred splice variants are splice variants of a nucleic acid represented by any of SEQ ID NO: 1, SEQ ID NO: 3, SEQ ID NO: 5 or SEQ ID NO: 7, or a splice variant of a nucleic acid encoding an orthologue or paralogue of any of SEQ ID NO: 2, SEQ ID NO: 4, SEQ ID NO: 6 or SEQ ID NO: 8. Preferably, the amino acid sequence encoded by the splice variant, when used in the construction of a phylogenetic tree, clusters with the group of COX VIIa subunit polypeptides comprising the amino acid sequence represented by SEQ ID NO: 2, SEQ ID NO: 4, SEQ ID NO: 6 or SEQ ID NO: 8 rather than with any other group.

Concerning YLD-ZnF polypeptides, preferred splice variants are splice variants of a nucleic acid represented by SEQ ID NO: 18, or a splice variant of a nucleic acid encoding an orthologue or paralogue of SEQ ID NO: 19. Preferably, the amino acid sequence encoded by the splice variant, when used in the construction of a phylogenetic tree, such as the one depicted in FIG. 4, clusters with the group of YLD-ZnF polypeptides comprising the amino acid sequence represented by SEQ ID NO: 19 (TA25762) rather than with any other group.

Concerning PKT polypeptides, preferred splice variants are splice variants of a nucleic acid represented by any of SEQ ID NO: 51 or SEQ ID NO: 53, or a splice variant of a nucleic acid encoding an orthologue or paralogue of any of SEQ ID NO: 52 or SEQ ID NO: 54. Preferably, the amino acid sequence encoded by the splice variant, when used in the construction of a phylogenetic tree, clusters with the group of PKT polypeptides comprising the amino acid sequence represented by SEQ ID NO: 52 or SEQ ID NO: 54 rather than with any other group.

Concerning NOA polypeptides, preferred splice variants are splice variants of a nucleic acid represented by SEQ ID NO: 58, or a splice variant of a nucleic acid encoding an orthologue or paralogue of SEQ ID NO: 59. Preferably, the amino acid sequence encoded by the splice variant, when used in the construction of a phylogenetic tree, such as the one depicted in FIG. 9, clusters with the group of NOA-like or NOA polypeptides, preferably with the NOA polypeptides comprising the amino acid sequence represented by SEQ ID NO: 59 (AT3G47450) rather than with any other group.

Concerning ASF1-like polypeptides, preferred splice variants are splice variants of a nucleic acid represented by SEQ ID NO: 134 or SEQ ID NO: 136, or a splice variant of a nucleic acid encoding an orthologue or paralogue of SEQ ID NO: 135 or SEQ ID NO: 137. Preferably, the amino acid sequence encoded by the splice variant, when used in the construction of a phylogenetic tree, such as the one depicted in FIG. 11, clusters with the group of ASF1-like polypeptides comprising the amino acid sequence represented by SEQ ID NO: 135 or SEQ ID NO: 137 rather than with any other group.

Concerning PHDF polypeptides, preferred splice variants are splice variants of a nucleic acid represented by any of SEQ ID NO: 175 or SEQ ID NO: 177, or a splice variant of a nucleic acid encoding an orthologue or paralogue of any of SEQ ID NO: 176 or SEQ ID NO: 177. Preferably, the amino acid sequence encoded by the splice variant, when used in the construction of a phylogenetic tree, clusters with the group of PHDF polypeptides comprising the amino acid sequence represented by SEQ ID NO: 176 or SEQ ID NO: 177 rather than with any other group.

Concerning group I MBF1 polypeptides, preferred splice variants are splice variants of a nucleic acid sequence represented by SEQ ID NO: 188, or a splice variant of a nucleic acid sequence encoding an orthologue or paralogue of SEQ ID NO: 189. Preferably, the splice variant is a splice variant of a nucleic acid sequence encoding a polypeptide sequence comprising (i) in increasing order of preference at least 70%, 75%, 80%, 85%, 90%, 95%, 98%, 99% or more amino acid sequence identity to an N-terminal multibridging domain with an InterPro entry IPR0013729 (PFAM entry PF08523 MBF1) as represented by SEQ ID NO: 250; and (ii) in increasing order of preference at least 70%, 75%, 80%, 85%, 90%, 95%, 98%, 99% or more amino acid sequence identity to a helix-turn-helix 3 domain with an InterPro entry IPR001387 (PFAM ENTRY PF01381 HTH_{—}3). More preferably, the splice variant is a splice variant of a nucleic acid sequence encoding a polypeptide sequence having in increasing order of preference at least 70%, 75%, 80%, 85%, 90%, 95%, 98%, 99% or more amino acid sequence identity to a group I MBF1 polypeptide as represented by SEQ ID NO: 189 or to any of the polypeptide sequences given in Table A7 herein. Most preferably, the splice variant is a splice variant of a nucleic acid sequence as represented by SEQ ID NO: 188, or of SEQ ID NO: 190, or of SEQ ID NO: 192, or of SEQ ID NO: 194, or of a nucleic acid sequence encoding a polypeptide sequence as represented respectively by SEQ ID NO: 189, by SEQ ID NO: 190, by SEQ ID NO: 192, by SEQ ID NO: 194.

Another nucleic acid variant useful in performing the methods of the invention is an allelic variant of a nucleic acid encoding a COX VIIa subunit polypeptide, or a YLD-ZnF polypeptide, or a PKT polypeptide, or a NOA polypeptide, or an ASF1-like polypeptide, or a PHDF polypeptide, or a group I MBF1 polypeptide, as defined hereinabove, an allelic variant being as defined herein.

According to the present invention, there is provided a method for enhancing abiotic stress tolerance and/or enhancing yield-related traits in plants, comprising introducing and expressing in a plant an allelic variant of any one of the nucleic acids given in Table A1 to A7 in the Examples Section, or comprising introducing and expressing in a plant an allelic variant of a nucleic acid encoding an orthologue, paralogue or homologue of any of the amino acid sequences given in Table A1 to A7 in the Examples Section.

Concerning COX VIIa subunit polypeptides, the polypeptides encoded by allelic variants useful in the methods of the present invention have substantially the same biological activity as the COX VIIa subunit polypeptide of any of SEQ ID NO: 2 or any of the amino acids depicted in Table A1 of the Examples section. Allelic variants exist in nature, and encompassed within the methods of the present invention is the use of these natural alleles. Preferably, the allelic variant is an allelic variant of any of SEQ ID NO: 1, SEQ ID NO: 3, SEQ ID NO: 5 or SEQ ID NO: 7 or an allelic variant of a nucleic acid encoding an orthologue or paralogue of SEQ ID NO: 2, SEQ ID NO: 4, SEQ ID NO: 6 or SEQ ID NO: 8. Preferably, the amino acid sequence encoded by the allelic variant, clusters with the COX VIIa subunit polypeptides comprising the amino acid sequence represented by SEQ ID NO: 2, SEQ ID NO: 4, SEQ ID NO: 6 or SEQ ID NO: 8 rather than with any other group.

Concerning YLD-ZnF polypeptides, the polypeptides encoded by allelic variants useful in the methods of the present invention have substantially the same biological activity as the YLD-ZnF polypeptide of SEQ ID NO: 19 and any of the amino acids depicted in Table A2 of the Examples section. Allelic variants exist in nature, and encompassed within the methods of the present invention is the use of these natural alleles. Preferably, the allelic variant is an allelic variant of SEQ ID NO: 18 or an allelic variant of a nucleic acid encoding an orthologue or paralogue of SEQ ID NO: 19. Preferably, the amino acid sequence encoded by the allelic variant, when used in the construction of a phylogenetic tree, such as the one depicted in FIG. 4, clusters with the group of YLD-ZnF polypeptides comprising the amino acid sequence represented by SEQ ID NO: 19 (TA25762) rather than with any other group.

Concerning PKT polypeptides, the polypeptides encoded by allelic variants useful in the methods of the present invention have substantially the same biological activity as the PKT polypeptide of any of SEQ ID NO: 52 or any of the amino acids depicted in Table A3 of the Examples section. Allelic variants exist in nature, and encompassed within the methods of the present invention is the use of these natural alleles. Preferably, the allelic variant is an allelic variant of any of SEQ ID NO: 51 or SEQ ID NO: 53 or an allelic variant of a nucleic acid encoding an orthologue or paralogue of SEQ ID NO: 52 or SEQ ID NO: 54. Preferably, the amino acid sequence encoded by the allelic variant, clusters with the PKT polypeptides comprising the amino acid sequence represented by SEQ ID NO: 52 or SEQ ID NO: 54 rather than with any other group.

Concerning NOA polypeptides, the polypeptides encoded by allelic variants useful in the methods of the present invention have substantially the same biological activity as the NOA polypeptide of SEQ ID NO: 59 and any of the amino acids depicted in Table A4 of the Examples section. Allelic variants exist in nature, and encompassed within the methods of the present invention is the use of these natural alleles. Preferably, the allelic variant is an allelic variant of SEQ ID NO: 58 or an allelic variant of a nucleic acid encoding an orthologue or paralogue of SEQ ID NO: 59. Preferably, the amino acid sequence encoded by the allelic variant, when used in the construction of a phylogenetic tree, such as the one depicted in FIG. 9, clusters with the group of NOA-like or NOA polypeptides, preferably with the NOA polypeptides comprising the amino acid sequence represented by SEQ ID NO: 59 (AT3G47450) rather than with any other group.

Concerning ASF1-like polypeptides, the polypeptides encoded by allelic variants useful in the methods of the present invention have substantially the same biological activity as the ASF1-like polypeptide of SEQ ID NO: 135 or SEQ ID NO: 137 and any of the amino acids depicted in Table A5 of Example 1. Allelic variants exist in nature, and encompassed within the methods of the present invention is the use of these natural alleles. Preferably, the allelic variant is an allelic variant of SEQ ID NO: 134 or SEQ ID NO: 136 or an allelic variant of a nucleic acid encoding an orthologue or paralogue of SEQ ID NO: 135 or SEQ ID NO: 137. Preferably, the amino acid sequence encoded by the allelic variant, when used in the construction of a phylogenetic tree, such as the one depicted in FIG. 11, clusters with the ASF1-like polypeptides comprising the amino acid sequence represented by SEQ ID NO: 135 or SEQ ID NO: 137 rather than with any other group.

Concerning PHDF polypeptides, the polypeptides encoded by allelic variants useful in the methods of the present invention have substantially the same biological activity as the PHDF polypeptide of any of SEQ ID NO: 176 or any of the amino acids depicted in Table A6 of the Examples section. Allelic variants exist in nature, and encompassed within the methods of the present invention is the use of these natural alleles. Preferably, the allelic variant is an allelic variant of any of SEQ ID NO: 175 or SEQ ID NO: 177 or an allelic variant of a nucleic acid encoding an orthologue or paralogue of SEQ ID NO: 176 or SEQ ID NO: 178. Preferably, the amino acid sequence encoded by the allelic variant, clusters with the PHDF polypeptides comprising the amino acid sequence represented by SEQ ID NO: 176 or SEQ ID NO: 178 rather than with any other group.

Concerning group I MBF1 polypeptides, the allelic variants useful in the methods of the present invention have substantially the same biological activity as a group I MBF1 polypeptide of SEQ ID NO: 189 and any of the polypeptide sequences depicted in Table A7 of Example 1. Allelic variants exist in nature, and encompassed within the methods of the present invention is the use of these natural alleles. Preferably, the allelic variant is an allelic variant of a polypeptide sequence comprising (i) in increasing order of preference at least 70%, 75%, 80%, 85%, 90%, 95%, 98%, 99% or more amino acid sequence identity to an N-terminal multibridging domain with an InterPro entry IPR0013729 (PFAM entry PF08523 MBF1) as represented by SEQ ID NO: 250; and (ii) in increasing order of preference at least 70%, 75%, 80%, 85%, 90%, 95%, 98%, 99% or more amino acid sequence identity to a helix-turn-helix 3 domain with an InterPro entry IPR001387 (PFAM ENTRY PF01381 HTH_{—}3). More preferably the allelic variant is an allelic variant encoding a polypeptide sequence having in increasing order of preference at least 70%, 75%, 80%, 85%, 90%, 95%, 98%, 99% or more amino acid sequence identity to a group I MBF1 polypeptide as represented by SEQ ID NO: 189 or to any of the polypeptide sequences given in Table A herein. Most preferably, the allelic variant is an allelic variant of SEQ ID NO: 188, or of SEQ ID NO: 190, or of SEQ ID NO: 192, or of SEQ ID NO: 194 or an allelic variant of a nucleic acid sequence encoding a polypeptide sequence as represented respectively by SEQ ID NO: 189, by SEQ ID NO: 191, by SEQ ID NO: 193, by SEQ ID NO: 195.

Gene shuffling or directed evolution may also be used to generate variants of nucleic acids encoding COX VIIa subunit polypeptides, or YLD-ZnF polypeptides, or PKT polypeptides, or NOA polypeptides, or ASF1-like polypeptides, or PHDF polypeptides, or group I MBF1 polypeptides, as defined above; the term “gene shuffling” being as defined herein.

According to the present invention, there is provided a method for enhancing abiotic stress tolerance and/or enhancing yield-related traits in plants, comprising introducing and expressing in a plant a variant of any one of the nucleic acid sequences given in Table A1 to A7 of the Examples Section, or comprising introducing and expressing in a plant a variant of a nucleic acid encoding an orthologue, paralogue or homologue of any of the amino acid sequences given in Table A1 to A7 of the Examples Section, which variant nucleic acid is obtained by gene shuffling.

Concerning COX VIIa subunit polypeptides, preferably, the amino acid sequence encoded by the variant nucleic acid obtained by gene shuffling, when used in the construction of a phylogenetic tree, clusters with the group of COX VIIa subunit polypeptides comprising the amino acid sequence represented by SEQ ID NO: 2, SEQ ID NO: 4, SEQ ID NO: 6 or SEQ ID NO: 8 rather than with any other group.

Concerning YLD-ZnF polypeptides, preferably, the amino acid sequence encoded by the variant nucleic acid obtained by gene shuffling, when used in the construction of a phylogenetic tree, such as the one depicted in FIG. 4, clusters with the group of YLD-ZnF polypeptides comprising the amino acid sequence represented by SEQ ID NO: 19 (TA25762) rather than with any other group.

Concerning PKT polypeptides, preferably, the amino acid sequence encoded by the variant nucleic acid obtained by gene shuffling, when used in the construction of a phylogenetic tree, clusters with the group of PKT polypeptides comprising the amino acid sequence represented by SEQ ID NO: 52 or SEQ ID NO: 54 rather than with any other group.

Concerning NOA polypeptides, preferably, the amino acid sequence encoded by the variant nucleic acid obtained by gene shuffling, when used in the construction of a phylogenetic tree such as the one depicted in FIG. 9, clusters with the group of NOA-like or NOA polypeptides, preferably with the NOA polypeptides comprising the amino acid sequence represented by SEQ ID NO: 59 (AT3G47450) rather than with any other group.

Concerning ASF1-like polypeptides, preferably, the amino acid sequence encoded by the variant nucleic acid obtained by gene shuffling, when used in the construction of a phylogenetic tree such as the one depicted in FIG. 11, clusters with the group of ASF1-like polypeptides comprising the amino acid sequence represented by SEQ ID NO: 135 or SEQ ID NO: 137 rather than with any other group.

Concerning PHDF polypeptides, preferably, the amino acid sequence encoded by the variant nucleic acid obtained by gene shuffling, when used in the construction of a phylogenetic tree, clusters with the group of PHDF polypeptides comprising the amino acid sequence represented by SEQ ID NO: 176 or SEQ ID NO: 178 rather than with any other group.

Concerning group I MBF1 polypeptides, preferably, the variant nucleic acid sequence obtained by gene shuffling encodes a polypeptide sequence (i) in increasing order of preference at least 70%, 75%, 80%, 85%, 90%, 95%, 98%, 99% or more amino acid sequence identity to an N-terminal multibridging domain with an InterPro entry IPR0013729 (PFAM entry PF08523 MBF1) as represented by SEQ ID NO: 250; and (ii) in increasing order of preference at least 70%, 75%, 80%, 85%, 90%, 95%, 98%, 99% or more amino acid sequence identity to a helix-turn-helix 3 domain with an InterPro entry IPR001387 (PFAM ENTRY PF01381 HTH_{—}3). More preferably, the variant nucleic acid sequence obtained by gene shuffling encodes a polypeptide sequence having in increasing order of preference at least 50%, 55%, 60%, 65%, 70%, 75%, 80%, 85%, 90%, 95%, 98%, 99% or more amino acid sequence identity to a group I MBF1 polypeptide as represented by SEQ ID NO: 189 or to any of the polypeptide sequences given in Table A7 herein. Most preferably, the nucleic acid sequence obtained by gene shuffling encodes a polypeptide sequence as represented by SEQ ID NO: 189, or by SEQ ID NO: 191, or by SEQ ID NO: 193, or by SEQ ID NO: 195.

Furthermore, nucleic acid variants may also be obtained by site-directed mutagenesis. Several methods are available to achieve site-directed mutagenesis, the most common being PCR based methods (Current Protocols in Molecular Biology. Wiley Eds.).

Nucleic acids encoding COX VIIa subunit polypeptides may be derived from any natural or artificial source. The nucleic acid may be modified from its native form in composition and/or genomic environment through deliberate human manipulation. Preferably the COX VIIa subunit polypeptide-encoding nucleic acid is from a plant, further preferably from a monocotyledonous or dicotyledonous plant, more preferably from the family Physcomitrella, Solanum, Hordeum or Populus.

Nucleic acids encoding YLD-ZnF polypeptides may be derived from any natural or artificial source. The nucleic acid may be modified from its native form in composition and/or genomic environment through deliberate human manipulation. Preferably the YLD-ZnF polypeptide-encoding nucleic acid is from a plant, further preferably from a dicotyledonous plant, more preferably from the family Fabaceae, most preferably the nucleic acid is from Medicago truncatula.

Nucleic acids encoding PKT polypeptides may be derived from any natural or artificial source. The nucleic acid may be modified from its native form in composition and/or genomic environment through deliberate human manipulation. Preferably the PKT polypeptide-encoding nucleic acid is from a plant, further preferably from a monocotyledonous or dicotyledonous plant, more preferably from the family Populus or Hordeum.

Nucleic acids encoding NOA polypeptides may be derived from any natural or artificial source. The nucleic acid may be modified from its native form in composition and/or genomic environment through deliberate human manipulation. Preferably the NOA polypeptide-encoding nucleic acid is from a plant, further preferably from a dicotyledonous plant, more preferably from the family Brassicaceae, most preferably the nucleic acid is from Arabidopsis thaliana.

Furthermore, the present invention also provides a hitherto unknown NOA polypeptide and NOA encoding nucleic acids. Therefore, according to one aspect of the invention there is provided an isolated nucleic acid molecule comprising:
(a) a nucleic acid represented by SEQ ID NO: 125;
(b) the complement of a nucleic acid represented by SEQ ID NO: 125;
(c) a nucleic acid encoding a NOA polypeptide having, in increasing order of preference, at least 50%, 55%, 60%, 65%, 70%, 75%, 80%, 85%, 90%, 95%, 96%, 97%, 98%, 99% or more sequence identity to the amino acid sequence represented by SEQ ID NO: 94; and an isolated polypeptide comprising:
(i) an amino acid sequence represented by SEQ ID NO: 94;
(ii) an amino acid sequence having, in increasing order of preference, at least 50%, 55%, 60%, 65%, 70%, 75%, 80%, 85%, 90%, 95%, 96%, 97%, 98%, 99% or more sequence identity to the amino acid sequence represented by SEQ ID NO: 94;
(iii) derivatives of any of the amino acid sequences given in (i) or (ii) above.

Nucleic acids encoding ASF1-like polypeptides may be derived from any natural or artificial source. The nucleic acid may be modified from its native form in composition and/or genomic environment through deliberate human manipulation. Preferably the ASF1-LIKE polypeptide-encoding nucleic acid is from a plant, further preferably from a monocotyledonous plant or a dicotyledonous plant, more preferably from the family Poaceae or Brassicacae, most preferably the nucleic acid is from Oryza sativa or Arbidopsis thaliana.

Nucleic acids encoding PHDF polypeptides may be derived from any natural or artificial source. The nucleic acid may be modified from its native form in composition and/or genomic environment through deliberate human manipulation. Preferably the PHDF polypeptide-encoding nucleic acid is from a plant, further preferably from a monocotyledonous or dicotyledonous plant, more preferably from the family Populus or Solanum.

Nucleic acid sequences encoding group I MBF1 polypeptides may be derived from any natural or artificial source. The nucleic acid sequence may be modified from its native form in composition and/or genomic environment through deliberate human manipulation. The nucleic acid sequence encoding a group I MBF1 polypeptide is from a plant, further preferably from a dicotyledonous plant, more preferably from the nucleic acid sequence is from Arabidopsis thaliana, or Medicago truncatula. Alternatively, the nucleic acid sequence encoding a group I MBF1 polypeptide is from a moncotyledonous plant, more preferably from the nucleic acid sequence is from Triticum aestivum.

Concerning COX VIIa polypeptides, or PKT polypeptides, or PHDF polypeptides, performance of the methods of the invention gives plants having enhanced tolerance to abiotic stress.

Concerning YLD-ZnF polypeptides, performance of the methods of the invention gives plants having enhanced yield-related traits. In particular performance of the methods of the invention gives plants having increased yield, especially increased seed yield relative to control plants, and/or increased early vigour. The terms “yield”, “seed yield” and “early vigour” are described in more detail in the “definitions” section herein.

Reference herein to enhanced yield-related traits is taken to mean an increase in biomass (weight) of one or more parts of a plant, which may include aboveground (harvestable) parts and/or (harvestable) parts below ground. In particular, such harvestable parts are seeds, and performance of the methods of the invention results in plants having increased seed yield relative to the seed yield of control plants. The term enhanced yield-related traits also encompasses early vigour.

Taking corn as an example, a yield increase may be manifested as one or more of the following: increase in the number of plants established per square meter, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per square meter, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others.

Concerning NOA polypeptides, or ASF1-like polypeptides, performance of the methods as described herein gives plants having enhanced yield-related traits. In particular performance of the methods of the invention gives plants having increased yield, especially increased seed yield relative to control plants. The terms “yield” and “seed yield” are described in more detail in the “definitions” section herein.

Reference herein to enhanced yield-related traits is taken to mean an increase in biomass (weight) of one or more parts of a plant, which may include aboveground (harvestable) parts and/or (harvestable) parts below ground. In particular, such harvestable parts are seeds, and performance of the methods of the invention results in plants having increased seed yield relative to the seed yield of control plants.

Concerning group I MBF1 polypeptides, performance of the methods of the invention gives plants having increased yield-related traits relative to control plants. The terms “yield” and “seed yield” are described in more detail in the “definitions” section herein.

Concerning abiotic stress tolerance, the present invention provides a method for enhancing stress tolerance in plants, relative to control plants, which method comprises modulating expression in a plant of a nucleic acid encoding a COX VIIa subunit polypeptide, a PKT polypeptide, a PHDF polypeptide, as defined herein.

Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35%, 30% or 25%, more preferably less than 20% or 15% in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, fungi, nematodes and insects.

In particular, the methods of the present invention may be performed under conditions of (mild) drought to give plants having increased yield relative to control plants. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location. Plants with optimal growth conditions, (grown under non-stress conditions) typically yield in increasing order of preference at least 97%, 95%, 92%, 90%, 87%, 85%, 83%, 80%, 77% or 75% of the average production of such plant in a given environment. Average production may be calculated on harvest and/or season basis. Persons skilled in the art are aware of average yield productions of a crop.

In particular, the methods of the present invention may be performed under conditions of (mild) drought to give plants having enhanced drought tolerance relative to control plants, which might manifest itself as an increased yield relative to control plants. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location. Plants with optimal growth conditions, (grown under non-stress conditions) typically yield in increasing order of preference at least 97%, 95%, 92%, 90%, 87%, 85%, 83%, 80%, 77% or 75% of the average production of such plant in a given environment. Average production may be calculated on harvest and/or season basis. Persons skilled in the art are aware of average yield productions of a crop.

Performance of the methods of the invention gives plants grown under (mild) drought conditions enhanced drought tolerance relative to control plants grown under comparable conditions. Therefore, according to the present invention, there is provided a method for enhancing drought tolerance in plants grown under (mild) drought conditions, which method comprises modulating expression in a plant of a nucleic acid encoding a COX VIIa subunit polypeptide, or a PKT polypeptide, or a PHDF polypeptide.

Performance of the methods of the invention gives plants grown under conditions of nutrient deficiency, particularly under conditions of nitrogen deficiency, enhanced tolerance to nutrient deficient conditions relative to control plants grown under comparable conditions. Therefore, according to the present invention, there is provided a method for enhancing tolerance to nutrient deficiency in plants grown under conditions of nutrient deficiency, which method comprises modulating expression in a plant of a nucleic acid encoding a COX VIIa subunit polypeptide, or a YLD-ZnF polypeptide, or a PKT polypeptide, or a NOA polypeptide, or an ASF1-like polypeptide, or a PHDF polypeptide, or a group I MBF1 polypeptide. Nutrient deficiency may result from a lack of nutrients such as nitrogen, phosphates and other phosphorous-containing compounds, potassium, calcium, magnesium, manganese, iron and boron, amongst others.

Performance of the methods of the invention gives plants grown under conditions of salt stress, enhanced tolerance to salt relative to control plants grown under comparable conditions. Therefore, according to the present invention, there is provided a method for enhancing salt tolerance in plants grown under conditions of salt stress, which method comprises modulating expression in a plant of a nucleic acid encoding a COX VIIa subunit polypeptide, or a YLD-ZnF polypeptide, or a PKT polypeptide, or a NOA polypeptide, or an ASF1-like polypeptide, or a PHDF polypeptide, or a group I MBF1 polypeptide. The term salt stress is not restricted to common salt (NaCl), but may be any one or more of: NaCl, KCl, LiCl, MgCl₂, CaCl₂, amongst others.

Concerning yield-related traits, the present invention provides a method for increasing yield, especially seed yield of plants, relative to control plants, which method comprises modulating expression in a plant of a nucleic acid encoding a YLD-ZnF polypeptide, or a NOA polypeptide, or an ASF1-like polypeptide, as defined herein.

The present invention also provides a method for increasing yield-related traits of plants relative to control plants, which method comprises increasing expression in a plant of a nucleic acid sequence encoding a group I MBF1 polypeptide as defined herein.

Since the transgenic plants according to the present invention have increased yield and/or increased yield-related traits, it is likely that these plants exhibit an increased growth rate (during at least part of their life cycle), relative to the growth rate of control plants at a corresponding stage in their life cycle.

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect increased (early) vigour. The increase in growth rate may alter the harvest cycle of a plant allowing plants to be sown later and/or harvested sooner than would otherwise be possible (a similar effect may be obtained with earlier flowering time; delayed flowering is usually not a desired trait in crops). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

According to a preferred feature of the present invention, performance of the methods of the invention gives plants having an increased growth rate relative to control plants. Therefore, according to the present invention, there is provided a method for increasing the growth rate of plants, which method comprises modulating or increasing expression in a plant of a nucleic acid encoding a YLD-ZnF polypeptide, or a NOA polypeptide, or an ASF1-like polypeptide, or a group I MBF1 polypeptide as defined herein.

Performance of the methods of the invention gives plants grown under non-stress conditions or under mild drought conditions increased yield relative to control plants grown under comparable conditions. Therefore, according to the present invention, there is provided a method for increasing yield in plants grown under non-stress conditions or under mild drought conditions, which method comprises modulating expression in a plant of a nucleic acid encoding a YLD-ZnF polypeptide, or a NOA polypeptide, or an ASF1-like polypeptide, or a group I MBF1 polypeptide.

The present invention encompasses plants or parts thereof (including seeds) obtainable by the methods according to the present invention. The plants or parts thereof comprise a nucleic acid transgene encoding a COX VIIa subunit polypeptide, or a YLD-ZnF polypeptide, or a PKT polypeptide, or a NOA polypeptide, or an ASF1-like polypeptide, or a PHDF polypeptide, or a group I MBF1 polypeptide, as defined above.

The invention also provides genetic constructs and vectors to facilitate introduction and/or expression in plants of nucleic acids encoding COX VIIa subunit polypeptides, or YLD-ZnF polypeptides, or PKT polypeptide, or NOA polypeptides, or ASF1-like polypeptides, or PHDF polypeptides, or group I MBF1 polypeptides. The gene constructs may be inserted into vectors, which may be commercially available, suitable for transforming into plants and suitable for expression of the gene of interest in the transformed cells. The invention also provides use of a gene construct as defined herein in the methods of the invention.

More specifically, the present invention provides a construct comprising:
(a) a nucleic acid encoding a COX VIIa subunit polypeptide, or a YLD-ZnF polypeptide, or a PKT polypeptide, or a NOA polypeptide, or an ASF1-like polypeptide, or a PHDF polypeptide, or a group I MBF1 polypeptide, as defined above;
(b) one or more control sequences capable of driving expression of the nucleic acid sequence of (a); and optionally
(c) a transcription termination sequence.

Preferably, the nucleic acid encoding a COX VIIa subunit polypeptide, or a YLD-ZnF polypeptide, or a PKT polypeptide, or a NOA polypeptide, or an ASF1-like polypeptide, or a PHDF polypeptide, or a group I MBF1 polypeptide, is as defined above. The term “control sequence” and “termination sequence” are as defined herein.

Concerning group I MBF1 polypeptides, preferably, one of the control sequences of a construct is a constitutive promoter isolated from a plant genome. An example of a constitutive promoter is a GOS2 promoter, preferably a GOS2 promoter from rice, most preferably a GOS2 sequence as represented by SEQ ID NO: 254. Alternatively, a constitutive promoter is an HMG promoter, preferably an HMG promoter from rice, most preferably an HMG promoter as represented by SEQ ID NO: 253.

Plants are transformed with a vector comprising any of the nucleic acids described above. The skilled artisan is well aware of the genetic elements that must be present on the vector in order to successfully transform, select and propagate host cells containing the sequence of interest. The sequence of interest is operably linked to one or more control sequences (at least to a promoter).

Advantageously, any type of promoter, whether natural or synthetic, may be used to drive expression of the nucleic acid sequence, but preferably the promoter is of plant origin. A constitutive promoter is particularly useful in the methods. Preferably the constitutive promoter is also a ubiquitous promoter of medium strength. See the “Definitions” section herein for definitions of the various promoter types.

Concerning group I MBF1 polypeptides, advantageously, any type of promoter, whether natural or synthetic, may be used to increase expression of the nucleic acid sequence. A constitutive promoter is particularly useful in the methods, preferably a constitutive promoter isolated from a plant genome. The plant constitutive promoter drives expression of a coding sequence at a level that is in all instances below that obtained under the control of a 35S CaMV viral promoter. An example of such a promoter is a GOS2 promoter as represented by SEQ ID NO: 254. Another example of such a promoter is an HMG promoter as represented by SEQ ID NO: 253.

In the case of group I MBF1 genes, organ-specific promoters, for example for preferred expression in leaves, stems, tubers, meristems, seeds, are useful in performing the methods of the invention. Developmentally-regulated and inducible promoters are also useful in performing the methods of the invention. See the “Definitions” section herein for definitions of the various promoter types.

Concerning COX VIIa subunit polypeptides, it should be clear that the applicability of the present invention is not restricted to the COX VIIa subunit polypeptide-encoding nucleic acid represented by SEQ ID NO: 1, SEQ ID NO: 3, SEQ ID NO: 5 or SEQ ID NO: 7, nor is the applicability of the invention restricted to expression of a COX VIIa subunit polypeptide-encoding nucleic acid when driven by a constitutive promoter.

The constitutive promoter is preferably a medium strength promoter, more preferably selected from a plant derived promoter, such as a GOS2 promoter, more preferably is the promoter GOS2 promoter from rice. Further preferably the constitutive promoter is represented by a nucleic acid sequence substantially similar to SEQ ID NO: 9, most preferably the constitutive promoter is as represented by SEQ ID NO: 9. See the “Definitions” section herein for further examples of constitutive promoters.

Optionally, one or more terminator sequences may be used in the construct introduced into a plant. Preferably, the construct comprises an expression cassette comprising a (GOS2) promoter, substantially similar to SEQ ID NO: 9, and the nucleic acid encoding the COX VIIa subunit polypeptide.

Concerning YLD-ZnF polypeptides, it should be clear that the applicability of the present invention is not restricted to the YLD-ZnF polypeptide-encoding nucleic acid represented by SEQ ID NO: 18, nor is the applicability of the invention restricted to expression of a YLD-ZnF polypeptide-encoding nucleic acid when driven by a constitutive promoter.

The constitutive promoter is preferably a medium strength promoter, more preferably selected from a plant derived promoter, such as a GOS2 promoter, more preferably is the GOS2 promoter from rice. Further preferably the constitutive promoter is represented by a nucleic acid sequence substantially similar to SEQ ID NO: 26, most preferably the constitutive promoter is as represented by SEQ ID NO: 26. See the “Definitions” section herein for further examples of constitutive promoters.

Optionally, one or more terminator sequences may be used in the construct introduced into a plant. Preferably, the construct comprises an expression cassette comprising a GOS2 promoter, substantially similar to SEQ ID NO: 26, and the nucleic acid encoding the YLD-ZnF polypeptide.

Concerning PKT polypeptides, it should be clear that the applicability of the present invention is not restricted to the PKT polypeptide-encoding nucleic acid represented by SEQ ID NO: 51 or SEQ ID NO: 53, nor is the applicability of the invention restricted to expression of a PKT polypeptide-encoding nucleic acid when driven by a constitutive promoter.

The constitutive promoter is preferably a medium strength promoter, more preferably selected from a plant derived promoter, such as a GOS2 promoter, more preferably is the GOS2 promoter from rice. Further preferably the constitutive promoter is represented by a nucleic acid sequence substantially similar to SEQ ID NO: 55, most preferably the constitutive promoter is as represented by SEQ ID NO: 55. See the “Definitions” section herein for further examples of constitutive promoters.

Optionally, one or more terminator sequences may be used in the construct introduced into a plant. Preferably, the construct comprises an expression cassette comprising a (GOS2) promoter, substantially similar to SEQ ID NO: 55, and the nucleic acid encoding the PKT polypeptide.

Concerning NOA polypeptides, it should be clear that the applicability of the present invention is not restricted to the NOA polypeptide-encoding nucleic acid represented by SEQ ID NO: 58, nor is the applicability of the invention restricted to expression of a NOA polypeptide-encoding nucleic acid when driven by a constitutive promoter.

The constitutive promoter is preferably a medium strength promoter, more preferably selected from a plant derived promoter, such as a GOS2 promoter, more preferably is the GOS2 promoter from rice. Further preferably the constitutive promoter is represented by a nucleic acid sequence substantially similar to SEQ ID NO: 71, most preferably the constitutive promoter is as represented by SEQ ID NO: 71. See the “Definitions” section herein for further examples of constitutive promoters.

Optionally, one or more terminator sequences may be used in the construct introduced into a plant. Preferably, the construct comprises an expression cassette comprising a rice GOS2 promoter, substantially similar to SEQ ID NO: 71, and the nucleic acid encoding the NOA polypeptide.

Concerning ASF1-like polypeptides, it should be clear that the applicability of the present invention is not restricted to the ASF1-like polypeptide-encoding nucleic acid represented by SEQ ID NO: 134 or SEQ ID NO: 136, nor is the applicability of the invention restricted to expression of an ASF1-like polypeptide-encoding nucleic acid when driven by a constitutive promoter.

The constitutive promoter is preferably a medium strength promoter, such as a GOS2 promoter, preferably the promoter is a GOS2 promoter from rice. Further preferably the constitutive promoter is represented by a nucleic acid sequence substantially similar to SEQ ID NO: 174, most preferably the constitutive promoter is as represented by SEQ ID NO: 174. See the “Definitions” section herein for further examples of constitutive promoters.

Optionally, one or more terminator sequences may be used in the construct introduced into a plant. Preferably, the construct comprises an expression cassette comprising a GOS2 promoter, substantially similar to SEQ ID NO: 174, and the nucleic acid encoding the ASF1-like polypeptide.

Concerning PHDF polypeptides, it should be clear that the applicability of the present invention is not restricted to the PHDF polypeptide-encoding nucleic acid represented by SEQ ID NO: 175 or SEQ ID NO: 177, nor is the applicability of the invention restricted to expression of a PHDF polypeptide-encoding nucleic acid when driven by a constitutive promoter.

The constitutive promoter is preferably a medium strength promoter, more preferably selected from a plant derived promoter, such as a GOS2 promoter, more preferably is the GOS2 promoter from rice. Further preferably the constitutive promoter is represented by a nucleic acid sequence substantially similar to SEQ ID NO: 181, most preferably the constitutive promoter is as represented by SEQ ID NO: 181. See the “Definitions” section herein for further examples of constitutive promoters.

Optionally, one or more terminator sequences may be used in the construct introduced into a plant. Preferably, the construct comprises an expression cassette comprising a (GOS2) promoter, substantially similar to SEQ ID NO: 181, and the nucleic acid encoding the PHDF polypeptide.

Concerning group I MBF1 polypeptides, it should be clear that the applicability of the present invention is not restricted to a nucleic acid sequence encoding a group I MBF1 polypeptide, as represented by SEQ ID NO: 188, or by SEQ ID NO: 190, or by SEQ ID NO: 192, or by SEQ ID NO: 194, nor is the applicability of the invention restricted to expression of a group I MBF1 polypeptide-encoding nucleic acid sequence when driven by a constitutive promoter.

Optionally, one or more terminator sequences may be used in the construct introduced into a plant.

Additional regulatory elements may include transcriptional as well as translational enhancers. Those skilled in the art will be aware of terminator and enhancer sequences that may be suitable for use in performing the invention. An intron sequence may also be added to the 5′ untranslated region (UTR) or in the coding sequence to increase the amount of the mature message that accumulates in the cytosol, as described in the definitions section. Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acids, it is advantageous to use marker genes (or reporter genes). Therefore, the genetic construct may optionally comprise a selectable marker gene. Selectable markers are described in more detail in the “definitions” section herein. The marker genes may be removed or excised from the transgenic cell once they are no longer needed. Techniques for marker removal are known in the art, useful techniques are described above in the definitions section.

It is known that upon stable or transient integration of nucleic acid sequences into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can for example be used in mutants in which these genes are not functional by, for example, deletion by conventional methods. Furthermore, nucleic acid sequence molecules encoding a selectable marker can be introduced into a host cell on the same vector that comprises the sequence encoding the polypeptides of the invention or used in the methods of the invention, or else in a separate vector. Cells which have been stably transfected with the introduced nucleic acid sequence can be identified for example by selection (for example, cells which have integrated the selectable marker survive whereas the other cells die). The marker genes may be removed or excised from the transgenic cell once they are no longer needed. Techniques for marker gene removal are known in the art, useful techniques are described above in the definitions section.

The invention also provides a method for the production of transgenic plants having enhanced abiotic stress tolerance and/or enhanced yield-related traits relative to control plants, comprising introduction and expression in a plant of any nucleic acid encoding a COX VIIa subunit polypeptide, or a YLD-ZnF polypeptide, or a PKT polypeptide, or a NOA polypeptide, or an ASF1-like polypeptide, or a PHDF polypeptide, or a group I MBF1 polypeptide, as defined hereinabove.

More specifically, the present invention provides a method for the production of transgenic plants having enhanced abiotic stress tolerance, particularly increased (mild) drought tolerance, which method comprises:
(i) introducing and expressing in a plant or plant cell a nucleic acid encoding a COX VIIa subunit polypeptide, or a PKT polypeptide, or a PHDF polypeptide; and
(ii) cultivating the plant cell under abiotic stress conditions.

The nucleic acid of (i) may be any of the nucleic acids capable of encoding a COX VIIa subunit polypeptide, or a PKT polypeptide, or a PHDF polypeptide, as defined herein.

More specifically, the present invention also provides a method for the production of transgenic plants having enhanced yield-related traits, particularly increased (seed) yield and/or early vigour, which method comprises:
(i) introducing and expressing in a plant or plant cell a nucleic acid encoding a YLD-ZnF polypeptide, or an ASF1-like polypeptide; and
(ii) cultivating the plant cell under conditions promoting plant growth and development.

The nucleic acid of (i) may be any of the nucleic acids capable of encoding a YLD-ZnF polypeptide, or an ASF1-like polypeptide, as defined herein.

More specifically, the present invention also provides a method for the production of transgenic plants having enhanced yield-related traits, particularly increased yield, which method comprises:
(i) introducing and expressing in a plant or plant cell a nucleic acid encoding a NOA polypeptide; and
(ii) cultivating the plant cell under conditions promoting plant growth and development.

The nucleic acid of (i) may be any of the nucleic acids capable of encoding a NOA polypeptide as defined herein.

More specifically, the present invention also provides a method for the production of transgenic plants having increased yield-related traits relative to control plants, which method comprises:
(i) introducing and expressing in a plant, plant part, or plant cell a nucleic acid sequence encoding a group I MBF1 polypeptide; and
(ii) cultivating the plant cell, plant part or plant under conditions promoting plant growth and development.

The nucleic acid sequence of (i) may be any of the nucleic acid sequences capable of encoding a group I MBF1 polypeptide as defined herein.

The nucleic acid may be introduced directly into a plant cell or into the plant itself (including introduction into a tissue, organ or any other part of a plant). According to a preferred feature of the present invention, the nucleic acid is preferably introduced into a plant by transformation. The term “transformation” is described in more detail in the “definitions” section herein.

The genetically modified plant cells can be regenerated via all methods with which the skilled worker is familiar. Suitable methods can be found in the above-mentioned publications by S. D. Kung and R. Wu, Potrykus or Höfgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated nucleic acid encoding a COX VIIa subunit polypeptide, or a YLD-ZnF polypeptide, or a PKT polypeptide, or a NOA polypeptide, or an ASF1-like polypeptide, or a PHDF polypeptide, or a group I MBF1 polypeptide, as defined hereinabove. Preferred host cells according to the invention are plant cells. Host plants for the nucleic acids or the vector used in the method according to the invention, the expression cassette or construct or vector are, in principle, advantageously all plants, which are capable of synthesizing the polypeptides used in the inventive method.

The methods of the invention are advantageously applicable to any plant. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant.

Examples of crop plants include soybean, sunflower, canola, alfalfa, rapeseed, linseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum, emmer, spelt, secale, einkorn, teff, milo and oats.

The methods of the invention are advantageously applicable to any plant. Plants that are particularly useful in the methods of the invention include all plants which belong to the superfamily Viridiplantae, in particular monocotyledonous and dicotyledonous plants including fodder or forage legumes, ornamental plants, food crops, trees or shrubs.

According to a preferred embodiment of the present invention, the plant is a crop plant. Examples of crop plants include soybean, sunflower, canola, alfalfa, rapeseed, linseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum, emmer, spelt, secale, einkorn, teff, milo and oats.

The invention also extends to harvestable parts of a plant such as, but not limited to seeds, leaves, fruits, flowers, stems, roots, rhizomes, tubers and bulbs, which harvestable parts comprise a recombinant nucleic acid encoding a COX VIIa subunit polypeptide, or a YLD-ZnF polypeptide, or a PKT polypeptide, or a NOA polypeptide, or an ASF1-like polypeptide, or a PHDF polypeptide, or a group I MBF1 polypeptide. The invention furthermore relates to products derived, preferably directly derived, from a harvestable part of such a plant, such as dry pellets or powders, oil, fat and fatty acids, starch or proteins.

According to a preferred feature of the invention, the modulated expression is increased expression. Methods for increasing expression of nucleic acids or genes, or gene products, are well documented in the art and examples are provided in the definitions section.

As mentioned above, a preferred method for modulating expression of a nucleic acid encoding a COX VIIa subunit polypeptide, or a YLD-ZnF polypeptide, or a PKT polypeptide, or a NOA polypeptide, or an ASF1-like polypeptide, or a PHDF polypeptide, or a group I MBF1 polypeptide, is by introducing and expressing in a plant a nucleic acid encoding a COX VIIa subunit polypeptide, or a YLD-ZnF polypeptide, or a PKT polypeptide, or a NOA polypeptide, or an ASF1-like polypeptide, or a PHDF polypeptide, or a group I MBF1 polypeptide; however the effects of performing the method, i.e. enhancing abiotic stress tolerance may also be achieved using other well known techniques, including but not limited to T-DNA activation tagging, TILLING, homologous recombination. A description of these techniques is provided in the definitions section.

The present invention also encompasses use of nucleic acids encoding COX VIIa subunit polypeptides, or PKT polypeptides, or PHDF polypeptides, as described herein and use of these COX VIIa subunit polypeptides, or PKT polypeptides, or PHDF polypeptides, in enhancing any of the aforementioned abiotic stresses in plants.

The present invention also encompasses use of nucleic acids encoding YLD-ZnF polypeptides, or NOA polypeptides, or ASF1-like polypeptides, as described herein and use of these YLD-ZnF polypeptides, or NOA polypeptides, or ASF1-like polypeptides, in enhancing any of the aforementioned yield-related traits in plants.

The present invention also encompasses use of nucleic acid sequences encoding group I MBF1 polypeptides as described herein and use of these group I MBF1 polypeptides in increasing any of the aforementioned yield-related traits in plants, under normal growth conditions, under abiotic stress growth (preferably osmotic stress growth conditions) conditions, and under growth conditions of reduced nutrient availability, preferably under conditions of reduced nitrogen availability.

Nucleic acids encoding COX VIIa subunit polypeptide, or YLD-ZnF polypeptide, or PKT polypeptide, or NOA polypeptide, or ASF1-like polypeptide, or PHDF polypeptide, or group I MBF1 polypeptide, described herein, or the COX VIIa subunit polypeptides, or YLD-ZnF polypeptides, or PKT polypeptides, or NOA polypeptides, or ASF1-like polypeptides, or PHDF polypeptides, or group I MBF1 polypeptides themselves, may find use in breeding programmes in which a DNA marker is identified which may be genetically linked to a gene encoding COX VIIa subunit polypeptide, or YLD-ZnF polypeptide, or PKT polypeptide, or NOA polypeptide, or ASF1-like polypeptide, or PHDF polypeptide, or group I MBF1 polypeptide. The nucleic acids/genes, or the COX VIIa subunit polypeptides, or YLD-ZnF polypeptides, or PKT polypeptides, or NOA polypeptides, or ASF1-like polypeptides, or PHDF polypeptides, or group I MBF1 polypeptides themselves, may be used to define a molecular marker. This DNA or protein marker may then be used in breeding programmes to select plants having enhanced abiotic stress tolerance and/or enhanced yield-related traits as defined hereinabove in the methods of the invention.

Allelic variants of a nucleic acid/gene encoding a COX VIIa subunit polypeptide, or a YLD-ZnF polypeptide, or a PKT polypeptide, or a NOA polypeptide, or an ASF1-like polypeptide, or a PHDF polypeptide, or a group I MBF1 polypeptide, may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

Nucleic acids encoding COX VIIa subunit polypeptides, or YLD-ZnF polypeptides, or PKT polypeptides, or NOA polypeptides, or ASF1-like polypeptides, or PHDF polypeptides, or group I MBF1 polypeptides, may also be used as probes for genetically and physically mapping the genes that they are a part of, and as markers for traits linked to those genes. Such information may be useful in plant breeding in order to develop lines with desired phenotypes. Such use of nucleic acids encoding a COX VIIa subunit polypeptide, or a YLD-ZnF polypeptide, or a PKT polypeptide, or a NOA polypeptide, or an ASF1-like polypeptide, or a PHDF polypeptide, or a group I MBF1 polypeptide, requires only a nucleic acid sequence of at least 15 nucleotides in length. The nucleic acids encoding a COX VIIa subunit polypeptide, or a YLD-ZnF polypeptide, or a PKT polypeptide, or a NOA polypeptide, or an ASF1-like polypeptide, or a PHDF polypeptide, or a group I MBF1 polypeptide, may be used as restriction fragment length polymorphism (RFLP) markers. Southern blots (Sambrook J, Fritsch EF and Maniatis T (1989) Molecular Cloning, A Laboratory Manual) of restriction-digested plant genomic DNA may be probed with the nucleic acids encoding a COX VIIa subunit polypeptide, or a YLD-ZnF polypeptide, or a PKT polypeptide, or a NOA polypeptide, or an ASF1-like polypeptide, or a PHDF polypeptide, or a group I MBF1 polypeptide. The resulting banding patterns may then be subjected to genetic analyses using computer programs such as MapMaker (Lander et al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the encoding nucleic acid a COX VIIa subunit polypeptide, or a YLD-ZnF polypeptide, or a PKT polypeptide, or a NOA polypeptide, or an ASF1-like polypeptide, or a PHDF polypeptide, or a group I MBF1 polypeptide, in the genetic map previously obtained using this population (Botstein et al. (1980) Am. J. Hum. Genet. 32:314-331).

The production and use of plant gene-derived probes for use in genetic mapping is described in Bernatzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art. The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridisation (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples include allele-specific amplification (Kazazian (1989) J. Lab. Clin. Med 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having enhanced abiotic stress tolerance and/or enhanced yield-related traits, as described hereinbefore. These traits may also be combined with other economically advantageous traits, such as further abiotic or biotic stress tolerance-enhancing traits and/or yield-enhancing traits, enhanced yield-related traits and/or tolerance to other abiotic and biotic stresses, traits modifying various architectural features and/or biochemical and/or physiological features.

### Items

### 1. COX VIIa Subunit Polypeptides

6. Method for enhancing abiotic stress tolerance in plants by modulating expression in a plant of a nucleic acid encoding a cytochrome c oxidase (COX) VIIa subunit polypeptide (COX VIIa subunit) or an orthologue or paralogue thereof.
7. Method according to item 1, wherein said modulated expression is effected by introducing and expressing in a plant a nucleic acid encoding cytochrome c oxidase (COX) VIIa subunit polypeptide.
8. Method according to items 2 or 3, wherein said nucleic acid encoding a COX VIIa subunit polypeptide encodes any one of the proteins listed in Table A1 or is a portion of such a nucleic acid, or a nucleic acid capable of hybridising with such a nucleic acid.
9. Method according to any one of items 1 to 4, wherein said nucleic acid sequence encodes an orthologue or paralogue of any of the proteins given in Table A1.
10. Method according to items 3 or 4, wherein said nucleic acid is operably linked to a constitutive promoter, preferably to a GOS2 promoter, most preferably to a GOS2 promoter from rice.
11. Method according to any one of items 1 to 5, wherein said nucleic acid encoding a COX VIIa subunit polypeptide is of Physcomitrella patens.
12. Plant or part thereof, including seeds, obtainable by a method according to any one of items 1 to 6, wherein said plant or part thereof comprises a recombinant nucleic acid encoding a COX VIIa subunit polypeptide.
13. Construct comprising:
   (i) nucleic acid encoding a COX VIIa subunit polypeptide as defined in items 1 or 2;
   (ii) one or more control sequences capable of driving expression of the nucleic acid sequence of (a); and optionally
   (iii) a transcription termination sequence.
14. Construct according to item 9, wherein one of said control sequences is a constitutive promoter, preferably a GOS2 promoter, most preferably a GOS2 promoter from rice.
15. Use of a construct according to item 8 or 9 in a method for making plants having increased abiotic stress tolerance relative to control plants.
16. Plant, plant part or plant cell transformed with a construct according to item 8 or 9.
17. Method for the production of a transgenic plant having increased abiotic stress tolerance relative to control plants, comprising:
   (i) introducing and expressing in a plant a nucleic acid encoding a COX VIIa subunit polypeptide; and
   (ii) cultivating the plant cell under conditions promoting abiotic stress.
18. Transgenic plant having abiotic stress tolerance, relative to control plants, resulting from modulated expression of a nucleic acid encoding a COX VIIa subunit polypeptide, or a transgenic plant cell derived from said transgenic plant.
19. Transgenic plant according to item 7, 11 or 13, or a transgenic plant cell derived thereof, wherein said plant is a crop plant or a monocot or a cereal, such as rice, maize, wheat, barley, millet, rye, triticale, sorghum, sugarcane, emmer, spelt, secale, einkorn, teff, milo and oats.
20. Harvestable parts of a plant according to item 14, wherein said harvestable parts are preferably shoot biomass and/or seeds.
21. Products derived from a plant according to item 14 and/or from harvestable parts of a plant according to item 15.
22. Use of a nucleic acid encoding a COX VIIa subunit polypeptide in increasing yield, particularly in increasing abiotic stress tolerance, relative to control plants.

### 2. YLD-ZnF Polypeptides

1. A method for enhancing yield-related traits in plants relative to control plants, comprising modulating expression in a plant of a nucleic acid encoding a YLD-ZnF polypeptide, wherein said YLD-ZnF polypeptide comprises a zf-DNL domain.
2. Method according to item 1, wherein said YLD-ZnF polypeptide comprises one or more of the following motifs:
   (i) Motif 1, SEQ ID NO: 20,
   (ii) Motif 2, SEQ ID NO: 21,
   (iii) Motif 3, SEQ ID NO: 22,
   (iv) Motif 4, SEQ ID NO: 23.
3. Method according to item 1 or 2, wherein said modulated expression is effected by introducing and expressing in a plant a nucleic acid encoding a YLD-ZnF polypeptide.
4. Method according to any one of items 1 to 3, wherein said nucleic acid encoding a YLD-ZnF polypeptide encodes any one of the proteins listed in Table A2 or is a portion of such a nucleic acid, or a nucleic acid capable of hybridising with such a nucleic acid.
5. Method according to any one of items 1 to 4, wherein said nucleic acid sequence encodes an orthologue or paralogue of any of the proteins given in Table A2.
6. Method according to any preceding item, wherein said enhanced yield-related traits comprise increased yield, preferably increased seed yield, and/or increased early vigour relative to control plants.
7. Method according to any one of items 1 to 6, wherein said enhanced yield-related traits are obtained under non-stress conditions.
8. Method according to any one of items 1 to 6, wherein said enhanced yield-related traits are obtained under conditions of nitrogen deficiency.
9. Method according to any one of items 3 to 8, wherein said nucleic acid is operably linked to a constitutive promoter, preferably to a GOS2 promoter, most preferably to a GOS2 promoter from rice.
10. Method according to any one of items 1 to 9, wherein said nucleic acid encoding a YLD-ZnF polypeptide is of plant origin, preferably from a dicotyledonous plant, further preferably from the family Fabaceae, more preferably from the genus Medicago, most preferably from Medicago truncatula.
11. Plant or part thereof, including seeds, obtainable by a method according to any one of items 1 to 10, wherein said plant or part thereof comprises a recombinant nucleic acid encoding a YLD-ZnF polypeptide.
12. Construct comprising:
   (i) nucleic acid encoding a YLD-ZnF polypeptide as defined in items 1 or 2;
   (ii) one or more control sequences capable of driving expression of the nucleic acid sequence of (a); and optionally
   (iii) a transcription termination sequence.
13. Construct according to item 12, wherein one of said control sequences is a constitutive promoter, preferably a GOS2 promoter, most preferably a GOS2 promoter from rice.
14. Use of a construct according to item 12 or 13 in a method for making plants having increased yield, particularly increased seed yield, and/or increased early vigour relative to control plants.
15. Plant, plant part or plant cell transformed with a construct according to item 12 or 13.
16. Method for the production of a transgenic plant having increased yield, particularly increased biomass and/or increased seed yield relative to control plants, comprising:
   (i) introducing and expressing in a plant a nucleic acid encoding a YLD-ZnF polypeptide as defined in item 1 or 2; and
   (ii) cultivating the plant cell under conditions promoting plant growth and development.
17. Transgenic plant having increased yield, particularly increased seed yield, and/or increased early vigour, relative to control plants, resulting from modulated expression of a nucleic acid encoding a YLD-ZnF polypeptide as defined in item 1 or 2, or a transgenic plant cell derived from said transgenic plant.
18. Transgenic plant according to item 11, 15 or 17, or a transgenic plant cell derived thereof, wherein said plant is a crop plant or a monocot or a cereal, such as rice, maize, wheat, barley, millet, rye, triticale, sorghum emmer, spelt, secale, einkorn, teff, milo and oats.
19. Harvestable parts of a plant according to item 18, wherein said harvestable parts are preferably shoot biomass and/or seeds.
20. Products derived from a plant according to item 18 and/or from harvestable parts of a plant according to item 19.
21. Use of a nucleic acid encoding a YLD-ZnF polypeptide in increasing yield, particularly in increasing seed yield, and/or early vigour in plants, relative to control plants.

### 3. PKT Polypeptides

1. Method for enhancing abiotic stress tolerance in plants by modulating expression in a plant of a nucleic acid encoding a PKT polypeptide or an orthologue or paralogue thereof.
2. Method according to item 1, wherein said modulated expression is effected by introducing and expressing in a plant a nucleic acid encoding PKT polypeptide.
3. Method according to items 2 or 3, wherein said nucleic acid encoding a PKT polypeptide encodes any one of the proteins listed in Table A3 or is a portion of such a nucleic acid, or a nucleic acid capable of hybridising with such a nucleic acid.
4. Method according to any one of items 1 to 4, wherein said nucleic acid sequence encodes an orthologue or paralogue of any of the proteins given in Table A3.
5. Method according to items 3 or 4, wherein said nucleic acid is operably linked to a constitutive promoter, preferably to a GOS2 promoter, most preferably to a GOS2 promoter from rice.
6. Method according to any one of items 1 to 5, wherein said nucleic acid encoding a PKT polypeptide is of Populus trichocarpa.
7. Plant or part thereof, including seeds, obtainable by a method according to any one of items 1 to 6, wherein said plant or part thereof comprises a recombinant nucleic acid encoding a PKT polypeptide.
8. Construct comprising:
   (i) nucleic acid encoding a PKT polypeptide as defined in items 1 or 2;
   (ii) one or more control sequences capable of driving expression of the nucleic acid sequence of (a); and optionally
   (iii) a transcription termination sequence.
9. Construct according to item 9, wherein one of said control sequences is a constitutive promoter, preferably a GOS2 promoter, most preferably a GOS2 promoter from rice.
10. Use of a construct according to item 8 or 9 in a method for making plants having increased abiotic stress tolerance relative to control plants.
11. Plant, plant part or plant cell transformed with a construct according to item 8 or 9.
12. Method for the production of a transgenic plant having increased abiotic stress tolerance relative to control plants, comprising:
   (i) introducing and expressing in a plant a nucleic acid encoding a PKT polypeptide; and
   (ii) cultivating the plant cell under conditions promoting abiotic stress.
13. Transgenic plant having abiotic stress tolerance, relative to control plants, resulting from modulated expression of a nucleic acid encoding a PKT polypeptide, or a transgenic plant cell derived from said transgenic plant.
14. Transgenic plant according to item 7, 11 or 13, or a transgenic plant cell derived thereof, wherein said plant is a crop plant or a monocot or a cereal, such as rice, maize, wheat, barley, millet, rye, triticale, sorghum, sugarcane, emmer, spelt, secale, einkorn, teff, milo and oats.
15. Harvestable parts of a plant according to item 14, wherein said harvestable parts are preferably shoot biomass and/or seeds.
16. Products derived from a plant according to item 14 and/or from harvestable parts of a plant according to item 15.
17. Use of a nucleic acid encoding a PKT polypeptide in increasing yield, particularly in increasing abiotic stress tolerance, relative to control plants.

### 4. NOA Polypeptides

1. A method for enhancing yield-related traits in plants relative to control plants, comprising modulating expression in a plant of a nucleic acid encoding a nitric oxide associated (NOA) polypeptide, wherein said nitric oxide associated polypeptide comprises a PTHR11089 domain.
2. Method according to item 1, wherein said NOA polypeptide comprises one or more of the following motifs: Motif 5 (SEQ ID NO: 60), Motif 6 (SEQ ID NO: 61), Motif 7 (SEQ ID NO 62), Motif 8 (SEQ ID NO: 63), Motif 9 (SEQ ID NO: 64), and Motif 10 (SEQ ID NO: 65).
3. Method according to item 1 or 2, wherein said modulated expression is effected by introducing and expressing in a plant a nucleic acid encoding a NOA polypeptide.
4. Method according to any one of items 1 to 3, wherein said nucleic acid encoding a NOA polypeptide encodes any one of the proteins listed in Table A4 or is a portion of such a nucleic acid, or a nucleic acid capable of hybridising with such a nucleic acid.
5. Method according to any one of items 1 to 4, wherein said nucleic acid sequence encodes an orthologue or paralogue of any of the proteins given in Table A4.
6. Method according to any preceding item, wherein said enhanced yield-related traits comprise increased yield, preferably increased biomass and/or increased seed yield relative to control plants.
7. Method according to any one of items 1 to 6, wherein said enhanced yield-related traits are obtained under non-stress conditions.
8. Method according to any one of items 3 to 7, wherein said nucleic acid is operably linked to a constitutive promoter, preferably to a GOS2 promoter, most preferably to a GOS2 promoter from rice.
9. Method according to any one of items 1 to 8, wherein said nucleic acid encoding a NOA polypeptide is of plant origin, preferably from a dicotyledonous plant, further preferably from the family Brassicaceae, more preferably from the genus Arabidopsis, most preferably from Arabidopsis thaliana.
10. Plant or part thereof, including seeds, obtainable by a method according to any one of items 1 to 9, wherein said plant or part thereof comprises a recombinant nucleic acid encoding a NOA polypeptide.
11. Construct comprising:
   (i) nucleic acid encoding a NOA polypeptide as defined in items 1 or 2;
   (ii) one or more control sequences capable of driving expression of the nucleic acid sequence of (a); and optionally
   (iii) a transcription termination sequence.
12. Construct according to item 11, wherein one of said control sequences is a constitutive promoter, preferably a GOS2 promoter, most preferably a GOS2 promoter from rice.
13. Use of a construct according to item 11 or 12 in a method for making plants having increased yield, particularly increased biomass and/or increased seed yield relative to control plants.
14. Plant, plant part or plant cell transformed with a construct according to item 11 or 12.
15. Method for the production of a transgenic plant having increased yield, particularly increased biomass and/or increased seed yield relative to control plants, comprising:
   (i) introducing and expressing in a plant a nucleic acid encoding a NOA polypeptide as defined in item 1 or 2; and
   (ii) cultivating the plant cell under conditions promoting plant growth and development.
16. Transgenic plant having increased yield, particularly increased biomass and/or increased seed yield, relative to control plants, resulting from modulated expression of a nucleic acid encoding a NOA polypeptide as defined in item 1 or 2, or a transgenic plant cell derived from said transgenic plant.
17. Transgenic plant according to item 10, 14 or 16, or a transgenic plant cell derived thereof, wherein said plant is a crop plant or a monocot or a cereal, such as rice, maize, wheat, barley, millet, rye, triticale, sorghum emmer, spelt, secale, einkorn, teff, milo and oats.
18. Harvestable parts of a plant according to item 17, wherein said harvestable parts are preferably shoot biomass and/or seeds.
19. Products derived from a plant according to item 17 and/or from harvestable parts of a plant according to item 18.
20. Use of a nucleic acid encoding a NOA polypeptide in increasing yield, particularly in increasing seed yield and/or shoot biomass in plants, relative to control plants.
21. An isolated nucleic acid molecule comprising:
   (i) a nucleic acid represented by SEQ ID NO: 125;
   (ii) the complement of a nucleic acid represented by SEQ ID NO: 125;
   (iii) a nucleic acid encoding a NOA polypeptide having, in increasing order of preference, at least 50%, 55%, 60%, 65%, 70%, 75%, 80%, 85%, 90%, 95%, 96%, 97%, 98%, 99% or more sequence identity to the amino acid sequence represented by SEQ ID NO: 94.
22. An isolated polypeptide comprising:
   (i) an amino acid sequence represented by SEQ ID NO: 94;
   (ii) an amino acid sequence having, in increasing order of preference, at least 50%, 55%, 60%, 65%, 70%, 75%, 80%, 85%, 90%, 95%, 96%, 97%, 98%, 99% or more sequence identity to the amino acid sequence represented by SEQ ID NO: 94;
   (iii) derivatives of any of the amino acid sequences given in (i) or (ii) above.
      5. ASF1-like Polypeptides
1. A method for enhancing yield-related traits in plants relative to control plants, comprising modulating expression in a plant of a nucleic acid encoding an ASF1-like polypeptide.
2. Method according to item 1, wherein said ASF1-like polypeptide comprises one or more of the following motifs:

MOTIF I:
DLEWKL I/T YVGSA,
MOTIF II:
S/P P D/E P/V/T S/L/A/N K/R I R/P/Q E/A/D E/A D/E
I/V I/L GVTV L/I LLTC S/A Y,
MOTIF III:
Q/R EF V/I/L/M R V/I GYYV N/S/Q N/Q,
MOTIF IV:
V/I/L Q/R RNIL A/T/S/V D/E KPRVT K/R F P/A I,

or a motif having in increasing order of preference at least 50%, 51%, 52%, 53%, 54%, 55%, 56%, 57%, 58%, 59%, 60%, 81%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more sequence identity to any one or more of Motifs I to IV.
3. Method according to item 1 or 2, wherein said modulated expression is effected by introducing and expressing in a plant a nucleic acid encoding an ASF1-like polypeptide.
4. Method according to any preceding item, wherein said nucleic acid encoding an ASF1-like polypeptide encodes any one of the proteins listed in Table A5 or is a portion of such a nucleic acid, or a nucleic acid capable of hybridising with such a nucleic acid.
5. Method according to any preceding item, wherein said nucleic acid sequence encodes an orthologue or paralogue of any of the proteins given in Table A5.
6. Method according to any preceding item, wherein said enhanced yield-related traits comprise increased yield, preferably increased biomass and/or increased seed yield relative to control plants.
7. Method according to any one of items 1 to 6, wherein said enhanced yield-related traits are obtained under non-stress conditions.
8. Method according to any one of items 3 to 8, wherein said nucleic acid is operably linked to a constitutive promoter, preferably to a GOS2 promoter, most preferably to a GOS2 promoter from rice.
9. Method according to any preceding item, wherein said nucleic acid encoding an ASF1-like polypeptide is of plant origin, preferably from a monocotyledonous or dicotyledonous plant, further preferably from the family Poaceae or Brassicaceae, more preferably from the genus Arabidopsis, most preferably from Arabidopsis thaliana or from the genus Oryza or Oryza sativa.
10. Plant or part thereof, including seeds, obtainable by a method according to any preceding item, wherein said plant or part thereof comprises a recombinant nucleic acid encoding an ASF1-like polypeptide.
11. Construct comprising:
   (iv) nucleic acid encoding an ASF1-like polypeptide as defined in items 1 or 2;
   (v) one or more control sequences capable of driving expression of the nucleic acid sequence of (i); and optionally
   (vi) a transcription termination sequence.
12. Construct according to item 11, wherein one of said control sequences is a constitutive promoter, preferably a GOS2 promoter, most preferably a GOS2 promoter from rice.
13. Use of a construct according to item 11 or 12 in a method for making plants having increased yield, particularly increased biomass and/or increased seed yield relative to control plants.
14. Plant, plant part or plant cell transformed with a construct according to item 11 or 12.
15. Method for the production of a transgenic plant having increased yield, particularly increased biomass and/or increased seed yield relative to control plants, comprising:
   (i) introducing and expressing in a plant a nucleic acid encoding an ASF1-like polypeptide as defined in item 1 or 2; and
   (ii) cultivating the plant cell under conditions promoting plant growth and development.
16. Transgenic plant having increased yield, particularly increased biomass and/or increased seed yield, relative to control plants, resulting from modulated expression of a nucleic acid encoding an ASF1-like polypeptide as defined in item 1 or 2, or a transgenic plant cell derived from said transgenic plant.
17. Transgenic plant according to item 10, 14 or 16, or a transgenic plant cell derived thereof, wherein said plant is a crop plant or a monocot or a cereal, such as rice, maize, wheat, barley, millet, rye, triticale, sorghum emmer, spelt, secale, einkorn, teff, milo and oats.
18. Harvestable parts of a plant according to item 17, wherein said harvestable parts are preferably shoot biomass and/or seeds.
19. Products derived from a plant according to item 17 and/or from harvestable parts of a plant according to item 18.
20. Use of a nucleic acid encoding an ASF1-like polypeptide in increasing yield, particularly in increasing seed yield and/or shoot biomass in plants, relative to control plants.

### 6. PHDF Polypeptides

1. Method for enhancing abiotic stress tolerance in plants by modulating expression in a plant of a nucleic acid encoding a PHDF polypeptide or an orthologue or paralogue thereof.
2. Method according to item 1, wherein said modulated expression is effected by introducing and expressing in a plant a nucleic acid encoding PHDF polypeptide.
3. Method according to items 2 or 3, wherein said nucleic acid encoding a PHDF polypeptide encodes any one of the proteins listed in Table A6 or is a portion of such a nucleic acid, or a nucleic acid capable of hybridising with such a nucleic acid.
4. Method according to any one of items 1 to 4, wherein said nucleic acid sequence encodes an orthologue or paralogue of any of the proteins given in Table A6.
5. Method according to items 3 or 4, wherein said nucleic acid is operably linked to a constitutive promoter, preferably to a GOS2 promoter, most preferably to a GOS2 promoter from rice.
6. Method according to any one of items 1 to 5, wherein said nucleic acid encoding a PHDF polypeptide is of Solanum lycopersicum.
7. Plant or part thereof, including seeds, obtainable by a method according to any one of items 1 to 6, wherein said plant or part thereof comprises a recombinant nucleic acid encoding a PHDF polypeptide.
8. Construct comprising:
   (i) nucleic acid encoding a PHDF polypeptide as defined in items 1 or 2;
   (ii) one or more control sequences capable of driving expression of the nucleic acid sequence of (a); and optionally
   (iii) a transcription termination sequence.
9. Construct according to item 9, wherein one of said control sequences is a constitutive promoter, preferably a GOS2 promoter, most preferably a GOS2 promoter from rice.
10. Use of a construct according to item 8 or 9 in a method for making plants having increased abiotic stress tolerance relative to control plants.
11. Plant, plant part or plant cell transformed with a construct according to item 8 or 9.
12. Method for the production of a transgenic plant having increased abiotic stress tolerance relative to control plants, comprising:
   (i) introducing and expressing in a plant a nucleic acid encoding a PHDF polypeptide; and
   (ii) cultivating the plant cell under conditions promoting abiotic stress.
13. Transgenic plant having abiotic stress tolerance, relative to control plants, resulting from modulated expression of a nucleic acid encoding a PHDF polypeptide, or a transgenic plant cell derived from said transgenic plant.
14. Transgenic plant according to item 7, 11 or 13, or a transgenic plant cell derived thereof, wherein said plant is a crop plant or a monocot or a cereal, such as rice, maize, wheat, barley, millet, rye, triticale, sorghum, sugarcane, emmer, spelt, secale, einkorn, teff, milo and oats.
15. Harvestable parts of a plant according to item 14, wherein said harvestable parts are preferably shoot biomass and/or seeds.
16. Products derived from a plant according to item 14 and/or from harvestable parts of a plant according to item 15.
17. Use of a nucleic acid encoding a PHDF polypeptide in increasing yield, particularly in increasing abiotic stress tolerance, relative to control plants.
   7. group I MBF1 polypeptides
1. A method for increasing yield-related traits in plants relative to control plants, comprising increasing expression in a plant of a nucleic acid sequence encoding a group I multiprotein bridging factor 1 (MBF1) polypeptide, which group I MBF1 polypeptide comprises (i) in increasing order of preference at least 70%, 75%, 80%, 85%, 90%, 95%, 98%, 99% or more amino acid sequence identity to an N-terminal multibridging domain with an InterPro entry IPR0013729 (PFAM entry PF08523 MBF1) as represented by SEQ ID NO: 250; and (ii) in increasing order of preference at least 70%, 75%, 80%, 85%, 90%, 95%, 98%, 99% or more amino acid sequence identity to a helix-turn-helix 3 domain with an InterPro entry IPR001387 (PFAM ENTRY PF01381 HTH_{—}3).
2. Method according to item 1, wherein said group I MBF1 polypeptide comprises in increasing order of preference at least 50%, 55%, 60%, 65%, 70%, 75%, 80%, 85%, 90%, 95%, 98%, 99% or more amino acid sequence identity to a polypeptide as represented by SEQ ID NO: 189, or as represented by SEQ ID NO: 191, or as represented by SEQ ID NO: 193, or as represented by SEQ ID NO: 195.
3. Method according to item 1, wherein said group I MBF1 polypeptide comprises in increasing order of preference at least at least 50%, 55%, 60%, 65%, 70%, 75%, 80%, 85%, 90%, 95%, 98%, 99% or more amino acid sequence identity to any of the polypeptide sequences given in Table A7 herein.
4. Method according to any preceding item, wherein said group I MBF1 polypeptide, which when used in the construction of an MBF1 phylogenetic tree, such as the one depicted in FIG. 15, clusters with the group I MBF1 polypeptides comprising the polypeptide sequences as represented by SEQ ID NO: 189, SEQ ID NO: 191, SEQ ID NO: 193, and SEQ ID NO: 195, rather than with any other group.
5. Method according to any preceding item, wherein said group I MBF1 polypeptide complements a yeast strain deficient for MBF1 activity.
6. Method according to any preceding item, wherein said nucleic acid sequence encoding a group I MBF1 polypeptide is represented by any one of the nucleic acid sequence SEQ ID NOs given in Table A7 or a portion thereof, or a sequence capable of hybridising with any one of the nucleic acid sequences SEQ ID NOs given in Table A7, or to a complement thereof.
7. Method according to any preceding item, wherein said nucleic acid sequence encodes an orthologue or paralogue of any of the polypeptide sequence SEQ ID NOs given in Table A7.
8. Method according to any preceding item, wherein said increased expression is effected by any one or more of: T-DNA activation tagging, TILLING, or homologous recombination.
9. Method according to any preceding item, wherein said increased expression is effected by introducing and expressing in a plant a nucleic acid sequence encoding a group I MBF1 polypeptide.
10. Method according to any preceding item, wherein said increased yield-related trait is one or more of: increased aboveground biomass, increased early vigor, increased seed yield per plant, increased seed fill rate, increased number of filled seeds, or increased number of primary panicles.
11. Method according to any preceding item, wherein said increased yield-related traits are obtained in plants grown under conditions of reduced nutrient availablity, preferably reduced nitrogen availability.
12. Method according to any preceding item, wherein said nucleic acid sequence is operably linked to a constitutive promoter.
13. Method according to item 12, wherein said constitutive promoter is a GOS2 promoter, preferably a GOS2 promoter from rice, most preferably a GOS2 sequence as represented by SEQ ID NO: 254.
14. Method according to item 12, wherein said constitutive promoter is an HMG promoter, preferably an HMG promoter from rice, most preferably an HMG sequence as represented by SEQ ID NO: 253.
15. Method according to any preceding item, wherein said nucleic acid sequence encoding a group I MBF1 polypeptide is from a plant.
16. Method according to 15, wherein said nucleic acid sequence encoding a group I MBF1 polypeptide is from a dicotyledonous plant, more preferably from Arabidopsis thaliana, or Medicago truncatula.
17. Method according to 15, wherein said nucleic acid sequence encoding a group I MBF1 polypeptide is from a monocotyledonous plant, more preferably from Triticum aestivum.
18. Plants, parts thereof (including seeds), or plant cells obtainable by a method according to any preceding item, wherein said plant, part or cell thereof comprises an isolated nucleic acid transgene encoding a group I MBF1 polypeptide.
19. Construct comprising:
   (a) a nucleic acid sequence encoding a group I MBF1 polypeptide as defined in any one of items 1 to 7;
   (b) one or more control sequences capable of driving expression of the nucleic acid sequence of (a); and optionally
   (c) a transcription termination sequence.
20. Construct according to item 19 wherein said control sequence is a constitutive promoter.
21. Construct according to item 20 wherein said constitutive promoter is a GOS2 promoter, preferably a GOS2 promoter from rice, most preferably a GOS2 sequence as represented by SEQ ID NO: 254.
22. Construct according to item 20 wherein said constitutive promoter is an HMG promoter, preferably an HMG promoter from rice, most preferably an HMG sequence as represented by SEQ ID NO: 254.
23. Use of a construct according to any one of items 19 to 22 in a method for making plants having increased yield-related traits relative to control plants, which increased yield-related traits are one or more of: increased aboveground biomass, increased early vigor, increased seed yield per plant, increased seed fill rate, increased number of filled seeds, or increased number of primary panicles.
24. Plant, plant part or plant cell transformed with a construct according to any one of items 19 to 22.
25. Method for the production of transgenic plants having increased yield-related traits relative to control plants, comprising:
   (i) introducing and expressing in a plant, plant part, or plant cell, a nucleic acid sequence encoding a group I MBF1 polypeptide as defined in any one of items 1 to 7; and
   (ii) cultivating the plant cell, plant part, or plant under conditions promoting plant growth and development.
26. Transgenic plant having increased yield-related traits relative to control plants, resulting from increased expression of an isolated nucleic acid sequence encoding a group I MBF1 polypeptide as defined in any one of items 1 to 7, or a transgenic plant cell or transgenic plant part derived from said transgenic plant.
27. Transgenic plant according to item 18, 24, or 26, wherein said plant is a crop plant or a monocot or a cereal, such as rice, maize, wheat, barley, millet, rye, triticale, sorghum and oats, or a transgenic plant cell derived from said transgenic plant.
28. Harvestable parts comprising an isolated nucleic acid sequence encoding a group I MBF1 polypeptide, of a plant according to item 27, wherein said harvestable parts are preferably seeds.
29. Products derived from a plant according to item 27 and/or from harvestable parts of a plant according to item 28.
30. Use of a nucleic acid sequence encoding a group I MBF1 polypeptide as defined in any one of items 1 to 7, in increasing yield-related traits, comprising one or more of: increased aboveground biomass, increased early vigor, increased seed yield per plant, increased seed fill rate, increased number of filled seeds, or increased number of primary panicles.

### DESCRIPTION OF FIGURES

The present invention will now be described with reference to the following figures in which:

FIG. 1 represents the binary vector used for increased expression in Oryza sativa of a COX VIIa subunit-encoding nucleic acid under the control of a rice GOS2 promoter (pGOS2)

FIG. 2 represents the domain structure of SEQ ID NO: 19 with the zf-DNL domain (Pfam PF05180 shown in bold. The motifs 1 to 4 are underlined.

FIG. 3 represents a multiple alignment of various YLD-ZnF protein sequences.

FIG. 4 shows a phylogenetic tree of various YLD-ZnF protein sequences. The identifiers correspond to those used in FIG. 3.

FIG. 5 represents the binary vector used for increased expression in Oryza sativa of a YLD-ZnF-encoding nucleic acid under the control of a rice GOS2 promoter (pGOS2).

FIG. 6 represents the binary vector used for increased expression in Oryza sativa of a PKT-encoding nucleic acid under the control of a rice GOS2 promoter (pGOS2)

FIG. 7 represents SEQ ID NO: 59 with conserved motifs 11 to 15 shown in bold underlined

FIG. 8 represents a multiple alignment of various NOA polypeptides. SEQ ID NO: 59 is represented by At3g47450.

FIG. 9 shows a phylogenetic tree of various NOA polypeptides.

FIG. 10 represents the binary vector used for increased expression in Oryza sativa of a NOA-encoding nucleic acid under the control of a rice GOS2 promoter (pGOS2).

FIG. 11 shows a phylogenetic tree comprising the sequences represented by SEQ ID NO: 135 and SEQ ID NO: 137. The tree was made as described in Example 2. Query sequences clustering with either SEQ ID NO: 135 or 137 are suitable for use in the methods of the present invention.

FIG. 12 represents a multiple alignment of ASF1-like polypeptide sequences with Motifs I to IV boxed. The multiple alignment was made as described in Example 2.

FIG. 13 represents the binary vector for increased expression in Oryza sativa of an ASF1-like polypeptide encoding nucleic acid under the control of a rice GOS2 promoter (pGOS2)

FIG. 14 represents the binary vector used for increased expression in Oryza sativa of a PHDF-encoding nucleic acid under the control of a rice GOS2 promoter (pGOS2)

FIG. 15 represents an unrooted phylogenic tree for deduced amino acid sequences of MBF1s from 30 organisms and comparisons of amino acid sequences of plant MBF1 polypeptides, as described in Tsuda and Yamazaki (2004) Biochem Biophys Acta 1680: 1-10. Deduced amino acid sequences of MBF1s were aligned using the ClustaiX program, the tree was constructed using the neighbor-joining method, and the TreeView program. The scale bar indicates the genetic distance for 0.1 amino acid substitutions per site. Polypeptides useful in performing the methods of the invention cluster with group I MBF1, marked by a black arrow.

FIG. 16 represents a cartoon of a group I MBF1 polypeptide as represented by SEQ ID NO: 189, which comprises the following features: (i) an N-terminal multibridging factor 1 (MBF1) domain with an InterPro entry IPR013729 (and PFAM entry PF08523 MBF1); (ii) a Helix-turn-helix type 3 domain with an InterPro entry IPR001387 (and PFAM entry PF01381 HTH_{—}3).

FIG. 17 shows an AlignX (from Vector NTI 10.3, Invitrogen Corporation) multiple sequence alignment of a group I MBF1 polypeptides from Table A. An N-terminal multibridging factor 1 (MBF1) domain with an InterPro entry IPR013729 (and PFAM entry PF08523 MBF1), and a Helix-turn-helix type 3 domain with an InterPro entry IPR001387 (and PFAM entry PF01381 HTH_{—}3), are marked with X's below the consensus sequence. SEQ ID NO: 250 represents the polypeptide sequence corresponding to PF08523 of SEQ ID NO: 189, SEQ ID NO: 251 represents the polypeptide sequence corresponding to PF01381 of SEQ ID NO: 189.

FIG. 18 shows the binary vector for increased expression in Oryza sativa plants of a nucleic acid sequence encoding a group I MBF1 polypeptide under the control of a constitutive promoter functioning in plants.

### EXAMPLES

The present invention will now be described with reference to the following examples, which are by way of illustration alone. The following examples are not intended to completely define or otherwise limit the scope of the invention.

DNA manipulation: unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al. (1994), Current Protocols in Molecular Biology, Current Protocols. Standard materials and methods for plant molecular work are described in Plant Molecular Biology Labfax (1993) by R. D. D. Croy, published by BIOS Scientific Publications Ltd (UK) and Blackwell Scientific Publications (UK).

### Example 1

### Identification of Sequences Related to the Nucleic Acid Sequence Used in the Methods of the Invention

1.1. COX VIIa Subunit polypeptides

Sequences (full length cDNA, ESTs or genomic) related to the nucleic acid sequence used in the methods of the present invention are identified amongst those maintained in the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid or polypeptide sequences to sequence databases and by calculating the statistical significance of matches. For example, the polypeptide encoded by the nucleic acid of SEQ ID NO: 1, SEQ ID NO: 3, SEQ ID NO: 5, SEQ ID NO: 7 is used for the TBLASTN algorithm, with default settings and the filter to ignore low complexity sequences set off. The output of the analysis was viewed by pairwise comparison, and ranked according to the probability score (E-value), where the score reflects the probability that a particular alignment occurs by chance (the lower the E-value, the more significant the hit). In addition to E-values, comparisons are also scored by percentage identity. Percentage identity refers to the number of identical nucleotides (or amino acids) between the two compared nucleic acid (or polypeptide) sequences over a particular length. In some instances, the default parameters are adjusted to modify the stringency of the search. For example the E-value is increased to show less stringent matches. This way, short nearly exact matches are identified.

Table A1 provides a list of COX VIIa subunit nucleic acid sequences.

TABLE A1
Examples of COX Vlla subunit polypeptides:

| | | Nucleic acid | Polypeptide |
|---|---|---|---|
| Name | Organism | SEQ ID NO | SEQ ID NO |
| CoxVIIa-containing | Physcomitrella patens | 1 | 2 |
| polypeptide | | | |
| CoxVIIa-containing | Solanum lycopersicum | 3 | 4 |
| polypeptide | | | |
| CoxVIIa-containing | Hordeum vulgare | 5 | 6 |
| polypeptide | | | |
| CoxVIIa-containing | Populus trichocarpa | 7 | 8 |
| polypeptide | | | |

In some instances, related sequences are tentatively assembled and publicly disclosed by research institutions, such as The Institute for Genomic Research (TIGR; beginning with TA). The Eukaryotic Gene Orthologs (EGO) database is used to identify such related sequences, either by keyword search or by using the BLAST algorithm with the nucleic acid sequence or polypeptide sequence of interest. In other instances, special nucleic acid sequence databases are created for particular organisms, such as by the Joint Genome Institute.

### 1.2. YLD-ZnF Polypeptides

Sequences (full length cDNA, ESTs or genomic) related to the nucleic acid sequence used in the methods of the present invention were identified amongst those maintained in the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid or polypeptide sequences to sequence databases and by calculating the statistical significance of matches. For example, the polypeptide encoded by the nucleic acid used in the present invention was used for the TBLASTN algorithm, with default settings and the filter to ignore low complexity sequences set off. The output of the analysis was viewed by pairwise comparison, and ranked according to the probability score (E-value), where the score reflect the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid (or polypeptide) sequences over a particular length. In some instances, the default parameters may be adjusted to modify the stringency of the search. For example the E-value may be increased to show less stringent matches. This way, short nearly exact matches may be identified.

Table A2 provides a list of nucleic acid sequences related to the nucleic acid sequence used in the methods of the present invention.

TABLE A2
Examples of YLD-ZnF polypeptides:

| | Nucleic acid | Polypeptide |
|---|---|---|
| Plant Source | SEQ ID NO: | SEQ ID NO: |
| Medicago truncatula | 18 | 19 |
| Arabidopsis thaliana | 27 | 39 |
| Arabidopsis thaliana | 28 | 40 |
| Arabidopsis thaliana | 29 | 41 |
| Glycine max | 30 | 42 |
| Hordeum vulgare | 31 | 43 |
| Oryza sativa | 32 | 44 |
| Populus trichocarpa | 33 | 45 |
| Triticum aestivum | 34 | 46 |
| Triticum aestivum | 35 | 47 |
| Triticum aestivum | 36 | 48 |
| Zea mays | 37 | 49 |
| Zea mays | 38 | 50 |

In some instances, related sequences have tentatively been assembled and publicly disclosed by research institutions, such as The Institute for Genomic Research (TIGR; beginning with TA). The Eukaryotic Gene Orthologs (EGO) database may be used to identify such related sequences, either by keyword search or by using the BLAST algorithm with the nucleic acid sequence or polypeptide sequence of interest. In other instances, special nucleic acid sequence databases have been created for particular organisms, such as by the Joint Genome Institute. Further, access to proprietary databases, has allowed the identification of novel nucleic acid and polypeptide sequences.

### 1.3. PKT Polypeptides

Sequences (full length cDNA, ESTs or genomic) related to SEQ ID NO: 51 and SEQ ID NO: 53 are identified amongst those maintained in the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid or polypeptide sequences to sequence databases and by calculating the statistical significance of matches. For example, the polypeptide encoded by the nucleic acid of SEQ ID NO: 51 and SEQ ID NO: 53 is used in the TBLASTN algorithm, with default settings and the filter to ignore low complexity sequences set off. The output of the analysis was viewed by pairwise comparison, and ranked according to the probability score (E-value), where the score reflects the probability that a particular alignment occurs by chance (the lower the E-value, the more significant the hit). In addition to E-values, comparisons are also scored by percentage identity. Percentage identity refers to the number of identical nucleotides (or amino acids) between the two compared nucleic acid (or polypeptide) sequences over a particular length. In some instances, the default parameters are adjusted to modify the stringency of the search. For example the E-value is increased to show less stringent matches. This way, short nearly exact matches are identified.

Table A3 provides a list of PKT nucleic acid sequences.

TABLE A3
Examples of PKT polypeptides:

| | | Nucleic acid | Polypeptide |
|---|---|---|---|
| Name | Organism | SEQ ID NO | SEQ ID NO |
| Pt_PKT | Populus trichocarpa | 51 | 52 |
| Hv_PKT | Hordeum vulgare | 53 | 54 |

In some instances, related sequences are tentatively assembled and publicly disclosed by research institutions, such as The Institute for Genomic Research (TIGR; beginning with TA). The Eukaryotic Gene Orthologs (EGO) database is used to identify such related sequences, either by keyword search or by using the BLAST algorithm with the nucleic acid sequence or polypeptide sequence of interest. In other instances, special nucleic acid sequence databases are created for particular organisms, such as by the Joint Genome Institute.

### 1.4. NOA Polypeptides

Sequences (full length cDNA, ESTs or genomic) related to the nucleic acid sequence used in the methods of the present invention were identified amongst those maintained in the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid or polypeptide sequences to sequence databases and by calculating the statistical significance of matches. For example, the polypeptide encoded by the nucleic acid used in the present invention was used for the TBLASTN algorithm, with default settings and the filter to ignore low complexity sequences set off. The output of the analysis was viewed by pairwise comparison, and ranked according to the probability score (E-value), where the score reflect the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid (or polypeptide) sequences over a particular length. In some instances, the default parameters may be adjusted to modify the stringency of the search. For example the E-value may be increased to show less stringent matches. This way, short nearly exact matches may be identified.

Table A4 provides a list of nucleic acid sequences related to the nucleic acid sequence used in the methods of the present invention.

TABLE A4
Examples of NOA polypeptides:

| | Nucleic acid | Polypeptide |
|---|---|---|
| Name | SEQ ID NO: | SEQ ID NO: |
| AT3G47450.1#1 | 58 | 59 |
| AC195570 4.4#1 | 74 | 104 |
| Os02g0104700#1 | 75 | 105 |
| scaff 29.361#1 | 76 | 106 |
| 5283689#1 | 77 | 107 |
| 164227#1 | 78 | 108 |
| GSVIVT00029948001#1 | 79 | 109 |
| 8258#1 | 80 | 110 |
| 139489#1 | 81 | 111 |
| 49745#1 | 82 | 112 |
| 18820#1 | 83 | 113 |
| 17927#1 | 84 | 114 |
| 118673#1 | 85 | 115 |
| 194176#1 | 86 | 116 |
| 40200#1 | 87 | 117 |
| AT3G57180.1#1 | 88 | 118 |
| AC158502 36.4#1 | 89 | 119 |
| Os06g0498900#1 | 90 | 120 |
| scaff VI.400#1 | 91 | 121 |
| 5285494#1 | 92 | 122 |
| GSVIVT00025325001#1 | 93 | 123 |
| ZM07MC05087 62006489@5076#1 | 94 | 124 |
| AT4G10620.1#1 | 95 | 125 |
| Gm0053x00104#1 | 96 | 126 |
| LOC Os09g19980.1#1 | 97 | 127 |
| 5280283#1 | 98 | 128 |
| GSVIVT00024730001#1 | 99 | 129 |
| 141029#1 | 100 | 130 |
| 448312#1 | 101 | 131 |
| 27995#1 | 102 | 132 |
| 46935#1 | 103 | 133 |

In some instances, related sequences have tentatively been assembled and publicly disclosed by research institutions, such as The Institute for Genomic Research (TIGR; beginning with TA). The Eukaryotic Gene Orthologs (EGO) database may be used to identify such related sequences, either by keyword search or by using the BLAST algorithm with the nucleic acid sequence or polypeptide sequence of interest. In other instances, special nucleic acid sequence databases have been created for particular organisms, such as by the Joint Genome Institute. Further, access to proprietary databases, has allowed the identification of novel nucleic acid and polypeptide sequences.

1.5. ASF1-like Polypeptides

Sequences (full length cDNA, ESTs or genomic) related to ASF1-like nucleic acid sequence of SEQ ID NO: 134 and SEQ ID NO: 136 were identified from the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid or polypeptide sequences to sequence databases and by calculating the statistical significance of matches. For example, the polypeptides of SEQ ID NO: 135 and SEQ ID NO: 137 were used for the TBLASTN algorithm, with default settings and the filter to ignore low complexity sequences set off. The output of the analysis was viewed by pairwise comparison, and ranked according to the probability score (E-value), where the score reflect the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid (or polypeptide) sequences over a particular length. In some instances, the default parameters may be adjusted to modify the stringency of the search. For example the E-value may be increased to show less stringent matches. This way, short nearly exact matches may be identified.

Table A5 provides a list of nucleic acid sequences related to the ASF1-like sequences of SEQ ID NO: 134 and SEQ ID NO: 136

TABLE A5
Examples of ASF1-like nucleic acid and polypeptide sequences:

| | Nucleic acid | Polypeptide |
|---|---|---|
| Plant Source | SEQ ID NO: | SEQ ID NO: |
| Oryza sativa | 134 | 135 |
| Arabidopsis thaliana | 136 | 137 |
| Arabidopsis thaliana | 138 | 154 |
| Glycine max | 139 | 155 |
| Hordeum vulgare | 140 | 156 |
| Hordeum vulgare | 141 | 157 |
| Hordeum vulgare | 142 | 158 |
| Hordeum vulgare | 143 | 159 |
| Medicago truncatula | 144 | 160 |
| Medicago truncatula | 145 | 161 |
| Physcomitrella | 146 | 162 |
| patents | | |
| Physcomitrella | 147 | 163 |
| patents | | |
| Populus trichocarpa | 148 | 164 |
| Solanum lycopersicon | 149 | 165 |
| Solanum lycopersicon | 150 | 166 |
| Triticum aestivum | 151 | 167 |
| Zea mays | 152 | 168 |
| Zea mays | 153 | 169 |

In some instances, related sequences were tentatively assembled and publicly disclosed by research institutions, such as The Institute for Genomic Research (TIGR). The Eukaryotic Gene Orthologs (EGO) database may be used to identify such related sequences, either by keyword search or by using the BLAST algorithm with the nucleic acid or polypeptide sequence of interest.

### 1.6. PHDF Polypeptides

Sequences (full length cDNA, ESTs or genomic) related to SEQ ID NO: 175 and SEQ ID NO: 177 are identified amongst those maintained in the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid or polypeptide sequences to sequence databases and by calculating the statistical significance of matches. For example, the polypeptide encoded by the nucleic acid of SEQ ID NO: 175 and SEQ ID NO: 177 is used in the TBLASTN algorithm, with default settings and the filter to ignore low complexity sequences set off. The output of the analysis was viewed by pairwise comparison, and ranked according to the probability score (E-value), where the score reflects the probability that a particular alignment occurs by chance (the lower the E-value, the more significant the hit). In addition to E-values, comparisons are also scored by percentage identity. Percentage identity refers to the number of identical nucleotides (or amino acids) between the two compared nucleic acid (or polypeptide) sequences over a particular length. In some instances, the default parameters are adjusted to modify the stringency of the search. For example the E-value is increased to show less stringent matches. This way, short nearly exact matches are identified.

Table A6 provides a list of PHDF nucleic acid sequences.

TABLE A6
Examples PHDF polypeptides:

| | | Nucleic acid | Polypeptide |
|---|---|---|---|
| Name | Organism | SEQ ID NO | SEQ ID NO |
| Le_PHDF | Solanum lycopersicum | 175 | 176 |
| Pt_PHDF | Populus trichocarpa | 177 | 178 |
| Os_PHDF | Oryza sativa | 179 | 180 |

In some instances, related sequences are tentatively assembled and publicly disclosed by research institutions, such as The Institute for Genomic Research (TIGR; beginning with TA). The Eukaryotic Gene Orthologs (EGO) database is used to identify such related sequences, either by keyword search or by using the BLAST algorithm with the nucleic acid sequence or polypeptide sequence of interest. In other instances, special nucleic acid sequence databases are created for particular organisms, such as by the Joint Genome Institute.

1.7. group I MBF1 Polypeptides

Sequences (full length cDNA, ESTs or genomic) related to the nucleic acid sequence used in the methods of the present invention were identified amongst those maintained in the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid sequence or polypeptide sequences to sequence databases and by calculating the statistical significance of matches. For example, the polypeptide encoded by the nucleic acid sequence of the present invention was used for the TBLASTN algorithm, with default settings and the filter to ignore low complexity sequences set off. The output of the analysis was viewed by pairwise comparison, and ranked according to the probability score (E-value), where the score reflect the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid sequence (or polypeptide) sequences over a particular length. In some instances, the default parameters may be adjusted to modify the stringency of the search. For example the E-value may be increased to show less stringent matches. This way, short nearly exact matches may be identified.

Table A7 provides a list of nucleic acid sequences related to the nucleic acid sequence used in the methods of the present invention.

TABLE A7
Examples of group I MBF1 polypeptide sequences, and encoding
nucleic acid sequences

| | Public database | Nucleic acid | Polypeptide |
|---|---|---|---|
| Name | accession number | SEQ ID NO: | SEQ ID NO: |
| Arath_MBF1b | At3g58680 | 188 | 189 |
| Arath_MBF1a | At2g42680 | 190 | 191 |
| Medtr_group I MBF1 | BG452607.1 | 192 | 193 |
| Triae_group I MBF1 | CJ580790.1 | 194 | 195 |
| Elagu_MBF1 | EU284884.1 | 196 | 197 |
| Elagu_MBF1bis | EU284896.1 | 198 | 199 |
| Glyma_MBF1 | AK244428.1 | 200 | 201 |
| Gymco_MBF1 | EF051328.1 | 202 | 203 |
| Horvu_MBF1 | AK250323.1 | 204 | 205 |
| Horvu_group I MBF1 | CA020129.1 | 206 | 207 |
| Linus_MBF1 | EU830239.1 | 208 | 209 |
| Nicta_MBF1 | AB072698.1 | 210 | 211 |
| Orysa_MBF1 | AK120339.1 | 212 | 213 |
| Picsi_MBF1bis | EF084509.1 | 214 | 215 |
| Poptr_MBF1 | scaff_182.33 | 216 | 217 |
| Poptr_MBF1bis | EF146354.1 | 218 | 219 |
| Ricco_MBF1 | Z49698.1 | 220 | 221 |
| Soltu_MBF1 | AF232062 | 222 | 223 |
| Zeama_MBF1 | BT036744.1 | 224 | 225 |
| Zeama_MBF1bis | FL067563 | 226 | 227 |

In some instances, related sequences have tentatively been assembled and publicly disclosed by research institutions, such as The Institute for Genomic Research (TIGR; beginning with TA). The Eukaryotic Gene Orthologs (EGO) database may be used to identify such related sequences, either by keyword search or by using the BLAST algorithm with the nucleic acid sequence or polypeptide sequence of interest. In other instances, special nucleic acid sequence databases have been created for particular organisms, such as by the Joint Genome Institute. Further, access to proprietary databases, has allowed the identification of novel nucleic acid and polypeptide sequences.

### Example 2

### Alignment of Sequences Related to the Polypeptide Sequences Used in the Methods of the Invention

### 2.1. COX VIIa Subunit Polypeptides

Alignment of polypeptide sequences is performed using the ClustalW 2.0 algorithm of progressive alignment (Thompson et al. (1997) Nucleic Acids Res 25:4876-4882; Chenna et al. (2003). Nucleic Acids Res 31:3497-3500) with standard setting (slow alignment, similarity matrix: Gonnet (or Blosum 62 (if polypeptides are aligned), gap opening penalty 10, gap extension penalty: 0.2). Minor manual editing is done to further optimise the alignment.

A phylogenetic tree of COX VIIA SUBUNIT polypeptides is constructed using a neighbour-joining clustering algorithm as provided in the AlignX programme from the Vector NTI (Invitrogen).

Alignment of polypeptide sequences is performed using the ClustalW 2.0 algorithm of progressive alignment (Thompson et al. (1997) Nucleic Acids Res 25:4876-4882; Chenna et al. (2003). Nucleic Acids Res 31:3497-3500) with standard setting (slow alignment, similarity matrix: Gonnet, gap opening penalty 10, gap extension penalty: 0.2). Minor manual editing is done to further optimise the alignment.

### 2.2. YLD-ZnF Polypeptides

Alignment of polypeptide sequences was performed using the ClustalW 2.0 algorithm of progressive alignment (Thompson et al. (1997) Nucleic Acids Res 25:4876-4882; Chenna et al. (2003). Nucleic Acids Res 31:3497-3500) with standard setting (slow alignment, similarity matrix: Gonnet, gap opening penalty 10, gap extension penalty: 0.2). Minor manual editing was done to further optimise the alignment. The YLD-ZnF polypeptides are aligned in FIG. 3.

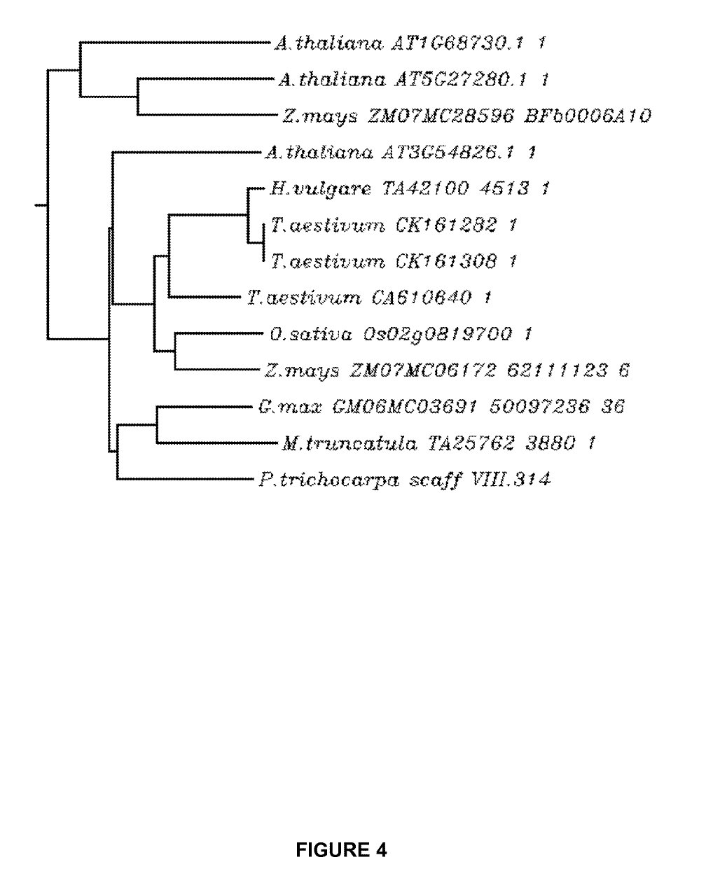

A phylogenetic tree of YLD-ZnF polypeptides (FIG. 4) was constructed using a neighbour-joining clustering algorithm as provided in the AlignX programme from the Vector NTI (Invitrogen).

### 2.3. PKT Polypeptides

Alignment of polypeptide sequences is performed using the ClustalW 2.0 algorithm of progressive alignment (Thompson et al. (1997) Nucleic Acids Res 25:4876-4882; Chenna et al. (2003). Nucleic Acids Res 31:3497-3500) with standard setting (slow alignment, similarity matrix: Gonnet (or Blosum 62 (if polypeptides are aligned), gap opening penalty 10, gap extension penalty: 0.2). Minor manual editing is done to further optimise the alignment.

A phylogenetic tree of PKT polypeptides is constructed using a neighbour-joining clustering algorithm as provided in the AlignX programme from the Vector NTI (Invitrogen).

Alignment of polypeptide sequences is performed using the ClustalW 2.0 algorithm of progressive alignment (Thompson et al. (1997) Nucleic Acids Res 25:4876-4882; Chenna et al. (2003). Nucleic Acids Res 31:3497-3500) with standard setting (slow alignment, similarity matrix: Gonnet, gap opening penalty 10, gap extension penalty: 0.2). Minor manual editing is done to further optimise the alignment.

### 2.4. NOA Polypeptides

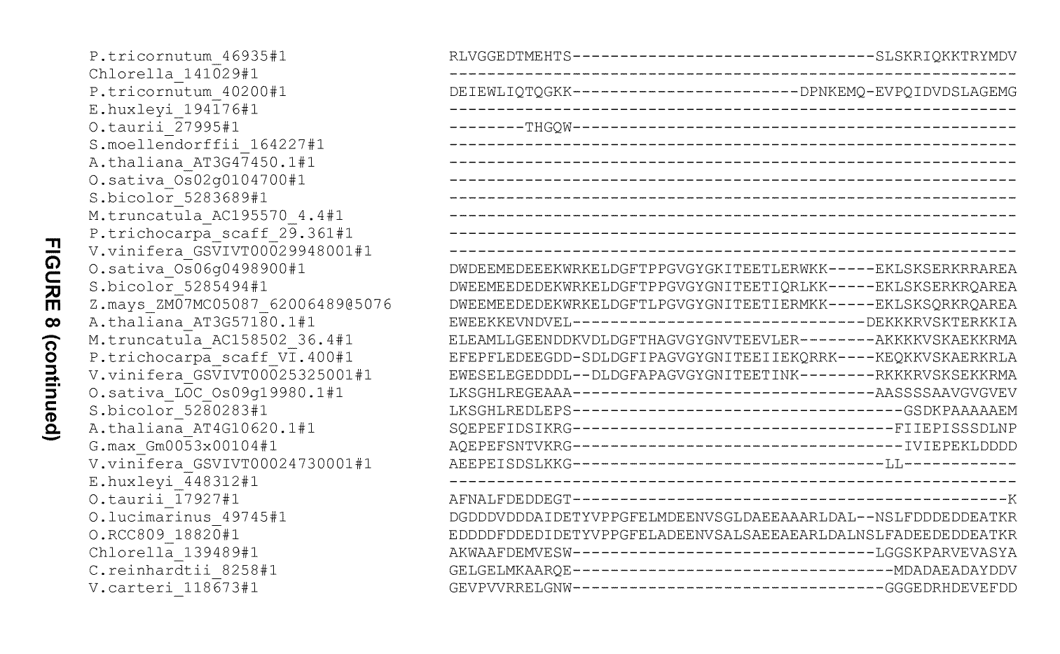

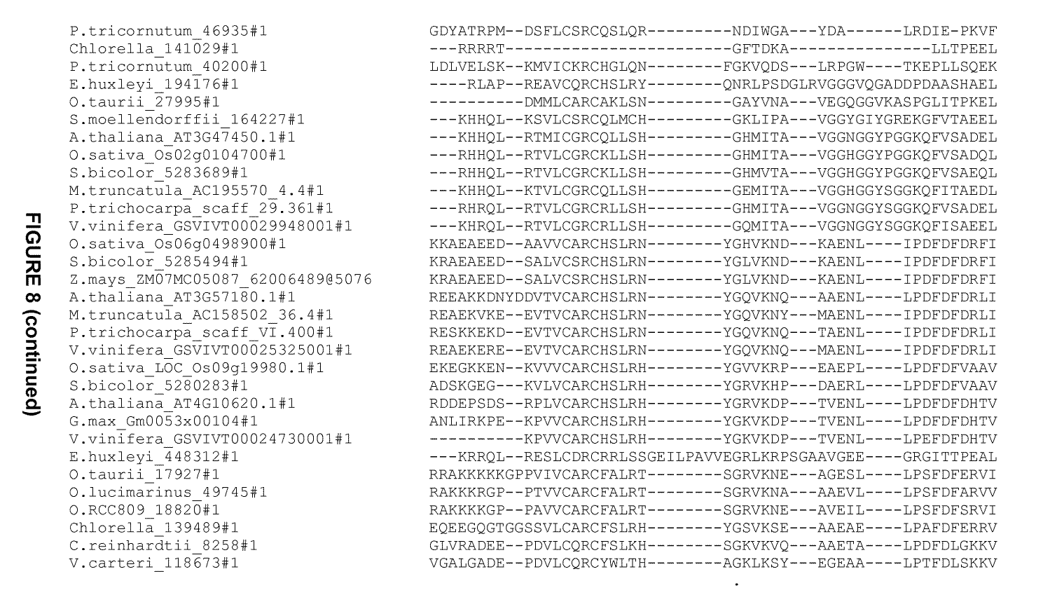

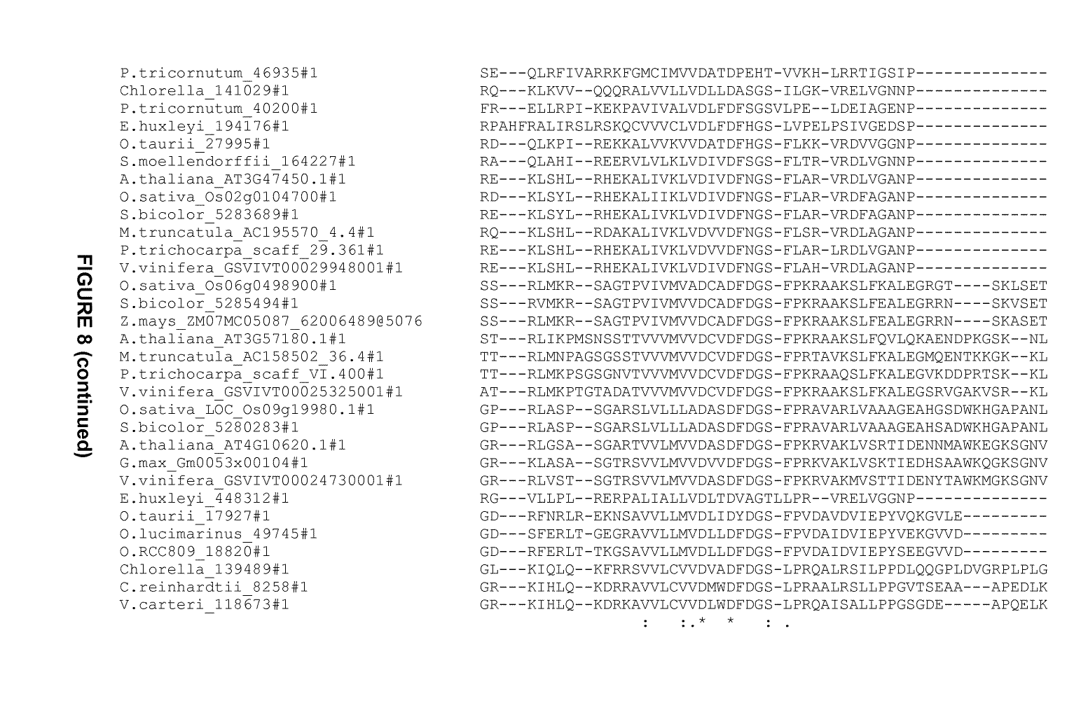

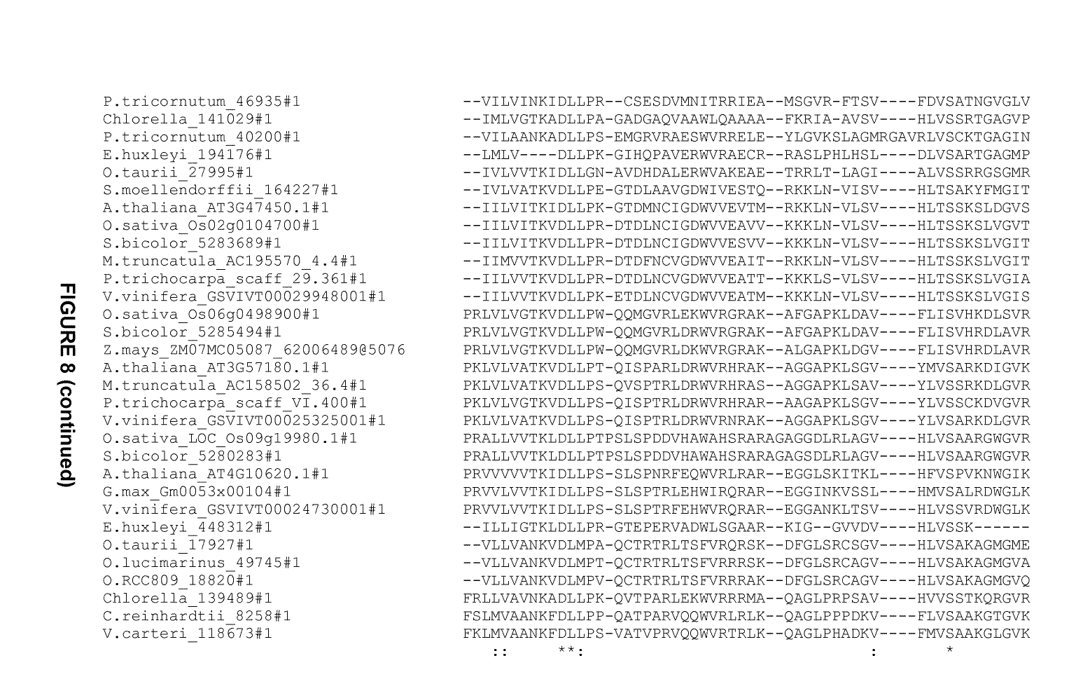

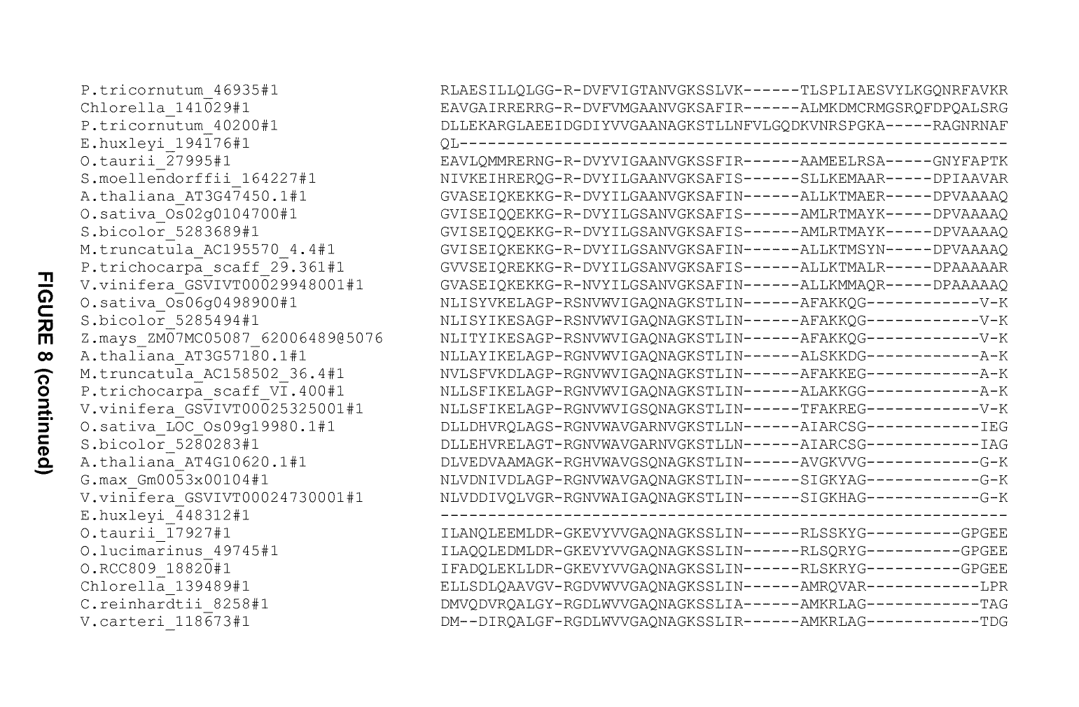

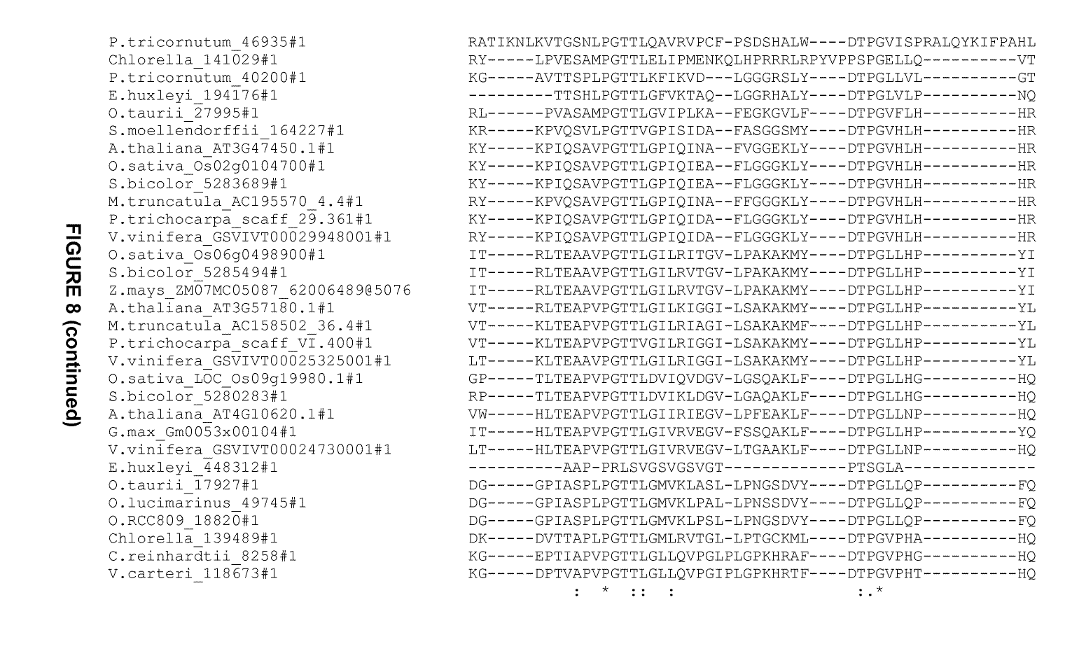

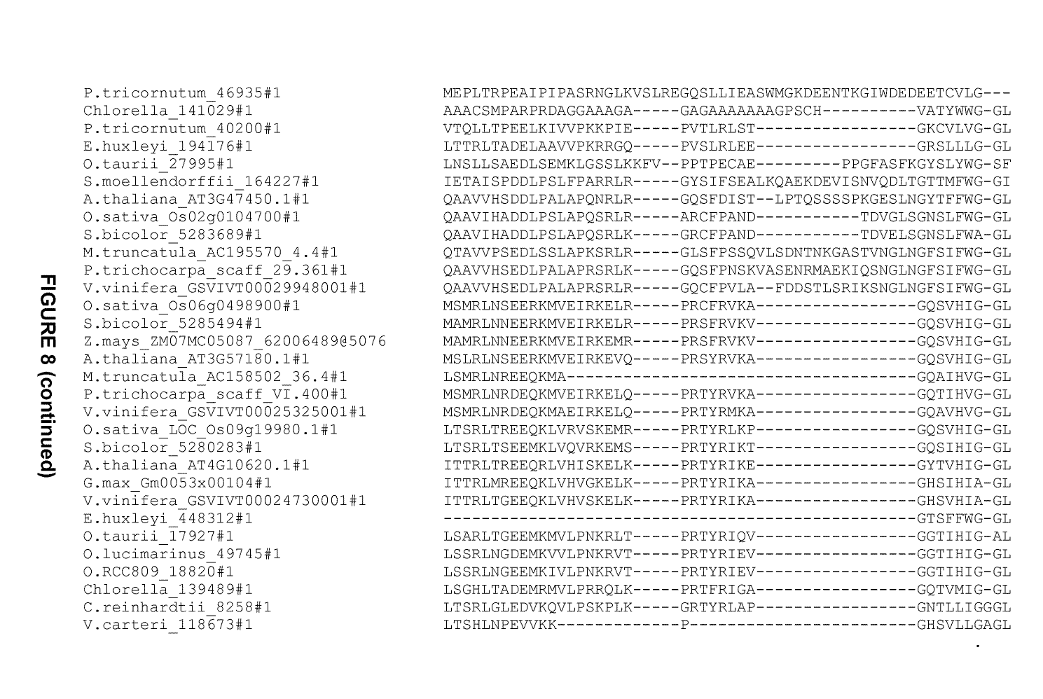

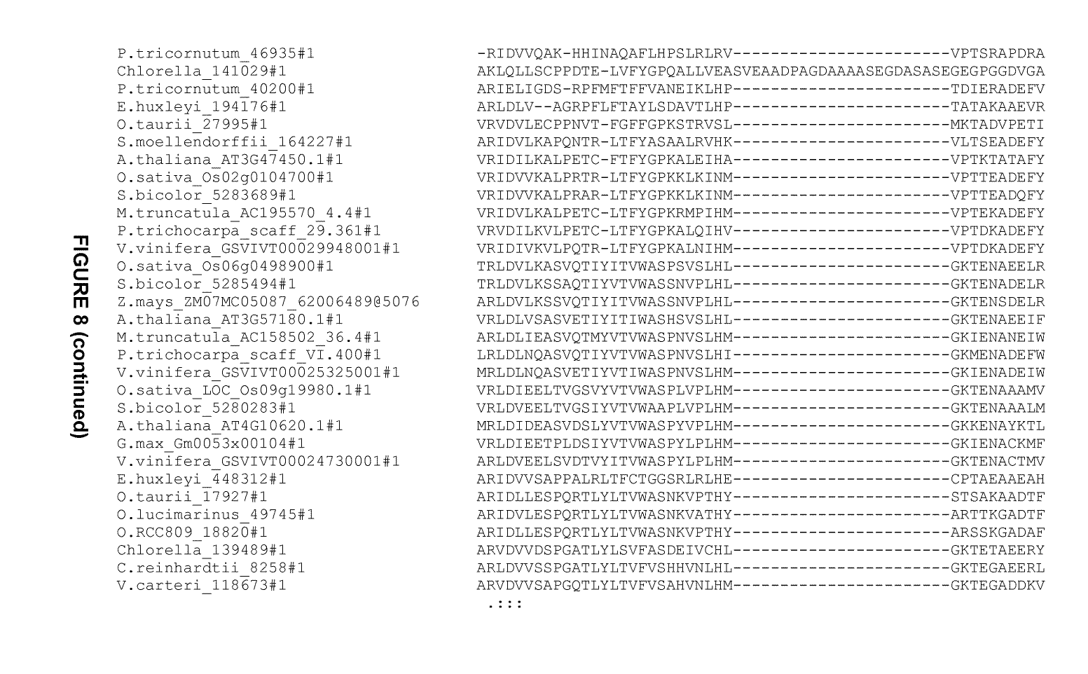

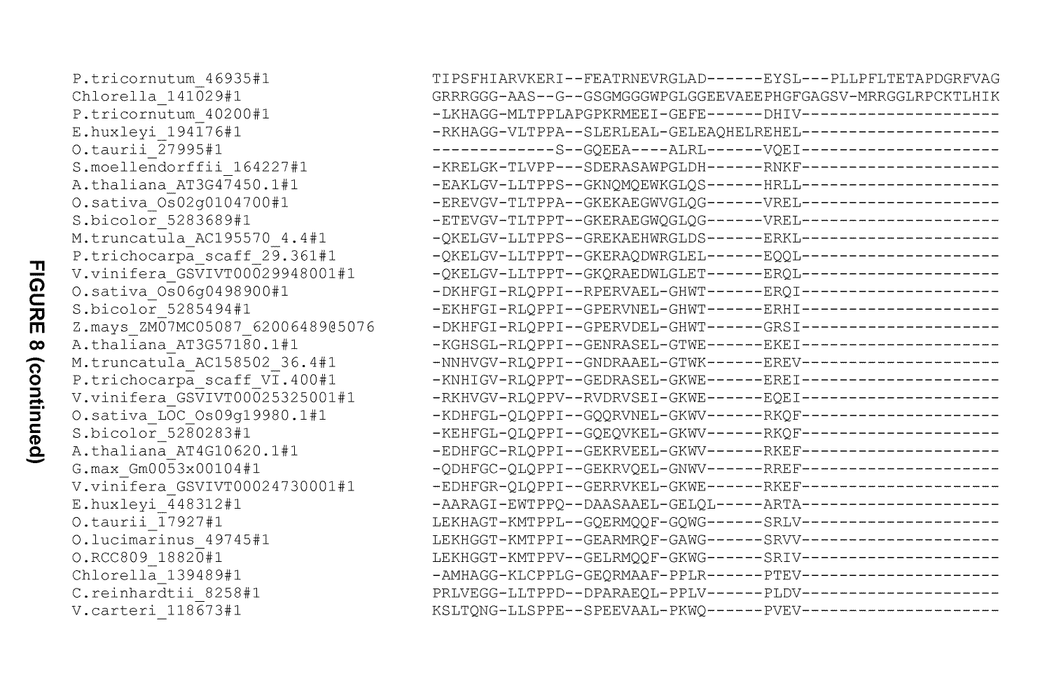

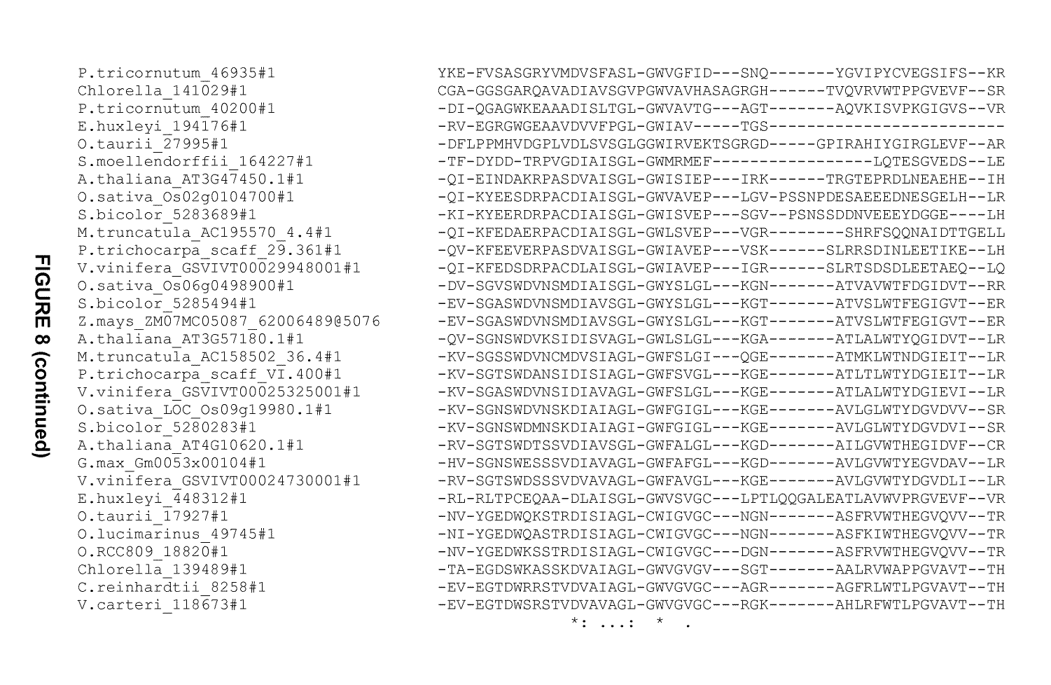

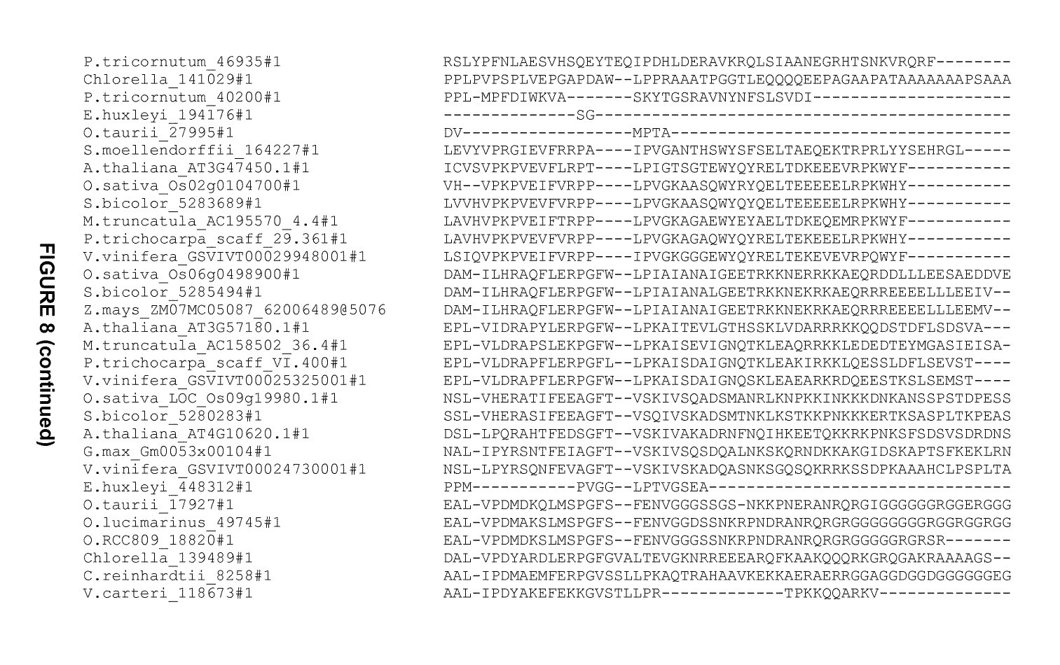

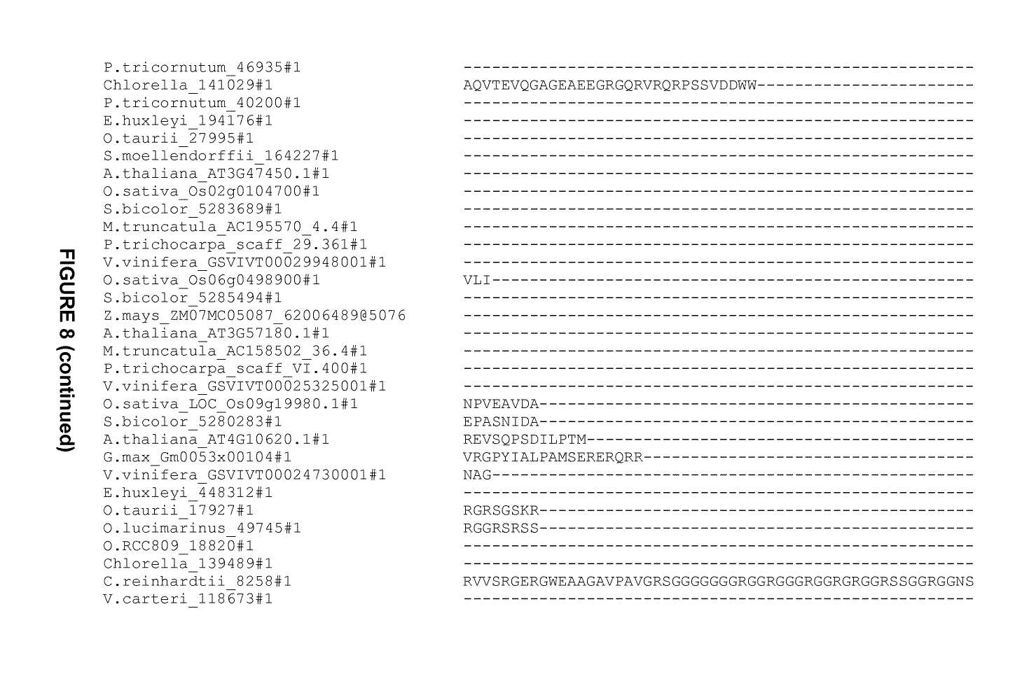

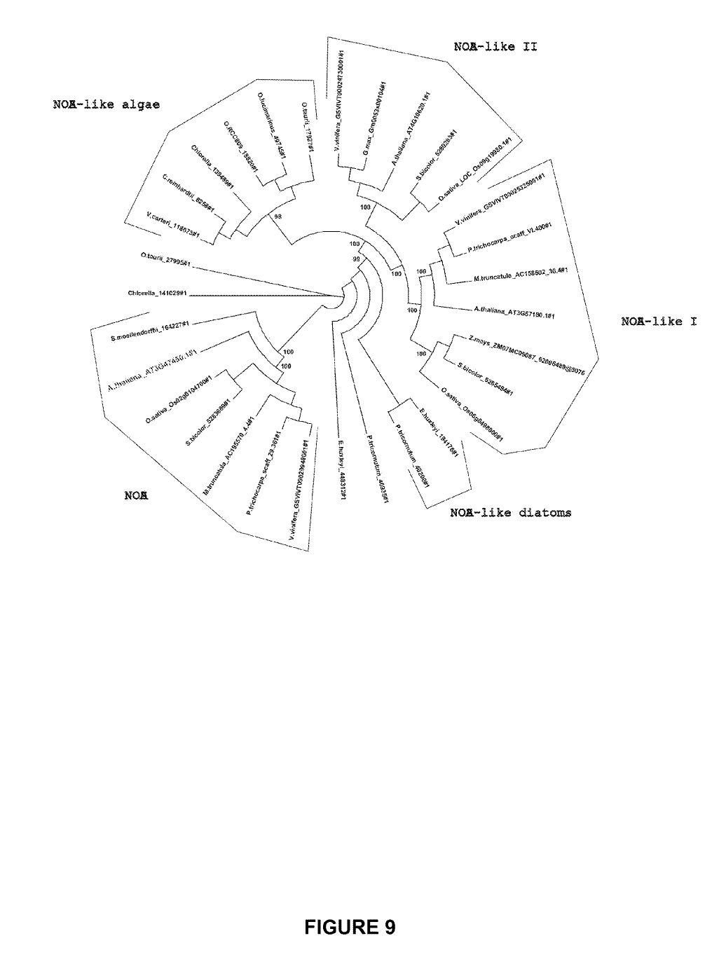

The proteins were aligned using MUSCLE (Edgar (2004), Nucleic Acids Research 32(5): 1792-97). A Neighbour-Joining tree was calculated using QuickTree (Howe et al. (2002), Bioinformatics 18(11): 1546-7). Support of the major branching after 100 bootstrap repetitions is indicated. A circular phylogram was drawn using Dendroscope (Huson et al. (2007), BMC Bioinformatics 8(1):460). The alignment is shown is FIG. 8, the phylogenetic tree is shown in FIG. 9.

2.5. ASF1-like Polypeptides

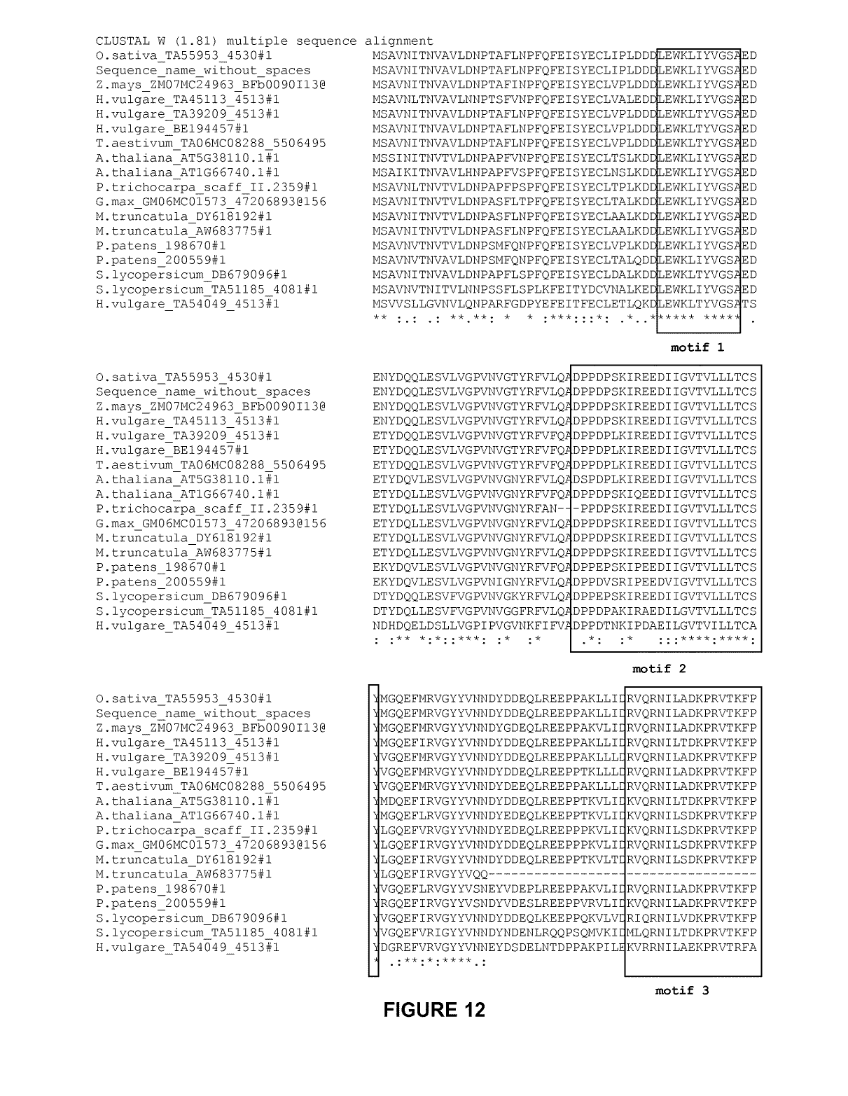

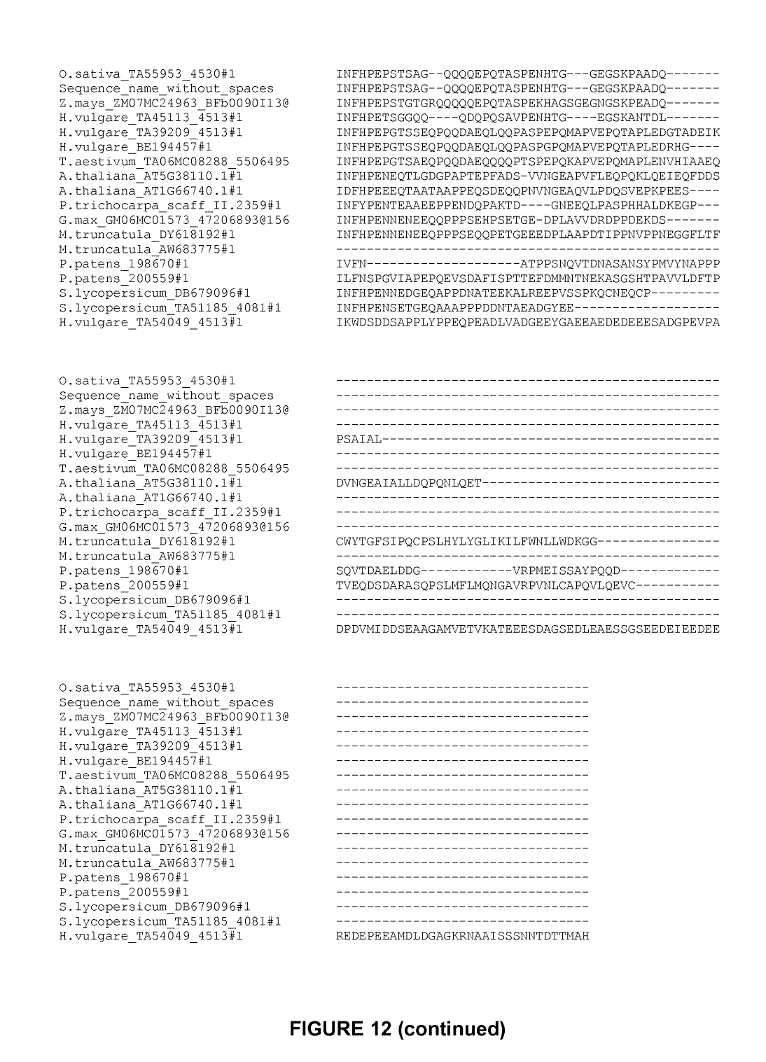

Alignment of polypeptide sequences was performed using the AlignX programme from the Vector NTI (Invitrogen) which is based on the popular Clustal W algorithm of progressive alignment (Thompson et al. (1997) Nucleic Acids Res 25:4876-4882; Chenna et al. (2003). Nucleic Acids Res 31:3497-3500). Default values are for the gap open penalty of 10, for the gap extension penalty of 0.1 and the selected weight matrix is Blosum 62 (if polypeptides are aligned). Minor manual editing was done to further optimise the alignment. Sequence conservation among ASF1-like polypeptides is essentially in the N-terminal domain of the polypeptides, the C-terminal domain usually being more variable in sequence length and composition. The ASF1-like polypeptides are aligned in FIG. 12.

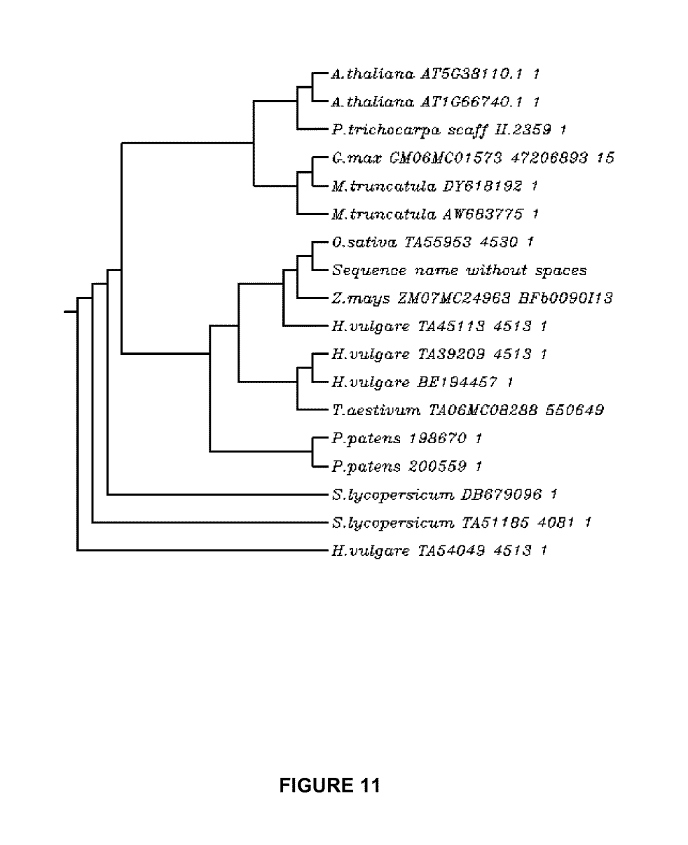

A phylogenetic tree of ASF1-like polypeptides (FIG. 11) was constructed using a neighbour-joining clustering algorithm as provided in the AlignX programme from the Vector NTI (Invitrogen).

### 2.6. PHDF Polypeptides

Alignment of polypeptide sequences is performed using the ClustalW 2.0 algorithm of progressive alignment (Thompson et al. (1997) Nucleic Acids Res 25:4876-4882; Chenna et al. (2003). Nucleic Acids Res 31:3497-3500) with standard setting (slow alignment, similarity matrix: Gonnet (or Blosum 62 (if polypeptides are aligned), gap opening penalty 10, gap extension penalty: 0.2). Minor manual editing is done to further optimise the alignment.

A phylogenetic tree of PHDF polypeptides is constructed using a neighbour-joining clustering algorithm as provided in the AlignX programme from the Vector NTI (Invitrogen).

Alignment of polypeptide sequences is performed using the ClustalW 2.0 algorithm of progressive alignment (Thompson et al. (1997) Nucleic Acids Res 25:4876-4882; Chenna et al. (2003). Nucleic Acids Res 31:3497-3500) with standard setting (slow alignment, similarity matrix: Gonnet, gap opening penalty 10, gap extension penalty: 0.2). Minor manual editing is done to further optimise the alignment.

### 2.7. Group I MBF1 Polypeptides

Multiple sequence alignment of all of a group I MBF1 polypeptide sequences in Table A7, as well as a few group II MBF1 sequences, was performed using the AlignX algorithm (from Vector NTI 10.3, Invitrogen Corporation). Results of the alignment are shown in FIG. 3 of the present application. An N-terminal multibridging factor 1 (MBF1) domain with an InterPro entry IPR013729 (and PFAM entry PF08523 MBF1), and a Helix-turn-helix type 3 domain with an InterPro entry IPR001387 (and PFAM entry PF01381 HTH_{—}3), are marked with X's below the consensus sequence. SEQ ID NO: 250 represents the polypeptide sequence corresponding to PF08523 of SEQ ID NO: 189, SEQ ID NO: 251 represents the polypeptide sequence corresponding to PF01381 of SEQ ID NO: 189.

### Example 3

### Calculation of Global Percentage Identity Between Polypeptide Sequences Useful in Performing the Methods of the Invention

### 3.1. COX VIIa Subunit Polypeptides

Global percentages of similarity and identity between full length polypeptide sequences is determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

Parameters used in the comparison are:
Scoring matrix: Blosum 62
First Gap: 12
Extending gap: 2

A MATGAT table for local alignment of a specific domain, or data on % identity/similarity between specific domains may also be performed.

### 3.2. YLD-ZnF Polypeptides

Global percentages of similarity and identity between full length polypeptide sequences useful in performing the methods of the invention were determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

Parameters used in the comparison were:
Scoring matrix: Blosum 62
First Gap: 12
Extending gap: 2

Results of the software analysis are shown in Table B1 for the global similarity and identity over the full length of the polypeptide sequences. Percentage identity is given above the diagonal in bold and percentage similarity is given below the diagonal (normal face).

The percentage identity between the YLD-ZnF polypeptide sequences useful in performing the methods of the invention can be as low as 19% amino acid identity compared to SEQ ID NO: 19 (TA25762).

TABLE B1
MatGAT results for global similarity and identity over the full length of the polypeptide sequences.
A MATGAT table for local alignment of a specific domain, or data on
% identity/similarity between specific domains may also be included.

| | | 1 | 2 | 3 | 4 | 5 | 6 | 7 | 8 | 9 | 10 | 11 | 12 |
|---|---|---|---|---|---|---|---|---|---|---|---|---|---|
| 1. | AT1G68730.1 | | 20.4 | 26.4 | 22.8 | 21.7 | 20.8 | 24.8 | 20.9 | 20.2 | 13.5 | 21.0 | 27.5 |
| 2. | AT3G54826.1 | 34.5 | | 21.3 | 42.2 | 39.4 | 37.8 | 43.7 | 43.4 | 39.4 | 24.0 | 40.6 | 19.6 |
| 3. | AT5G27280.1 | 40.6 | 39.0 | | 20.1 | 22.1 | 19.0 | 21.0 | 20.1 | 21.2 | 14.6 | 21.4 | 53.4 |
| 4. | GM06MC03691 | 35.6 | 56.1 | 35.4 | | 47.0 | 61.2 | 47.7 | 53.5 | 43.9 | 26.2 | 45.5 | 18.2 |
| 5. | TA42100 | 37.2 | 55.6 | 37.3 | 63.9 | | 41.1 | 68.2 | 46.1 | 94.2 | 37.5 | 67.7 | 18.8 |
| 6. | TA25762 | 39.2 | 53.8 | 34.0 | 72.4 | 55.8 | | 43.2 | 44.7 | 41.1 | 24.1 | 41.3 | 22.7 |
| 7. | Os02g0819700 | 41.0 | 52.5 | 37.7 | 60.6 | 81.7 | 59.8 | | 48.3 | 68.2 | 33.2 | 69.8 | 21.7 |
| 8. | Pt_scaff_VIII.314 | 34.7 | 54.7 | 39.2 | 66.3 | 64.3 | 61.3 | 59.8 | | 44.7 | 26.6 | 47.8 | 23.5 |
| 9. | CK161282 | 34.6 | 54.3 | 36.3 | 59.2 | 95.3 | 56.3 | 81.2 | 62.8 | | 38.0 | 66.8 | 19.7 |
| 10. | CA610640 | 22.9 | 33.2 | 24.1 | 36.7 | 41.9 | 34.2 | 43.1 | 35.2 | 42.4 | | 34.5 | 12.3 |
| 11. | ZM07MC06172 | 37.4 | 53.4 | 36.8 | 63.3 | 77.0 | 57.8 | 80.9 | 62.3 | 77.0 | 42.2 | | 22.9 |
| 12. | ZM07MC28596 | 38.9 | 32.3 | 62.7 | 30.3 | 30.8 | 36.5 | 34.1 | 35.5 | 31.8 | 22.3 | 35.1 | |

### 3.3. PKT Polypeptides

Global percentages of similarity and identity between full length polypeptide sequences is determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

Parameters used in the comparison are:
Scoring matrix: Blosum 62
First Gap: 12
Extending gap: 2

A MATGAT table for local alignment of a specific domain, or data on % identity/similarity between specific domains may also be performed.

### 3.4. NOA Polypeptides

Global percentages of similarity and identity between full length polypeptide sequences useful in performing the methods of the invention were determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

Parameters used in the comparison were:
Scoring matrix: Blosum 62
First Gap: 12
Extending gap: 2

Results of the software analysis are shown in Table B2 for the global similarity and identity over the full length of the polypeptide sequences. Percentage identity is given above the diagonal in bold and percentage similarity is given below the diagonal (normal face).

The percentage identity between the NOA polypeptide sequences useful in performing the methods of the invention can be as low as yy % amino acid identity compared to SEQ ID NO: 59.

TABLE B2
MatGAT results for global similarity and identity over the full length of the polypeptide sequences.
A MATGAT table for local alignment of a specific domain, or data on % identity/similarity between specific domains may also be included.

| | | 1 | 2 | 3 | 4 | 5 | 6 | 7 | 8 | 9 | 10 | 11 | 12 | 13 | 14 | 15 | 16 |
|---|---|---|---|---|---|---|---|---|---|---|---|---|---|---|---|---|---|
| 1. | AT3G47450.1#1 | | 63.4 | 59.8 | 66.4 | 60.5 | 43.8 | 65.4 | 18.9 | 20.4 | 21.4 | 21.4 | 20.9 | 18.1 | 20.7 | 20.6 | 20 |
| 2. | AC195570_4.4#1 | 77.5 | | 64.8 | 71.5 | 63.2 | 44.3 | 69 | 20.6 | 21.3 | 21.8 | 22.7 | 22.7 | 20.7 | 20.9 | 20.8 | 21 |
| 3. | Os02g0104700#1 | 75.6 | 80.3 | | 66.1 | 85.8 | 44.2 | 64.3 | 21.1 | 22 | 23.5 | 22.5 | 22 | 20.7 | 21.7 | 21.1 | 20 |
| 4. | scaff_29.361#1 | 82.3 | 84.6 | 80.1 | | 66.5 | 44.7 | 75 | 21.4 | 20.6 | 21.1 | 22.6 | 22.3 | 19.8 | 21.6 | 21.7 | 20.5 |
| 5. | 5283689#1 | 75.8 | 79.5 | 92.3 | 80 | | 43.9 | 64.9 | 21.4 | 21.3 | 23.8 | 22.9 | 22.4 | 20.3 | 21.2 | 21.8 | 19 |
| 6. | 164227#1 | 60.4 | 61.3 | 61.1 | 63.4 | 60.4 | | 44.7 | 20.6 | 22.2 | 22.2 | 23.5 | 20.8 | 19.8 | 20.1 | 20.1 | 21.9 |
| 7. | GSVIVT00029948001#1 | 78.8 | 81.5 | 79.4 | 86.5 | 79.7 | 61.9 | | 21.9 | 20 | 22 | 23.2 | 22.8 | 19 | 21.7 | 22.4 | 21 |
| 8. | 8258#1 | 34.2 | 35.2 | 34.7 | 35.3 | 34.9 | 35.3 | 36 | | 38.4 | 33.8 | 29.1 | 28.5 | 45.8 | 20.1 | 22.4 | 28.1 |
| 9. | 139489#1 | 38 | 37.7 | 38.6 | 38.4 | 38 | 40.3 | 38 | 52 | | 37.1 | 34.2 | 31.8 | 35.1 | 21.9 | 22.9 | 34.1 |
| 10. | 49745#1 | 40.2 | 37.9 | 39.4 | 40.6 | 38.9 | 41.6 | 39.7 | 45.2 | 53.4 | | 75.4 | 67.1 | 26.2 | 21.4 | 23.5 | 31.8 |
| 11. | 18820#1 | 41.5 | 40.6 | 40.8 | 41.3 | 41.4 | 42.1 | 41.1 | 39.4 | 49.1 | 81.5 | | 74.5 | 25.5 | 21.8 | 24 | 32.6 |
| 12. | 17927#1 | 39.9 | 42.2 | 41.1 | 40.8 | 40.3 | 38.6 | 41.6 | 38.5 | 47.1 | 74.5 | 83.9 | | 22 | 21.2 | 22.5 | 28.7 |
| 13. | 118673#1 | 32.7 | 34.1 | 34.5 | 35.3 | 33.8 | 36.6 | 35.2 | 61.9 | 48.6 | 39.8 | 38.2 | 35.7 | | 21.6 | 24 | 26 |
| 14. | 194176#1 | 35.3 | 35.6 | 34.7 | 34.6 | 33.2 | 35.6 | 34.7 | 29.7 | 33.3 | 31.6 | 35.1 | 35.4 | 31.9 | | 24.4 | 20.2 |
| 15. | 40200#1 | 36.9 | 34.8 | 35.6 | 38 | 34.9 | 40 | 38.5 | 41.1 | 41.1 | 38.7 | 39.2 | 37.6 | 41.4 | 33.8 | | 23.7 |
| 16. | AT3G57180.1#1 | 41.3 | 37.7 | 39.3 | 41.8 | 36.8 | 41.5 | 40.4 | 43.2 | 53.1 | 51.1 | 48.3 | 45.8 | 42.8 | 28.9 | 43.7 | |
| 17. | AC158502_36.4#1 | 38.1 | 40.6 | 39.8 | 40.6 | 36.5 | 41.2 | 41.4 | 42.3 | 52.8 | 50.5 | 47.1 | 45.9 | 42.4 | 30 | 40.4 | 74.4 |
| 18. | Os06g0498900#1 | 37.2 | 35.8 | 37.2 | 39.8 | 37.3 | 38.3 | 37.6 | 44.3 | 50.8 | 47.7 | 44.9 | 42.7 | 42.8 | 29.4 | 40.3 | 66.5 |
| 19. | scaff_VI.400#1 | 36.3 | 38.9 | 38.3 | 39.8 | 37.2 | 39.5 | 39.2 | 44.6 | 50.9 | 50.3 | 48 | 44.3 | 42.9 | 29.6 | 43 | 79 |
| 20. | 5285494#1 | 38.6 | 38.3 | 38.7 | 39.6 | 39.3 | 39.5 | 38.7 | 43.4 | 53 | 48 | 46.5 | 42.8 | 43.2 | 30.6 | 40.6 | 68.2 |
| 21. | GSVIVT00025325001#1 | 39.4 | 41.5 | 37.9 | 42 | 37.5 | 41.2 | 42.2 | 40.8 | 51.9 | 51.5 | 48.9 | 48.1 | 39.8 | 33.5 | 41.3 | 74.1 |
| 22. | ZM07MC05087 | 37.4 | 38.2 | 38.8 | 39.1 | 38.3 | 36.4 | 38 | 42.8 | 51.5 | 48.1 | 45.7 | 42.5 | 43.8 | 29.9 | 41.1 | 66.9 |
| | 62006489@5076#1 | | | | | | | | | | | | | | | | |
| 23. | AT4G10620.1#1 | 39.5 | 38.7 | 40.4 | 42 | 37.4 | 41.1 | 40 | 44.2 | 48.9 | 49 | 46.9 | 46.7 | 41.1 | 32 | 39.4 | 56.8 |
| 24. | Gm0053x00104#1 | 39.5 | 39.8 | 39 | 40.8 | 38.7 | 43.1 | 40.2 | 44.3 | 50.3 | 50.2 | 49.6 | 46.8 | 42.1 | 30.4 | 38 | 58.2 |
| 25. | LOC_Os09g19980.1#1 | 39.7 | 38.4 | 40.4 | 40.7 | 40.2 | 36.8 | 39.6 | 43.2 | 48.6 | 47.5 | 44.7 | 43.4 | 40.6 | 33.1 | 37.7 | 56.1 |
| 26. | 5280283#1 | 41.2 | 40.2 | 40.4 | 41.4 | 39.9 | 39.2 | 40.9 | 45.2 | 49.4 | 48.5 | 46.6 | 45.4 | 41.3 | 34.5 | 37 | 56.7 |
| 27. | GSVIVT00024730001#1 | 39.2 | 41.2 | 40.6 | 42 | 40.5 | 42.1 | 41.9 | 42.8 | 48.3 | 48.8 | 50.5 | 49.1 | 41.6 | 33 | 37.9 | 55.3 |
| 28. | 141029#1 | 44.9 | 43.4 | 41.8 | 42.7 | 40.2 | 41.4 | 42.7 | 29.3 | 31.2 | 32.8 | 38.9 | 38 | 26 | 26.6 | 26.8 | 28.7 |
| 29. | 448312#1 | 36.2 | 37.5 | 37.5 | 37.6 | 37.9 | 33.1 | 38.6 | 25.2 | 25.3 | 26.2 | 27.5 | 30.3 | 23.8 | 40.3 | 25.8 | 28.6 |
| 30. | 27995#1 | 45.1 | 47.9 | 46.6 | 47 | 47.8 | 43.2 | 45 | 30.2 | 34.4 | 34.4 | 36.5 | 37.9 | 29.9 | 40.7 | 32.4 | 33.2 |
| 31. | 46935#1 | 36.3 | 35 | 33.8 | 36.1 | 33.6 | 37.3 | 37.1 | 34.9 | 35.6 | 34.2 | 32.4 | 30.3 | 37.4 | 27.3 | 36.6 | 34 |

| | | 17 | 18 | 19 | 20 | 21 | 22 | 23 | 24 | 25 | 26 | 27 | 28 | 29 | 30 | 31 |
|---|---|---|---|---|---|---|---|---|---|---|---|---|---|---|---|---|
| 1. | AT3G47450.1#1 | 22.4 | 20.8 | 20.1 | 21.3 | 20.9 | 21.6 | 20.8 | 20 | 22.3 | 20.9 | 20.8 | 23.9 | 25.1 | 31.3 | 20.2 |
| 2. | AC195570_4.4#1 | 21.9 | 21.2 | 23.3 | 22.6 | 23.8 | 22 | 21.2 | 22.3 | 22.6 | 22.9 | 23.2 | 23.6 | 28.1 | 31.5 | 20.5 |
| 3. | Os02g0104700#1 | 22.8 | 23.2 | 21.7 | 23.3 | 22.1 | 22.2 | 21.6 | 21.3 | 23.1 | 21.5 | 22.6 | 23.5 | 28.4 | 30.3 | 20.3 |
| 4. | scaff_29.361#1 | 22 | 21.1 | 22.5 | 23.9 | 22.1 | 23.1 | 21.7 | 22 | 23.2 | 21.9 | 22.1 | 24.3 | 25.8 | 32.2 | 20 |
| 5. | 5283689#1 | 21.9 | 21.7 | 22 | 23.8 | 22 | 23 | 21.5 | 21.4 | 23.3 | 21.5 | 23.1 | 22.6 | 27.6 | 32 | 19.3 |
| 6. | 164227#1 | 23.5 | 20.8 | 24 | 21.6 | 21.9 | 21.5 | 20.9 | 21.6 | 20.1 | 21.1 | 22.2 | 23 | 24.5 | 28.5 | 20.7 |
| 7. | GSVIVT00029948001#1 | 23.5 | 23.6 | 22.4 | 23.2 | 21.4 | 22.3 | 21.8 | 22.6 | 23.7 | 22.8 | 23.5 | 23.3 | 27.3 | 30.4 | 20.6 |
| 8. | 8258#1 | 28.1 | 29 | 27.6 | 28.9 | 26.5 | 28.4 | 28.9 | 29.4 | 31.4 | 30.6 | 29.4 | 20.1 | 18.6 | 19.6 | 19.6 |
| 9. | 139489#1 | 33.5 | 33.7 | 33.1 | 33.4 | 33.5 | 32.7 | 31.5 | 30.5 | 31.5 | 31.8 | 31.2 | 19.9 | 17.9 | 20.6 | 19.8 |
| 10. | 49745#1 | 29.4 | 29.8 | 31.3 | 30.2 | 31.8 | 30.2 | 29.2 | 29.1 | 29 | 29.5 | 28.9 | 17.1 | 16.9 | 22 | 19.1 |
| 11. | 18820#1 | 29.4 | 29.2 | 30.9 | 31 | 31.5 | 29.7 | 30.3 | 31.6 | 29.3 | 28.6 | 31.9 | 20.1 | 17.5 | 21.6 | 18.4 |
| 12. | 17927#1 | 28.8 | 27.5 | 27.6 | 28.3 | 28.9 | 26.9 | 28.9 | 29.2 | 28 | 29.3 | 29.4 | 21 | 17.5 | 22.3 | 18.4 |
| 13. | 118673#1 | 26.4 | 25.6 | 25.8 | 26.3 | 24.4 | 26.9 | 25.8 | 25.4 | 26.7 | 25.7 | 26.9 | 16.2 | 16.7 | 18.7 | 20.3 |
| 14. | 194176#1 | 19.7 | 19.7 | 18.8 | 20.5 | 20.7 | 19.6 | 21.2 | 19.7 | 23 | 23 | 22.3 | 15.8 | 24.3 | 23 | 16.4 |
| 15. | 40200#1 | 23.6 | 22.8 | 24.6 | 22.9 | 23.7 | 23.7 | 22.9 | 20.8 | 22.4 | 22.2 | 23.1 | 17.7 | 17 | 20.2 | 18.8 |
| 16. | AT3G57180.1#1 | 55.9 | 50.1 | 60.6 | 48.9 | 59.8 | 49 | 38.4 | 38.5 | 38 | 36.7 | 39.5 | 15.2 | 17.4 | 20 | 17.1 |
| 17. | AC158502_36.4#1 | | 51.7 | 63.8 | 50.4 | 64.9 | 49.9 | 36.9 | 38.1 | 38.1 | 36.5 | 38 | 14.3 | 17.5 | 20.9 | 19.3 |
| 18. | Os06g0498900#1 | 67.1 | | 53.8 | 79 | 53.4 | 78.1 | 36.7 | 35.9 | 37.3 | 36.7 | 36.8 | 16.3 | 17.6 | 18.8 | 18.3 |
| 19. | scaff_VI.400#1 | 76.7 | 70.6 | | 52.7 | 66.8 | 51.8 | 37.3 | 38.6 | 37.4 | 37 | 39.4 | 14.5 | 17.2 | 19.9 | 18 |
| 20. | 5285494#1 | 67.6 | 87.2 | 69.7 | | 53.8 | 91.2 | 36.4 | 36 | 36.4 | 36.2 | 36.5 | 16.8 | 18.3 | 19.7 | 19.3 |
| 21. | GSVIVT00025325001#1 | 80.2 | 69 | 77.9 | 68.9 | | 53.2 | 38.7 | 40.3 | 38.2 | 37.5 | 41.8 | 14.7 | 16.9 | 20.7 | 17.1 |
| 22. | ZM07MC05087 | 67.1 | 85.8 | 70.1 | 94.5 | 68.1 | | 35.5 | 35.6 | 35.9 | 36.8 | 36.7 | 16.1 | 18.2 | 20.1 | 18.1 |
| | 62006489@5076#1 | | | | | | | | | | | | | | | |
| 23. | AT4G10620.1#1 | 58 | 52.4 | 56.2 | 53 | 59.8 | 53.4 | | 60.7 | 48.2 | 48.1 | 61.7 | 16.9 | 17.5 | 20.3 | 17.1 |
| 24. | Gm0053x00104#1 | 59.3 | 52.7 | 58.4 | 54.4 | 62 | 54.2 | 77.5 | | 50.7 | 48.4 | 65.7 | 19 | 17.2 | 19.3 | 19 |
| 25. | LOC_Os09g19980.1#1 | 56.2 | 52.3 | 55.1 | 53.5 | 59.4 | 53.4 | 67.9 | 65.8 | | 78.8 | 51.1 | 16.2 | 19.5 | 22.1 | 20 |
| 26. | 5280283#1 | 55.7 | 51.8 | 54.1 | 52.4 | 60.1 | 53.4 | 68.7 | 65.1 | 86.4 | | 49.1 | 15.6 | 20.4 | 22.7 | 19.2 |
| 27. | GSVIVT00024730001#1 | 57.6 | 51.7 | 55.6 | 52.6 | 59.8 | 52.2 | 76.4 | 77 | 64.9 | 65.7 | | 20.1 | 19.3 | 22.4 | 20.2 |
| 28. | 141029#1 | 28.2 | 25.1 | 28.2 | 28.1 | 27 | 27.8 | 30.7 | 36.4 | 28.8 | 28.1 | 36.4 | | 21.4 | 21.8 | 16.6 |
| 29. | 448312#1 | 28.6 | 26.3 | 26.7 | 26.3 | 27.8 | 27.1 | 27.3 | 26.8 | 27 | 28.5 | 29 | 33.2 | | 24.6 | 14.1 |
| 30. | 27995#1 | 33.8 | 30.7 | 33.2 | 31.1 | 34 | 32.6 | 39 | 34.5 | 35.1 | 37.2 | 37.6 | 36.8 | 37 | | 19.5 |
| 31. | 46935#1 | 36.4 | 35.2 | 35.7 | 34.9 | 33.9 | 34.7 | 33.2 | 34.5 | 35.4 | 33.3 | 34 | 29.8 | 24.6 | 31 | |

3.5. ASF1-like Polypeptides

Global percentages of similarity and identity between full length ASF1-like polypeptide sequences was determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix.

Parameters used in the comparison are:
Scoring matrix: Blosum 62
First Gap: 12
Extending gap: 2

A MATGAT table for local alignment of a specific domain, or data on % identity/similarity between specific domains may also be made.

### 3.6. PHDF Polypeptides

Global percentages of similarity and identity between full length polypeptide sequences is determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

Parameters used in the comparison are:
Scoring matrix: Blosum 62
First Gap: 12
Extending gap: 2

A MATGAT table for local alignment of a specific domain, or data on % identity/similarity between specific domains may also be performed.

### 3.7. Group I MBF1 Polypeptides

Global percentages of similarity and identity between full length polypeptide sequences useful in performing the methods of the invention were determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

Parameters used in the comparison were:
Scoring matrix: Blosum 62
First Gap: 12
Extending gap: 2

Results of the software analysis are shown in Table B3 for the global similarity and identity over the full length of the polypeptide sequences (excluding the partial polypeptide sequences).

The percentage identity between the full length polypeptide sequences useful in performing the methods of the invention can be as low as 74% amino acid identity compared to SEQ ID NO: 189.

TABLE B3
MatGAT results for global similarity and identity over the
full length of the polypeptide sequences of Table A7.

| | | 1 | 2 | 3 | 4 | 5 | 6 | 7 | 8 | 9 | 10 | 11 | 12 | 13 | 14 | 15 | 16 |
|---|---|---|---|---|---|---|---|---|---|---|---|---|---|---|---|---|---|
| 1. | Arath_MBF1b | | 92 | 85 | 78 | 82 | 81 | 84 | 84 | 78 | 78 | 75 | 80 | 80 | 74 | 82 | 82 |
| 2. | Arath_MBF1a | 97 | | 86 | 80 | 81 | 80 | 85 | 82 | 79 | 80 | 75 | 80 | 82 | 74 | 82 | 82 |
| 3. | Medtr_MBF1a_b | 95 | 93 | | 80 | 83 | 80 | 88 | 82 | 78 | 85 | 81 | 84 | 81 | 75 | 87 | 87 |
| 4. | Triae_MBF1a/b | 92 | 91 | 92 | | 82 | 79 | 82 | 78 | 99 | 82 | 73 | 75 | 92 | 74 | 78 | 78 |
| 5. | Elagu_MBF1 | 93 | 92 | 94 | 90 | | 87 | 83 | 88 | 80 | 80 | 80 | 82 | 84 | 78 | 85 | 85 |
| 6. | Elagu_MBF1bis | 92 | 90 | 93 | 90 | 95 | | 82 | 86 | 78 | 77 | 81 | 82 | 80 | 78 | 86 | 85 |
| 7. | Glyma_MBF1 | 97 | 95 | 97 | 93 | 94 | 94 | | 85 | 82 | 82 | 82 | 85 | 87 | 76 | 85 | 85 |
| 8. | Gymco_MBF1 | 94 | 93 | 94 | 92 | 97 | 94 | 96 | | 76 | 75 | 79 | 83 | 80 | 77 | 86 | 85 |
| 9. | Horvu_MBF1 | 92 | 91 | 92 | 99 | 90 | 90 | 93 | 92 | | 80 | 72 | 73 | 91 | 72 | 77 | 77 |
| 10. | Horvu_MBF1a_b | 89 | 89 | 92 | 89 | 89 | 88 | 92 | 89 | 89 | | 78 | 78 | 85 | 74 | 82 | 82 |
| 11. | Linus_MBF1 | 89 | 87 | 89 | 86 | 89 | 90 | 91 | 89 | 86 | 87 | | 83 | 78 | 75 | 88 | 87 |
| 12. | Nicta_MBF1 | 92 | 90 | 94 | 88 | 92 | 91 | 95 | 91 | 88 | 90 | 91 | | 79 | 76 | 89 | 87 |
| 13. | Orysa_MBF1 | 94 | 92 | 94 | 95 | 93 | 92 | 97 | 94 | 95 | 92 | 89 | 92 | | 78 | 82 | 81 |
| 14. | Picsi_MBF1bis | 85 | 83 | 85 | 84 | 87 | 86 | 87 | 88 | 84 | 82 | 82 | 83 | 87 | | 80 | 80 |
| 15. | Poptr_MBF1 | 93 | 92 | 94 | 89 | 94 | 93 | 94 | 94 | 89 | 92 | 92 | 94 | 92 | 85 | | 94 |
| 16. | Poptr_MBF1bis | 93 | 92 | 94 | 90 | 94 | 94 | 94 | 94 | 90 | 92 | 93 | 94 | 92 | 84 | 97 | |
| 17. | Ricco_MBF1 | 94 | 92 | 94 | 90 | 94 | 92 | 95 | 94 | 90 | 90 | 93 | 92 | 94 | 84 | 94 | 96 |
| 18. | Soltu_MBF1 | 94 | 92 | 95 | 89 | 93 | 92 | 96 | 93 | 89 | 90 | 92 | 99 | 92 | 84 | 94 | 96 |
| 19. | Zeama_MBF1 | 94 | 92 | 93 | 94 | 93 | 93 | 97 | 94 | 94 | 91 | 91 | 92 | 99 | 85 | 92 | 92 |
| 20. | Zeama_MBF1bis | 95 | 93 | 95 | 94 | 94 | 92 | 98 | 96 | 94 | 92 | 89 | 93 | 99 | 86 | 92 | 92 |
| 21. | Allce_MBF1c | 70 | 69 | 69 | 68 | 72 | 71 | 71 | 73 | 68 | 68 | 70 | 69 | 69 | 66 | 69 | 72 |
| 22. | Arath_MBF1c | 70 | 69 | 70 | 70 | 71 | 71 | 72 | 72 | 70 | 70 | 68 | 69 | 71 | 72 | 70 | 70 |
| 23. | Chlre_MBF1a/b | 71 | 71 | 73 | 72 | 70 | 69 | 73 | 71 | 72 | 73 | 71 | 74 | 73 | 67 | 73 | 73 |
| 24. | Lyces_MBF1c | 68 | 67 | 68 | 68 | 69 | 70 | 70 | 69 | 68 | 67 | 71 | 69 | 70 | 65 | 69 | 71 |
| 25. | Orysa_MBF1c | 64 | 63 | 67 | 63 | 65 | 65 | 66 | 65 | 63 | 62 | 64 | 66 | 65 | 67 | 66 | 67 |
| 26. | Phypa_MBF1 | 87 | 85 | 89 | 86 | 89 | 87 | 90 | 89 | 86 | 85 | 86 | 89 | 90 | 85 | 87 | 87 |
| 27. | Phypa_MBF1bis | 79 | 80 | 78 | 78 | 76 | 77 | 81 | 80 | 78 | 78 | 77 | 78 | 80 | 72 | 78 | 80 |
| 28. | Picsi_MBF1c | 72 | 72 | 71 | 72 | 74 | 74 | 75 | 74 | 72 | 72 | 72 | 73 | 73 | 70 | 72 | 74 |
| 29. | Retra_MBF1 | 72 | 71 | 71 | 70 | 72 | 70 | 75 | 74 | 70 | 68 | 70 | 72 | 73 | 70 | 70 | 72 |
| 30. | Triae_MBF1c | 63 | 62 | 66 | 61 | 64 | 64 | 65 | 64 | 61 | 61 | 63 | 65 | 63 | 67 | 65 | 66 |
| 31. | Zeama_MBF1c | 61 | 60 | 65 | 60 | 64 | 63 | 63 | 63 | 60 | 61 | 63 | 65 | 62 | 64 | 64 | 65 |

| | | 17 | 18 | 19 | 20 | 21 | 22 | 23 | 24 | 25 | 26 | 27 | 28 | 29 | 30 | 31 |
|---|---|---|---|---|---|---|---|---|---|---|---|---|---|---|---|---|
| 1. | Arath_MBF1b | 82 | 82 | 82 | 81 | 49 | 51 | 57 | 44 | 49 | 71 | 60 | 56 | 49 | 48 | 48 |
| 2. | Arath_MBF1a | 81 | 81 | 84 | 83 | 48 | 49 | 58 | 44 | 48 | 70 | 61 | 54 | 47 | 47 | 48 |
| 3. | Medtr_MBF1a_b | 85 | 85 | 82 | 85 | 48 | 49 | 57 | 45 | 47 | 72 | 58 | 54 | 46 | 47 | 47 |
| 4. | Triae_MBF1a/b | 78 | 75 | 90 | 91 | 44 | 47 | 57 | 44 | 44 | 70 | 58 | 48 | 45 | 44 | 43 |
| 5. | Elagu_MBF1 | 85 | 82 | 84 | 85 | 50 | 50 | 57 | 47 | 49 | 72 | 61 | 54 | 49 | 49 | 50 |
| 6. | Elagu_MBF1bis | 85 | 83 | 82 | 80 | 50 | 49 | 57 | 46 | 49 | 71 | 62 | 56 | 49 | 49 | 50 |
| 7. | Glyma_MBF1 | 85 | 87 | 87 | 87 | 46 | 47 | 60 | 46 | 47 | 77 | 58 | 53 | 47 | 46 | 47 |
| 8. | Gymco_MBF1 | 85 | 83 | 81 | 81 | 48 | 49 | 56 | 45 | 47 | 73 | 58 | 57 | 47 | 46 | 47 |
| 9. | Horvu_MBF1 | 77 | 75 | 89 | 89 | 44 | 47 | 56 | 44 | 43 | 70 | 58 | 48 | 44 | 44 | 43 |
| 10. | Horvu_MBF1a_b | 78 | 76 | 86 | 87 | 48 | 49 | 58 | 46 | 44 | 73 | 60 | 54 | 45 | 45 | 45 |
| 11. | Linus_MBF1 | 87 | 84 | 79 | 78 | 50 | 47 | 59 | 46 | 46 | 73 | 59 | 56 | 47 | 46 | 48 |
| 12. | Nicta_MBF1 | 85 | 91 | 80 | 80 | 49 | 47 | 58 | 46 | 50 | 76 | 59 | 57 | 47 | 50 | 51 |
| 13. | Orysa_MBF1 | 82 | 78 | 97 | 96 | 47 | 48 | 60 | 44 | 46 | 76 | 60 | 50 | 47 | 47 | 47 |
| 14. | Picsi_MBF1bis | 79 | 75 | 78 | 77 | 48 | 49 | 56 | 43 | 47 | 76 | 56 | 53 | 48 | 49 | 47 |
| 15. | Poptr_MBF1 | 91 | 87 | 83 | 82 | 50 | 49 | 59 | 46 | 49 | 74 | 61 | 57 | 49 | 49 | 50 |
| 16. | Poptr_MBF1bis | 92 | 87 | 82 | 82 | 51 | 49 | 60 | 47 | 50 | 75 | 62 | 56 | 48 | 50 | 50 |
| 17. | Ricco_MBF1 | | 85 | 83 | 83 | 52 | 49 | 60 | 48 | 49 | 74 | 60 | 54 | 49 | 49 | 48 |
| 18. | Soltu_MBF1 | 94 | | 80 | 80 | 47 | 47 | 60 | 45 | 47 | 77 | 57 | 53 | 46 | 47 | 49 |
| 19. | Zeama_MBF1 | 94 | 92 | | 97 | 48 | 48 | 60 | 44 | 46 | 77 | 60 | 52 | 47 | 46 | 47 |
| 20. | Zeama_MBF1bis | 94 | 94 | 99 | | 47 | 47 | 59 | 44 | 46 | 77 | 60 | 53 | 47 | 46 | 45 |
| 21. | Allce_MBF1c | 72 | 70 | 70 | 71 | | 68 | 46 | 66 | 59 | 47 | 60 | 63 | 70 | 60 | 60 |
| 22. | Arath_MBF1c | 69 | 70 | 70 | 70 | 79 | | 46 | 67 | 57 | 50 | 60 | 64 | 74 | 58 | 58 |
| 23. | Chlre_MBF1a/b | 72 | 76 | 73 | 73 | 65 | 62 | | 42 | 42 | 58 | 51 | 41 | 44 | 43 | 44 |
| 24. | Lyces_MBF1c | 71 | 69 | 71 | 69 | 75 | 77 | 58 | | 56 | 46 | 55 | 58 | 70 | 56 | 57 |
| 25. | Orysa_MBF1c | 66 | 67 | 64 | 65 | 67 | 70 | 57 | 67 | | 44 | 55 | 53 | 62 | 90 | 83 |
| 26. | Phypa_MBF1 | 87 | 89 | 90 | 92 | 69 | 71 | 71 | 67 | 63 | | 53 | 52 | 47 | 43 | 43 |
| 27. | Phypa_MBF1bis | 78 | 78 | 79 | 80 | 78 | 78 | 66 | 73 | 68 | 74 | | 67 | 66 | 54 | 54 |
| 28. | Picsi_MBF1c | 72 | 72 | 73 | 74 | 79 | 82 | 59 | 76 | 69 | 70 | 85 | | 63 | 52 | 52 |
| 29. | Retra_MBF1 | 70 | 70 | 72 | 72 | 81 | 85 | 63 | 81 | 75 | 70 | 83 | 83 | | 62 | 64 |
| 30. | Triae_MBF1c | 65 | 66 | 62 | 63 | 69 | 71 | 58 | 66 | 94 | 62 | 67 | 68 | 74 | | 81 |
| 31. | Zeama_MBF1c | 64 | 65 | 62 | 62 | 68 | 72 | 58 | 69 | 88 | 61 | 69 | 68 | 76 | 87 | |

The percentage amino acid identity can be significantly increased if the most conserved region of the polypeptides are compared. For example, when comparing the amino acid sequence of an N-terminal multibridging factor 1 (MBF1) domain with an InterPro entry IPR013729 (and PFAM entry PF08523 MBF1) as represented by SEQ ID NO: 250, or of a Helix-turn-helix type 3 domain with an InterPro entry IPR001387 (and PFAM entry PF01381 HTH_{—}3) as represented by SEQ ID NO: 251, with the respective corresponding domains of the polypeptides of Table A7, the percentage amino acid identity increases significantly (in order of preference at least 75%, 80%, 85%, 90%, 95%, 98%, 99% or more amino acid sequence identity).

### Example 4

### Identification of Domains Comprised in Polypeptide Sequences Useful in Performing the Methods of the Invention

### 4.1. COX VIIa Subunit Polypeptides

The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, ProDom and Pfam, Smart and TIGRFAMs. Pfam is a large collection of multiple sequence alignments and hidden Markov models covering many common protein domains and families. Pfam is hosted at the Sanger Institute server in the United Kingdom. Interpro is hosted at the European Bioinformatics Institute in the United Kingdom.

### 4.2. YLD-ZnF Polypeptides

The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, ProDom and Pfam, Smart and TIGRFAMs. Pfam is a large collection of multiple sequence alignments and hidden Markov models covering many common protein domains and families. Pfam is hosted at the Sanger Institute server in the United Kingdom. Interpro is hosted at the European Bioinformatics Institute in the United Kingdom.

The results of the InterPro scan of the polypeptide sequence as represented by SEQ ID NO: 19 are presented in Table C1.

TABLE C1
InterPro scan results (major accession numbers) of the polypeptide sequence as
represented by SEQ ID NO: 19.

| | | | amino acid coordinates on |
|---|---|---|---|
| Database | accession number | accession name | SEQ ID NO: 19 |
| InterPro | IPR007853 | Zinc finger, Zim17-type | |
| Method | AccNumber | shortName | location |
| HMMPanther | PTHR20922 | UNCHARACTERIZED | T[115-193] 6.5e−24 |
| HMMPfam | PF05180 | zf-DNL | T[106-170] 4.2e−27 |
| InterPro | NULL | NULL | |
| Method | AccNumber | shortName | location |
| HMMPanther | PTHR20922:SF13 | UNCHARACTERIZED | T[115-193] 6.5e−24 |

4.3. PKT polypeptides—ASF1-like Polypeptides—PHDF Polypeptides

The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, ProDom and Pfam, Smart and TIGRFAMs. Pfam is a large collection of multiple sequence alignments and hidden Markov models covering many common protein domains and families. Pfam is hosted at the Sanger Institute server in the United Kingdom. Interpro is hosted at the European Bioinformatics Institute in the United Kingdom.

### 4.4. NOA Polypeptides

The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, ProDom and Pfam, Smart and TIGRFAMs. Pfam is a large collection of multiple sequence alignments and hidden Markov models covering many common protein domains and families. Pfam is hosted at the Sanger Institute server in the United Kingdom. Interpro is hosted at the European Bioinformatics Institute in the United Kingdom.

The results of the InterPro scan of the polypeptide sequence as represented by SEQ ID NO: 59 are presented in Table C2.

TABLE C2
InterPro scan results (major accession numbers) of the polypeptide sequence as
represented by SEQ ID NO: 59.

| Method | AccNumber | shortName | location |
|---|---|---|---|
| Gene3D | G3DSA:3.40.50.300 | no description | T[177-352] 3.2e−17 |
| HMMPanther | PTHR11089 | GTP-BINDING PROTEIN- | T[195-494] 2.3e−49 |
| | | RELATED | |
| HMMPanther | PTHR11089:SF3 | GTP-BINDING PROTEIN- | T[195-494] 2.3e−49 |
| | | RELATED | |
| | | PLANT/BACTERIA | |
| Superfamily | SSF52540 | P-loop containing | T[174-349] 4.6e−18 |
| | | nucleoside triphosphate | |
| | | hydrolases | |

### 4.5. Group I MBF1 Polypeptides

The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, ProDom and Pfam, Smart and TIGRFAMs. Interpro is hosted at the European Bioinformatics Institute in the United Kingdom.

The results of the InterPro scan of the polypeptide sequence as represented by SEQ ID NO: 189 are presented in Table C3.

TABLE C3
InterPro scan results of the polypeptide sequence as represented by SEQ ID NO: 189

| InterPro accession | Integrated database | Integrated database | Integrated database |
|---|---|---|---|
| number and name | name | accession number | accession name |
| IPR001387 | PFAM | PF01381 | HTH_3 |
| Helix-turn-helix type 3 | | | |
| domain | | | |
| | SMART | SM00530 | HTH_XRE |
| | Profile | PS50943 | HTH_CROC1 |
| IPR010982 | SuperFamily | SSF47413 | Lambda_like_DNA |
| Lambda repressor-like, | | | |
| DNA binding domain | | | |
| IPR013729 | PFAM | PF08523 | MBF1 |
| Multibridging factor 1, | | | |
| N-terminal domain | | | |
| No IPR unintegrated | GENE3D | G3DSA:1.10.260.40 | G3DSA:1.10.260.40 |
| No IPR unintegrated | PANTHER | PTHR10245 | PTHR10245 |
| No IPR unintegrated | PANTHER | PTHR10245:SF1 | PTHR10245:SF1 |

### Example 5

### Topology Prediction of the Polypeptide Sequences Useful in Performing the Methods of the Invention

### 5.1. COX VIIa Subunit Polypeptides—PKT Polypeptides—PHDF Polypeptides

TargetP 1.1 predicts the subcellular location of eukaryotic proteins. The location assignment is based on the predicted presence of any of the N-terminal pre-sequences: chloroplast transit peptide (cTP), mitochondrial targeting peptide (mTP) or secretory pathway signal peptide (SP). Scores on which the final prediction is based are not really probabilities, and they do not necessarily add to one. However, the location with the highest score is the most likely according to TargetP, and the relationship between the scores (the reliability class) may be an indication of how certain the prediction is. The reliability class (RC) ranges from 1 to 5, where 1 indicates the strongest prediction. TargetP is maintained at the server of the Technical University of Denmark. For the sequences predicted to contain an N-terminal presequence a potential cleavage site can also be predicted.

A number of parameters are selected, such as organism group (non-plant or plant), cutoff sets (none, predefined set of cutoffs, or user-specified set of cutoffs), and the calculation of prediction of cleavage sites (yes or no).

Many other algorithms can be used to perform such analyses, including:
ChloroP 1.1 hosted on the server of the Technical University of Denmark;
Protein Prowler Subcellular Localisation Predictor version 1.2 hosted on the server of the Institute for Molecular Bioscience, University of Queensland, Brisbane, Australia;
PENCE Proteome Analyst PA-GOSUB 2.5 hosted on the server of the University of Alberta, Edmonton, Alberta, Canada;
TMHMM, hosted on the server of the Technical University of Denmark
PSORT (URL: psort.org)
PLOC (Park and Kanehisa, Bioinformatics, 19, 1656-1663, 2003).

### 5.2. YLD-ZnF Polypeptides

TargetP 1.1 predicts the subcellular location of eukaryotic proteins. The location assignment is based on the predicted presence of any of the N-terminal pre-sequences: chloroplast transit peptide (cTP), mitochondrial targeting peptide (mTP) or secretory pathway signal peptide (SP). Scores on which the final prediction is based are not really probabilities, and they do not necessarily add to one. However, the location with the highest score is the most likely according to TargetP, and the relationship between the scores (the reliability class) may be an indication of how certain the prediction is. The reliability class (RC) ranges from 1 to 5, where 1 indicates the strongest prediction. TargetP is maintained at the server of the Technical University of Denmark.

For the sequences predicted to contain an N-terminal presequence a potential cleavage site can also be predicted.

A number of parameters were selected, such as organism group (non-plant or plant), cutoff sets (none, predefined set of cutoffs, or user-specified set of cutoffs), and the calculation of prediction of cleavage sites (yes or no).

The results of TargetP 1.1 analysis of the polypeptide sequence as represented by SEQ ID NO: 19 are presented Table D1. The “plant” organism group has been selected, no cutoffs defined, and the predicted length of the transit peptide requested. The subcellular localization of the polypeptide sequence as represented by SEQ ID NO: 2 may be the mitochondrion.

TABLE D1
TargetP 1.1 analysis of the polypeptide
sequence as represented by SEQ ID NO: 19.

| Name | Len | cTP | mTP | SP | other | Loc | RC | TPlen |
|---|---|---|---|---|---|---|---|---|
| SEQIDNO: 19 | 199 | 0.186 | 0.890 | 0.001 | 0.040 | M | 2 | 13 |
| cutoff | | 0.000 | 0.000 | 0.000 | 0.000 | | | |

Abbreviations: Len, Length; cTP, Chloroplastic transit peptide; mTP, Mitochondrial transit peptide, SP, Secretory pathway signal peptide, other, Other subcellular targeting, Loc, Predicted Location; RC, Reliability class; TPlen, Predicted transit peptide length.

Many other algorithms can be used to perform such analyses, including:
ChloroP 1.1 hosted on the server of the Technical University of Denmark;
Protein Prowler Subcellular Localisation Predictor version 1.2 hosted on the server of the Institute for Molecular Bioscience, University of Queensland, Brisbane, Australia;
PENCE Proteome Analyst PA-GOSUB 2.5 hosted on the server of the University of Alberta, Edmonton, Alberta, Canada;
TMHMM, hosted on the server of the Technical University of Denmark
PSORT (URL: psort.org)
PLOC (Park and Kanehisa, Bioinformatics, 19, 1656-1663, 2003).

### 5.3. NOA Polypeptides

TargetP 1.1 predicts the subcellular location of eukaryotic proteins. The location assignment is based on the predicted presence of any of the N-terminal pre-sequences: chloroplast transit peptide (cTP), mitochondrial targeting peptide (mTP) or secretory pathway signal peptide (SP). Scores on which the final prediction is based are not really probabilities, and they do not necessarily add to one. However, the location with the highest score is the most likely according to TargetP, and the relationship between the scores (the reliability class) may be an indication of how certain the prediction is. The reliability class (RC) ranges from 1 to 5, where 1 indicates the strongest prediction. TargetP is maintained at the server of the Technical University of Denmark.

For the sequences predicted to contain an N-terminal presequence a potential cleavage site can also be predicted.

A number of parameters were selected, such as organism group (non-plant or plant), cutoff sets (none, predefined set of cutoffs, or user-specified set of cutoffs), and the calculation of prediction of cleavage sites (yes or no).

The results of TargetP 1.1 analysis of the polypeptide sequence as represented by SEQ ID NO: 59 are presented Table D2. The “plant” organism group has been selected, no cutoffs defined, and the predicted length of the transit peptide requested. The subcellular localization of the polypeptide sequence as represented by SEQ ID NO: 59 may be the mitochondrion. SEQ ID NO: 59 is described as mitochondrial protein (Guo & Crawford, Plant Cell 17, 3436-3450, 2005) and as a plastidial protein (Flores-Pérez et al., 2008).

TABLE D2
TargetP 1.1 analysis of the polypeptide
sequence as represented by SEQ ID NO: 59.

| Name | Len | cTP | mTP | SP | other | Loc | RC | TPlen |
|---|---|---|---|---|---|---|---|---|
| NOA1 | 561 | 0.398 | 0.779 | 0.010 | 0.025 | M | 4 | 6 |
| cutoff | | 0.000 | 0.000 | 0.000 | 0.000 | | | |

Abbreviations: Len, Length; cTP, Chloroplastic transit peptide; mTP, Mitochondrial transit peptide, SP, Secretory pathway signal peptide, other, Other subcellular targeting, Loc, Predicted Location; RC, Reliability class; TPlen, Predicted transit peptide length.

Many other algorithms can be used to perform such analyses, including:
ChloroP 1.1 hosted on the server of the Technical University of Denmark;
Protein Prowler Subcellular Localisation Predictor version 1.2 hosted on the server of the Institute for Molecular Bioscience, University of Queensland, Brisbane, Australia;
PENCE Proteome Analyst PA-GOSUB 2.5 hosted on the server of the University of Alberta, Edmonton, Alberta, Canada;
TMHMM, hosted on the server of the Technical University of Denmark
PSORT (URL: psort.org)
PLOC (Park and Kanehisa, Bioinformatics, 19, 1656-1663, 2003).
   5.4. ASF1-like Polypeptides

TargetP 1.1 predicts the subcellular location of eukaryotic proteins. The location assignment is based on the predicted presence of any of the N-terminal pre-sequences: chloroplast transit peptide (cTP), mitochondrial targeting peptide (mTP) or secretory pathway signal peptide (SP). Scores on which the final prediction is based are not really probabilities, and they do not necessarily add to one. However, the location with the highest score is the most likely according to TargetP, and the relationship between the scores (the reliability class) may be an indication of how certain the prediction is. The reliability class (RC) ranges from 1 to 5, where 1 indicates the strongest prediction. TargetP is maintained at the server of the Technical University of Denmark.

For the sequences predicted to contain an N-terminal presequence a potential cleavage site can also be predicted.

A number of parameters are selected, such as organism group (non-plant or plant), cutoff sets (none, predefined set of cutoffs, or user-specified set of cutoffs), and the calculation of prediction of cleavage sites (yes or no).

Many other algorithms can be used to perform such analyses, including:
ChloroP 1.1 hosted on the server of the Technical University of Denmark;
Protein Prowler Subcellular Localisation Predictor version 1.2 hosted on the server of the Institute for Molecular Bioscience, University of Queensland, Brisbane, Australia;
PENCE Proteome Analyst PA-GOSUB 2.5 hosted on the server of the University of Alberta, Edmonton, Alberta, Canada;
TMHMM, hosted on the server of the Technical University of Denmark
PSORT (URL: psort.org)
PLOC (Park and Kanehisa, Bioinformatics, 19, 1656-1663, 2003).

### Example 6

### Subcellular Localisation Prediction of the Polypeptide Sequences Useful in Performing the Methods of the Invention

### 6.1. Group I MBF1 Polypeptides

Experimental methods for protein localization range from immunolocalization to tagging of proteins using green fluorescent protein (GFP) or beta-glucuronidase (GUS). Such methods to identify subcellular compartmentalisation of group I MBF1 polypeptides are well known in the art.

Computational prediction of protein localisation from sequence data was performed. Among algorithms well known to a person skilled in the art are available at the ExPASy Proteomics tools hosted by the Swiss Institute for Bioinformatics, for example, PSort, TargetP, ChloroP, LocTree, Predotar, LipoP, MITOPROT, PATS, PTS1, SignalP, TMHMM, TMpred, and others.

TargetP 1.1 predicts the subcellular location of eukaryotic proteins. The location assignment is based on the predicted presence of any of the N-terminal pre-sequences: chloroplast transit peptide (cTP), mitochondrial targeting peptide (mTP) or secretory pathway signal peptide (SP). Scores on which the final prediction is based are not really probabilities, and they do not necessarily add to one. However, the location with the highest score is the most likely according to TargetP, and the relationship between the scores (the reliability class) may be an indication of how certain the prediction is. The reliability class (RC) ranges from 1 to 5, where 1 indicates the strongest prediction. TargetP is maintained at the server of the Technical University of Denmark.

For the sequences predicted to contain an N-terminal presequence a potential cleavage site can also be predicted.

A number of parameters were selected, such as organism group (non-plant or plant), cutoff sets (none, predefined set of cutoffs, or user-specified set of cutoffs), and the calculation of prediction of cleavage sites (yes or no).

The results of TargetP 1.1 analysis of the polypeptide sequence as represented by SEQ ID NO: 189 are presented in the Table below. The “plant” organism group has been selected, and no cutoffs defined. The predicted subcellular localization of the polypeptide sequence as represented by SEQ ID NO: 189 is not chloroplastic, not mitochondrial and not the secretory pathway, but most likely the nucleus.

Table showing TargetP 1.1 analysis of the polypeptide sequence as represented by SEQ ID NO: 189

| | |
|---|---|
| Length (AA) | 142 |
| Chloroplastic transit peptide | 0.395 |
| Mitochondrial transit peptide | 0.131 |
| Secretory pathway signal peptide | 0.063 |
| Other subcellular targeting | 0.670 |
| Predicted Location | Other |
| Reliability class | 4 |

### Example 7

### Assay Related to the Polypeptide Sequences Useful in Performing the Methods of the Invention

### 7.1. NOA Polypeptides

A GTPase assay for AtNOS1 is described in Moreau et al. (2008). En bref, 20 or 40 μM of AtNOS1 protein are incubated with 500 μM GTP, 2 mM MgCl₂, 200 mM KCl in buffer B (50 mM Tris HCl pH 7.5, 150 mM NaCl, 10% glycerol and 2 mM DTT) at 37° C. overnight. Samples are boiled for 5 minutes to stop the reaction and to precipitate the proteins and are then centrifuged for 5 minutes. The supernatant is analysed by reverse phase HPLC on a Waters Sunfire C18 5 μM (4.5×250 mm) column. Nucleotides are separated with an isocratic condition at 1 ml/min of 100 mM KH₂PO₄ at pH 6.5, 10 mM tetra-butyl ammonium bromide, 0.2 mM NaN₃ and 7.5% acetonitrile. Control reactions in the absence of protein are analysed following the same procedure.

Rates of GTP hydrolysis are quantified by measuring [³²P] phosphate release (Majumdar et al., J. Biol. Chem. 279, 40137-40145, 2004). Reactions containing 1 nM [γ-³²P]GTP (2 μCi) and varying amounts of cold GTP are prepared in 300 μl of buffer B supplemented with 5 mM MgCl₂ and 200 mM KCl. The reaction is started by addition of the protein. At various times, 50 μl aliquots are mixed with 1 ml of activated charcoal (5% in 50 mM NaH₂PO₄). After 1 min centrifugation, [γ³²-P] phosphates in the supernatant are counted on a liquid scintillation counter. Counts per min (cpm) are plotted as a function of time for the different GTP concentrations. Reactions in the absence of protein are conducted to control for spontaneous hydrolysis. Km and Vmax values are determined by plotting the initial velocity of GTP hydrolysis (v₀) as a function of the substrate concentration. Curves are fitted to the equation v₀=(Vmax×[GTP])/(Km+[GTP]) using Origin Pro 7.5 software.

### 7.2. Group I MBF1 Polypeptides

Group I MBF1 polypeptides useful in the methods of the present invention (at least in their native form) typically, but not necessarily, have transcriptional regulatory activity and capacity to interact with other proteins. DNA-binding activity and protein-protein interactions may readily be determined in vitro or in vivo using techniques well known in the art (for example in Current Protocols in Molecular Biology, Volumes 1 and 2, Ausubel et al. (1994), Current Protocols). Group I MBF1 polypeptides contain a Helix-turn-helix type 3 domain.

Furthermore, group I MBF1 polypeptides useful in performing the methods of the invention are capable of complementing a yeast mutant strain lacking MBF1 acitivity, as described in Tsuda et al. (2004) Plant Cell Physiol 45: 225-231.

### Example 8

### Cloning of the nucleic acid sequence used in the methods of the invention

### 8.1. COX VIIa Subunit Polypeptides

The nucleic acid sequence is amplified by PCR using as template a cDNA library (in pCMV Sport 6.0; Invitrogen, Paisley, UK). PCR is performed using Hifi Taq DNA polymerase in standard conditions, using 200 ng of template in a 50 μl PCR mix. The primers include the AttB sites for Gateway recombination. The amplified PCR fragment is purified also using standard methods. The first step of the Gateway procedure, the BP reaction, is then performed, during which the PCR fragment recombines in vivo with the pDONR201 plasmid to produce, according to the Gateway terminology, an “entry clone”. Plasmid pDONR201 is purchased from Invitrogen, as part of the Gateway® technology.

The entry clone comprising SEQ ID NO: 1, 3, 5 or 7 is then used in an LR reaction with a destination vector used for Oryza sativa transformation. This vector contains as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 9) for constitutive expression is located upstream of this Gateway cassette.

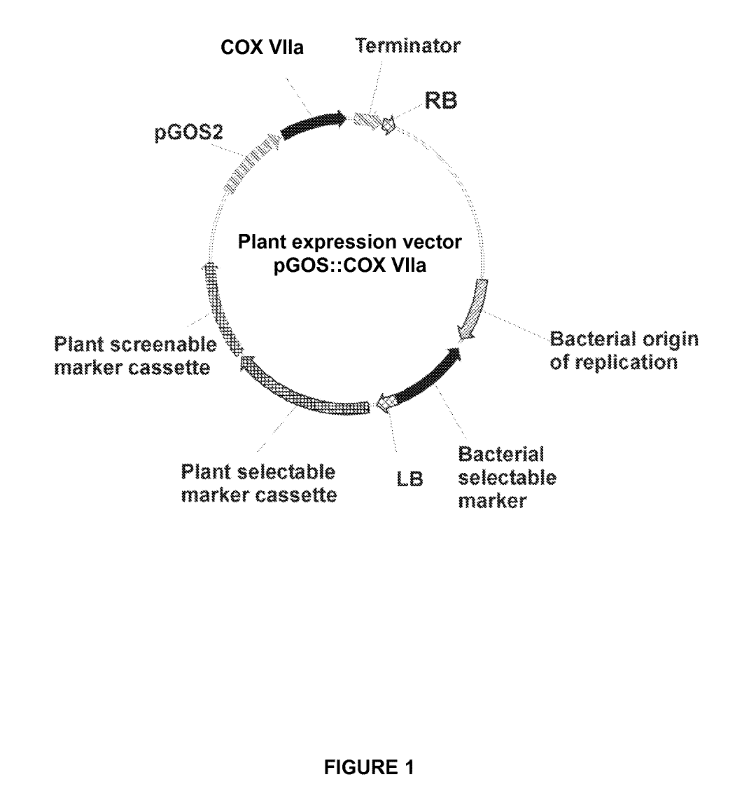

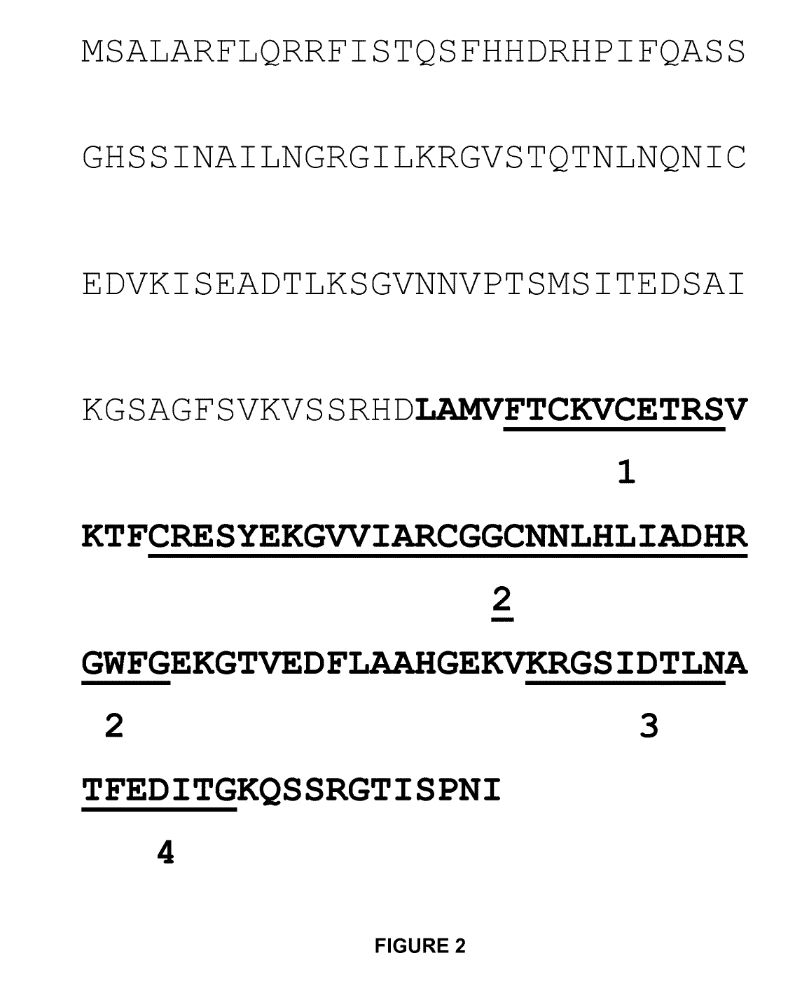

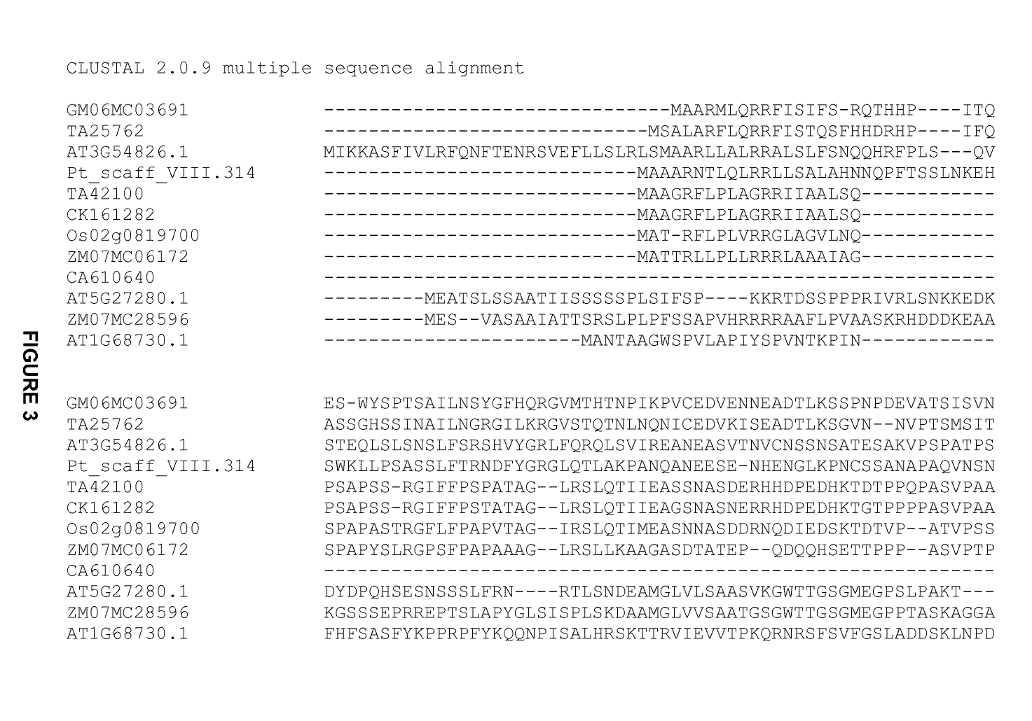

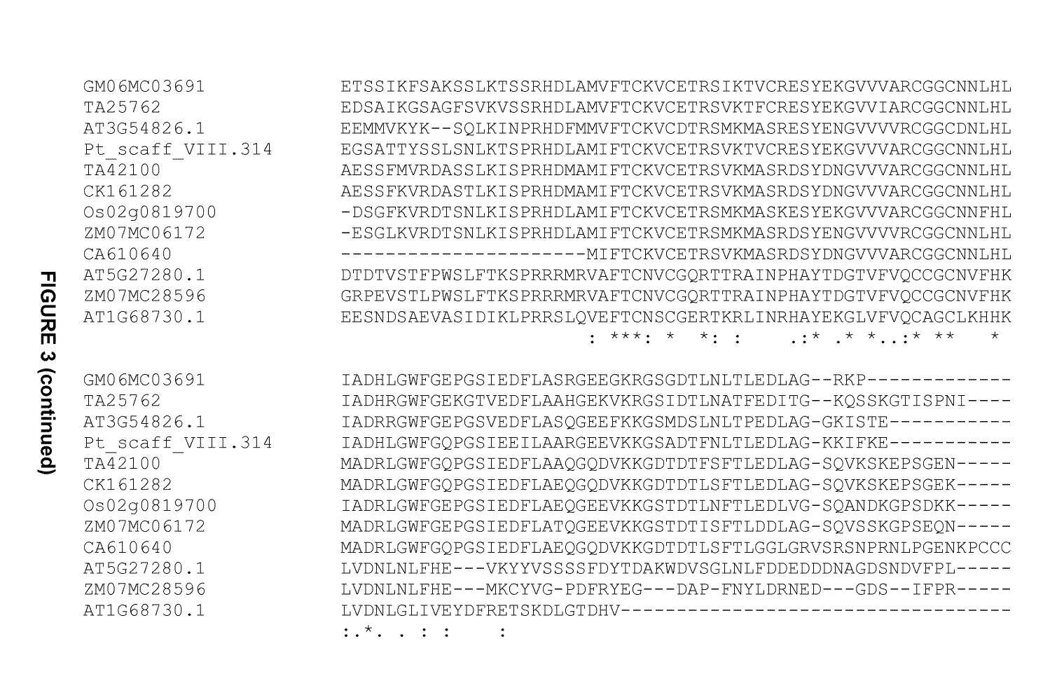

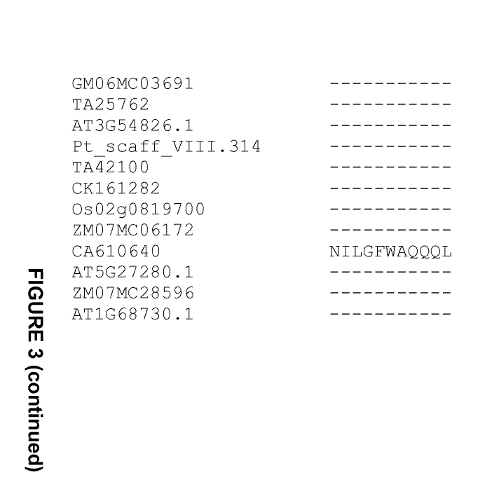

After the LR recombination step, the resulting expression vector pGOS2:COX VIIa subunit (FIG. 1) is transformed into Agrobacterium strain LBA4044 according to methods well known in the art.

### 8.2. YLD-ZnF Polypeptides

The nucleic acid sequence used in the methods of the invention was amplified by PCR using as template a custom-made Medicago truncatula seedlings cDNA library (in pCMV Sport 6.0; Invitrogen, Paisley, UK). PCR was performed using Hifi Taq DNA polymerase in standard conditions, using 200 ng of template in a 50 μl PCR mix. The primers used were prm11653 (SEQ ID NO: 24; sense, start codon in bold): 5′-ggggacaagtttgtacaaaaaagcaggc ttaaacaatgtcggcgttggcgagg-3′ and prm11654 (SEQ ID NO: 25; reverse, complementary): 5′-ggggaccactttgtacaagaaagctgggtcccttccaatatctcagtgctaccc-3′, which include the AttB sites for Gateway recombination. The amplified PCR fragment was purified also using standard methods. The first step of the Gateway procedure, the BP reaction, was then performed, during which the PCR fragment recombined in vivo with the pDONR201 plasmid to produce, according to the Gateway terminology, an “entry clone”, pYLD-ZnF. Plasmid pDONR201 was purchased from Invitrogen, as part of the Gateway® technology.

The entry clone comprising SEQ ID NO: 18 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 29) for constitutive specific expression was located upstream of this Gateway cassette.

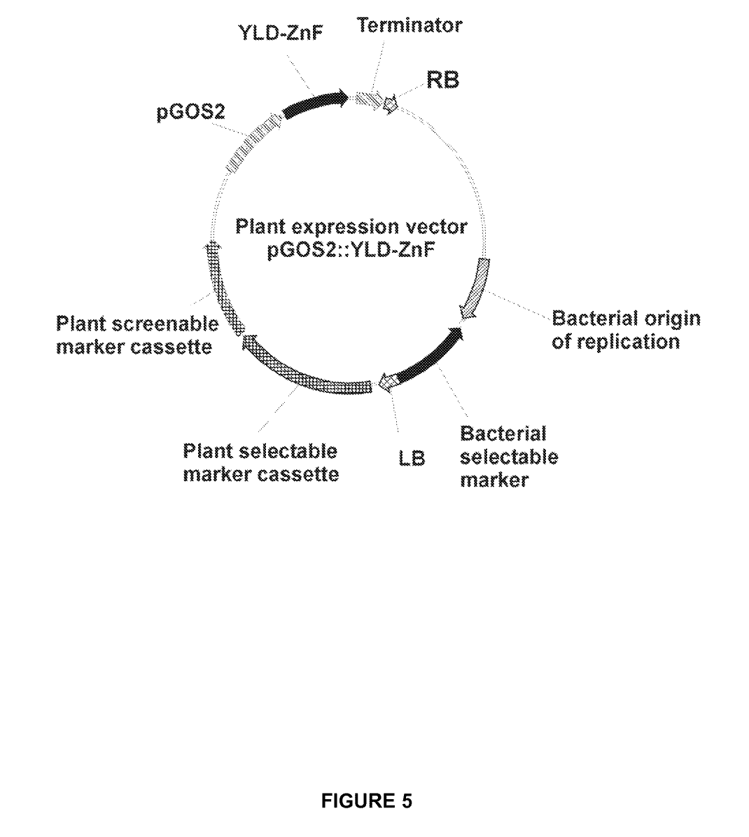

After the LR recombination step, the resulting expression vector pGOS2:YLD-ZnF (FIG. 5) was transformed into Agrobacterium strain LBA4044 according to methods well known in the art.

### 8.3. PKT Polypeptides

The nucleic acid sequence is amplified by PCR using as template a cDNA library (in pCMV Sport 6.0; Invitrogen, Paisley, UK). PCR is performed using Hifi Taq DNA polymerase in standard conditions, using 200 ng of template in a 50 μl PCR mix. The primers include the AttB sites for Gateway recombination. The amplified PCR fragment is purified also using standard methods. The first step of the Gateway procedure, the BP reaction, is then performed, during which the PCR fragment recombines in vivo with the pDONR201 plasmid to produce, according to the Gateway terminology, an “entry clone”. Plasmid pDONR201 is purchased from Invitrogen, as part of the Gateway® technology.

The entry clone comprising SEQ ID NO: 51 or SEQ ID NO: 53 is then used in an LR reaction with a destination vector used for Oryza sativa transformation. This vector contains as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 55) for constitutive expression is located upstream of this Gateway cassette.

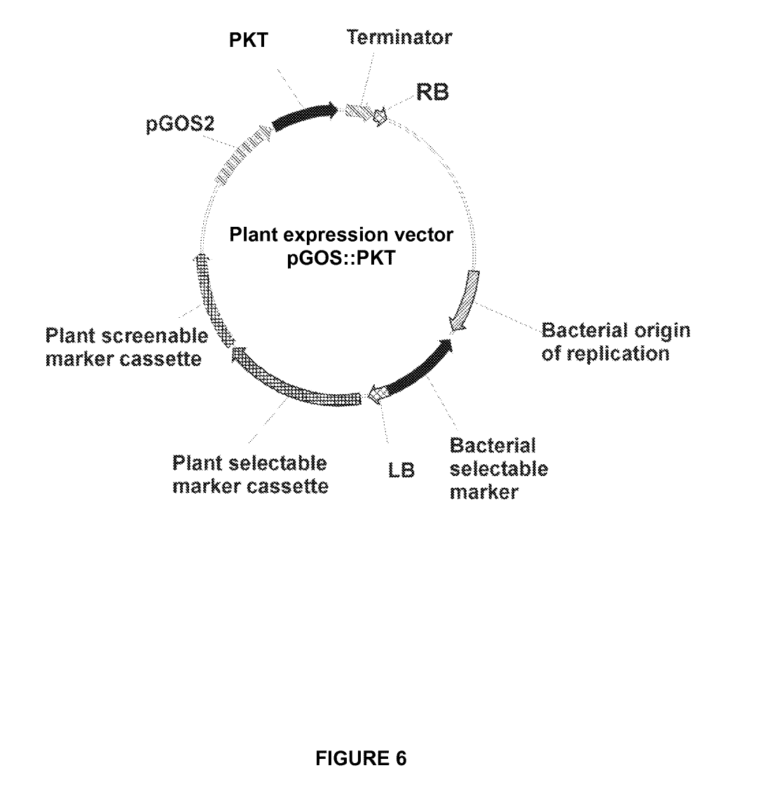

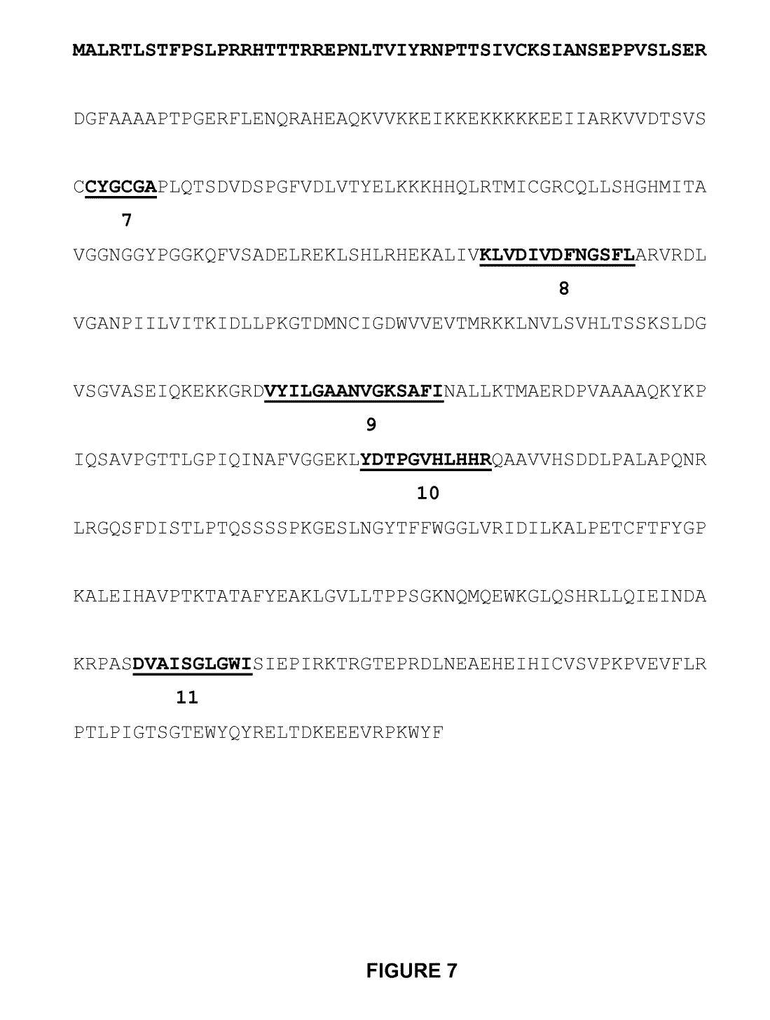

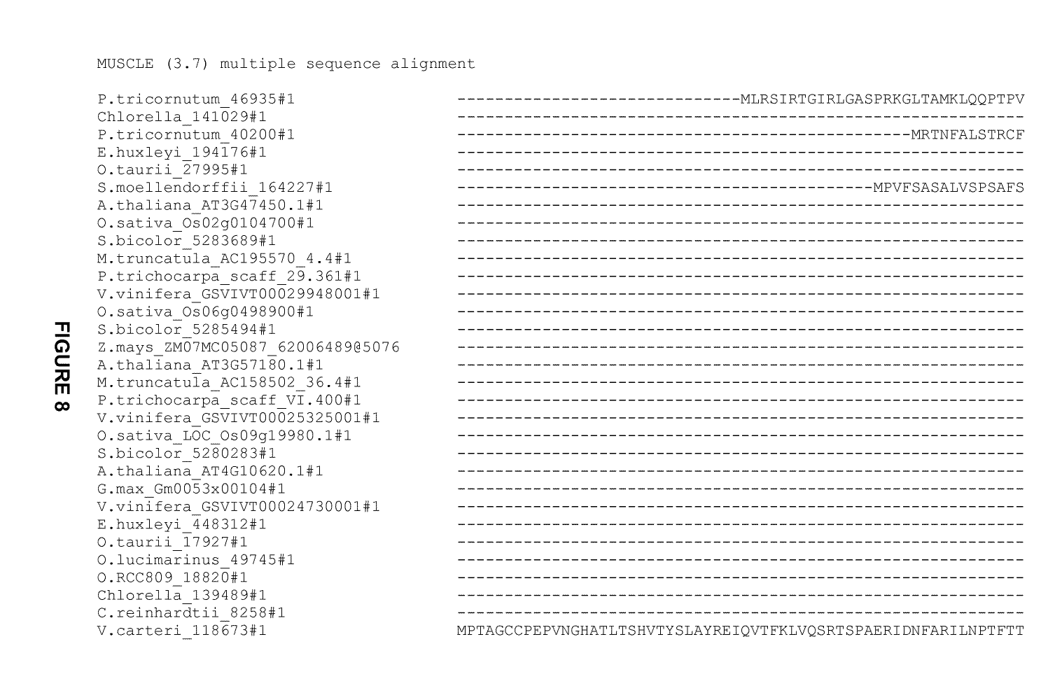

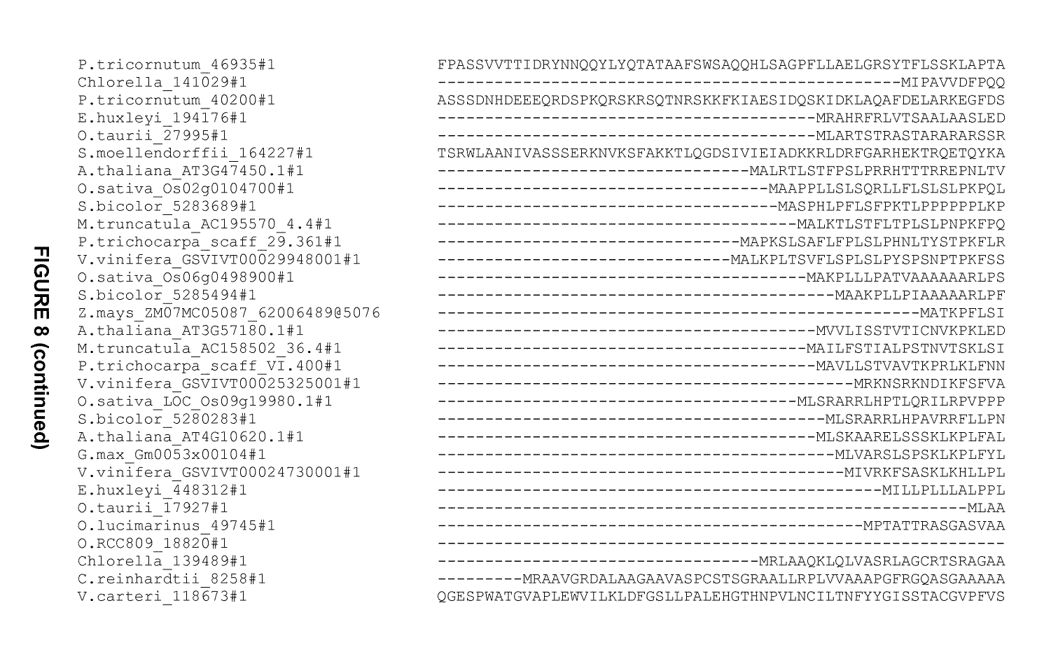

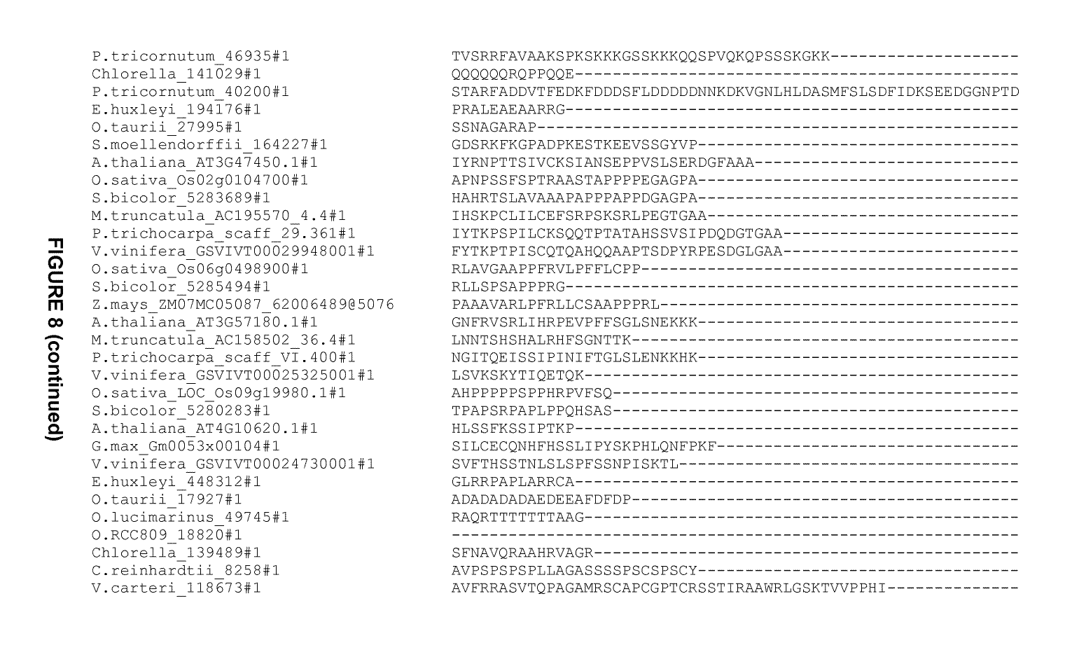

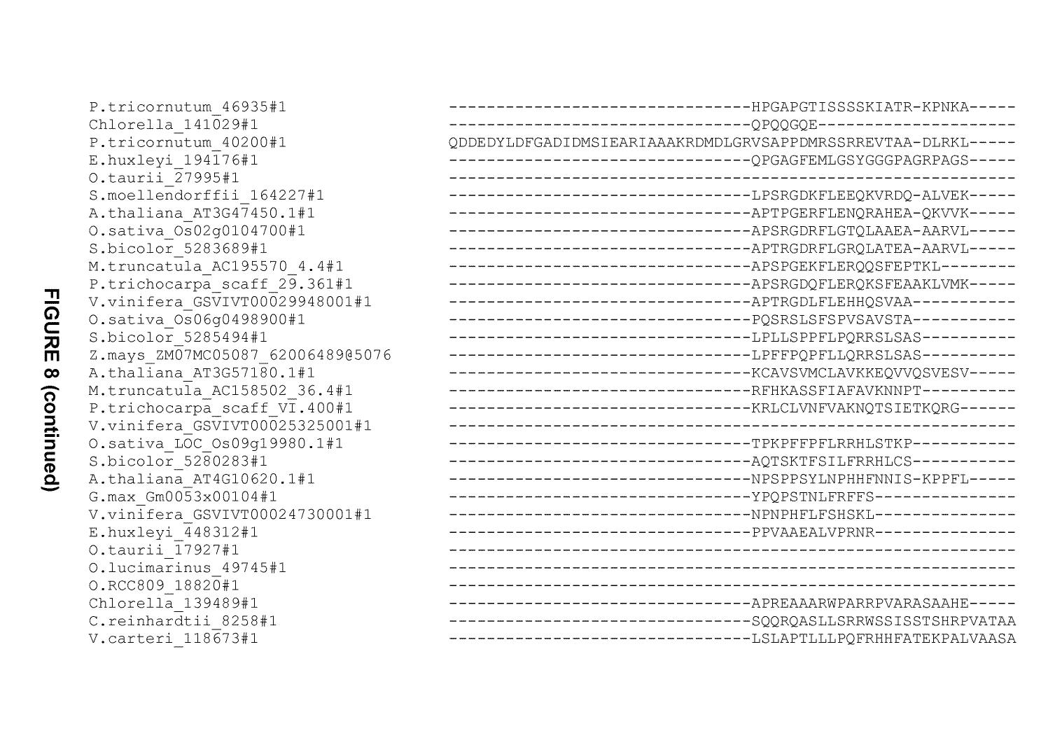

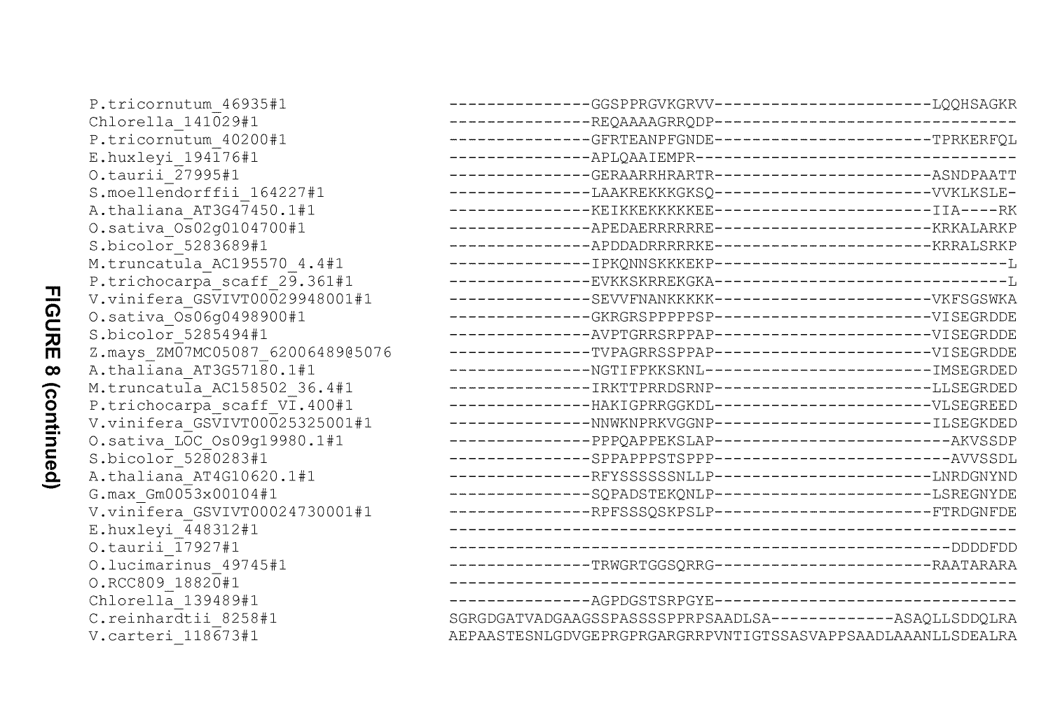

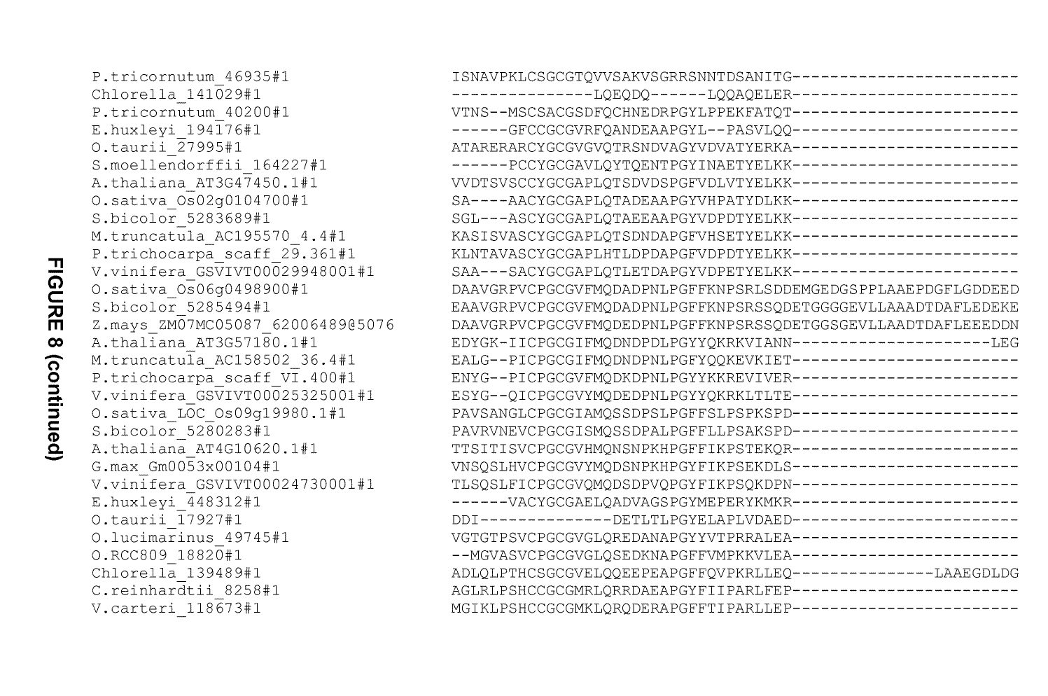

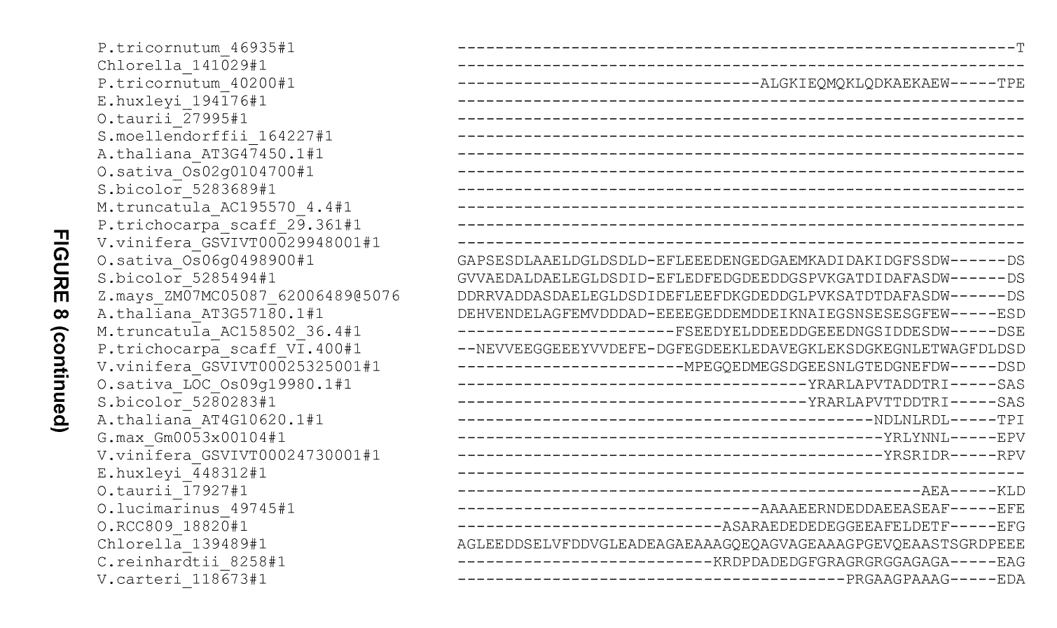

After the LR recombination step, the resulting expression vector pGOS2:PKT (FIG. 6) is transformed into Agrobacterium strain LBA4044 according to methods well known in the art.

8.4. NOA Polypeptides

The nucleic acid sequence used in the methods of the invention was amplified by PCR using as template a custom-made Arabidopsis thaliana seedlings cDNA library (in pCMV Sport 6.0; Invitrogen, Paisley, UK). PCR was performed using Hifi Taq DNA polymerase in standard conditions, using 200 ng of template in a 50 μl PCR mix. The primers used were prm09511 (SEQ ID NO: 72; sense, start codon in bold): 5′-ggggacaagtttgtacaaaaaagcaggct taaacaatggcgctacgaacactct-3′ and prm09512 (SEQ ID NO: 73; reverse, complementary): 5′-ggggaccactttgtacaagaaagctgggttaagccgatatttttgcatct-3′, which include the AttB sites for Gateway recombination. The amplified PCR fragment was purified also using standard methods. The first step of the Gateway procedure, the BP reaction, was then performed, during which the PCR fragment recombined in vivo with the pDONR201 plasmid to produce, according to the Gateway terminology, an “entry clone”, pNOA. Plasmid pDONR201 was purchased from Invitrogen, as part of the Gateway® technology.

The entry clone comprising SEQ ID NO: 58 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 71) for constitutive specific expression was located upstream of this Gateway cassette.

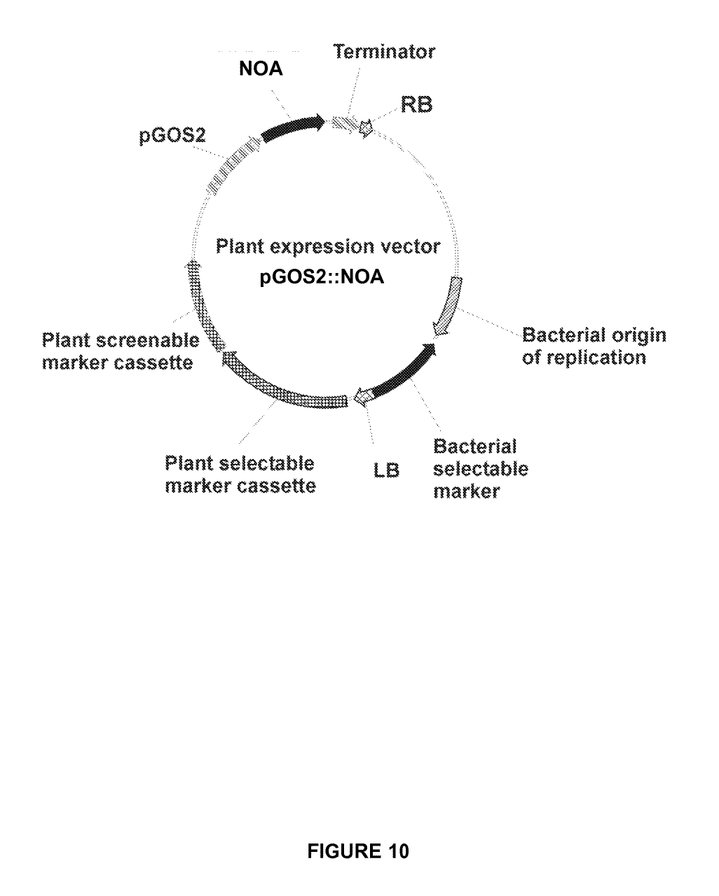

After the LR recombination step, the resulting expression vector pGOS2:NOA (FIG. 10) was transformed into Agrobacterium strain LBA4044 according to methods well known in the art.

8.5. ASF1-like Polypeptides

The ASF1-like nucleic acid sequence was amplified by PCR using as template a cDNA library (in pCMV Sport 6.0; Invitrogen, Paisley, UK). PCR was performed using Hifi Taq DNA polymerase in standard conditions, using 200 ng of template in a 50 μl PCR mix. For the rice ASF1-like sequence, the primers used were prm41 (SEQ ID NO: 170; sense, start codon in bold): 5′-aaaaagcaggctcacaatggagaatgggaaaagagac-3′ and prm41× (SEQ ID NO: 171; reverse, complementary): 5′-agaaagctgggttggttttaactagttccaccg-3′, which include the AttB sites for Gateway recombination. The amplified PCR fragment was purified also using standard methods. The first step of the Gateway procedure, the BP reaction, was then performed, during which the PCR fragment recombines in vivo with the pDONR201 plasmid to produce, according to the Gateway terminology, an “entry clone”, pASF1-like. Plasmid pDONR201 was purchased from Invitrogen, as part of the Gateway® technology.

For the Arabidopsis thaliana ASF1-like sequence, the primers used were prm41 (SEQ ID NO: 172; sense, start codon in bold): 5′-aaaaagcaggctcacaatggagaatgggaaaagagac-3′ and prm41× (SEQ ID NO: 173; reverse, complementary): 5′-agaaagctgggttggttttaac tagttccaccg-3′.

The entry clone comprising SEQ ID NO: 134 or SEQ ID NO: 136 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 174) for constitutive expression was located upstream of this Gateway cassette.

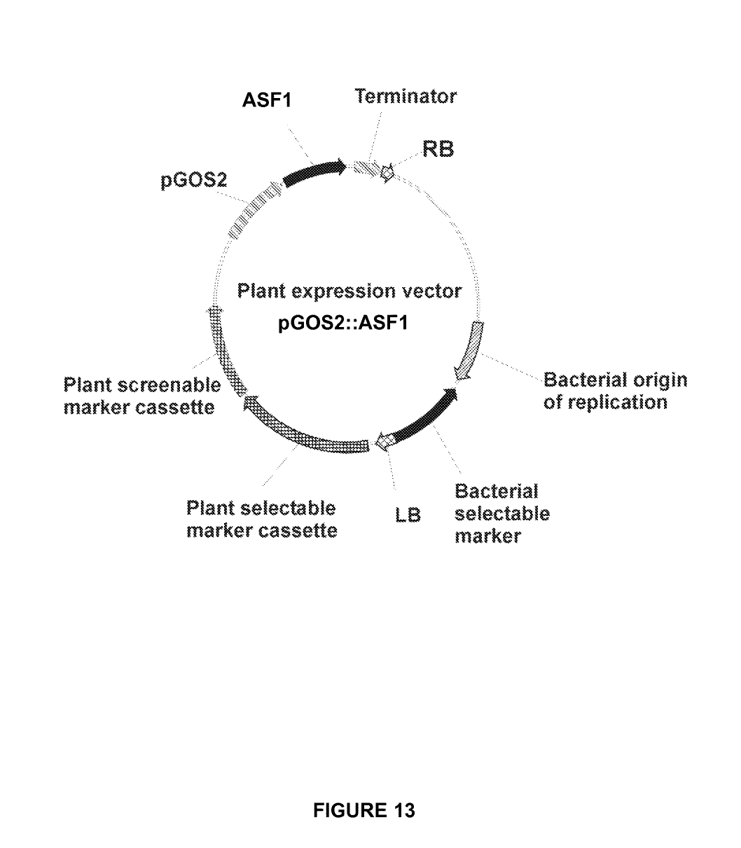

After the LR recombination step, the resulting expression vector pGOS2:ASF1-like (FIG. 13) was transformed into Agrobacterium strain LBA4044 according to methods well known in the art.

### 8.6. PHDF Polypeptides

The nucleic acid sequence is amplified by PCR using as template a cDNA library (in pCMV Sport 6.0; Invitrogen, Paisley, UK). PCR is performed using Hifi Taq DNA polymerase in standard conditions, using 200 ng of template in a 50 μl PCR mix. The primers include the AttB sites for Gateway recombination. The amplified PCR fragment is purified also using standard methods. The first step of the Gateway procedure, the BP reaction, is then performed, during which the PCR fragment recombines in vivo with the pDONR201 plasmid to produce, according to the Gateway terminology, an “entry clone”. Plasmid pDONR201 is purchased from Invitrogen, as part of the Gateway® technology.

The entry clone comprising SEQ ID NO: 175 or SEQ ID NO: 177 is then used in an LR reaction with a destination vector used for Oryza sativa transformation. This vector contains as functional elements within the T-DNA borders: a plant selectable marker; a screenable marker expression cassette; and a Gateway cassette intended for LR in vivo recombination with the nucleic acid sequence of interest already cloned in the entry clone.

A rice GOS2 promoter (SEQ ID NO: 181) for constitutive expression is located upstream of this Gateway cassette.

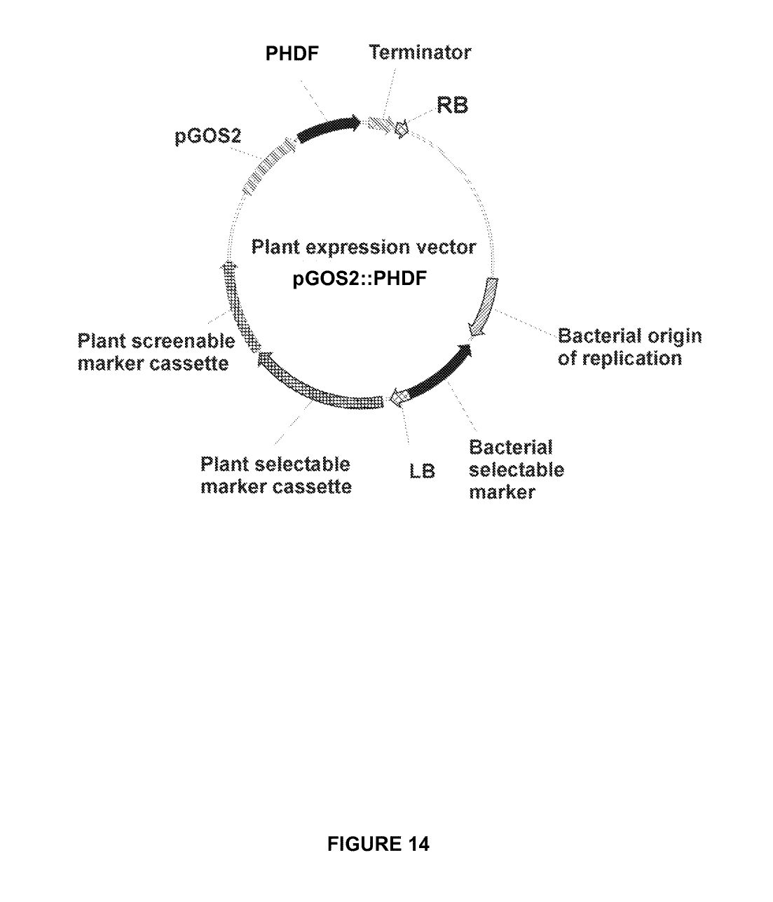

After the LR recombination step, the resulting expression vector pGOS2:PHDF (FIG. 14) is transformed into Agrobacterium strain LBA4044 according to methods well known in the art.

### 8.7. Group I MBF1 Polypeptides

Unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al. (1994), Current Protocols in Molecular Biology, Current Protocols. Standard materials and methods for plant molecular work are described in Plant Molecular Biology Labfax (1993) by R. D. D. Croy, published by BIOS Scientific Publications Ltd (UK) and Blackwell Scientific Publications (UK).

The following primers, which include the AttB sites for Gateway recombination, were used for PCR amplification, using as template a cDNA bank constructed using RNA from plants at different developmental staaes:

| Nucleic acid | Source | Forward primer | Reverse primer |
|---|---|---|---|
| sequence | organism | sequence | sequence |
| SEQ ID NO: 188 | Arabidopsis | SEQ ID NO: 255 | SEQ ID NO: 256 |
| | thaliana | | |
| SEQ ID NO: 190 | Arabidopsis | SEQ ID NO: 255 | SEQ ID NO: 257 |
| | thaliana | | |
| SEQ ID NO: 192 | Medicago | SEQ ID NO: 260 | SEQ ID NO: 261 |
| | truncatula | | |
| SEQ ID NO: 194 | Triticum | SEQ ID NO: 258 | SEQ ID NO: 259 |
| | aestivum | | |

SEQ ID NO: 255 prm09335 forward for SEQ ID NO: 188
and SEQ ID NO: 190
Ggggacaagtttgtacaaaaaagcaggcttaaacaatggccggaattgg
ac
SEQ ID NO: 256 prm09336 reverse for SEQ ID NO: 188
ggggaccactttgtacaagaaagctgggttgttgttacctttaagagctt
tg
SEQ ID NO: 257 prm09337 reverse for SEQ ID NO: 190
Ggggaccactttgtacaagaaagctgggtagaacttggctcacttctttc
SEQ ID NO: 258 prm10242 forward for SEQ ID NO: 194
ggggacaagtttgtacaaaaaagcaggcttaaacaatggctgggattggt
cc
SEQ ID NO: 259 prm10243 reverse for SEQ ID NO: 194
Ggggaccactttgtacaagaaagctgggtgtaaggcaaatagacagggct
SEQ ID NO: 260 prm10244 forward for SEQ ID NO: 192
Ggggacaagtttgtacaaaaaagcaggcttaaacaatgtcaggtctaggc
catatt
SEQ ID NO: 261 prm10245 reverse for SEQ ID NO: 192
ggggaccactttgtacaagaaagctgggtattaggtcttcatttcttgcc

PCR was performed using Hifi Taq DNA polymerase in standard conditions. A PCR fragment of the expected length (including attB sites) was amplified and purified also using standard methods. The first step of the Gateway procedure, the BP reaction, was then performed, during which the PCR fragment recombined in vivo with the pDONR201 plasmid to produce, according to the Gateway terminology, an “entry clone”. Plasmid pDONR201 was purchased from Invitrogen, as part of the Gateway® technology.

The entry clone comprising SEQ ID NO: 188 or SEQ ID NO: 190 or SEQ ID NO: 192 or SEQ ID NO: 194 was subsequently used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice constitutive promoter (SEQ ID NO: 253 or SEQ ID NO: 254) for constitutive expression was located upstream of this Gateway cassette.

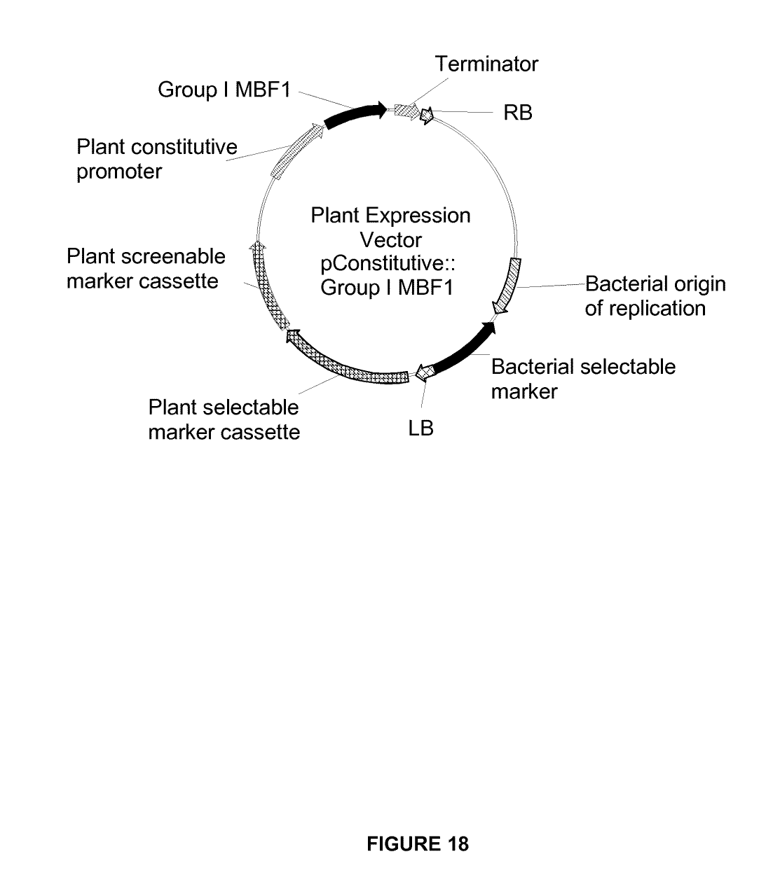

After the LR recombination step, the resulting expression vector pConstitutive:group I MBF1 (where pConstitutive is either SEQ ID NO: 253 or SEQ ID NO: 254; where group I MBF1 is either SEQ ID NO: 188 or SEQ ID NO: 190 or SEQ ID NO: 192 or SEQ ID NO: 194; FIG. 18) for constitutive expression, was transformed into Agrobacterium strain LBA4044 according to methods well known in the art.

### Example 9

### Plant Transformation

### Rice Transformation

The Agrobacterium containing the expression vector was used to transform Oryza sativa plants. Mature dry seeds of the rice japonica cultivar Nipponbare were dehusked. Sterilization was carried out by incubating for one minute in 70% ethanol, followed by 30 minutes in 0.2% HgCl₂, followed by a 6 times 15 minutes wash with sterile distilled water. The sterile seeds were then germinated on a medium containing 2,4-D (callus induction medium). After incubation in the dark for four weeks, embryogenic, scutellum-derived calli were excised and propagated on the same medium. After two weeks, the calli were multiplied or propagated by subculture on the same medium for another 2 weeks. Embryogenic callus pieces were sub-cultured on fresh medium 3 days before co-cultivation (to boost cell division activity).

Agrobacterium strain LBA4404 containing the expression vector was used for co-cultivation. Agrobacterium was inoculated on AB medium with the appropriate antibiotics and cultured for 3 days at 28° C. The bacteria were then collected and suspended in liquid co-cultivation medium to a density (OD₆₀₀) of about 1. The suspension was then transferred to a Petri dish and the calli immersed in the suspension for 15 minutes. The callus tissues were then blotted dry on a filter paper and transferred to solidified, co-cultivation medium and incubated for 3 days in the dark at 25° C. Co-cultivated calli were grown on 2,4-D-containing medium for 4 weeks in the dark at 28° C. in the presence of a selection agent. During this period, rapidly growing resistant callus islands developed. After transfer of this material to a regeneration medium and incubation in the light, the embryogenic potential was released and shoots developed in the next four to five weeks. Shoots were excised from the calli and incubated for 2 to 3 weeks on an auxin-containing medium from which they were transferred to soil. Hardened shoots were grown under high humidity and short days in a greenhouse.

Approximately 35 independent T0 rice transformants were generated for one construct. The primary transformants were transferred from a tissue culture chamber to a greenhouse. After a quantitative PCR analysis to verify copy number of the T-DNA insert, only single copy transgenic plants that exhibit tolerance to the selection agent were kept for harvest of T1 seed. Seeds were then harvested three to five months after transplanting. The method yielded single locus transformants at a rate of over 50% (Aldemita and Hodges 1996, Chan et al. 1993, Hiei et al. 1994).

### Corn Transformation

Transformation of maize (Zea mays) is performed with a modification of the method described by Ishida et al. (1996) Nature Biotech 14(6): 745-50. Transformation is genotype-dependent in corn and only specific genotypes are amenable to transformation and regeneration. The inbred line A188 (University of Minnesota) or hybrids with A188 as a parent are good sources of donor material for transformation, but other genotypes can be used successfully as well. Ears are harvested from corn plant approximately 11 days after pollination (DAP) when the length of the immature embryo is about 1 to 1.2 mm. Immature embryos are cocultivated with Agrobacterium tumefaciens containing the expression vector, and transgenic plants are recovered through organogenesis. Excised embryos are grown on callus induction medium, then maize regeneration medium, containing the selection agent (for example imidazolinone but various selection markers can be used). The Petri plates are incubated in the light at 25° C. for 2-3 weeks, or until shoots develop. The green shoots are transferred from each embryo to maize rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Wheat Transformation

Transformation of wheat is performed with the method described by Ishida et al. (1996) Nature Biotech 14(6): 745-50. The cultivar Bobwhite (available from CIMMYT, Mexico) is commonly used in transformation. Immature embryos are co-cultivated with Agrobacterium tumefaciens containing the expression vector, and transgenic plants are recovered through organogenesis. After incubation with Agrobacterium, the embryos are grown in vitro on callus induction medium, then regeneration medium, containing the selection agent (for example imidazolinone but various selection markers can be used). The Petri plates are incubated in the light at 25° C. for 2-3 weeks, or until shoots develop. The green shoots are transferred from each embryo to rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Soybean Transformation

Soybean is transformed according to a modification of the method described in the Texas A&M patent U.S. Pat. No. 5,164,310. Several commercial soybean varieties are amenable to transformation by this method. The cultivar Jack (available from the Illinois Seed foundation) is commonly used for transformation. Soybean seeds are sterilised for in vitro sowing. The hypocotyl, the radical and one cotyledon are excised from seven-day old young seedlings. The epicotyl and the remaining cotyledon are further grown to develop axillary nodes. These axillary nodes are excised and incubated with Agrobacterium tumefaciens containing the expression vector. After the cocultivation treatment, the explants are washed and transferred to selection media. Regenerated shoots are excised and placed on a shoot elongation medium. Shoots no longer than 1 cm are placed on rooting medium until roots develop. The rooted shoots are transplanted to soil in the greenhouse. T1 seeds are produced from plants that exhibit tolerance to the selection agent and that contain a single copy of the T-DNA insert.

### Rapeseed/Canola Transformation

Cotyledonary petioles and hypocotyls of 5-6 day old young seedling are used as explants for tissue culture and transformed according to Babic et al. (1998, Plant Cell Rep 17: 183-188). The commercial cultivar Westar (Agriculture Canada) is the standard variety used for transformation, but other varieties can also be used. Canola seeds are surface-sterilized for in vitro sowing. The cotyledon petiole explants with the cotyledon attached are excised from the in vitro seedlings, and inoculated with Agrobacterium (containing the expression vector) by dipping the cut end of the petiole explant into the bacterial suspension. The explants are then cultured for 2 days on MSBAP-3 medium containing 3 mg/l BAP, 3% sucrose, 0.7 Phytagar at 23° C., 16 hr light. After two days of co-cultivation with Agrobacterium, the petiole explants are transferred to MSBAP-3 medium containing 3 mg/l BAP, cefotaxime, carbenicillin, or timentin (300 mg/l) for 7 days, and then cultured on MSBAP-3 medium with cefotaxime, carbenicillin, or timentin and selection agent until shoot regeneration. When the shoots are 5-10 mm in length, they are cut and transferred to shoot elongation medium (MSBAP-0.5, containing 0.5 mg/l BAP). Shoots of about 2 cm in length are transferred to the rooting medium (MS0) for root induction. The rooted shoots are transplanted to soil in the greenhouse. T1 seeds are produced from plants that exhibit tolerance to the selection agent and that contain a single copy of the T-DNA insert.

### Alfalfa Transformation

A regenerating clone of alfalfa (Medicago sativa) is transformed using the method of (McKersie et al., 1999 Plant Physiol 119: 839-847). Regeneration and transformation of alfalfa is genotype dependent and therefore a regenerating plant is required. Methods to obtain regenerating plants have been described. For example, these can be selected from the cultivar Rangelander (Agriculture Canada) or any other commercial alfalfa variety as described by Brown DCW and A Atanassov (1985. Plant Cell Tissue Organ Culture 4: 111-112). Alternatively, the RA3 variety (University of Wisconsin) has been selected for use in tissue culture (Walker et al., 1978 Am J Bot 65:654-659). Petiole explants are cocultivated with an overnight culture of Agrobacterium tumefaciens C58C1 pMP90 (McKersie et al., 1999 Plant Physiol 119: 839-847) or LBA4404 containing the expression vector. The explants are cocultivated for 3 d in the dark on SH induction medium containing 288 mg/L Pro, 53 mg/L thioproline, 4.35 g/L K2SO4, and 100 μm acetosyringinone. The explants are washed in half-strength Murashige-Skoog medium (Murashige and Skoog, 1962) and plated on the same SH induction medium without acetosyringinone but with a suitable selection agent and suitable antibiotic to inhibit Agrobacterium growth. After several weeks, somatic embryos are transferred to BOi2Y development medium containing no growth regulators, no antibiotics, and 50 g/L sucrose. Somatic embryos are subsequently germinated on half-strength Murashige-Skoog medium. Rooted seedlings were transplanted into pots and grown in a greenhouse. T1 seeds are produced from plants that exhibit tolerance to the selection agent and that contain a single copy of the T-DNA insert.

### Cotton Transformation

Cotton is transformed using Agrobacterium tumefaciens according to the method described in U.S. Pat. No. 5,159,135. Cotton seeds are surface sterilised in 3% sodium hypochlorite solution during 20 minutes and washed in distilled water with 500 μg/ml cefotaxime. The seeds are then transferred to SH-medium with 50 μg/ml benomyl for germination. Hypocotyls of 4 to 6 days old seedlings are removed, cut into 0.5 cm pieces and are placed on 0.8% agar. An Agrobacterium suspension (approx. 108 cells per ml, diluted from an overnight culture transformed with the gene of interest and suitable selection markers) is used for inoculation of the hypocotyl explants. After 3 days at room temperature and lighting, the tissues are transferred to a solid medium (1.6 g/l Gelrite) with Murashige and Skoog salts with B5 vitamins (Gamborg et al., Exp. Cell Res. 50:151-158 (1968)), 0.1 mg/l 2,4-D, 0.1 mg/l 6-furfurylaminopurine and 750 μg/ml MgCL2, and with 50 to 100 μg/ml cefotaxime and 400-500 μg/ml carbenicillin to kill residual bacteria. Individual cell lines are isolated after two to three months (with subcultures every four to six weeks) and are further cultivated on selective medium for tissue amplification (30° C., 16 hr photoperiod). Transformed tissues are subsequently further cultivated on non-selective medium during 2 to 3 months to give rise to somatic embryos. Healthy looking embryos of at least 4 mm length are transferred to tubes with SH medium in fine vermiculite, supplemented with 0.1 mg/l indole acetic acid, 6 furfurylaminopurine and gibberellic acid. The embryos are cultivated at 30° C. with a photoperiod of 16 hrs, and plantlets at the 2 to 3 leaf stage are transferred to pots with vermiculite and nutrients. The plants are hardened and subsequently moved to the greenhouse for further cultivation.

### Example 10

### Phenotypic Evaluation Procedure

### 10.1 Evaluation Setup

Approximately 35 independent T0 rice transformants are generated. The primary transformants are transferred from a tissue culture chamber to a greenhouse for growing and harvest of T1 seed. Six events, of which the T1 progeny segregated 3:1 for presence/absence of the transgene, are retained. For each of these events, approximately 10 T1 seedlings containing the transgene (hetero- and homo-zygotes) and approximately 10 T1 seedlings lacking the transgene (nullizygotes) are selected by monitoring visual marker expression. The transgenic plants and the corresponding nullizygotes are grown side-by-side at random positions. Greenhouse conditions are for shorts days (12 hours light), 28° C. in the light and 22° C. in the dark, and a relative humidity of 70%.

Four T1 events were further evaluated in the T2 generation following the same evaluation procedure as for the T1 generation but with more individuals per event. From the stage of sowing until the stage of maturity the plants are passed several times through a digital imaging cabinet. At each time point digital images (2048×1536 pixels, 16 million colours) are taken of each plant from at least 6 different angles.

### Drought Screen

Plants from T2 seeds are grown in potting soil under normal conditions until they approached the heading stage. They are then transferred to a “dry” section where irrigation is withheld. Humidity probes are inserted in randomly chosen pots to monitor the soil water content (SWC). When SWC goes below certain thresholds, the plants are automatically re-watered continuously until a normal level is reached again. The plants are then re-transferred again to normal conditions. The rest of the cultivation (plant maturation, seed harvest) is the same as for plants not grown under abiotic stress conditions. Growth and yield parameters are recorded as detailed for growth under normal conditions.

### Nitrogen Use Efficiency Screen

Rice plants from T2 seeds were grown in potting soil under normal conditions except for the nutrient solution. The pots were watered from transplantation to maturation with a specific nutrient solution containing reduced N nitrogen (N) content, usually between 7 to 8 times less. The rest of the cultivation (plant maturation, seed harvest) was the same as for plants not grown under abiotic stress. Growth and yield parameters were recorded as detailed for growth under normal conditions.

### Salt Stress Screen

Plants are grown on a substrate made of coco fibers and argex (3 to 1 ratio). A normal nutrient solution is used during the first two weeks after transplanting the plantlets in the greenhouse. After the first two weeks, 25 mM of salt (NaCl) is added to the nutrient solution, until the plants are harvested. Seed-related parameters are then measured.

### 10.2 Statistical Analysis: F Test

A two factor ANOVA (analysis of variants) was used as a statistical model for the overall evaluation of plant phenotypic characteristics. An F test was carried out on all the parameters measured of all the plants of all the events transformed with the gene of the present invention. The F test was carried out to check for an effect of the gene over all the transformation events and to verify for an overall effect of the gene, also known as a global gene effect. The threshold for significance for a true global gene effect was set at a 5% probability level for the F test. A significant F test value points to a gene effect, meaning that it is not only the mere presence or position of the gene that is causing the differences in phenotype.

### 10.3 Parameters Measured

### Biomass-Related Parameter Measurement

From the stage of sowing until the stage of maturity the plants were passed several times through a digital imaging cabinet. At each time point digital images (2048×1536 pixels, 16 million colours) were taken of each plant from at least 6 different angles.

The plant aboveground area (or leafy biomass) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The above ground area is the area measured at the time point at which the plant had reached its maximal leafy biomass. The early vigour is the plant (seedling) aboveground area three weeks post-germination. Increase in root biomass is expressed as an increase in total root biomass (measured as maximum biomass of roots observed during the lifespan of a plant); or as an increase in the root/shoot index (measured as the ratio between root mass and shoot mass in the period of active growth of root and shoot).

Early vigour was determined by counting the total number of pixels from aboveground plant parts discriminated from the background. This value was averaged for the pictures taken on the same time point from different angles and was converted to a physical surface value expressed in square mm by calibration. The results described below are for plants three weeks post-germination.

### Seed-Related Parameter Measurements

The mature primary panicles were harvested, counted, bagged, barcode-labelled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed yield was measured by weighing all filled husks harvested from a plant. Total seed number per plant was measured by counting the number of husks harvested from a plant. Thousand Kernel Weight (TKW) is extrapolated from the number of filled seeds counted and their total weight. The Harvest Index (HI) in the present invention is defined as the ratio between the total seed yield and the above ground area (mm²), multiplied by a factor 10⁶. The total number of flowers per panicle as defined in the present invention is the ratio between the total number of seeds and the number of mature primary panicles. The seed fill rate as defined in the present invention is the proportion (expressed as a %) of the number of filled seeds over the total number of seeds (or florets).

### Examples 11

### Results of the Phenotypic Evaluation of the Transgenic Plants

### 11.1. YLD-ZnF Polypeptides

Transgenic rice plants expressing an YLD-ZnF nucleic acid and grown under non-stress conditions showed increased seed yield, in particular increased Thousand Kernel Weight. Four out of six lines had an overall increased TKW of 3.2% with a p value of 0.0000. In addition, when grown under nitrogen limitation, the transgenic rice plants expressing an YLD-ZnF nucleic acid showed increased early vigour: two lines out of six tested lines had an average increase of 8.2% (p-value 0.017).

### 11.2. NOA Polypeptides

The evaluation of transgenic rice plants expressing a NOA nucleic acid under non-stress conditions revealed an increase in yield compared to the control plants. An overall increase of 7.5% in total seed weight (p-value≦0.05) was observed for the T1 generation plants, and this yield increase was again observed for the T2 plants (9.2% overall increase in total seed weight, p-value≦0.05). In addition, there was also an increase in above ground biomass, harvest index and thousand kernel weight, in the number of filled seeds and in the number of flowers per panicle.

11.3. ASF1-like Polypeptides

The results of the evaluation of transgenic rice plants expressing an ASF1-like nucleic acid from rice or Arabidopsis thaliana under non-stress conditions are presented below. A percentage difference between the transgenic plants compared to the nulls (controls) is shown.

ASF1-like Sequence from Rice

| | % Overall (at | % Average of |
|---|---|---|
| Parameter | least 5 lines) | best lines |
| TKW | 4.7% | |
| Emergence Vigour | 1.5% | 20.1% |
| Total seed yield | 4.2% | 13.7% |
| No. filled seeds | −0.4% | 11.45% |
| No. flowers per panicle | 7.6% | 14.1% |
| Harvest Index | 4.7% | 12.77% |

ASF1-like Sequence from Arabidopsis thaliana

| | % Overall (at | % Average of |
|---|---|---|
| Parameter | least 5 lines) | best lines |
| Aboveground area | 1.7% | 19.9% |
| Root max | 3.3% | 13.2% |
| Total seed yield | 7.2% | 35.6% |
| Time to flower | 2.2% | 4.35% |
| No. filled seeds | 7.4% | 32% |
| Total number of seeds | 9.6% | 38.8% |
| No. first panicles | 1.4% | 27.15% |

The above results for the Arabidopsis thaliana ASF1-like sequence is for the T1 generation. Comparable results were seen in the T2 generation, further including a positive tendency for greenness index.

### 11.4. Group I MBF1 Polypeptides

The results of the evaluation of T1 or T2 generation transgenic rice plants expressing a nucleic acid sequence encoding a group I MBF1 polypeptide, under the control of a constitutive promoter, and grown under normal growth conditions, are presented in Table E1 below.

TABLE E1
Results of the evaluation of T1 or T2 generation transgenic rice
plants expressing the nucleic acid sequence encoding a group I MBF1
polypeptide, under the control of a promoter for constitutive
expression, and grown under normal growth conditions.

| Nucleic acid | Promoter | |
|---|---|---|
| sequence | sequence | Positive parameters |
| SEQ ID NO: 188 | SEQ ID NO: 253 | Total seed yield per plant, early |
| | | vigor |
| SEQ ID NO: 190 | SEQ ID NO: 254 | Total seed yield per plant, early |
| | | vigor, |
| | | seed fill rate, number of filled |
| | | seeds |
| SEQ ID NO: 192 | SEQ ID NO: 254 | Early vigor |

The results of the evaluation of T1 or T2 generation transgenic rice plants expressing a nucleic acid sequence encoding a group I MBF1 polypeptide, under the control of a constitutive promoter, and grown under reduced nutrient availability conditions, are presented in Table E2 below.

TABLE E2
Results of the evaluation of T1 or T2 generation transgenic rice
plants expressing the nucleic acid sequence encoding a group I MBF1
polypeptide, under the control of a promoter for constitutive
expression, and grown under reduced nutrient availability conditions.

| Nucleic acid | Promoter | |
|---|---|---|
| sequence | sequence | Positive parameters |
| SEQ ID NO: 190 | SEQ ID NO: 253 | Early vigor, aboveground biomass, |
| | | number of first panicles |
| SEQ ID NO: 194 | SEQ ID NO: 253 | Early vigor, aboveground biomass, |
| | | number of first panicles |

1-21. (canceled)

22. A method for enhancing abiotic stress tolerance and/or enhancing yield-related traits in a plant relative to a control plant, comprising modulating expression in a plant of a nucleic acid selected from the group consisting of:
(a) a nucleic acid encoding a cytochrome c oxidase (COX) VIIa subunit polypeptide
(COX VIIa subunit), or an orthologue or paralogue thereof;
(b) a nucleic acid encoding a YLD-ZnF polypeptide, wherein the YLD-ZnF polypeptide comprises a zf-DNL domain;
(c) a nucleic acid encoding a protein kinase with TPR repeat (PKT) polypeptide, or an orthologue or paralogue thereof;
(d) a nucleic acid encoding a nitric oxide associated (NOA) polypeptide, wherein said NOA polypeptide comprises a PTHR11089 domain;
(e) a nucleic acid encoding an Anti-silencing factor 1 (ASF1)-like polypeptide;
(f) a nucleic acid encoding a plant homeodomain finger (PHDF) polypeptide, or an orthologue or paralogue thereof; and
(g) a nucleic acid encoding a group I multiprotein bridging factor 1 (MBF1) polypeptide, wherein the group I MBF1 polypeptide comprises (i) an amino acid sequence having at least 70% or more amino acid sequence identity to an N-terminal multibridging domain with an InterPro entry IPR0013729 (PFAM entry PF08523 MBF1) as represented by SEQ ID NO: 250; and (ii) an amino acid sequence having at least 70% or more amino acid sequence identity to a helix-turn-helix 3 domain with an InterPro entry IPR001387 (PFAM ENTRY PF01381 HTH_{—}3).

23. The method of claim 22, wherein said modulated expression is effected by introducing and expressing in a plant said nucleic acid.

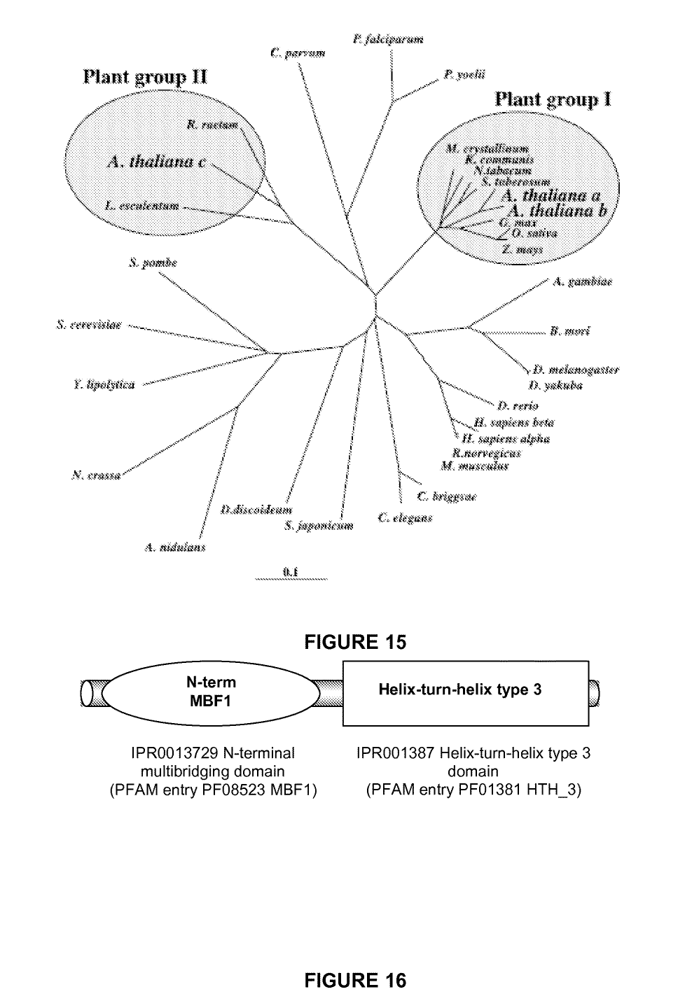

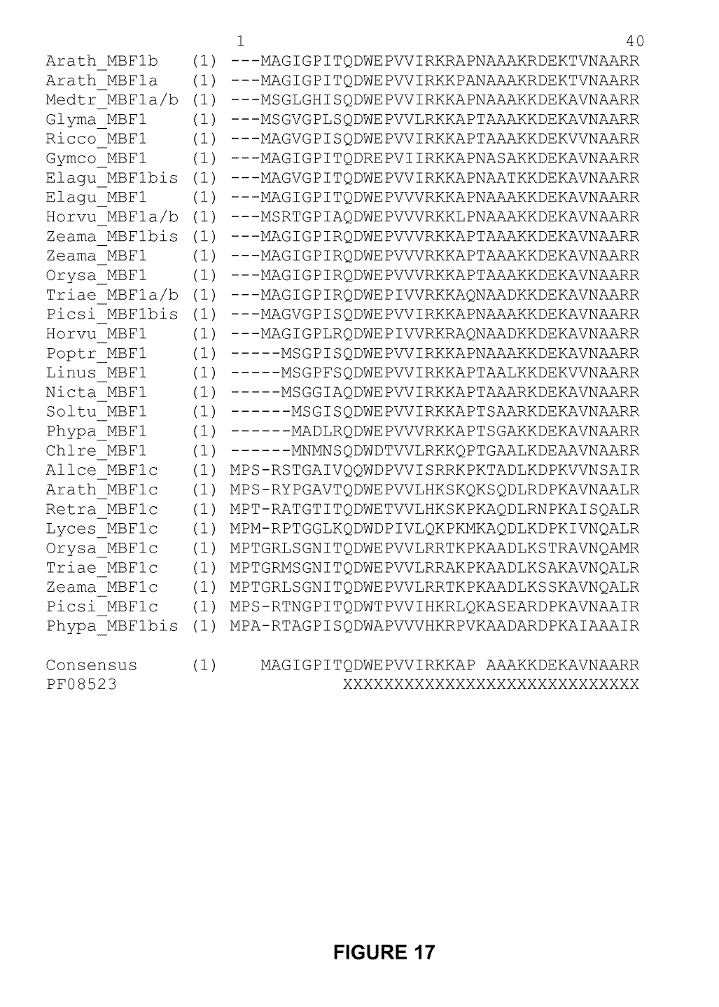

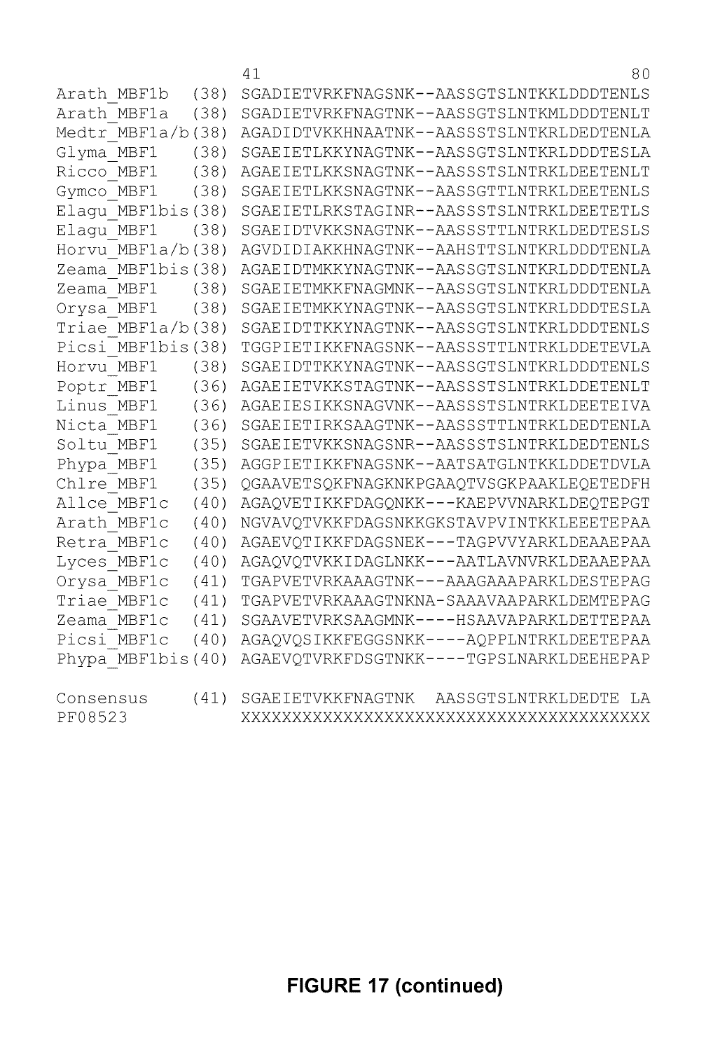

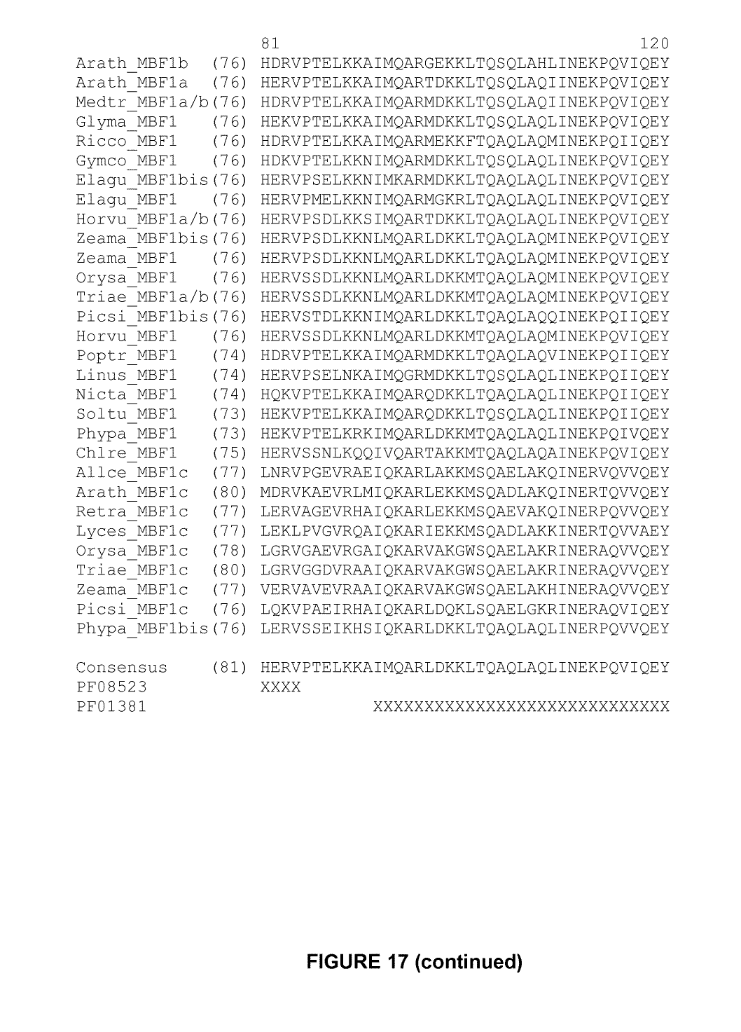

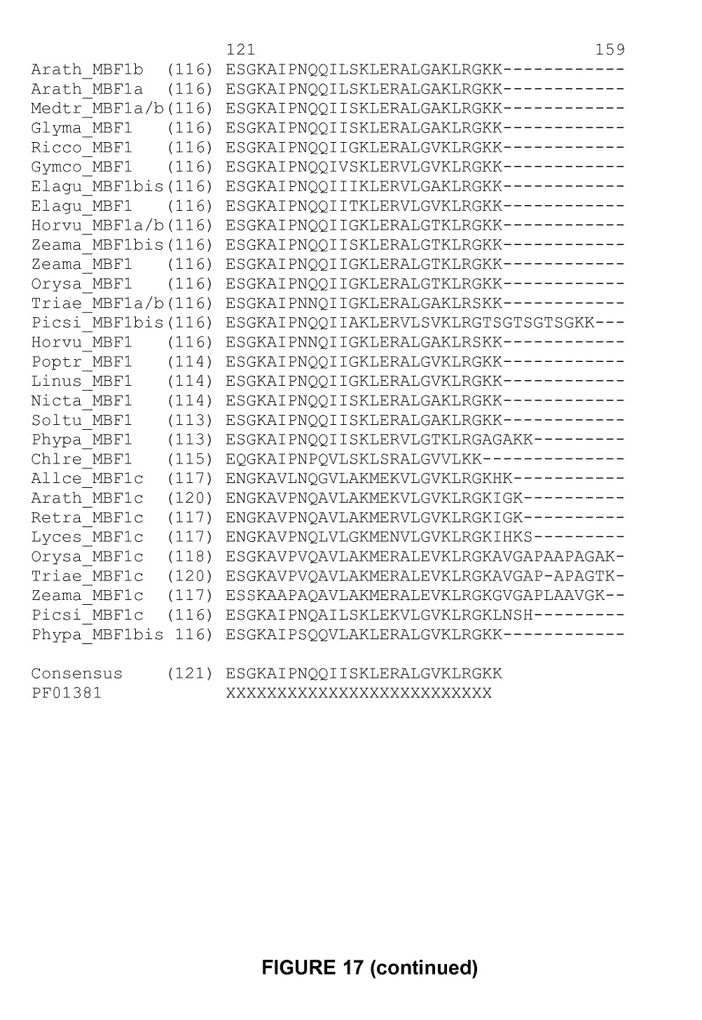

24. The method of claim 22, wherein said nucleic acid is selected from the group consisting of:
(a) a nucleic acid encoding a COX VIIa subunit polypeptide listed in Table A1 or an orthologue or paralogue thereof, or a portion of said nucleic acid, or a nucleic acid capable of hybridizing with said nucleic acid;
(b) a nucleic acid encoding a YLD-ZnF polypeptide, wherein the YLD-ZnF polypeptide comprises one or more of Motif 1 (SEQ ID NO: 20), Motif 2 (SEQ ID NO: 21), Motif 3 (SEQ ID NO: 22), or Motif 4 (SEQ ID NO: 23);
(c) a nucleic acid encoding a YLD-ZnF polypeptide listed in Table A2 or an orthologue or paralogue thereof, or a portion of said nucleic acid, or a nucleic acid capable of hybridizing with said nucleic acid;
(d) a nucleic acid encoding a PKT polypeptide listed in Table A3 or an orthologue or paralogue thereof, or a portion of said nucleic acid, or a nucleic acid capable of hybridizing with said nucleic acid;
(e) a nucleic acid encoding a NOA polypeptide, wherein the NOA polypeptide comprises one or more of Motif 5 (SEQ ID NO: 60), Motif 6 (SEQ ID NO: 61), Motif 7 (SEQ ID NO 62), Motif 8 (SEQ ID NO: 63), Motif 9 (SEQ ID NO: 64), or Motif 10 (SEQ ID NO: 65);
(f) a nucleic acid encoding a NOA polypeptide listed in Table A4 or an orthologue or paralogue thereof, or a portion of said nucleic acid, or a nucleic acid capable of hybridizing with said nucleic acid;
(g) a nucleic acid encoding an ASF1-like polypeptide, wherein the ASF1-like polypeptide comprises one or more of MOTIF I (SEQ ID NO: 262), MOTIF II (SEQ ID NO: 263), MOTIF III (SEQ ID NO: 264), MOTIF IV (SEQ ID NO: 265), or a motif having at least 50% more sequence identity to any one or more of MOTIFs I to IV;
(h) a nucleic acid encoding an ASF1-like polypeptide listed in Table A5 or an orthologue or paralogue thereof, or a portion of said nucleic acid, or a nucleic acid capable of hybridizing with said nucleic acid;
(i) a nucleic acid encoding a PHDF polypeptide listed in Table A6 or an orthologue or paralogue thereof, or a portion of said nucleic acid, or a nucleic acid capable of hybridizing with said nucleic acid;
(j) a nucleic acid encoding a group I MBF1 polypeptide, wherein the group I MBF1 polypeptide comprises at least 50% or more amino acid sequence identity to the polypeptide sequence of SEQ ID NO: 189, 191, 193, or 195;
(k) a nucleic acid encoding a group I MBF1 polypeptide, wherein the group I MBF1 polypeptide comprises at least 50% or more amino acid sequence identity to any of the polypeptides listed in Table A7;
(l) a nucleic acid encoding a group I MBF1 polypeptide, wherein the group I MBF1 polypeptide, when used in the construction of an MBF1 phylogenetic tree, such as the one depicted in FIG. 15, clusters with the group I MBF1 polypeptides comprising the polypeptide sequence of SEQ ID NO: 189, 191, 193 and 195, rather than with any other group;
(m) a nucleic acid encoding a group I MBF1 polypeptide, wherein the group I MBF1 polypeptide complements a yeast strain deficient for MBF1 activity; and
(n) a nucleic acid encoding a group I MBF1 polypeptide listed in Table A7 or an orthologue or paralogue thereof, or a portion of said nucleic acid, or a nucleic acid capable of hybridizing with said nucleic acid.

25. The method of claim 22, wherein said nucleic acid is operably linked to a constitutive promoter, a GOS2 promoter, or a GOS2 promoter from rice.

26. The method of claim 22, wherein said nucleic acid is selected from the group consisting of:
(a) a nucleic acid encoding a COX VIIa subunit polypeptide obtained from Physcomitrella patens;
(b) a nucleic acid encoding a YLD-ZnF polypeptide obtained from a plant, a dicotyledonous plant, a plant from the family Fabaceae, a plant from the genus Medicago, or Medicago truncatula;
(c) a nucleic acid encoding a PKT polypeptide obtained from Populus trichocarpa;
(d) a nucleic acid encoding a NOA polypeptide obtained from a plant, a dicotyledonous plant, a plant from the family Brassicaceae, a plant from the genus Arabidopsis, or Arabidopsis thaliana;
(e) a nucleic acid encoding an ASF1-like polypeptide obtained from a plant, a monocotyledonous or dicotyledonous plant, a plant from the family Poaceae or Brassicaceae, a plant from the genus Arabidopsis or Oryza, Arabidopsis thaliana, or Oryza sativa;
(f) a nucleic acid encoding a PHDF polypeptide obtained from Solanum lycopersicum; and
(g) a nucleic acid encoding a group I MBF1 polypeptide obtained from a plant, a monocotyledonous or dicotyledonous plant, Arabidopsis thaliana, Medicago truncatula, or Triticum aestivum.

27. The method of claim 22, wherein the enhanced yield-related traits comprise increased yield, increased seed yield, and/or increased early vigour relative to a control plant.

28. The method of claim 22, wherein the enhanced yield-related traits are obtained under non-stress conditions.

29. A plant or part thereof, including seeds, obtained from the method of claim 22, wherein said plant or part thereof comprises said nucleic acid.

30. The plant or part thereof of claim 29, wherein said plant is a crop plant or a monocot or a cereal selected from the group consisting of rice, maize, wheat, barley, millet, rye, triticale, sorghum, sugarcane, emmer, spelt, secale, einkom, teff, milo, and oats.

31. Harvestable parts of the plant of claim 29.

32. Harvestable parts of claim 31, which are shoot biomass and/or seeds.

33. Products derived from the plant or part thereof of claim 29 and/or harvestable parts of said plant.

34. A construct comprising:
(i) a nucleic acid;
(ii) one or more control sequences capable of driving expression of said nucleic acid; and optionally
(iii) a transcription termination sequence,
wherein said nucleic acid is selected from the group consisting of:
(a) a nucleic acid encoding a cytochrome c oxidase (COX) VIIa subunit polypeptide (COX VIIa subunit), or an orthologue or paralogue thereof;
(b) a nucleic acid encoding a YLD-ZnF polypeptide, wherein the YLD-ZnF polypeptide comprises a zf-DNL domain;
(c) a nucleic acid encoding a protein kinase with TPR repeat (PKT) polypeptide, or an orthologue or paralogue thereof;
(d) a nucleic acid encoding a nitric oxide associated (NOA) polypeptide, wherein said nitric oxide associated polypeptide comprises a PTHR11089 domain;
(e) a nucleic acid encoding an Anti-silencing factor 1 (ASF1)-like polypeptide;
(f) a nucleic acid encoding a plant homeodomain finger (PHDF) polypeptide, or an orthologue or paralogue thereof; and
(g) a nucleic acid encoding a group I multiprotein bridging factor 1 (MBF1) polypeptide, wherein the group I MBF1 polypeptide comprises (i) an amino acid sequence having at least 70% or more amino acid sequence identity to an N-terminal multibridging domain with an InterPro entry IPR0013729 (PFAM entry PF08523 MBF1) as represented by SEQ ID NO: 250; and (ii) an amino acid sequence having at least 70% or more amino acid sequence identity to a helix-turn-helix 3 domain with an InterPro entry IPR001387 (PFAM ENTRY PF01381 HTH_{—}3).

35. The construct of claim 34, wherein said one or more control sequences is a constitutive promoter, a GOS2 promoter, or a GOS2 promoter from rice.

36. A plant, plant part, or plant cell transformed with the construct of claim 34.

37. The plant, plant part, or plant cell of claim 36, wherein said plant is a crop plant or a monocot or a cereal selected from the group consisting of rice, maize, wheat, barley, millet, rye, triticale, sorghum, sugarcane, emmer, spelt, secale, einkorn, teff, milo, and oats.

38. Harvestable parts of the plant of claim 36.

39. Harvestable parts of claim 38, which are shoot biomass and/or seeds.

40. Products derived from the plant, plant part, or plant cell of claim 36 and/or harvestable parts of said plant.

41. A method for producing a transgenic plant with enhanced abiotic stress tolerance and/or enhanced yield-related traits relative to a control plant, comprising introducing the construct of claim 34 into a plant.

42. The method of claim 42, further comprising cultivating the plant under conditions promoting abiotic stress.

43. An isolated nucleic acid molecule comprising:
(a) the nucleotide sequence of SEQ ID NO: 125;
(b) the complement of the nucleotide sequence of SEQ ID NO: 125; or
(c) a nucleotide sequence encoding a NOA polypeptide having at least 50% or more sequence identity to the amino acid sequence of SEQ ID NO: 94.

44. An isolated polypeptide comprising:
(a) the amino acid sequence of SEQ ID NO: 94;
(b) an amino acid sequence having at least 50% or more sequence identity to the amino acid sequence of SEQ ID NO: 94; or
(c) derivatives of any of the amino acid sequences of (i) or (ii) above.
